(19) United States
(12) Patent Application Publication
(10) Pub. No.: US 2026/0308185 A1
(43) Pub. Date: Oct. 8, 2026

(54) STAT DEGRADERS AND USES THEREOF

(71) Applicant: Recludix Pharma, Inc., San Diego, CA (US)

(72) Inventors: Neil Bifulco, San Diego, CA (US); Howard Bregman, San Diego, CA (US); Giovanni Cianchetta, San Diego, CA (US); Brian Hodous, Arlington, MA (US); Samuel K. Reznik, Arlington, MA (US); Yong Tang, West Roxbury, MA (US); Andrew Tasker, San Diego, CA (US); Rishi G. Vaswani, San Diego, CA (US); Ernest Allen Sickmier, San Diego, CA (US); Xia Tian, San Diego, CA (US); John Yeoman, San Diego, CA (US)

(21) Appl. No.: 19/479,567

(22) PCT Filed: May 15, 2024
(86) PCT No.: PCT/US2024/029374

§ 371 Date: Oct. 28, 2025

Related U.S. Application Data
(60) Provisional application No. 63/466,572, filed on May 15, 2023.

(51) Int. Cl. C07F 9/6561; A61K 31/675

(52) U.S. Cl. CPC C07F 9/6561; A61K 31/675

(57) ABSTRACT

Provided are compounds of Formula (I): (I) and pharmaceutically acceptable salts and compositions thereof, which are useful for treating a variety of conditions associated with STAT3 and/or STAT6.

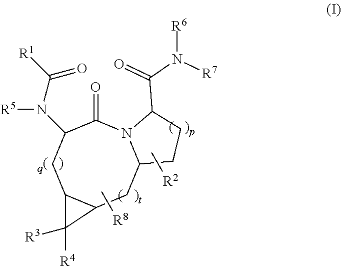

### I. FIELD OF THE INVENTION

The present invention relates to novel compounds for use as degraders of Signal Transducer and Activator of Transcription (STAT).

### I. BACKGROUND

The Signal Transducer and Activator of Transcription (STAT) family of proteins consists of transcription factors that play an essential role in the regulation of cell processes, such as proliferation, differentiation, apoptosis and angiogenesis. Seven STAT genes have been identified in the human genome: STAT1, STAT2, STAT3, STAT4, STAT5a, STAT5b, and STAT6.

STAT3 has received particular attention because it is strongly associated with the promotion of tumor growth and immune evasion, and the only STAT family member whose genetic deletion results in embryonic lethality. Indeed, aberrantly elevated STAT3 activity has been estimated to occur in more than 70% of human cancers. Activated STAT3 mediates critical gene expression changes and molecular events that dysregulate cell growth and apoptosis, promote angiogenesis, invasion, metastasis, and the development of resistance to apoptosis, and suppress the host's immune surveillance of the tumor, thereby making constitutively-active STAT3 a critical mediator of carcinogenesis and tumor progression.

Another STAT protein that has gained recent interest is STAT6. Recent studies have shown that STAT6 signaling is essential for IL-4- and IL-13-induced epithelial mesenchymal transition (EMT) and aggressiveness of colorectal cancer cells (CRC) cells. STAT6 is involved in several aspects of inflammatory disease and other related conditions. STAT6 is involved in several aspects of inflammatory disease and other related conditions.

Ubiquitin-Proteasome Pathway (UPP) or Ubiquitin-Proteasome System (UPS) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

### III. SUMMARY

Given their role in the regulation of cell processes, modulating the activity of one or more STAT proteins, particularly STAT3 and/or STAT6, represent a pivotal area of investigation for the treatment of cancer, inflammatory conditions, and other therapeutic needs.

Targeted protein degradation has become an advancing area as seen with rapid progression of PROTACs (proteolysis targeting chimeras) to clinic. PROTACs are protein degraders, which utilize the cell's own waste disposal machinery to eliminate, instead of inhibit, a target protein. PROTACs are bifunctional in that they simultaneously bind a target protein and an E3 ligase protein. This event subsequently ubiquitylates the target, marking it for proteasomal degradation. This, and related processes, represent a new therapeutic modality for the treatment of diseases.

The present invention provides novel bifunctional compounds for degradation of STAT3 and/or STAT6 by recruiting them to E3 ubiquitin ligase for degradation. The advantage of using the methods of the invention are that a broad range of targets that may be modulated by recruiting E3 ubiquitin ligase for degradation.

In certain aspects, the invention provides effective degraders of the STAT3 and/or STAT6. The degraders of STAT3 and/or STAT6 have general Formula A:

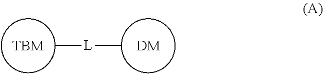

wherein, TBM is target binding moiety, DM is degradation moiety; and L is a linker between TBM and DM. A TBM is a moiety that binds to a target protein that should be degraded. In certain embodiments, the TBM is the moiety that binds to STAT3 and/or STAT6. The degradation moiety is responsible for including degradation of the target. In certain embodiments, the degradation moiety acts by recruitment of ubiquiting E3 ligase to the target attached to TBM, and thus results in degradation of the target.

In certain aspects, the compounds of the invention provide that the TBM moiety comprises a STAT3 and/or STAT6 binding moiety. The STAT3 and/or STAT6 binding moeity are inhibitors and/or modulators of STAT3 and/or STAT6. In certain embodiments, the STAT3 and/or STAT6 binding are compounds provided in WO 2023/192960, which is incorporated by reference in its entirety.

In certain embodiments, the STAT3 and/or STAT6 binding moiety is a compound of Formula (I′):

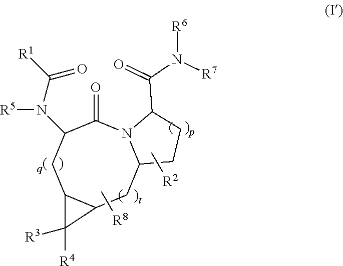

or a pharmaceutically acceptable salt thereof, wherein:
q is 0 or 1 and t is 0, 1, or 2, provided that at least one of q or t is 1;
p is 1 or 2;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], or —CR^{1a}R^{2a}P(O)[NH(AA)C(O)OR^{T}]][NH(AA)C(O)OR^{T}], —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, and (C₁-C₄)alkyl;
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
R⁸ is hydrogen or (C₁-C₄)alkyl;
AA is the residue of an alpha or beta natural or non-natural amino acid;
R^{T} and R^{Ty} are each independently selected from (C₁-C₄)alkyl, (C₁-C₄)alkyl-C(O)O(C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NR^{e}(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{X}, and R^{Z} are each independently selected from halo, cyano, (C₁-C₄)alkyl, cyano(C₁-C₄)alkyl, C₃-C₆cycloalkyl), halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, —(C₁-C₄)alkyl(C₁-C₄)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In certain embodiments from the present invention, the L and DM moieties are described herein. In certain embodiments, the L and DM moieties are provided in WO 2024/064080, which is incorporated by reference in its entirety.

In certain aspects, the invention provides that the compounds of the invention are compounds having the structural Formula I:

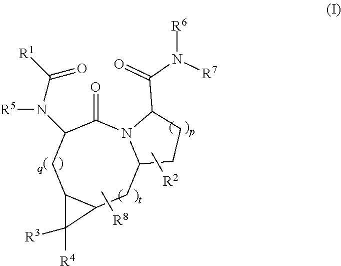

or a pharmaceutically acceptable salt thereof, wherein:
q is 0 or 1 and t is 0, 1, or 2, provided that at least one of q or t is 1;
p is 1 or 2;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)OR^{1b}][NH(AA)C(O)OR^{T}], —CR^{1a}R^{2a}P(O)[NHR^{Ty}][NH(AA)C(O)OR^{T}], —P(O)OR^{1a}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], or —CR^{1a}R^{2a}P(O)[NH(AA)C(O)OR^{T}]][NH(AA)C(O)OR^{T}], —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alky]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, and (C₁-C₄)alkyl;
R⁵ and R⁶ are each independently selected from hydrogen, phenyl, and (C₁-C₄)alkyl;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocycyl or a 5- to 12-membered monocyclic or bicyclic heteroaryl, each of which being optionally substituted with, as valency permits, R¹² and/or 1 to 3 groups selected from R^{Q};
R⁸ is hydrogen or (C₁-C₄)alkyl;
R^{10A}, R^{10B}, R^{10C}, and R^{10D} are each independently a chemical spacer unit;
R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10C}E, —NHR^{10C}E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
AA is the residue of an alpha or beta natural or non-natural amino acid;
R^{T} and R^{Ty} are each independently selected from (C₁-C₄)alkyl, (C₁-C₄)alkyl-C(O)O(C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{c}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NR^{e}(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F} and R^{X} are each independently selected from halo, cyano, (C₁-C₄)alkyl, cyano(C₁-C₄)alkyl, (C₃-C₆)cycloalkyl, halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, —(C₁-C₄)alkyl(C₁-C₄)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In one aspect, the disclosed compounds of Formula I and pharmaceutically acceptable salts thereof act as protein degraders of STAT3 and/or STAT6, and are useful in a variety of therapeutic applications such as, for example, in treating cancer and inflammatory conditions.

Pharmaceutical compositions comprising the compounds and pharmaceutically acceptable salts of Formula I, as well as methods for their preparation are also included.

Methods of treating conditions responsive to the degradation of STAT3 and/or STAT6 using the described compounds, pharmaceutically acceptable salts, and compositions thereof are also included.

### IV. DETAILED DESCRIPTION

### 1. General Description of Compounds

The present invention provides novel bifunctional compounds for degradation of STAT3 and/or STAT6 by recruiting them to E3 ubiquitin ligase for degradation. The advantage of using the methods of the invention are that a broad range of targets that may be modulated by recruiting E3 ubiquitin ligase for degradation.

In certain aspects, the invention provides effective degraders of the STAT3 and/or STAT6. The degraders of STAT3 and/or STAT6 have general Formula A:

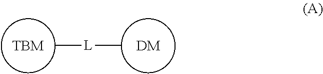

wherein, TBM is target binding moiety; DM is degradation moiety; and L is a linker between TBM and DM. A TBM is a moiety that binds to a target protein that should be degraded. In certain embodiments, the TBM is the moiety that binds to STAT3 and/or STAT6. The degradation moiety is responsible for including degradation of the target. In certain embodiments, the degradation moiety acts by recruitment of ubiquiting E3 ligase to the target attached to TBM, and thus results in degradation of the target.

In certain aspects, the compounds of the invention provide that the TBM moiety comprises a STAT3 and/or STAT6 binding moiety. The STAT3 and/or STAT6 binding moeity are inhibitors and/or modulators of STAT3 and/or STAT6. In certain embodiments, the STAT3 and/or STAT6 binding are compounds provided in WO 2023/192960, which is incorporated by reference in its entirety.

In a first embodiment, provided herein is a compound having the structural Formula I:

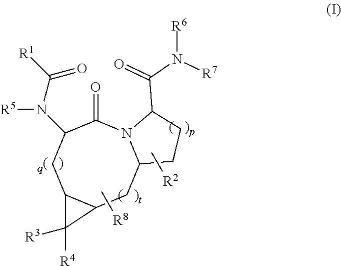

or a pharmaceutically acceptable salt thereof, wherein:
q is 0 or 1 and t is 0, 1, or 2, provided that at least one of q or t is 1;
p is 1 or 2;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{1b}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], or —CR^{1a}R^{2a}P(O)[NH(AA)C(O)OR^{T}]][NH(AA)C(O)OR^{T}], —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₅)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, and (C₁-C₄)alkyl;
R⁵ and R⁶ are each independently selected from hydrogen, phenyl, and (C₁-C₄)alkyl;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R⁸ is hydrogen or (C₁-C₄)alkyl;
R^{10A}, R^{10B}, R^{10C}, and R^{10D} are each independently a chemical spacer unit;
R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10C}E, —NHR^{10C}E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
AA is the residue of an alpha or beta natural or non-natural amino acid;
R^{T} and R^{Ty} are each independently selected from (C₁-C₄)alkyl-(C₁-C₄)alkyl-C(O)O(C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)—NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{X} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NR(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F} and R^{X} are each independently selected from halo, cyano, (C₁-C₄)alkyl, cyano(C₁-C₄)alkyl, (C₃-C₀)cycloalkyl, halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, —(C₁-C₄)alkyl(C₁-C₄)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₁-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

### 2. Definitions

When used in connection to describe a chemical group that may have multiple points of attachment, a hyphen (-) designates the point of attachment of that group to the variable to which it is defined. For example, —NR^{c}C(O)R^{e} means that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

Unless otherwise specified, the term “alkyl” when used alone or as part of a larger moiety, such as “haloalkyl”, and the like, means saturated straight-chain or branched monovalent hydrocarbon radical.

The term “haloalkyl” includes mono, poly, and perhaloalkyl groups where the halogens are independently selected from fluorine, chlorine, bromine, and iodine.

“Alkoxy” means an alkyl radical attached through an oxygen linking atom, represented by —O-alkyl. For example, “(C₁-C₄)alkoxy” includes methoxy, ethoxy, propoxy, and butoxy.

“Haloalkoxy” is a haloalkyl group which is attached to another moiety via an oxygen atom such as, e.g., —OCHF₂ or —OCF₃.

The term “oxo” means the group ═O.

The term “imino” means the group ═NH.

Unless otherwise specified, the term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiophenyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

Unless otherwise specified, the term “heterocyclyl” means a 4- to 12-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, tetrahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, and the like. It will be understood that when specified, optional substituents on a heterocyclyl group may be present on any substitutable position and, include, e.g., the position at which the heterocyclyl is attached (where valency permits).

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “aryl” refers to an aromatic carbocyclic single ring or two fused ring system containing 6 to 10 carbon atoms. Examples include phenyl, indanyl, tetrahydronaphthalene, and naphthyl. In one aspect, the aryl is phenyl or naphthyl.

The terms “cycloalkyl”, used alone or as part of a larger moiety, refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The term alkylene refers to a refers to a bivalent radical of a saturated, straight or branched chain hydrocarbon (e.g., CH₂) of the specified length.

The term alkynylene refers to an alkylene group that has at least one triple bond within the chain e.g., —(CH₂)₅CCH—.

The “residue of an amino acid” is the moiety remaining after formation of a bond between a reactive group in another compound (e.g., an amino group) and the carboxylic acid in the amino acid, after formation of a bond between a reactive group in another compound (e.g., a carboxylic acid) and the amino group in the amino acid, or both. As a consequence of the bond(s) formation, the carboxylic acid in the amino acid no longer has the OH group and instead has a bond between the carbonyl group and the reactive group in the compound; the amino group has only one hydrogen atom and instead has a bond between the reactive group in the other compound and the nitrogen of the amino group; or both. For example, the “residue of an alpha amino acid” can be depicted structurally as NH₂CR′R—C(O)—, —NHCR′R—C(O)OH or —NHCR′R—C(O)—; and the “residue of an beta amino acid” can be depicted structurally as or NH₂CR′RCH₂—C(O)—, —NHCR′RCH₂—C(O)OH or —NHCR′RCH₂—C(O)—, where R′ is H or C₁-C₆ alkyl and R is H or C₁-C₆ alkyl optionally substituted with 1 to 3 groups selected from halo, (C₁-C₃)alkoxy, OH, NH₂, —NH(C₁-C₄ alkyl), —N[(C₁-C₄ alkyl)]₁₂, SH, S(C₁-C₄ alkyl), imino, COOH, —COO(C₁-C₄ alkyl), —CO(C₁-C₄ alkyl), —CONH(C₁-C₄ alkyl)phenyl, phenyl, and 5- to 10-membered heteroaryl, wherein said C₁-C₆ alkyl may also be optionally interrupted by a sulfur or nitrogen heteroatom and wherein said phenyl is optionally substituted with 1 to 3 groups selected from OH, cyano, (C₁-C₄ alkyl), and halo(C₁-C₄ alkyl); or R is taken together with the nitrogen atoms from the alpha or beta amino acid residue to form a 4- to 6-membered heterocyclyl. For naturally occurring alpha amino acid (i.e., amino acids that occur in nature), R′ is H and R is selected from hydrogen, methyl, isopropyl, —CH₂CH(CH₃)₂, —(CH₂)₂SCH₃, —CH(CH₃)(CH₂CH₃), CH₂OH, —CH(OH)(CH₃), CH₂SH, —CH₂C(O)NH₂, —(CH₂)₂C(O)NH₂, benzyl, p-hydroxybenzyl, —CH₂(indolyl), —(CH₂)₄NH₂, —(CH₂)₃NHC(═NH₂)NH₂, —CH₂(imidazolyl), —(CH₂)COOH, and —(CH₂)₂COOH; or R taken together with the nitrogen atom of the alpha or beta amino acid residue forms a pyrrolidinyl ring.

Non-natural amino acids are known in the art and include e.g., alpha-alkyl amino acids (e.g., alpha methyl), alpha-alkylalkoxy amino acids (e.g., alpha —CH₂OCH₃), N-methyl amino acids, homo-amino acids, etc.

A chemical spacer unit refers to refers to a cleavable or non-cleavable that joins the E3 ligase binder to the remainder of the molecule described herein. In one aspect, the chemical spacer is uncleavable in vivo. In one aspect, the chemical spacer comprises one or more cyclic ring systems. In another aspect, the chemical spacer comprises an alkyl chain optionally substituted by and/or interrupted with one or more chemical groups. In one aspect, the chemical spacer comprises optimal spatial and chemical properties to effectuate optimal therapeutic activity.

A chemical moiety that binds to E3 ligase refers to a chemical structure that binds to the pocket or surface of E3 ligase. E3 ligase binders are known in the art and include, but are not limited to CRBN, VHL, IAP, or MDM2 based E3 binders. See e.g., Angewandte Chemie International Edition 59(36), May 2020. DOI:10.1002/anie.202004310; Medicinal Chemistry Communication 10(10), August 2019, DOI: 10.1039/C9MD00272C; and Molecules 2022, 27(19), 6515; https://doi.org/10.3390/molecules27196515.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

The terms “subject” and “patient” may be used interchangeably, and means a mammal in need of treatment, e.g., companion animals (e.g., dogs, cats, and the like), farm animals (e.g., cons, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, poly vinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that is sufficient to achieve the desired therapeutic effect (such as treatment of a condition recited herein) under the conditions of administration.

### 3. Compounds of the Invention

In certain embodiments, the STAT3 and/or STAT6 binding moiety is a compound of Formula (I′):

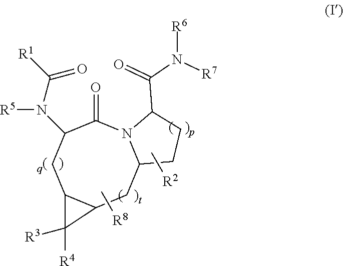

or a pharmaceutically acceptable salt thereof, % herein:
q is 0 or 1 and t is 0, 1, or 2, provided that at least one of q or t is 1;
p is 1 or 2;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NTH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, —CR^{1a}R^{2a}P(O)[OR^{1b}][NH(AA)C(O)OR^{T}], CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{T}][NH(AA)C(O)OR^{T}], or —CR^{1a}R^{2a}P(O)[NH(AA)C(O)OR^{T}][NH(AA)C(O)OR^{T}], —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}]][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl, —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, and (C₁-C₄)alkyl;
R⁵ and R⁶ are each independently selected from hydrogen, phenyl, and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits. 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits. 1 to 3 groups selected from R^{Q};
R⁸ is hydrogen or (C₁-C₄)alkyl;
AA is the residue of an alpha or beta natural or non-natural amino acid;
R^{T} and R^{Ty} are each independently selected from (C₁-C₄)alkyl, (C₁-C₄)alkyl-C(O)O(C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR, —C(O)R^{g}, —C(O)OR^{c}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NR^{e}(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{X}, and R^{Z} are each independently selected from halo, cyano, (C₁-C₄)alkyl, cyano(C₁-C₄)alkyl, C₃-C₆cycloalkyl), halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{e}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, —(C₁-C₄)alkyl(C₁-C₄)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

In certain embodiments from the present invention, the L and DM moieties are described herein. In certain embodiments, the L and DM moieties are provided in WO 2024/064080, which is incorporated by reference in its entirety.

In some embodiments, DM is an E3 ligase ligand well known to one of ordinary skill in the art including those described in M. Toure, C. M. Crews. Angew. Chem. Int. Ed., 2016, 55, 1966, T. Uehara et al. Nature Chemical Biology, 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, US 2019/0076540, WO 2017/197046, US 2019/0076542, WO 2017/197051, US 2019/0076539, WO 2017/197055, US 2019/0076541, and WO 2017/197056, the entirety of each of which is herein incorporated by reference.

In certain embodiments, L is attached to a modifiable carbon, oxygen, or nitro substitution or replacement group on one or both of DM and TBM.

In certain embodiments, the DM is a cereblon E3 ubiquitin ligase binding moiety (hereinafter also referred to as LBM).

In some embodiments, LBM is selected from the group consisting of

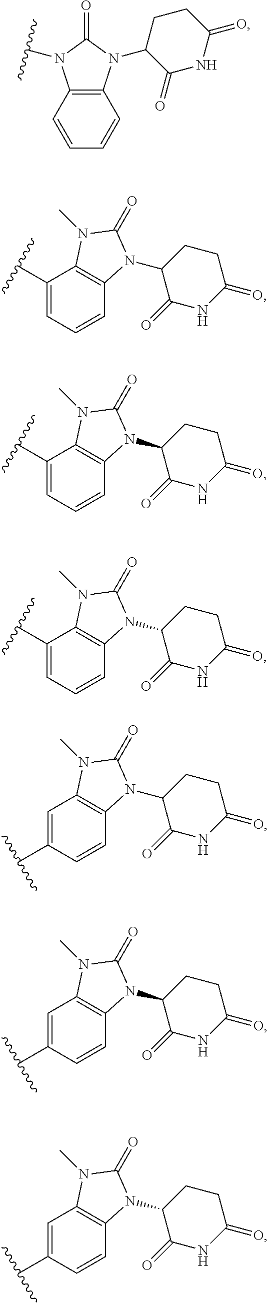

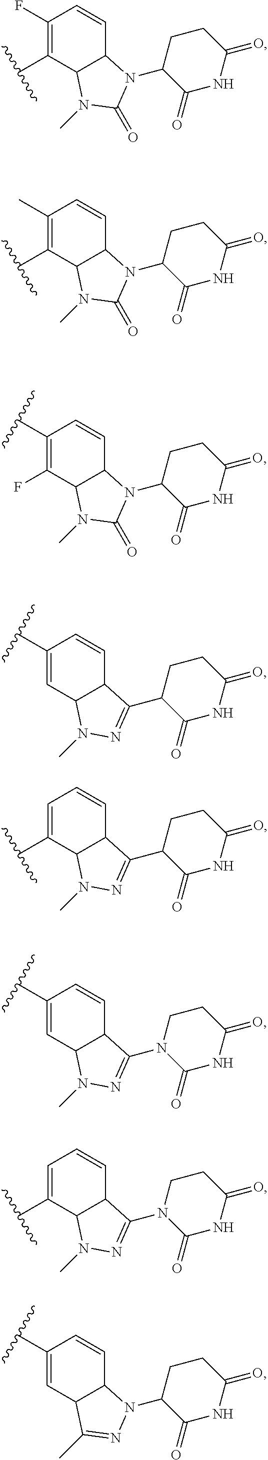

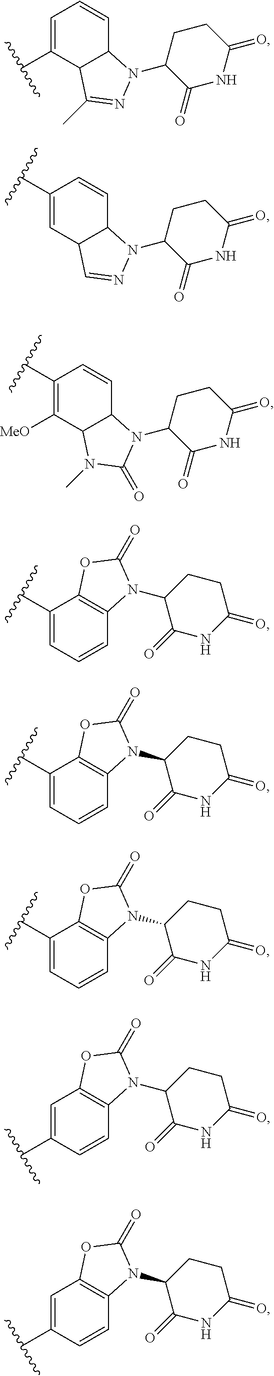

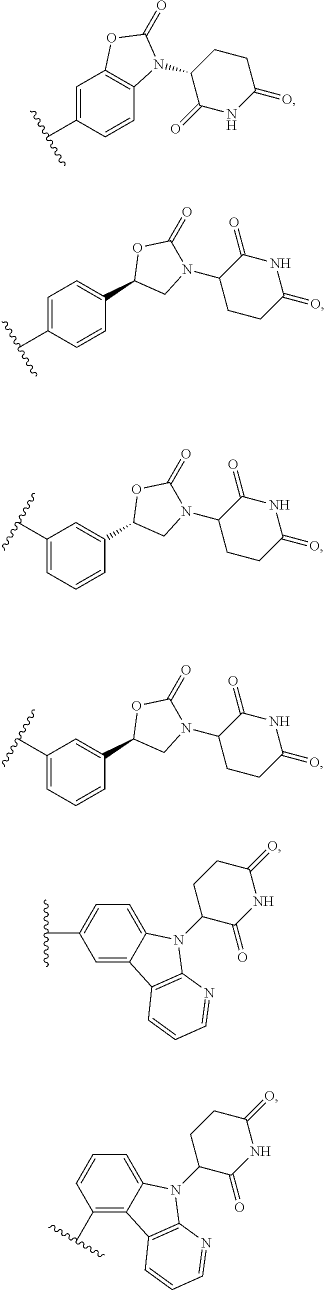 [SMILES: *c1ccc2c(c1)oc(=O)n2[C@@H]1CCC(=O)NC1=O ; *c1ccc([C@@H]2CN(C3CCC(=O)NC3=O)C(=O)O2)cc1 ; *c1cccc([C@H]2CN(C3CCC(=O)NC3=O)C(=O)O2)c1 ; *c1cccc([C@@H]2CN(C3CCC(=O)NC3=O)C(=O)O2)c1 ; *c1ccc2c(c1)c1cccnc1n2C1CCC(=O)NC1=O ; *c1cccc2c1c1cccnc1n2C1CCC(=O)NC1=O]

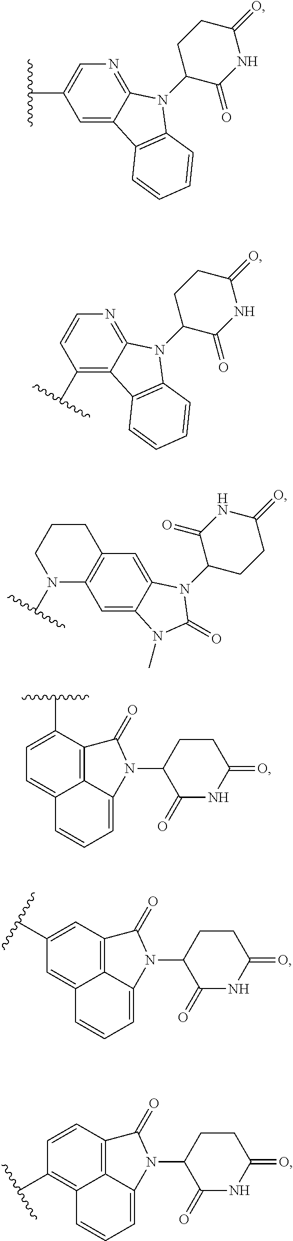

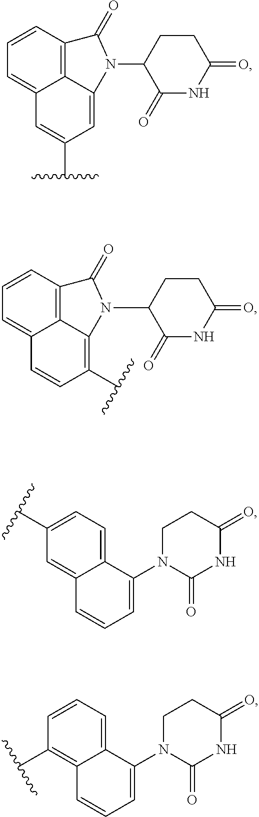 [SMILES: *c1cc2c3c(cccc3c1)C(=O)N2C1CCC(=O)NC1=O ; *c1ccc2cccc3c2c1N(C1CCC(=O)NC1=O)C3=O ; *c1ccc2c(N3CCC(=O)NC3=O)cccc2c1 ; *c1cccc2c(N3CCC(=O)NC3=O)cccc12]

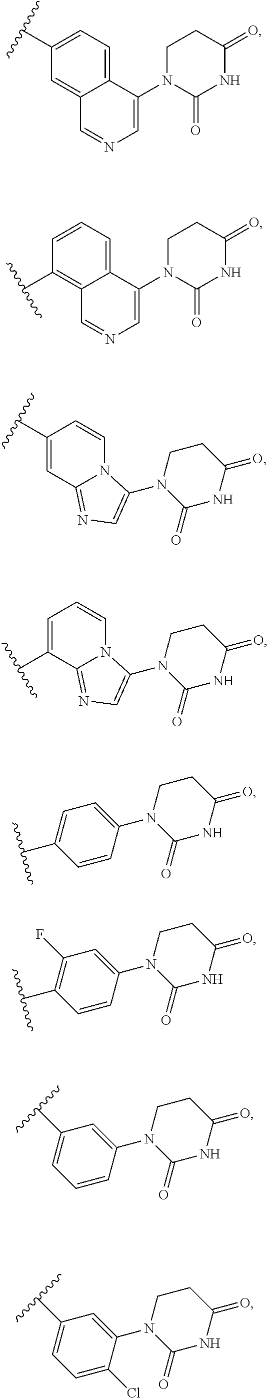

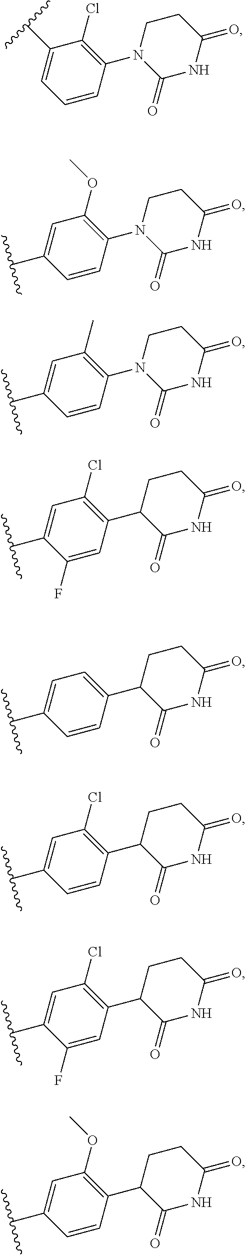

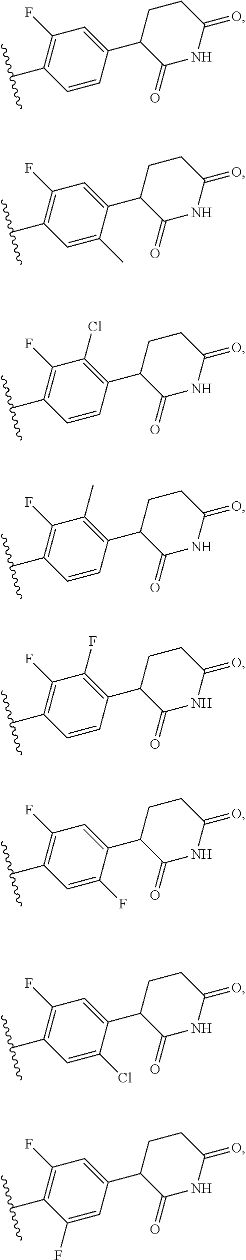

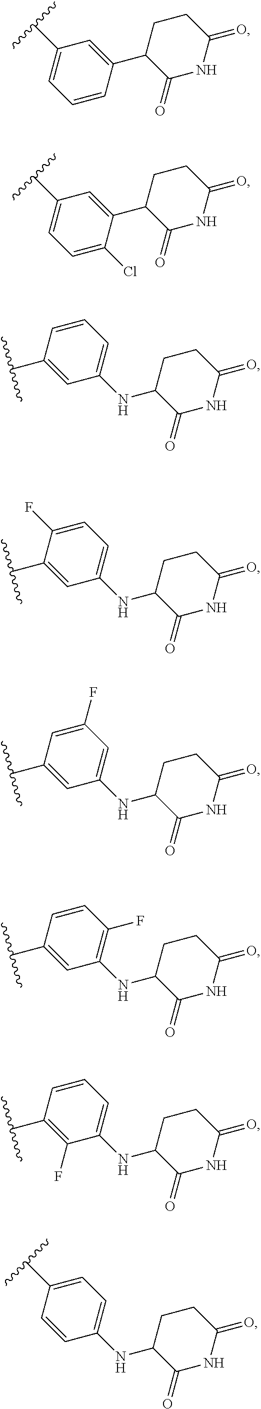

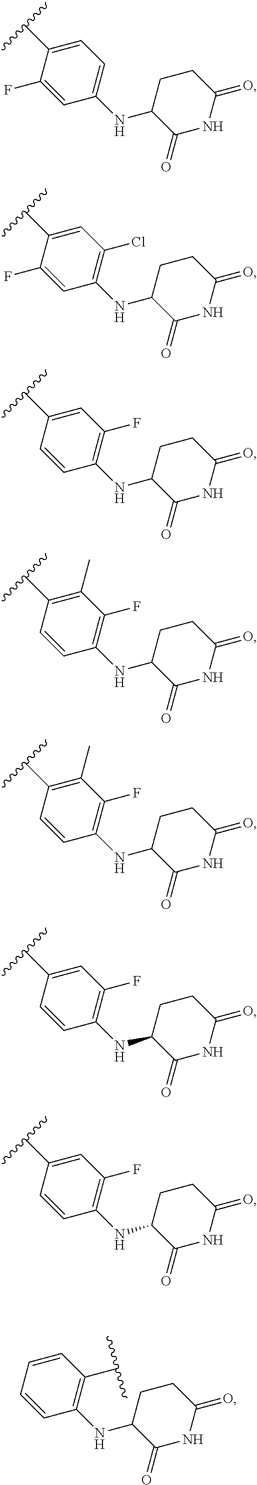

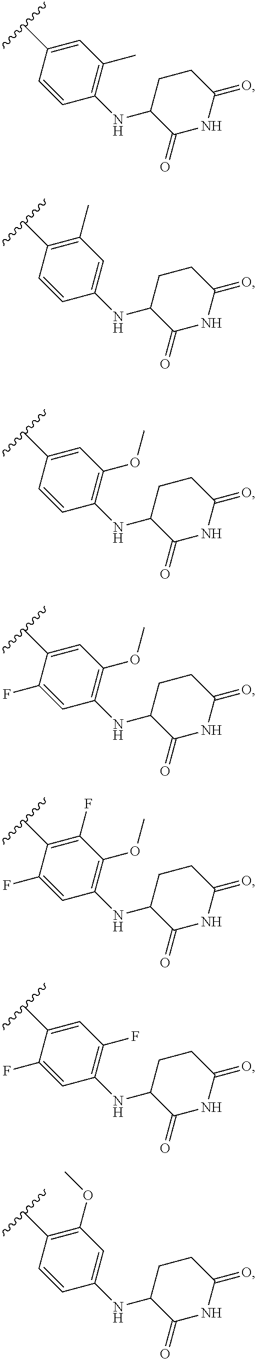

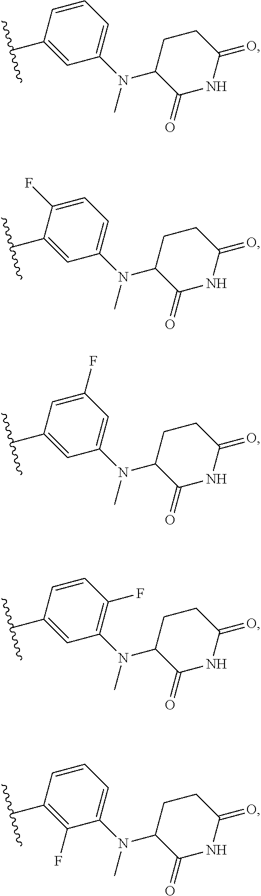 [SMILES: *c1cccc(N(C)C2CCC(=O)NC2=O)c1 ; *c1cc(N(C)C2CCC(=O)NC2=O)ccc1F ; *c1cc(F)cc(N(C)C2CCC(=O)NC2=O)c1 ; *c1ccc(F)c(N(C)C2CCC(=O)NC2=O)c1 ; *c1cccc(N(C)C2CCC(=O)NC2=O)c1F]

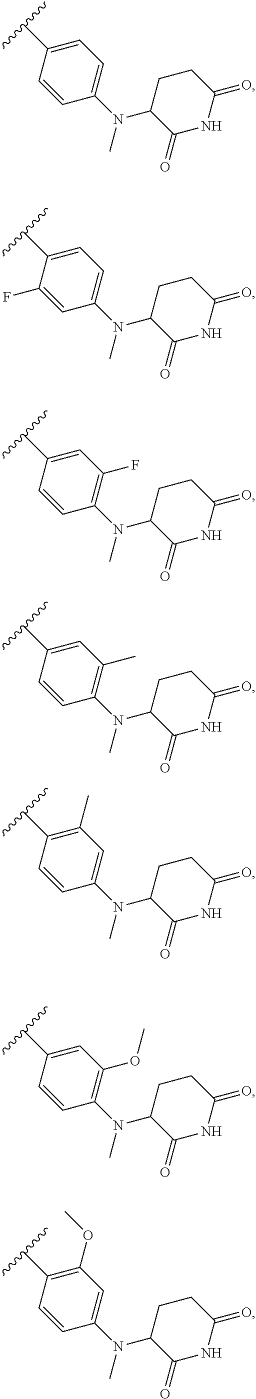

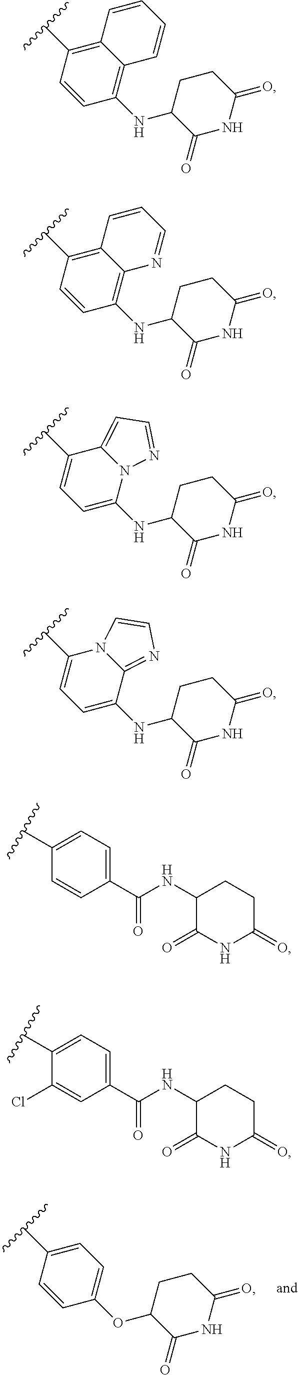

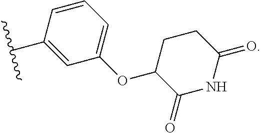 [SMILES: *c1cccc(OC2CCC(=O)NC2=O)c1]

In some embodiments, LBM is E3 Ubiquitin ligase binding moiety recited in Varfolomeev. E. et al., IAP Antagonists Induce Autoubiquitination of c-IAPs. NF-κB activation, and TNFα-Dependent Apoptosis. Cell, 2007, 131(4): 669-81, such as, for example:

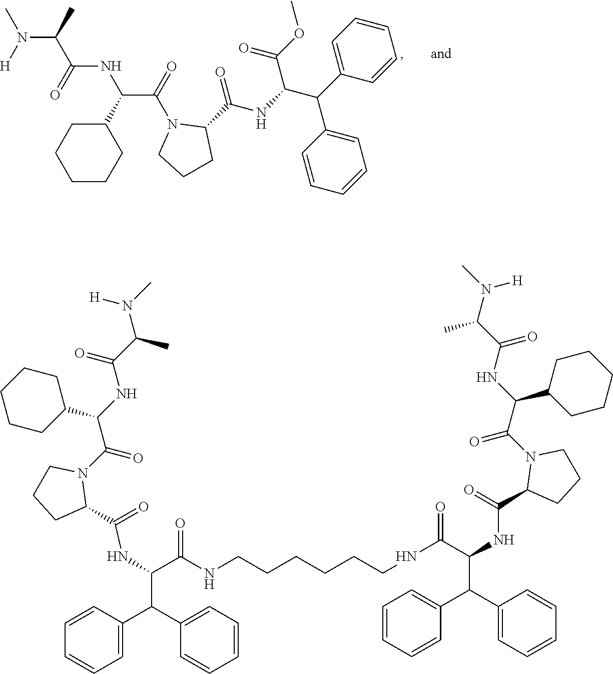

wherein L is attached to a modifiable carbon, oxygen, nitrogen or sulfur atom.

In certain embodiments, LBM is selected from the group consisting of:

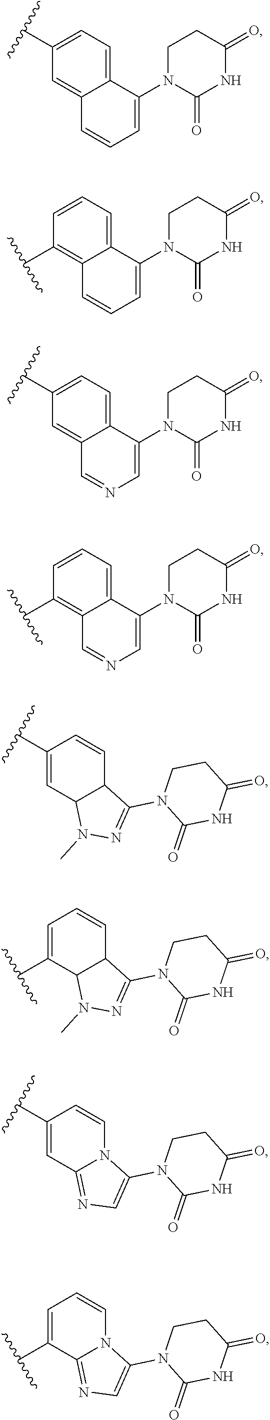

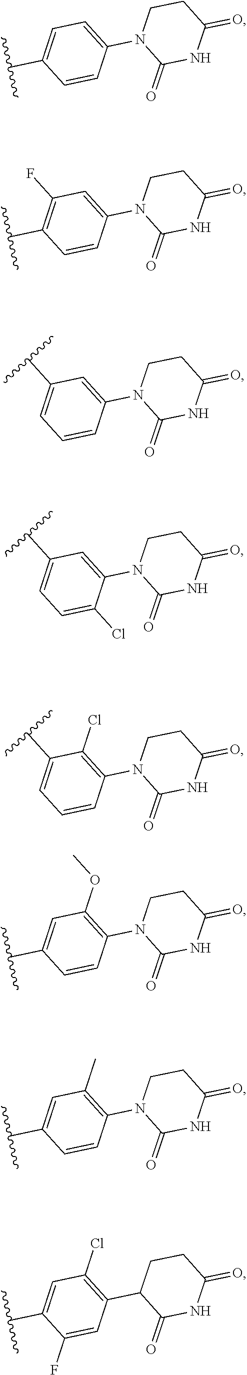

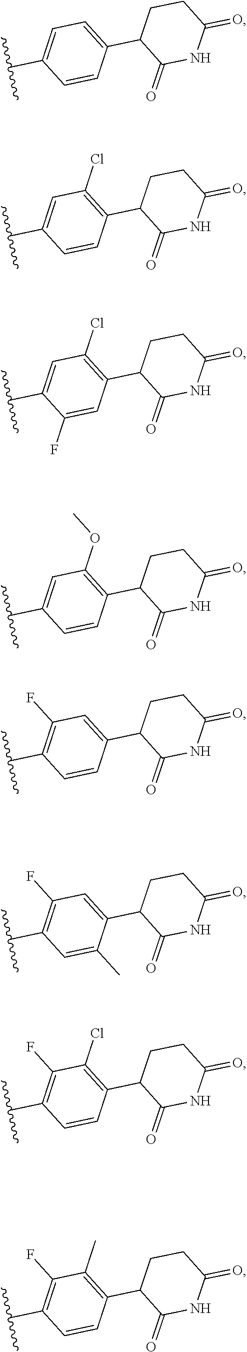

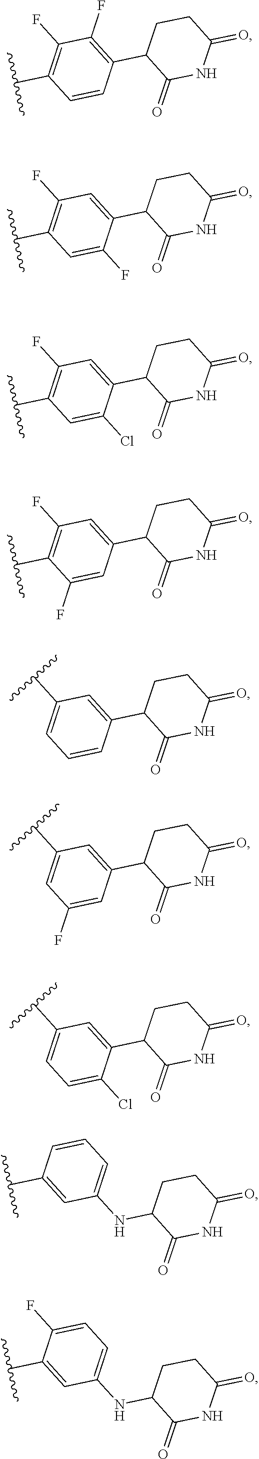

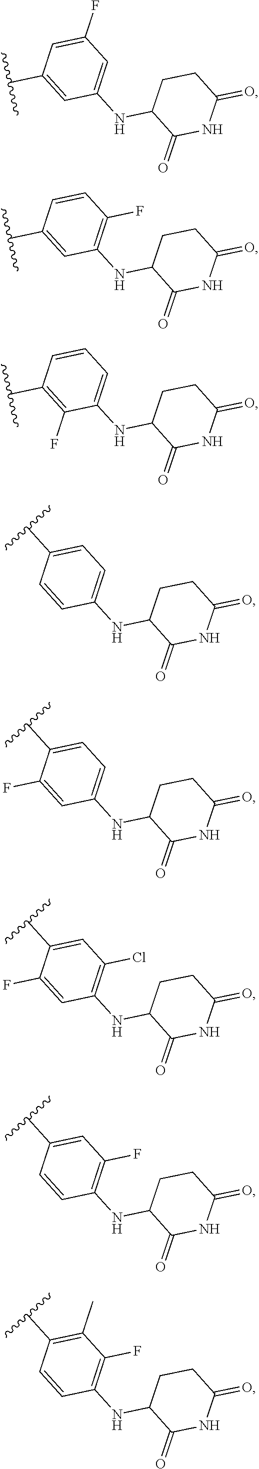

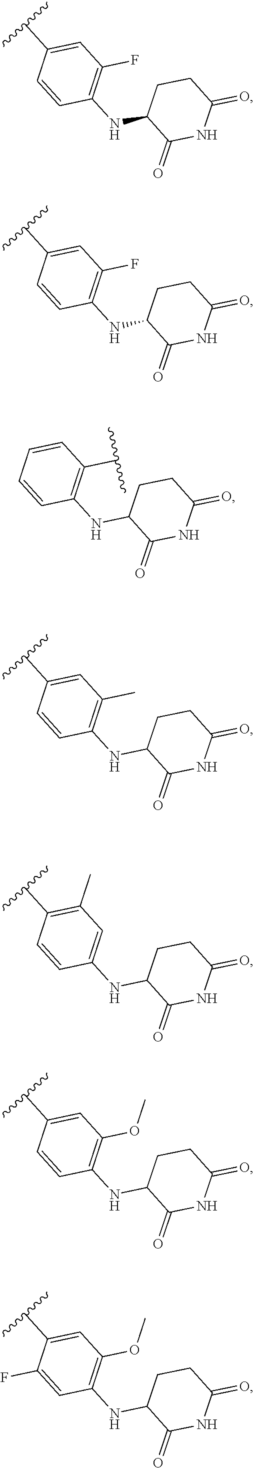

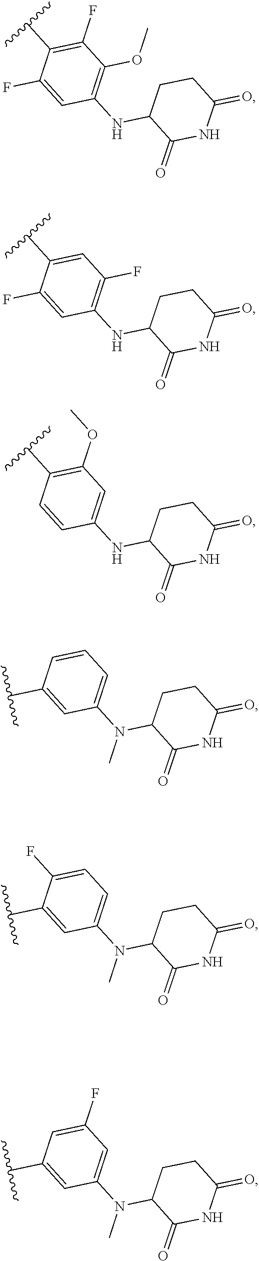 [SMILES: *c1c(F)cc(NC2CCC(=O)NC2=O)c(OC)c1F ; *c1cc(F)c(NC2CCC(=O)NC2=O)cc1F ; *c1ccc(NC2CCC(=O)NC2=O)cc1OC ; *c1cccc(N(C)C2CCC(=O)NC2=O)c1 ; *c1cc(N(C)C2CCC(=O)NC2=O)ccc1F ; *c1cc(F)cc(N(C)C2CCC(=O)NC2=O)c1]

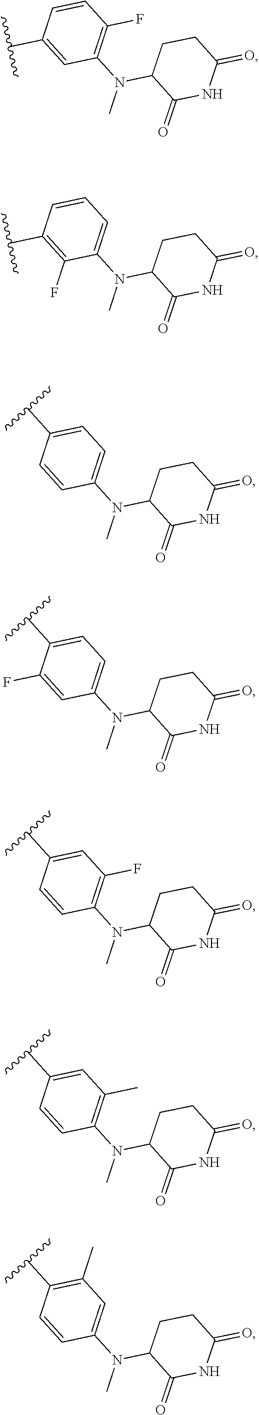

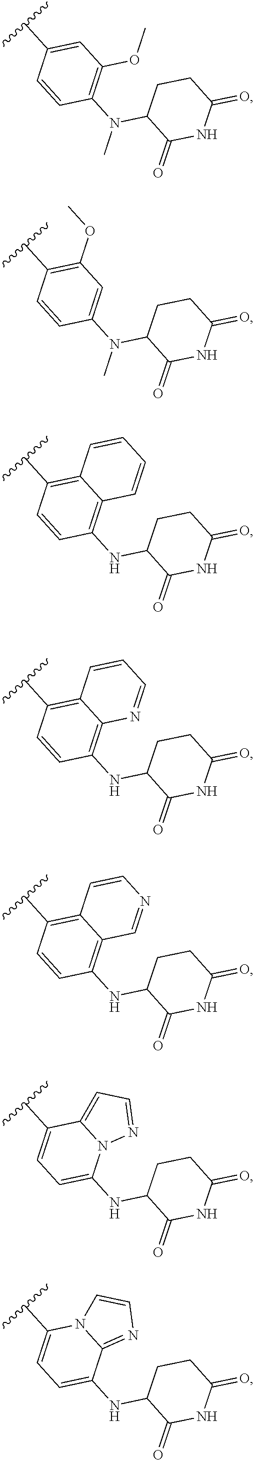

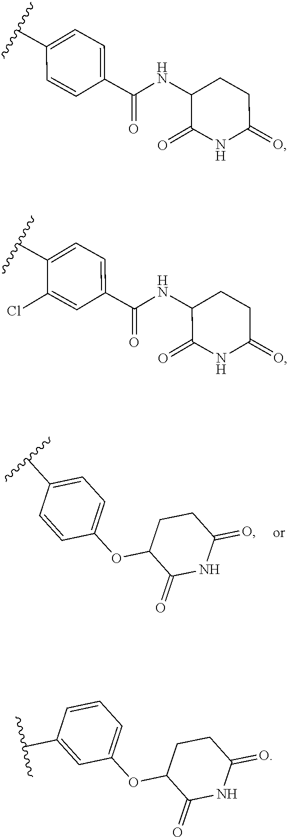 [SMILES: *c1ccc(C(=O)NC2CCC(=O)NC2=O)cc1 ; *c1ccc(C(=O)NC2CCC(=O)NC2=O)cc1Cl ; *c1ccc(OC2CCC(=O)NC2=O)cc1 ; "or" ; *c1cccc(OC2CCC(=O)NC2=O)c1]

In certain embodiments, the LBM is selected from the group consisting of:

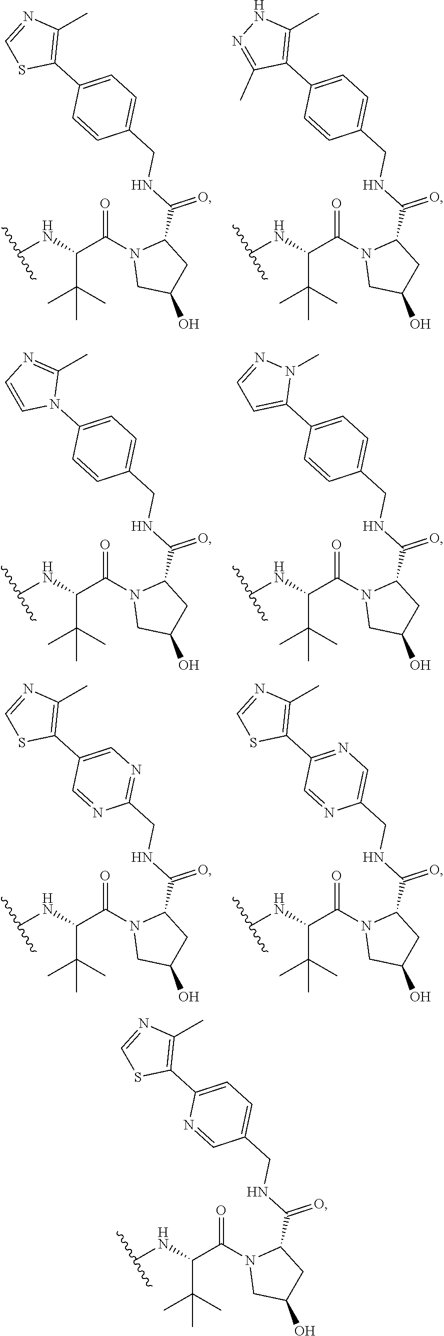

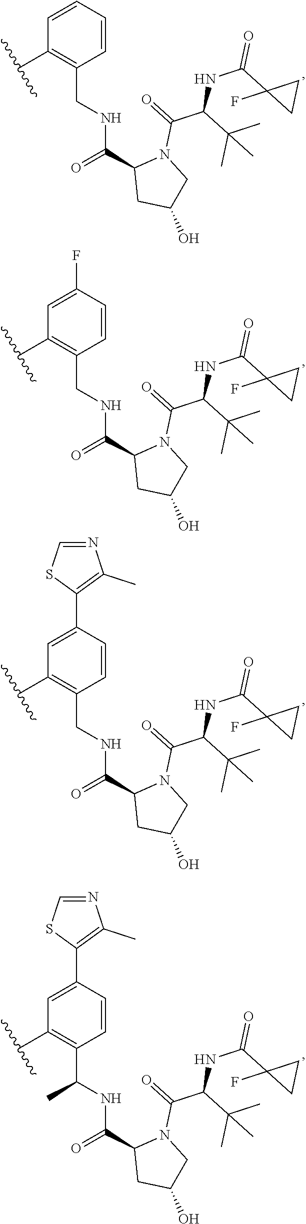 [SMILES: *c1ccccc1CNC(=O)[C@@H]1C[C@@H](O)CN1C(=O)[C@@H](NC(=O)C1(F)CC1)C(C)(C)C ; *c1cc(F)ccc1CNC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(F)CC1)C(C)(C)C ; *c1cc(-c2scnc2C)ccc1[C@H](C)NC(=O)[C@@H]1C[C@@H](O)CN1C(=O)[C@@H](NC(=O)C1(F)CC1)C(C)(C)C]

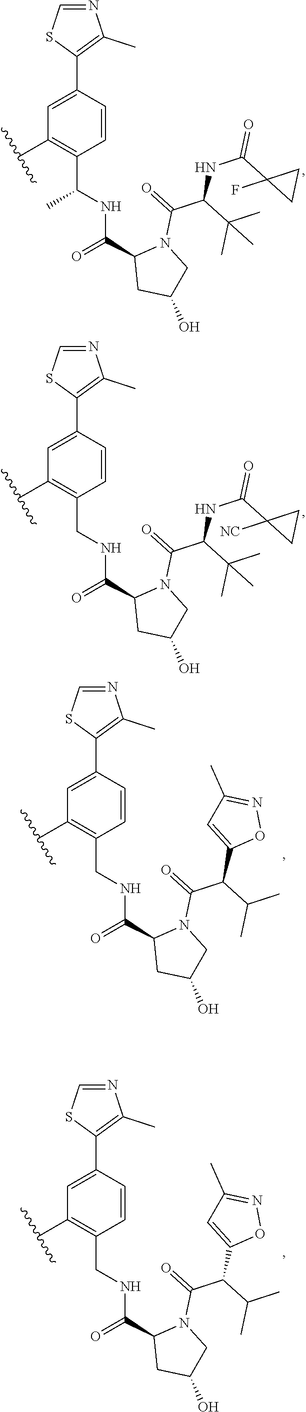 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C ; *c1cc(-c2scnc2C)ccc1CNC(=O)[C@@H]1C[C@@H](O)CN1C(=O)[C@@H](NC(=O)C1(C#N)CC1)C(C)(C)C ; *c1cc(-c2scnc2C)ccc1CNC(=O)[C@@H]1C[C@@H](O)CN1C(=O)[C@@H](c1cc(C)no1)C(C)C ; *c1cc(-c2scnc2C)ccc1CNC(=O)[C@@H]1C[C@@H](O)CN1C(=O)[C@H](c1cc(C)no1)C(C)C]

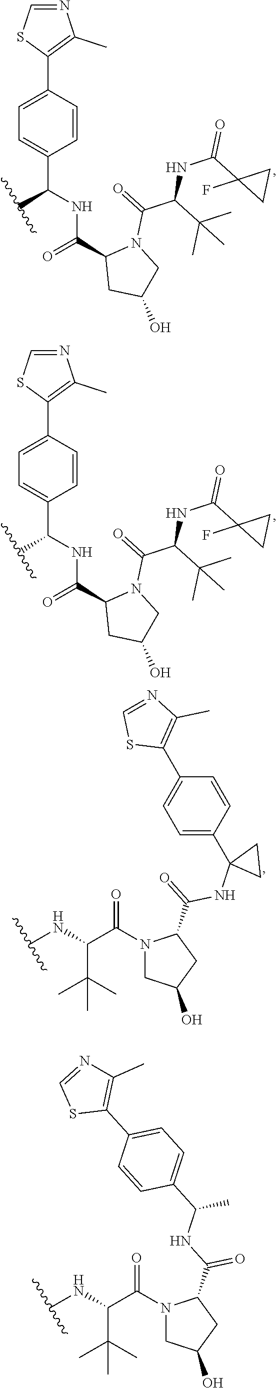

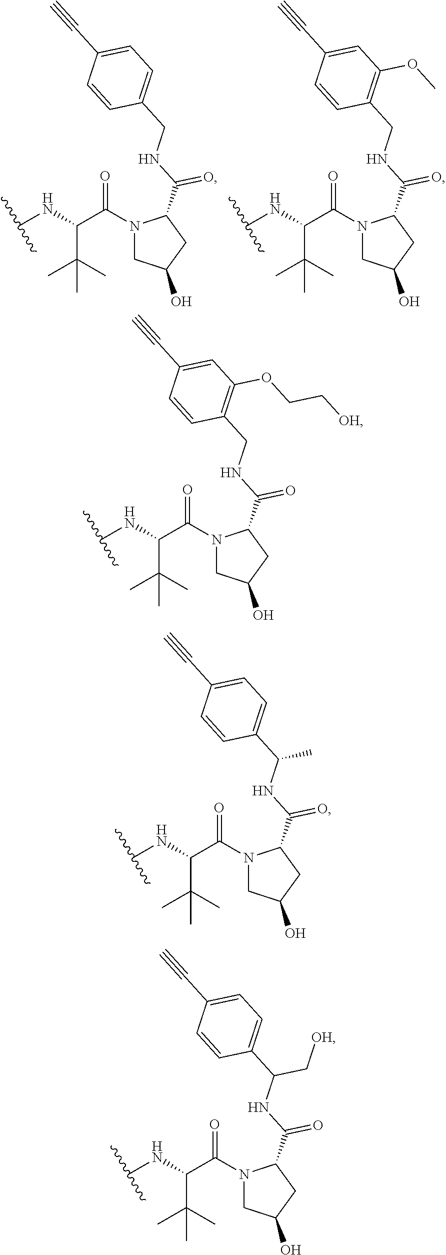

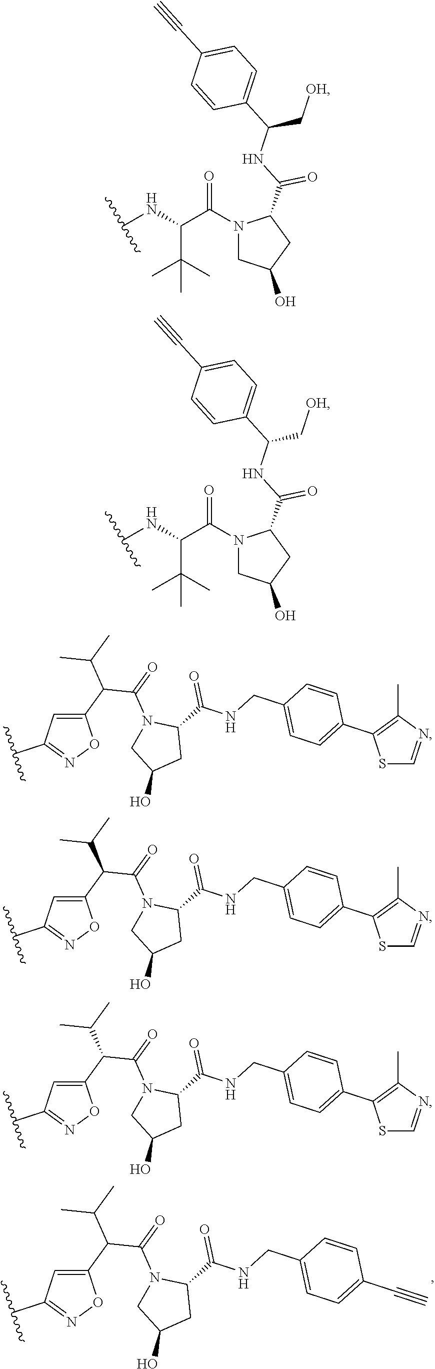

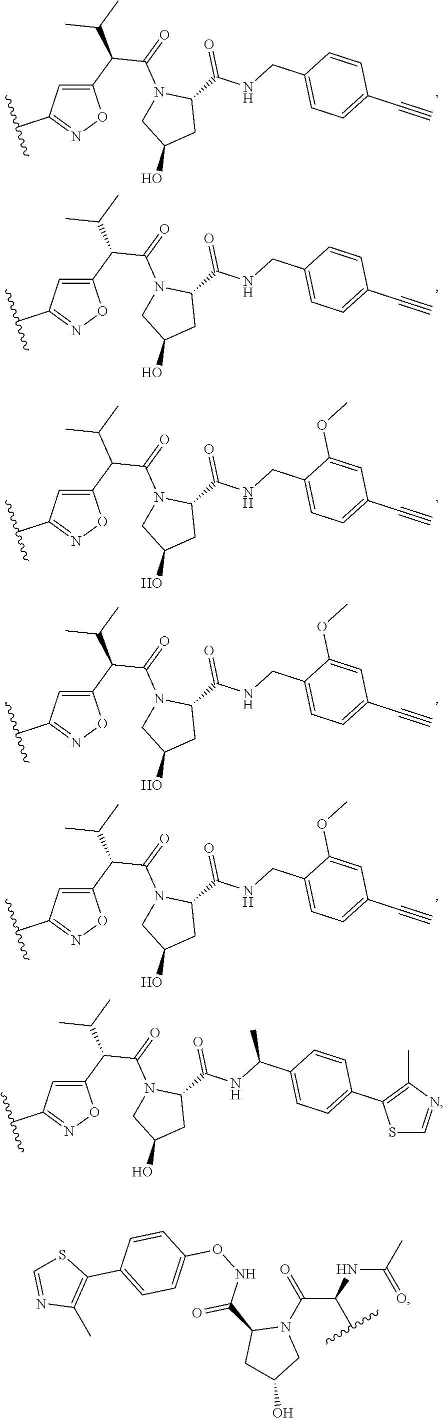

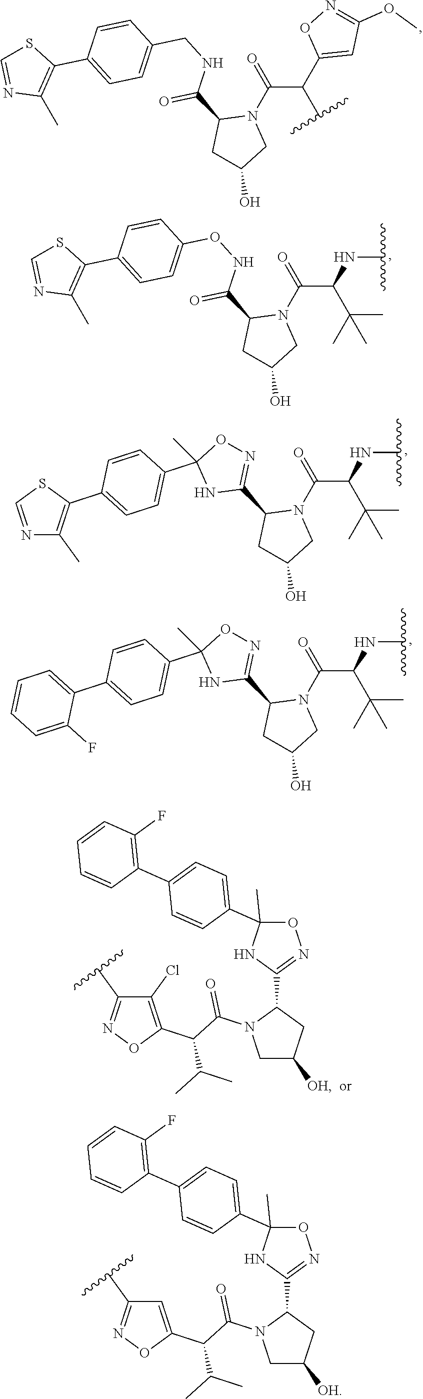

In certain embodiments, LBM is selected from the group consisting of:

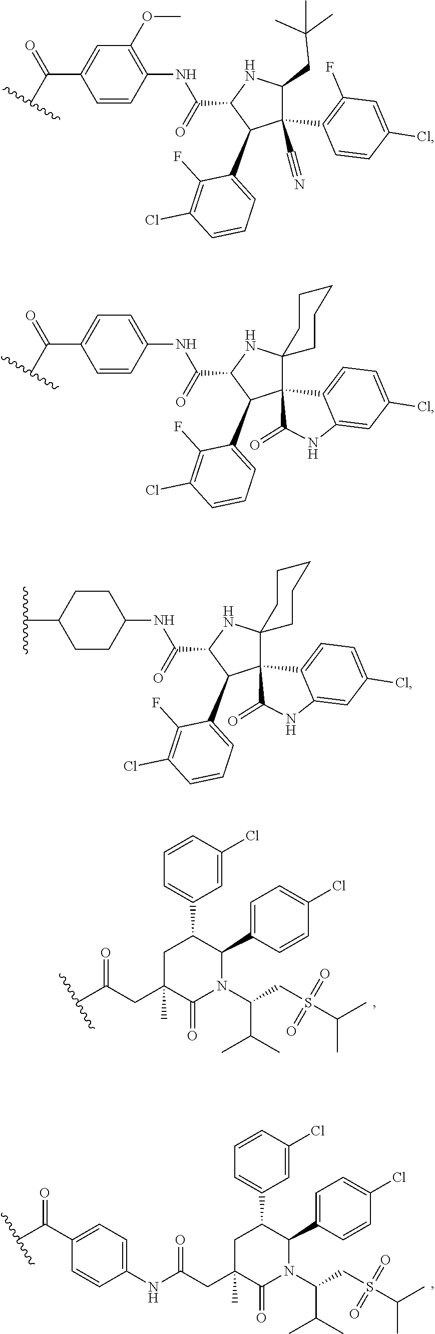 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *C(=O)c1ccc(NC(=O)[C@@H]2NC3(CCCCC3)[C@@]3(C(=O)Nc4cc(Cl)ccc43)[C@H]2c2cccc(Cl)c2F)cc1 ; *C1CCC(NC(=O)[C@@H]2NC3(CCCCC3)[C@@]3(C(=O)Nc4cc(Cl)ccc43)[C@H]2c2cccc(Cl)c2F)CC1 ; *C(=O)C[C@@]1(C)C[C@H](c2cccc(Cl)c2)[C@@H](c2ccc(Cl)cc2)N([C@H](CS(=O)(=O)C(C)C)C(C)C)C1=O ; *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

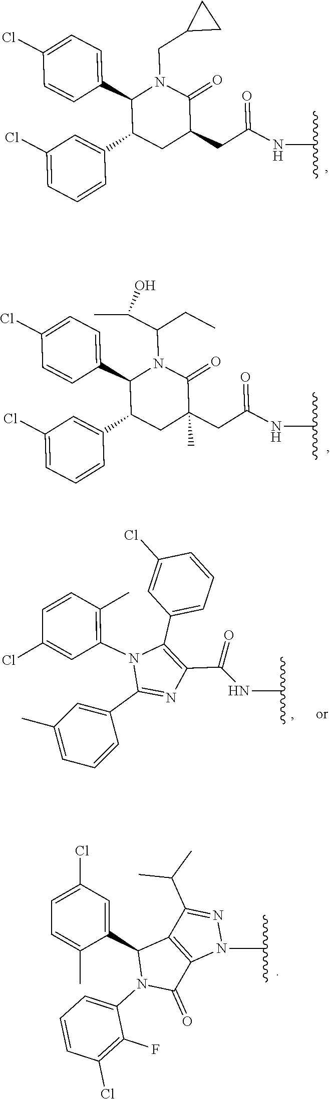 [SMILES: *NC(=O)C[C@H]1C[C@H](c2cccc(Cl)c2)[C@@H](c2ccc(Cl)cc2)N(CC2CC2)C1=O ; *NC(=O)C[C@@]1(C)C[C@H](c2cccc(Cl)c2)[C@@H](c2ccc(Cl)cc2)N(C(CC)[C@H](C)O)C1=O ; *NC(=O)c1nc(-c2cccc(C)c2)n(-c2cc(Cl)ccc2C)c1-c1cccc(Cl)c1 ; *n1nc(C(C)C)c2c1C(=O)N(c1cccc(Cl)c1F)[C@H]2c1cc(Cl)ccc1C]

As described herein, L is a bivalent moiety that connects TBM and DM.

In certain embodiments, L is a bivalent moiety that connects TBM to a lysine mimetic.

In certain embodiments, L is a covalent bond or a bivalent, saturated or partially unsaturated, straight or branched C₁₋₅₀ hydrocarbon chain, wherein 0-6 methylene units of L may optionally be replaced by -Cy-, —CHF—, —CF₂—, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, NRC(O)O—,

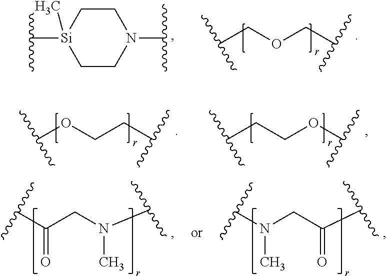

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
r is 0, 1, 2, 3, 4, 5, 6, 7, 8, 9, or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro-carbocyclylenyl. In some embodiments, each -Cy- is independently an optionally substituted 8-10 membered bicyclic saturated or partially unsaturated carbocyclylenyl. In some embodiments, each -Cy is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro-heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is substituted with C₁₋₆ alkyl (e.g., methyl, ethyl, isopropyl). In some embodiments, -Cy- is substituted with oxo. In some embodiments, -Cy- is substituted with halogen. In some embodiments, -Cy- is substituted with fluoro. In some embodiments, -Cy- is substituted with geminal difluoro. In some embodiments, -Cy- is substituted with —OH. In some embodiments, -Cy- is substituted with —NR₂.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, L is —NR—(C₁₋₁₀ aliphatic)-. In some embodiments, L is —(C₁₋₁₀ aliphatic)-NR—(C1-10aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L, is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH2CH2O)₁₋₁₀CH₂CH₂—. In some embodiments, L is -Cy-CONR—(C₁₋₁₀ aliphatic). In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy- CONR—(C₁₋₁₀ aliphatic)-. In some embodiments, L is -Cy-(C₁₋₁₀ aliphatic)-CONR-Cy-(C₁₋₁₀ aliphatic)-.

In certain embodiments, L is any bivalent linker provided in WO 2024/064080, incorporated herein by reference in its entirety.

### 4. Compounds

In a first embodiment, provided is a compound of Formula I:

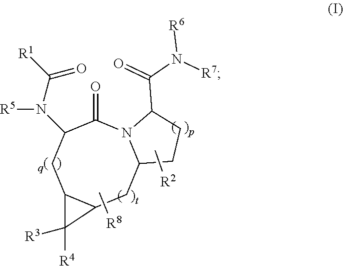

or a pharmaceutically acceptable salt thereof, wherein the variables are as described above. Alternatively, as part of the first embodiment, R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with R¹² and/or, as valency permits, optionally further substituted with 1 to 3 groups selected from R^{Q}, and the remaining variables are as described above.

In a second embodiment, the compound of Formula I is of the Formula II:

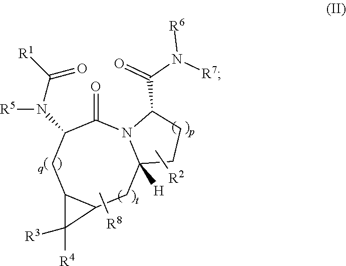

or a pharmaceutically acceptable salt thereof, wherein the variables are as described above for Formula I.

In a third embodiment, q in the compound of Formula I or II, or pharmaceutically acceptable salt thereof is 1, wherein the remaining variables are as described above for Formula I.

In a fourth embodiment, t in the compound of Formula I or II, or pharmaceutically acceptable salt thereof is 1, wherein the remaining variables are as described above for Formula I or the third embodiment.

In a fifth embodiment, p in the compound of Formula I or II, or pharmaceutically acceptable salt thereof is 1, wherein the remaining variables are as described above for Formula I or any one of the third or fourth embodiment.

In a sixth embodiment, R² in the compound of Formula I or II, or a pharmaceutically acceptable salt thereof is hydrogen, wherein the remaining variables are as described above for Formula I or any one of the third to fifth embodiment.

In a seventh embodiment, the compound of Formula I is of the Formula III or IV

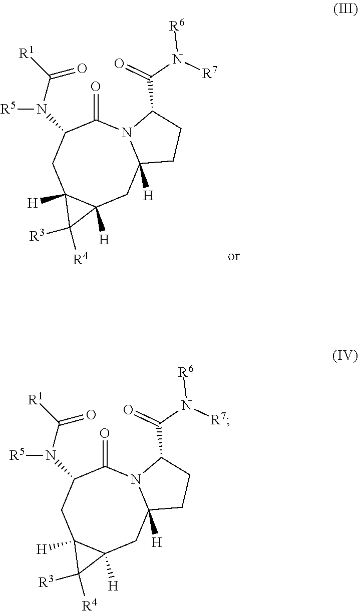

or a pharmaceutically acceptable salt thereof, wherein the variables are as described above for Formula I.

In a an eighth embodiment, R⁵ in the compound of Formula I, II, III, or IV, or pharmaceutically acceptable salt thereof, is hydrogen, wherein the remaining variables are as described for Formula I or any one of the third to sixth embodiment.

In a ninth embodiment, R³ and R⁴ in the compound of Formula I, II, III, or IV, or pharmaceutically acceptable salt thereof, are each independently selected from hydrogen and halo, wherein the remaining variables are as described for Formula I or any one of the third to sixth or eighth embodiments. Alternatively, as part of a ninth embodiment, R³ and R⁴ in the compound of Formula I, II, III, or IV, or pharmaceutically acceptable salt thereof, are each hydrogen, wherein the remaining variables are as described for Formula I or any one of the third to sixth or eighth embodiments. Alternatively, as part of a ninth embodiment, R³ and R⁴ in the compound of Formula I, II, III, or IV, or pharmaceutically acceptable salt thereof, are each fluoro, wherein the remaining variables are as described for Formula I or any one of the third to sixth or eighth embodiments.

In a tenth embodiment, R¹ in the compound of Formula I, II, III, or IV, or pharmaceutically acceptable salt thereof, is selected from 8- to 10-membered fused bicyclic heteroaryl and aryl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}], wherein the remaining variables are as described for Formula I or any one of the third to sixth, eighth or ninth embodiments. Alternatively, as part of a tenth embodiment, R¹ in the compound of Formula I, II, III, or IV, or pharmaceutically acceptable salt thereof, is selected from benzothiophenyl and naphthalenyl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}], wherein the remaining variables are as described for Formula I or any one of the third to sixth, eighth or ninth embodiments. Alternatively, as part of a tenth embodiment, R¹ in the compound of Formula I, II, III, or IV, or pharmaceutically acceptable salt thereof is selected from

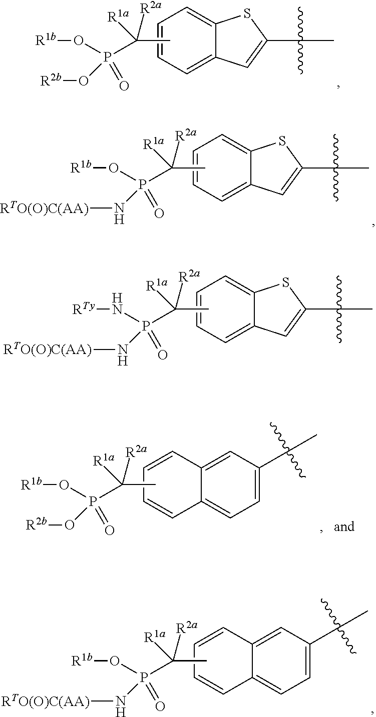

wherein the remaining variables are as described for Formula I or any one of the third to sixth, eighth or ninth embodiments. Alternatively, as part of a tenth embodiment, R¹ in the compound of Formula I, II, III, or IV, or pharmaceutically acceptable salt thereof, is

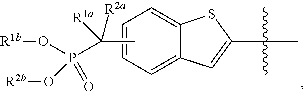

wherein the remaining variables are as described for Formula I or any one of the third to sixth, eighth or ninth embodiments.

In an eleventh embodiment, R^{1a} and R^{2a} in the compound of Formula I, II, III, or IV, or pharmaceutically acceptable salt thereof, are each independently selected from hydrogen and fluoro, wherein the remaining variables are as described for Formula I or any one of the third to sixth and eighth to tenth embodiments. Alternatively, as part of an eleventh embodiment, R^{1a} and R^{2a} in the compound of Formula I, II, III, or IV, or pharmaceutically acceptable salt thereof, are each hydrogen, wherein the remaining variables are as described for Formula I or any one of the third to sixth and eighth to tenth embodiments. Alternatively, as part of an eleventh embodiment, R^{1a} in the compound of Formula I, II, III, or IV, or pharmaceutically acceptable salt thereof, is hydrogen and R^{2a} is fluoro, wherein the remaining variables are as described for Formula I or any one of the third to sixth and eighth to tenth embodiments. Alternatively, as part of an eleventh embodiment, R^{1a} and R^{2a} in the compound of Formula I, II, III, or IV, or pharmaceutically acceptable salt thereof, are each fluoro, wherein the remaining variables are as described for Formula I or any one of the third to sixth and eighth to tenth embodiments.

In a twelfth embodiment, R^{1b} and R^{2b} in the compound of Formula I, II, III, or IV, or pharmaceutically acceptable salt thereof, are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-OC(O)—[(C₁-C₄)alkyl]-OH, —[(C₁-C₄)alkyl]-OC(O)O-[5- to 7-membered heterocyclyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)—[(C₁-C₄)alkyl]-OH, and phenyl, wherein the remaining variables are as described for Formula I or any one of the third to sixth and eighth to eleventh embodiments. Alternatively, as part of a twelfth embodiment, R^{1b} and R^{2b} in the compound of Formula I, II, III, or IV, or pharmaceutically acceptable salt thereof, are each independently selected from hydrogen, [(C₁-C₄)alkyl]-OC(O)—[(C₁-C₄)alkyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], and —[(C₁-C₄)alkyl]-SC(O)—[(C₁-C₄)alkyl], wherein the remaining variables are as described for Formula I or any one of the third to sixth and eighth to eleventh embodiments. Alternatively, as part of a twelfth embodiment, R^{1b} and R^{2b} in the compound of Formula I, II, III, or IV, or pharmaceutically acceptable salt thereof, are each —[(C₁-C₄)alkyl]-OC(O)—[(C₁-C₄)alkyl], wherein the remaining variables are as described for Formula I or any one of the third to sixth and eighth to eleventh embodiments. Alternatively, as part of a twelfth embodiment, R^{1b} and R^{2b} in the compound of Formula I, II, III, or IV, or pharmaceutically acceptable salt thereof, are each hydrogen, wherein the remaining variables are as described for Formula I or any one of the third to sixth and eighth to eleventh embodiments.

In a thirteenth embodiment, —CR^{1a}R^{2a}P(O)OR^{1b}OR^{2b} in the compound of Formula I, II, III, or IV, or pharmaceutically acceptable salt thereof, is selected from

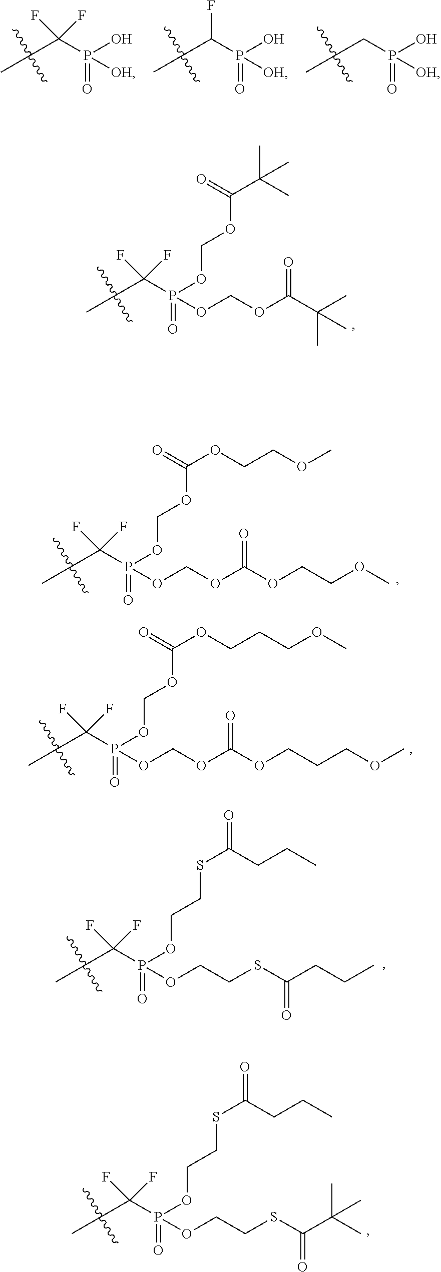

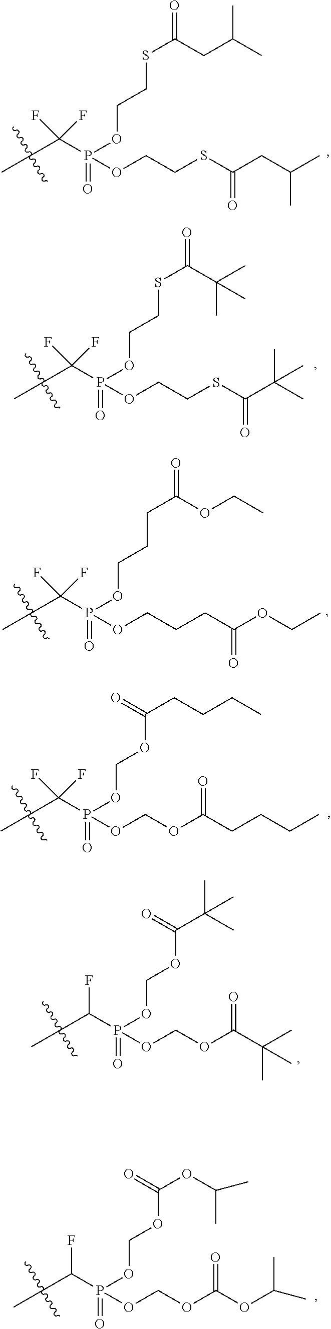

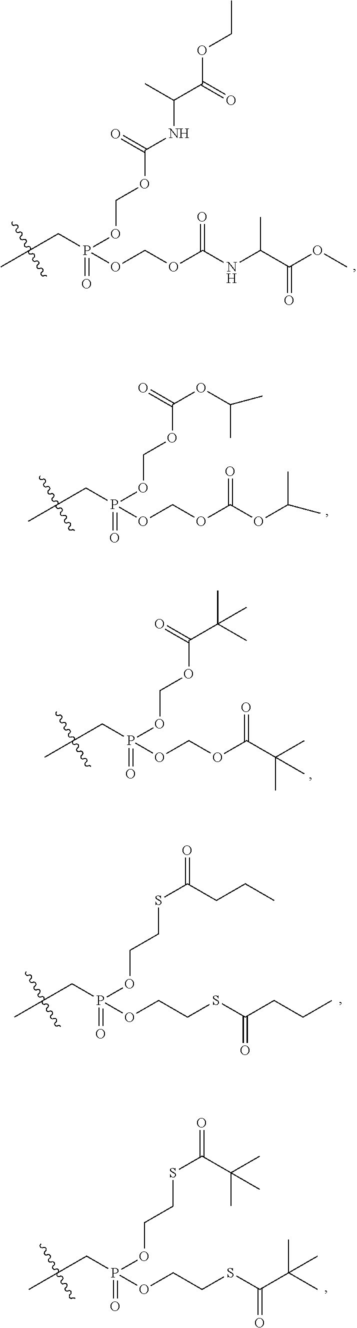 [SMILES: *CP(=O)(OCOC(=O)NC(C)C(=O)OC)OCOC(=O)NC(C)C(=O)OCC ; *CP(=O)(OCOC(=O)OC(C)C)OCOC(=O)OC(C)C ; *CP(=O)(OCOC(=O)C(C)(C)C)OCOC(=O)C(C)(C)C ; *CP(=O)(OCCSC(=O)CCC)OCCSC(=O)CCC ; *CP(=O)(OCCSC(=O)C(C)(C)C)OCCSC(=O)C(C)(C)C]

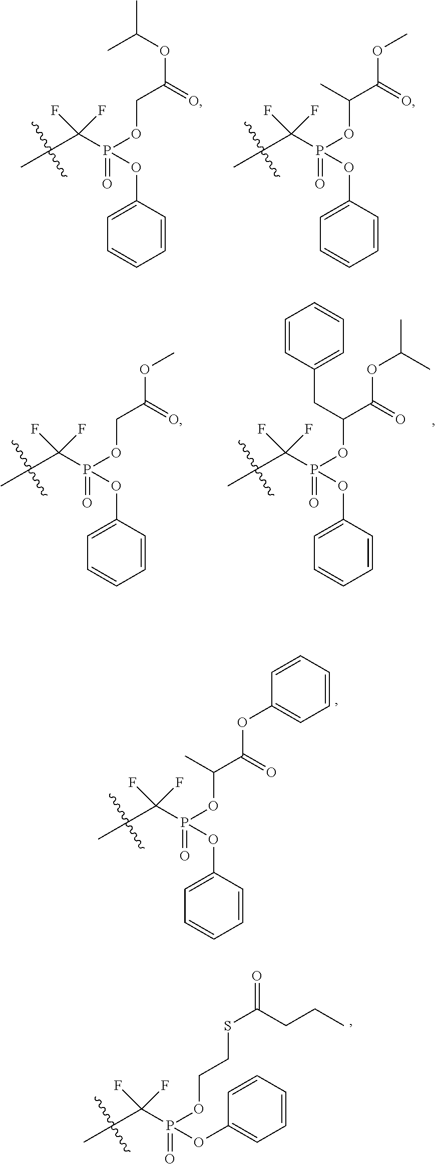 [SMILES: *C(F)(F)P(=O)(OCC(=O)OC(C)C)Oc1ccccc1 ; *C(F)(F)P(=O)(Oc1ccccc1)OC(C)C(=O)OC ; *C(F)(F)P(=O)(OCC(=O)OC)Oc1ccccc1 ; *C(F)(F)P(=O)(Oc1ccccc1)OC(Cc1ccccc1)C(=O)OC(C)C ; *C(F)(F)P(=O)(Oc1ccccc1)OC(C)C(=O)Oc1ccccc1 ; *C(F)(F)P(=O)(OCCSC(=O)CCC)Oc1ccccc1]

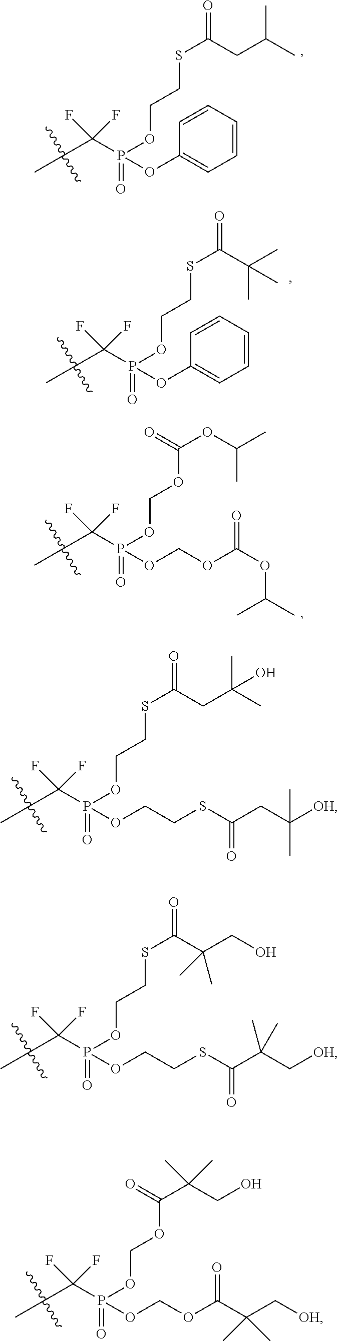

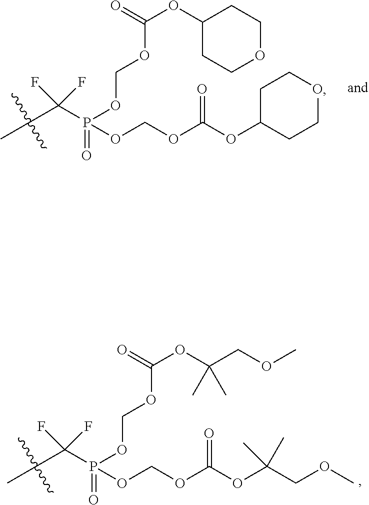 [SMILES: *C(F)(F)P(=O)(OCOC(=O)OC1CCOCC1)OCOC(=O)OC1CCOCC1 ; *C(F)(F)P(=O)(OCOC(=O)OC(C)(C)COC)OCOC(=O)OC(C)(C)COC]

wherein the remaining variables are as described for Formula I or any one of the third to sixth and eighth to twelfth embodiments. Alternatively, as part of a thirteenth embodiment, —CR^{1a}R^{2a}P(O)OR^{1b}OR^{2b} in the compound of Formula I, II, III, or IV, or pharmaceutically acceptable salt thereof, is

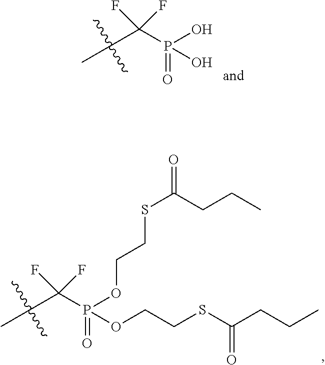 [SMILES: *C(F)(F)P(=O)(O)O ; *C(F)(F)P(=O)(OCCSC(=O)CCC)OCCSC(=O)CCC]

wherein the remaining variables are as described for Formula I or any one of the third to sixth and eighth to twelfth embodiments.

In a fourteenth embodiment, R⁶ and R⁷ in the compound of Formula I, II, III, or IV, or pharmaceutically acceptable salt thereof, together with the nitrogen atom to which they are attached form a 4- to 9-membered monocyclic or bicyclic heterocyclyl substituted with R¹² and/or, as valency permits, optionally further substituted with 1 to 3 groups selected from R^{Q}, wherein the remaining variables are as described for Formula I or any one of the third to sixth and eighth to thirteenth embodiments. Alternatively, as part of a fourteenth embodiment, R and R⁷ in the compound of Formula I, II, III, or IV, or pharmaceutically acceptable salt thereof, together with the nitrogen atom to which they are attached form pyrrolidinyl, azetidinyl, or 4-azaspiro[2.4]heptanyl, each substituted with R¹² and/or, as valency permits, optionally further substituted with 1 to 3 groups selected from R^{Q}, wherein the remaining variables are as described for Formula I or any one of the third to sixth and eighth to thirteenth embodiments.

In a fifteenth embodiment, R^{Q} in the compound of Formula I, II, III, or IV, or pharmaceutically acceptable salt thereof, is cyano or 5- to 7-membered heteroaryl, wherein the remaining variables are as described for Formula I or any one of the third to sixth and eighth to fourteenth embodiments. Alternatively, as part of a fifteenth embodiment, R^{Q} in the compound of Formula I, II, III, or IV, or pharmaceutically acceptable salt thereof, is cyano or pyridinyl, wherein the remaining variables are as described for Formula I or any one of the third to sixth and eighth to fourteenth embodiments.

In a sixteenth embodiment, R¹² in the compound of Formula I, II, III, or IV, or pharmaceutically acceptable salt thereof, is selected from —NHC(O)R^{10C}E, —OR^{10C}E, phenyl, pyridinyl, and pyridin-2(1H)-one, each substituted with R^{10C}E, —OR^{10C}E, —NHR^{10C}E, or —[N(C₁-C₄)alkyl]R^{10C}E, wherein the remaining variables are as described for Formula I or any one of the third to sixth and eighth to fifteenth embodiments.

In a seventeenth embodiment, R^{10C} in the compound of Formula I, II, III, or IV, or pharmaceutically acceptable salt thereof, is selected from (C₁-C₁₀)alkylene and (C₂-C₁₀)alkynelene each optionally substituted with one or more groups selected from halo and oxo, and wherein said (C₁-C₆)alkylene and (C₂-C₁₀)alkynelene may also be optionally interrupted by one or more heteroatoms selected from S, N, and O, and/or optionally interrupted by one or more rings selected from 5- to 7-membered heteroaryl, (C₃-C₆)cycloakyl, phenyl, and 4- to 7-membered heterocyclyl, wherein the remaining variables are as described for Formula I or any one of the third to sixth and eighth to sixteenth embodiments. Alternatively, as part of a seventeenth embodiment, R^{10C} in the compound of Formula I, II, III, or IV, or pharmaceutically acceptable salt thereof, is selected from (C₁-C₁₀)alkylene, (C₂-C₁₀)alkynelene, —(CH₂)ₘC(O)NH[(CH₂)O]ᵥ(CH₂)ₙ, —(CH₂)O[(CH₂)O](CH₂)ₙ, —(C₂-C₈)alkynelene[4- to 7-membered heterocyclyl], 4- to 7-membered heterocyclyl, [(CH₂)O]ᵥ(CH₂)ₙ, and —(CH₂)ₙC(O)NH[4- to 7-membered heterocyclyl], wherein m, n, and v are each independently selected from 0, 1, 2, 3, 4, 5, and 6, wherein the remaining variables are as described for Formula I or any one of the third to sixth and eighth to sixteenth embodiments. In another alternative, as part of a seventeenth embodiment, R^{10C} in the compound of Formula I, II, III or IV or pharmaceutically acceptable salt thereof, is selected from:

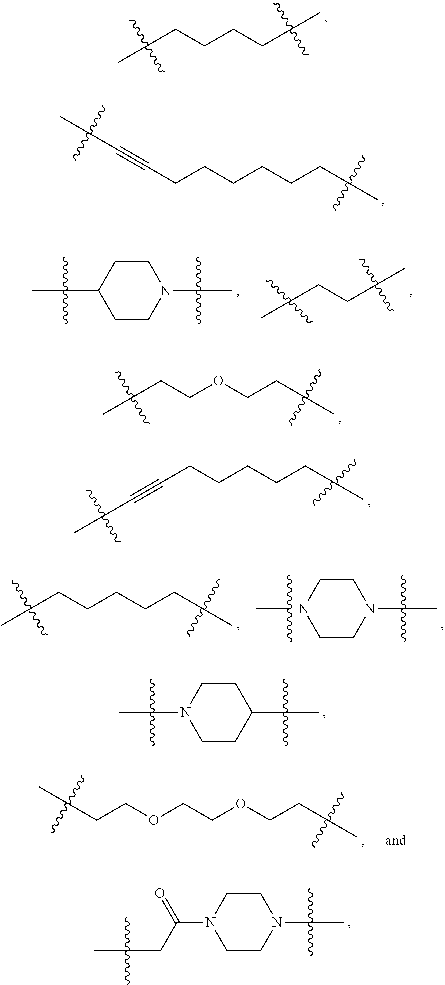

wherein the remaining variables are as described for Formula I or any one of the third to sixth and eighth to sixteenth embodiments.

In an eighteenth embodiment, E in the compound of Formula I, II, III, or IV, or pharmaceutically acceptable salt thereof, is selected from a CRBN. VHL, IAP, or MDM2 based E3 binder, wherein the remaining variables are as described for Formula I or any one of the third to sixth and eighth to seventeenth embodiments. Alternatively, as part of an eighteenth embodiment E in the compound of Formula I, II, III, or IV, or pharmaceutically acceptable salt thereof, is selected from

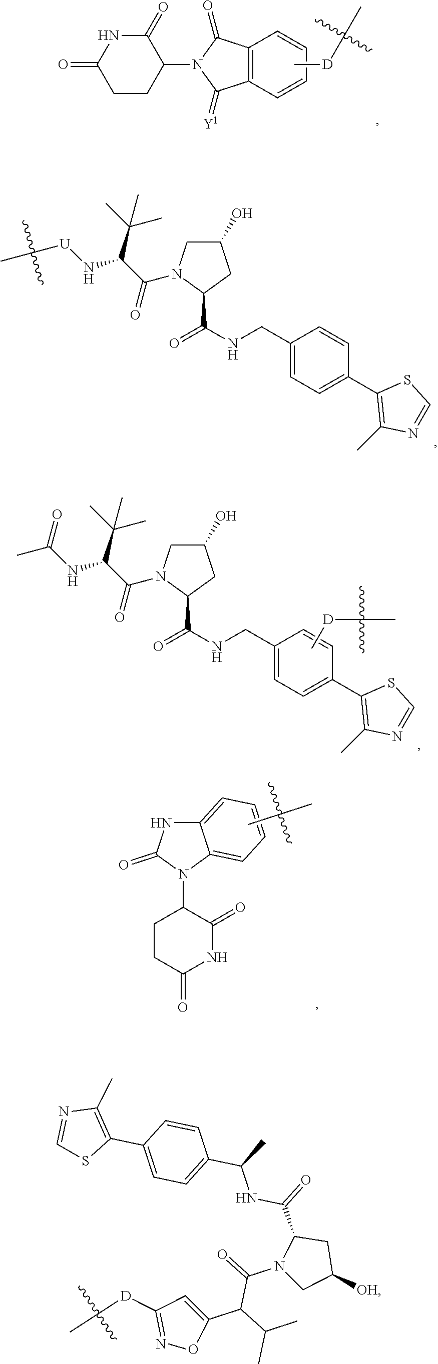

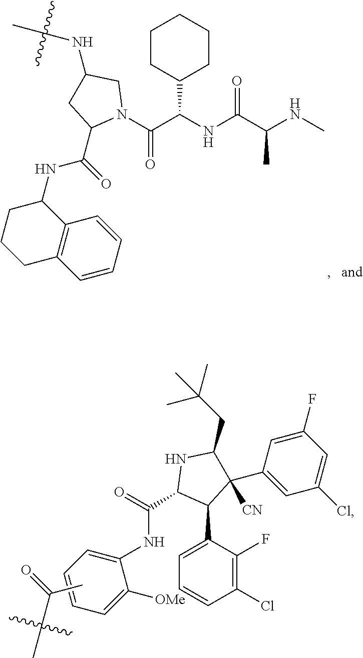

wherein:
Y and Y¹ are each independently is O or CH₂;
D is O, NH, or a bond;
U is absent or C(O); and
   R is halo or cyano, wherein the remaining variables are as described for Formula I or any one of the third to sixth and eighth to seventeenth embodiments. In another alternative, as part of an eighteenth embodiment E in the compound of Formula I, II, III, or IV, or pharmaceutically acceptable salt thereof, is

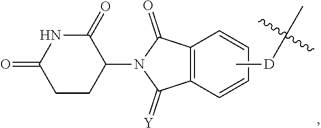

wherein the remaining variables are as described for Formula i or any one of the third to sixth and eighth to seventeenth embodiments.

Compounds having the Formula I are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of STAT proteins, in particular STAT3 and/or STAT6. In some aspects, the compounds, pharmaceutical acceptable salts, and pharmaceutical compositions described herein degrade STAT3 and/or STAT6.

In some aspects, the compounds and pharmaceutical compositions described herein are useful in a condition responsive to the modulation (or degradation) of STAT3 and/or STAT6. Thus, provided herein are methods of treating a condition responsive to the modulation (e.g., degradation) of STAT3 and/or STAT6 in a subject, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a condition responsive to the modulation (e.g., degradation) of STAT3 and/or STAT6. Also provided is a compound described herein, or a pharmaceutically acceptable salt thereof, or a pharmaceutical composition comprising a disclosed compound or pharmaceutically acceptable salt thereof, for use in treating a condition responsive to the modulation (e.g., degradation) of STAT3 and/or STAT6.

In one aspect, the condition responsive to the modulation (e.g., degradation) of STAT3 and/or STAT6 include, but are not limited to, cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In another aspect, the condition responsive to the modulation (e.g., degradation) of STAT3 and/or STAT6 include, but are not limited to, cancer (see, e.g., Turkson & Jove, Oncogene 2000, 19:6613-6626), diabetes (see, e.g., Gurzov et al., FEBS 2016, 283:3002), cardiovascular disease (see, e.g., Grote et al., Vasc. Pharmacol. 2005, 43:2005), viral disease (see, e.g., Gao et al., J Hepatol. 2012, 57(2):430), autoimmune diseases such as lupus (see, e.g., Goropevsek et al., Clin. Rev. Alleg. & Immun. 2017, 52(2):164), and rheumatoid arthritis (see, e.g., Walker & Smith, J. Rheumat. 2005, 32(9), 1650), autoinflammatory syndromes (see, e.g., Rauch et al., Jak-Stat 2013, 2(1):e23820), atherosclerosis (see, e.g., Ortiz-Munoz et al., Arterio., Thromho., Vase. Bio. 2009, 29:525), psoriasis (see, e.g., Andres et al., Exp. Derm. 2013, 22(5):323), allergic disorders (see, e.g., Oh et al., Eur. Respir. Rev. 2019, 19(115):46), inflammatory bowel disease (see, e.g., Sugimoto, World J Gastroenterol. 2008, 14(33):5110), inflammation (see, e.g., Tamiya et al., Arterio. Thrombo., Vasc. Bio. 2011, 31:980), acute and chronic gout and gouty arthritis, neurological disorders (see, e.g., Campbell. Brain Res. Rev. 2005, 48(2): 166), metabolic syndrome, immunodeficiency disorders such as AIDS and HIV (see, e.g., O'Shea et al., N. Engl. J. Med. 2013, 368:161), destructive bone disorders (see, e.g., Jatiani et al., Genes & Can. 2011, 1(10):979), osteoarthritis, proliferative disorders, Waldenstrom's Macroglobulinemia (see, e.g., Hodge et al., Blood 2014, 123(7):1055) infectious diseases, conditions associated with cell death, pathologic immune conditions involving T cell activation, and CNS disorders.

Proliferative disorders, include, but are not limited to a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-I driven disorder, an MyD88 driven disorder, Smoldering of indolent multiple myeloma, or hematological malignancies (including leukemia, diffuse large B-cell lymphoma (DLBCL). ABC DLBCL, chronic lymphocytic leukemia (CLL), chronic lymphocytic lymphoma, primary effusion lymphoma, Burkitt lymphoma/leukemia, acute lymphocytic leukemia. B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the cancer to be treated is selected from glioma, breast cancer, prostate cancer, head and neck squamous cell carcinoma, skin melanomas, ovarian cancer, malignant peripheral nerve sheath tumors (MPNST), and pancreatic cancer. In other embodiments, the cancer to be treated is cancer selected from glioma, breast cancer, prostate cancer, head and neck squamous cell carcinoma, skin melanomas, ovarian cancer, malignant peripheral nerve sheath tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes. In other embodiments, the cancer is selected from solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia Such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

Compounds, salts, and compositions described herein are also useful in the treatment of inflammatory or obstructive airways diseases, resulting, for example, in reduction of tissue damage, airways inflammation, bronchial hyperreactivity, remodeling or disease progression. Inflammatory or obstructive airways diseases include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds, salts, and compositions described herein are also useful in the treatment of heteroimmune diseases including, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Compounds, salts, and compositions described herein are also useful in the treatment of other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. Compounds, salts, and compositions described herein are also useful in the treatment of bronchitis including, but not limited to, acute, arachidic, catarrhal, croupus, chronic or phthinoid bronchitis. Compounds, salts, and compositions described herein are also useful in the treatment of pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds, salts, and compositions described herein are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds, salts, and compositions described herein are also useful in the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy. Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospirosis renal disease, glaucoma, retinal disease, ageing, headache, pain, complex regional pain syndrome, cardiac hypertrophy, musclewasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases. COPD (reduction of damage, airways inflammation, bronchial hyperreactivity, remodeling or disease progression), pulmonary disease, cystic fibrosis, acid induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis. Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, cardiovascular diseases which can be treated according to the present methods include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the present methods include, but are not limited to. Alzheimer's disease, Parkinson's disease, amyotrophic lateral sclerosis. Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of die specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Preparation of Compounds

The compounds claimed herein were prepared following the procedures outlined in the following schemes. Compound names were generated using the software built into ChemDraw. To the extent that there are discrepancies between the name or a compound and its depicted structure, the depicted chemical structure is to be taken as the appropriate compound.

The following intermediates can be prepared according to the protocols described in PCT/US2023/065171:
(3S,6S,7aS,8aR,9aR)-6-((tert-butoxycarbonyl)amino)-5-oxodecahydro-1H-cyclopropa[d]pyrrolo[1,2-a]azocine-3-carboxylic acid;
(difluoro(2-((4-nitrophenoxy)carbonyl)benzo[b]thiophen-5-yl)methyl)phosphonic acid; and
(difluoro(2-((perfluorophenoxy)carbonyl)benzo[b]thiophen-5-yl)methyl)phosphonic acid.

The following intermediates can be prepared according to the protocols described in PCT/US2023/010447:
tert-butyl (R)-6-(2-oxo-1,2-dihydropyridin-4-yl)-4-azaspiro[2.4]heptane-4-carboxylate; and
tert-butyl 3-(4-fluoropyridin-3-yl)azetidine-1-carboxylate.

### Preparation of ((2-(((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 1)

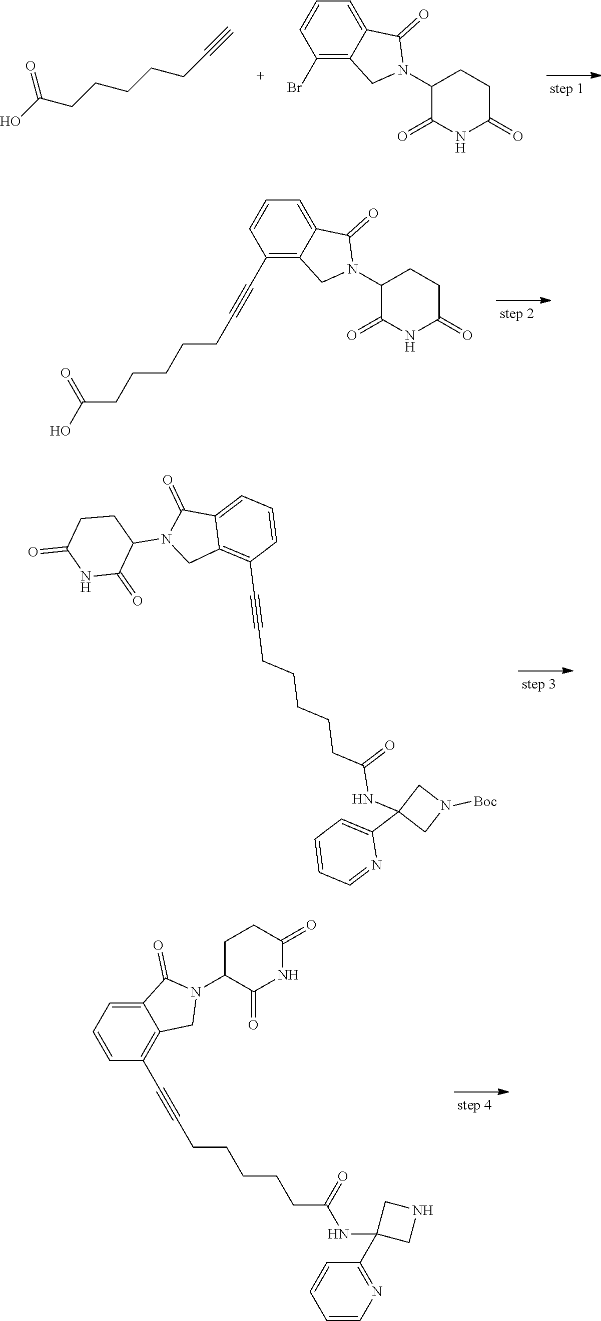

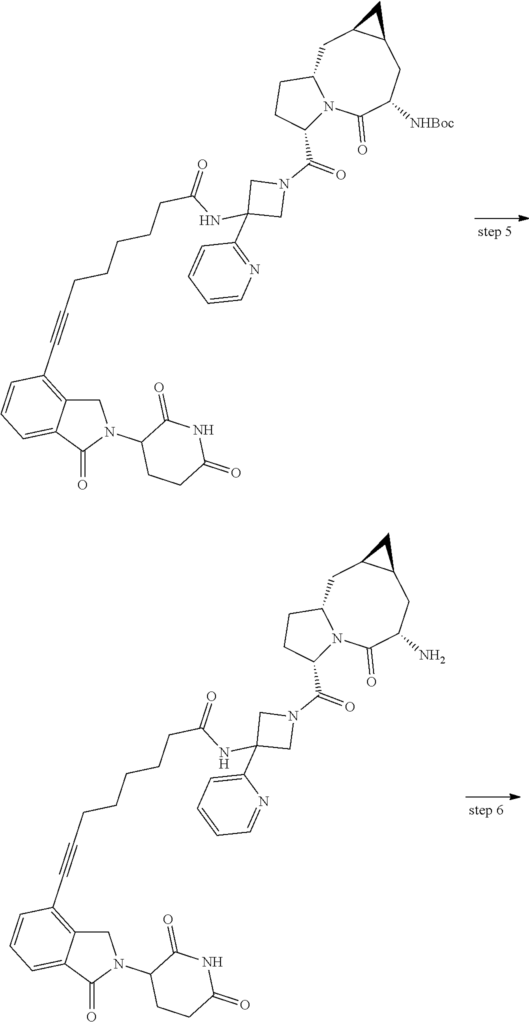 [SMILES: CC(C)(C)OC(=O)N[C@H]1C[C@@H]2C[C@@H]2C[C@H]2CC[C@@H](C(=O)N3CC(NC(=O)CCCCCC#Cc4cccc5c4CN(C4CCC(=O)NC4=O)C5=O)(c4ccccn4)C3)N2C1=O ; N[C@H]1C[C@@H]2C[C@@H]2C[C@H]2CC[C@@H](C(=O)N3CC(NC(=O)CCCCCC#Cc4cccc5c4CN(C4CCC(=O)NC4=O)C5=O)(c4ccccn4)C3)N2C1=O]

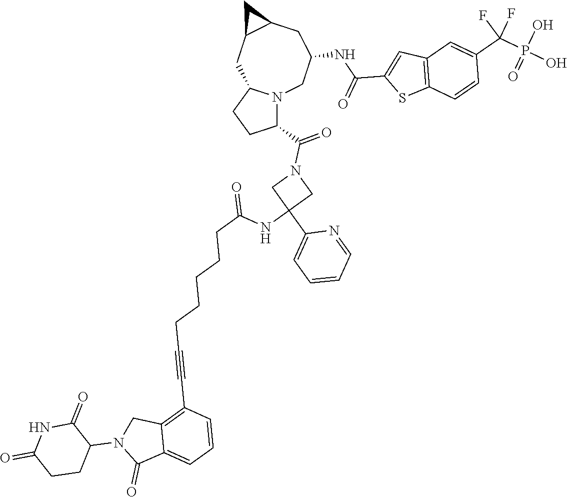 [SMILES: O=C1CCC(N2Cc3c(C#CCCCCCC(=O)NC4(c5ccccn5)CN(C(=O)[C@@H]5CC[C@@H]6C[C@H]7C[C@H]7C[C@H](NC(=O)c7cc8cc(C(F)(F)P(=O)(O)O)ccc8s7)CN65)C4)cccc3C2=O)C(=O)N1]

### Step 1: Preparation of 8-(2-(2,6-dioxopiperidin-3-yl)-1-oxoisoindolin-4-yl)oct-7-ynoic acid

To a solution of oct-7-ynoic acid (0.20 g, 1.42 mmol, 1.2 eq) in DMF (2 mL) was added 3-(4-bromo-1-oxo-2,3-dihydro-1H-isoindol-2-yl)piperidine-2,6-dione (0.40 g, 1.23 mmol, 1.0 eq), copper iodide (47 mg, 0.25 mmol, 0.2 eq), and then Pd(dppf)Cl₂ (90 mg, 0, 12 mmol, 0.1 eq) and triethylamine (0.37 g, 3.7 mmol, 3.0 eq) was added under N₂ atmosphere. The mixture was stirred at 80° C. for 3 hours to give a brown mixture. The reaction mixture was quenched by 20 mL water and extracted with ethyal acetate (20 mL×3). The combined organic layer was washed with brine 20 mL, dried over Na₂SO₄, filtered and concentrated under reduced pressure to give 8-(2-(2,6-dioxopiperidin-3-yl)-1-oxoisoindolin-4-yl)oct-7-ynoic acid (0.10 g, 14% yield) as a white solid. LCMS (ESI) m/z=383.0

### Step 2: Preparation of tert-butyl 3-(8-(2-(2,6-dioxopiperidin-3-yl)-1-oxoisoindolin-4-yl)oct-7-ynamido)-3-(pyridin-2-yl)azetidine-1-carboxylate

To a solution of 8-(2-(2,6-dioxopiperidin-3-yl)-1-oxoisoindolin-4-yl)oct-7-ynoic acid (90 mg, 0.24 mmol, 1.0 eq) in DCM (1 mL) was added HATU (80 mg, 0.21 mmol, 0.9 eq) and ethylbis(propan-2-yl)amine (45 mg, 0.35 mmol, 1.5 eq), the mixture was stirred at 25° C. for 10 minutes, after the mixture was added tert-butyl 3-amino-3-(pyridin-2-yl)azetidine-1-carboxylate (64 mg, 0.26 mmol, 1.1 eq), the mixture was stirred at 25° C. for 1 hour to give a brown mixture. The reaction solution is concentrated directly to give tert-butyl 3-(8-(2-(2,6-dioxopiperidin-3-yl)-1-oxoisoindolin-4-yl)oct-7-ynamido)-3-(pyridin-2-yl)azetidine-1-carboxylate (0.10 g, crude) as a brown oil. LCMS (ESI) m/z=614.5

### Step 3: Preparation of 8-(2-(2,6-dioxopiperidin-3-yl)-1-oxoisoindolin-4-yl)-N-(3-(pyridin-2-yl)azetidin-3-yl)oct-7-ynamide

To a solution of tert-butyl 3-(8-(2-(2,6-dioxopiperidin-3-yl)-1-oxoisoindolin-4-yl)oct-7-ynamido)-3-(pyridin-2-yl)azetidine-1-carboxylate (0.10 g, 0.16 mmol, 1.0 eq) in TFA (0.1 mL) and DCM (0.2 mL), the mixture was stirred at 25° C. for 30 minutes to give a brown mixture. The reaction mixture was concentrated under reduced pressure to give 8-(2-(2,6-dioxopiperidin-3-yl)-1-oxoisoindolin-4-yl)-N-(3-(pyridin-2-yl)azetidin-3-yl)oct-7-ynamide (0.1 g, crude) as a brown oil. LCMS (ESI) m/z=514.3

Step 4: Preparation of tert-butyl ((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (59 mg, 0.17 mmol, 0.9 eq) in DCM (1 mL) was added HATU (88 mg, 0.23 mmol, 1.2 eq) and ethylbis(propan-2-yl)amine (50 mg, 0.40 mmol, 2.0 eq), the mixture was stirred at 25° C. for 10 minutes, the mixture was added 8-(2-(2,6-dioxopiperidin-3-yl)-1-oxoisoindolin-4-yl)-N-(3-(pyridin-2-yl)azetidin-3-yl)oct-7-ynamide (0.10 g, 0.19 mmol, 1.0 eq), the mixture was stirred at 25° C. for 1 hour to give a brown mixture. The reaction solution is concentrated directly. The residue was purified by flash silica gel chromatography to give tert-butyl ((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamate (0.15 g, 91% yield) as a white solid. LCMS (ESI) m/z=834.4

### Step 5: Preparation of N-(1-((3S 6S,7aS,8aR,9aR)-6-amino-5-oxodecahydro-1H-cyclopropa[d]pyrrolo[1,2-a]azocine-3-carbonyl)-3-(pyridin-2-yl)azetidin-3-yl)-8-(2-(2,6-dioxopiperidin-3-yl)-1-oxoisoindolin-4-yl)oct-7-ynamide

To a solution of tert-butyl ((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamate (0.15 g, 0.18 mmol, 1.0 eq) in TFA (0.5 mL) and DCM (1 mL), the mixture was stirred at 25° C. for 30 minutes to give a brown mixture. The reaction mixture was concentrated under reduced pressure to give N-(1-((3S,6S,7aS,8aR,9aR)-6-amino-5-oxodecahydro-1H-cyclopropa[d]pyrrolo[1,2-a]azocine-3-carbonyl)-3-(pyridin-2-yl)azetidin-3-yl)-8-(2-(2,6-dioxopiperidin-3-yl)-1-oxoisoindolin-4-yl)oct-7-ynamide (0.15 g, crude) as a yellow oil. LCMS (ESI) m/z=734.4

### Step 6: Preparation of ((2-(((3S 6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of N-(1-((3S,6S,7aS,8aR,9aR)-6-amino-5-oxodecahydro-1H-cyclopropa[d]pyrrolo[1,2-a]azocine-3-carbonyl)-3-(pyridin-2-yl)azetidin-3-yl)-8-(2-(2,6-dioxopiperidin-3-yl)-1-oxoisoindolin-4-yl)oct-7-ynamide (0.15 g, 0.20 mmol, 1.0 eq) and (difluoro(2-((4-nitrophenoxy)carbonyl)benzo[b]thiophen-5-yl)methyl)phosphonic acid (88 mg, 0.20 mmol, 1.0 eq) in DCM (2 mL) were added 1H-1,2,3-benzotriazol-1-ol (32.90 mg, 0.24 mmol, 1.2 eq) and ethylbis(propan-2-yl)amine (79 mg, 0.61 mmol, 3.0 eq), the mixture was stirred at 25° C. for 10 minutes to give a brown mixture. The reaction solution is concentrated directly to give a residue. The residue was purified by prep-HPLC (column: Welch Xtimate C18 150×25 mm×5 um; mobile phase: [water (TFA)-ACN]; B %: 16%-46%, 10 min). The solution was lyophilization to give ((2-(((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 1, 31 mg, 15% yield) was obtained as a white solid. LCMS (ESI) m/z=1024.5. ¹H NMR (400 MHz, DMSO-d₆) δ 10.98 (s, 1H), 9.08-8.95 (m, 1H), 8.92-8.81 (m, 1H), 8.62-8.52 (m, 1H), 8.33 (d, J=2.8 Hz, 1H), 8.13 (d, J=8.4 Hz, 1H), 8.08 (s, 1H), 7.75-7.67 (m, 2H), 7.64-7.56 (m, 2H), 7.53-7.47 (m, 1H), 7.40-7.29 (m, 1H), 7.27-7.22 (m, 1H), 7.22-6.93 (m, 1H), 5.16-5.07 (m, 1H), 4.97-4.89 (m, 1H), 4.65-4.49 (m, 2H), 4.35 (s, 2H), 4.31-4.25 (m, 2H), 4.15 (d, J=10.0 Hz, 3H), 4.04 (s, 2H), 2.94-2.82 (m, 1H), 2.45-2.41 (m, 2H), 2.36-2.28 (m, 1H), 2.23 (t, J=7.2 Hz, 2H), 2.18-2.08 (m, 2H), 2.06-1.95 (m, 3H), 1.89-1.80 (m, 2H), 1.61-1.52 (m, 4H), 1.47-1.35 (m, 3H), 0.88-0.77 (m, 1H), 0.76-0.67 (m, 1H), 0.00 (d, J=2.0 Hz, 1H).

### Preparation of S,S′-(((((2-(((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 2)

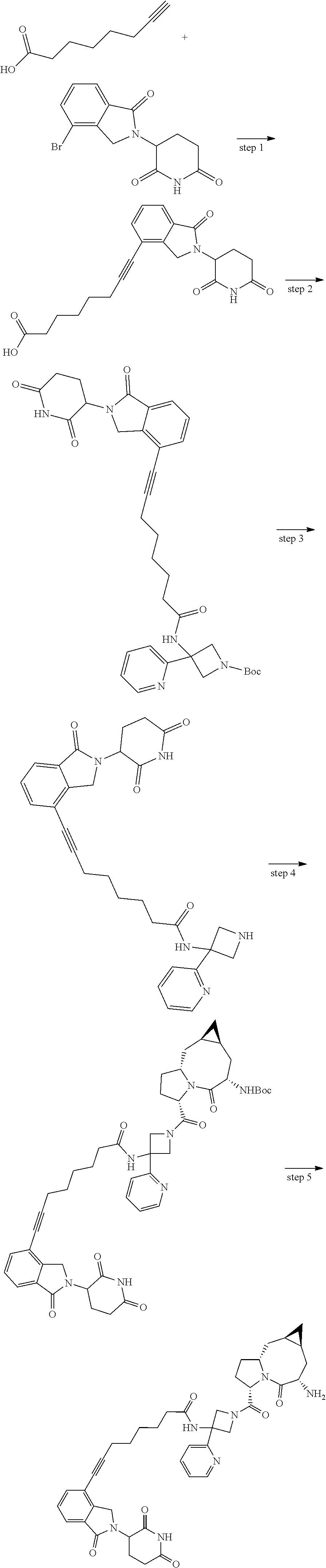

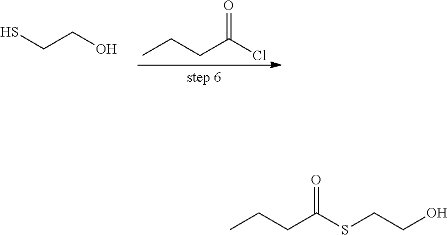

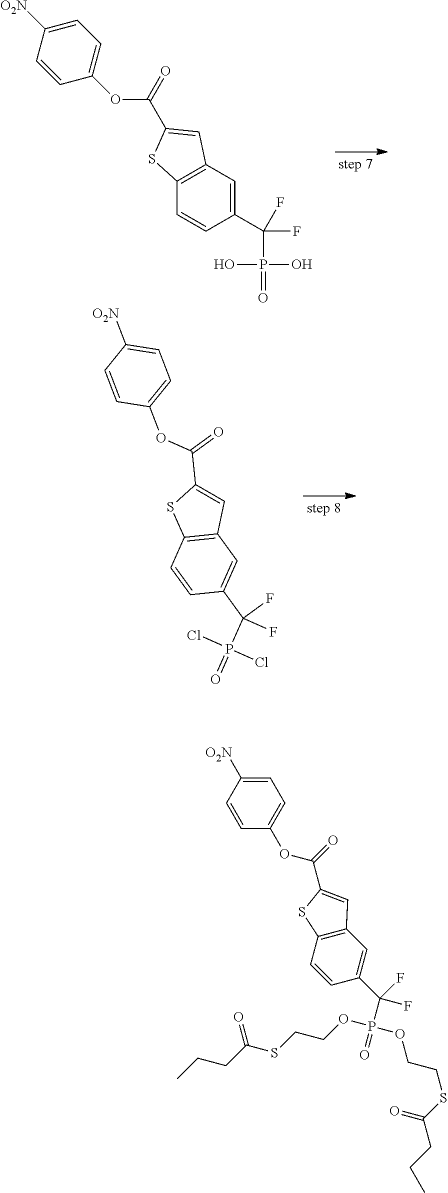 [SMILES: O=C(Oc1ccc([N+](=O)[O-])cc1)c1cc2cc(C(F)(F)P(=O)(O)O)ccc2s1 ; O=C(Oc1ccc([N+](=O)[O-])cc1)c1cc2cc(C(F)(F)P(=O)(Cl)Cl)ccc2s1 ; CCCC(=O)SCCOP(=O)(OCCSC(=O)CCC)C(F)(F)c1ccc2sc(C(=O)Oc3ccc([N+](=O)[O-])cc3)cc2c1]

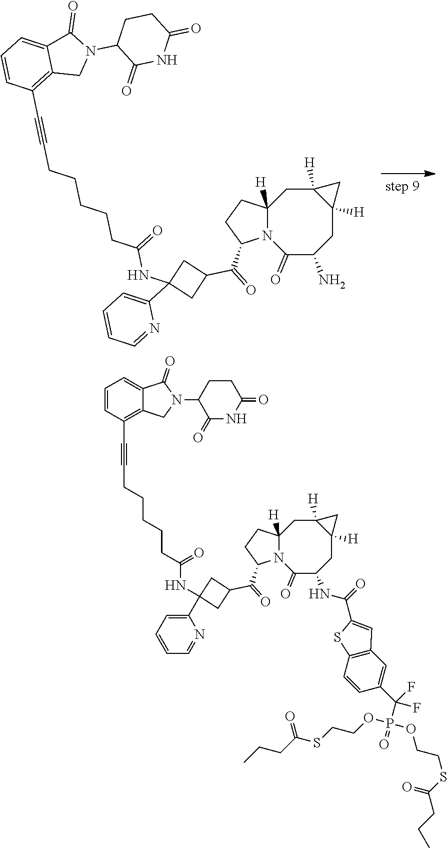 [SMILES: N[C@H]1C[C@@H]2C[C@@H]2C[C@H]2CC[C@@H](C(=O)C3CC(NC(=O)CCCCCC#Cc4cccc5c4CN(C4CCC(=O)NC4=O)C5=O)(c4ccccn4)C3)N2C1=O ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)C5CC(NC(=O)CCCCCC#Cc6cccc7c6CN(C6CCC(=O)NC6=O)C7=O)(c6ccccn6)C5)N4C3=O)cc2c1]

### Step 1: Preparation of 8-(2-(2,6-dioxopiperidin-3-yl)-1-oxoisoindolin-4-yl)oct-7-ynoic acid 8-(2-(2,6-dioxopiperidin-3-yl)-1-oxoisoindolin-4-yl)oct-7-ynoic acid

To a solution of oct-7-ynoic acid (0.10 g, 0.74 mmol, 1.20 eq) in DMF (2.00 mL) was added 3-(4-bromo-1-oxoisoindolin-2-yl)piperidine-2,6-dione (0.20 g, 0.60 mmol, 1.00 eq), copper iodide (27 mg, 0.12 mmol, 0.20 eq), Pd(dppf)₂Cl₂ (45 mg, 62 μmol, 0.10 eq), triethylamine (0.19 g, 1.9 mmol, 3.0 eq) under N₂ atmosphere. The mixture was stirred at 80° C. for 3 hours to give a brown solution. The residue was diluted with water (10 mL) and extracted with ethyl acetate (20 mL×3), the combined organic layers were washed with saturated brine (20 mL×2), dried over Na₂SO₄, filtered and concentrated under reduced pressure to give 8-(2-(2,6-dioxopiperidin-3-yl)-1-oxoisoindolin-4-yl)oct-7-ynoic acid 8-(2-(2,6-dioxopiperidin-3-yl)-1-oxoisoindolin-4-yl)oct-7-ynoic acid (0.17 g, 72% yield) as yellow oil. LCMS (ESI) m/z=382.8

### Step 2: Preparation of tert-butyl 3-(8-(2-(2,6-dioxopiperidin-3-yl)-1-oxoisoindolin-4-yl)oct-7-ynamido)-3-(pyridin-2-yl)azetidine-1-carboxylate

To a solution of 8-(2-(2,6-dioxopiperidin-3-yl)-1-oxoisoindolin-4-yl)oct-7-ynoic acid 8-(2-(2,6-dioxopiperidin-3-yl)-1-oxoisoindolin-4-yl)oct-7-ynoic acid (0.17 g, 0.27 mmol, 1.0 eq) in DCM (3.0 mL) was added HATU (0.12 g, 0.32 mmol, 1.20 eq) and ethylbis(propan-2-yl)amine (0.10 g, 0.80 mmol, 3.0 eq), then stirred at 25° C. for 5 minutes. tert-butyl 3-amino-3-(pyridin-2-yl)azetidine-1-carboxylate (66 mg, 0.27 mmol, 1.0 eq) was added to the mixture. The reaction was stirred at 25° C. for 1 hour to give a black suspension solution. The reaction mixture was concentrated under reduced pressure to give a residue. The residue was purified by flash silica gel chromatography to give tert-butyl 3-(8-(2-(2,6-dioxopiperidin-3-yl)-1-oxoisoindolin-4-yl)oct-7-ynamido)-3-(pyridin-2-yl)azetidine-1-carboxylate (0.16 g, 98% yield) as a yellow oil. LCMS (ESI) m/z=614.2. ¹H NMR (400 MHz, CHLOROFORM-d) δ 7.77 (m, 2H) 7.61-7.67 (m, 2H) 7.50-7.56 (m, 2H) 7.40-7.47 (m, 1H) 4.43-4.47 (m, 1H) 4.30 (s, 2H) 4.14 (s, 4H) 2.36-2.49 (m, 4H) 2.18-2.33 (m, 10H) 1.41 (s, 9H)

### Step 3: Preparation of 8-(2-(2,6-dioxopiperidin-3-yl)-1-oxoisoindolin-4-yl)-N-(3-(pyridin-2-yl)azetidin-3-yl)oct-7-ynamide

The tert-butyl 3-(8-(2-(2,6-dioxopiperidin-3-yl)-1-oxoisoindolin-4-yl)oct-7-ynamido)-3-(pyridin-2-yl)azetidine-1-carboxylate (0.16 g, 0.26 mmol, 1.0 eq) was dissolved in DCM (3.0 mL) and TFA (1.0 mL). Then the mixture was stirred at 25° C. for 1 hour to give a brown solution. The reaction mixture was concentrated under reduced pressure to give 8-(2-(2,6-dioxopiperidin-3-yl)-1-oxoisoindolin-4-yl)-N-(3-(pyridin-2-yl)azetidin-3-yl)oct-7-ynamide (0.16 g, crude) as a yellow oil. LCMS (ESI) m/z=514.2

### Step 4: Preparation of tert-butyl ((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (90 mg, 0.27 mmol, 1.0 eq) in DCM (1.0 mL) was added HATU (0.10 g, 0.27 mmol, 1.00 eq) and ethylbis(propan-2-yl)amine (0.14 g, 1.05 mmol, 4.00 eq), then stirred at 25° C. for 5 minutes. A solution of 8-(2-(2,6-dioxopiperidin-3-yl)-1-oxoisoindolin-4-yl)-N-(3-(pyridin-2-yl)azetidin-3-yl)oct-7-ynamide (0, 14 g, 0.27 mmol, 1.00 eq) and ethylbis(propan-2-yl)amine (0.14 g, 1.05 mmol, 4.00 eq) in DCM (1.00 mL) was added to the mixture, then the mixture was stirred at 25° C. for 1 hour to give a yellow solution. The reaction mixture was concentrated under reduced pressure to give a residue. The residue was purified by flash silica gel chromatography to give tert-butyl ((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamate (0.12 g, 54% yield) as a yellow oil. LCMS (ESI) m/z=834.5

### Step 5: Preparation of N-(1-((3S,6S,7aS,8aR,9aR)-6-amino-5-oxodecahydro-1H-cyclopropa[d]pyrrolo[1,2-a]azocine-3-carbonyl)-3-(pyridin-2-yl)azetidin-3-yl)-8-(2-(2,6-dioxopiperidin-3-yl)-1-oxoisoindolin-4-yl)oct-7-ynamide

The tert-butyl ((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamate (0.10 g, 0.12 mmol, 1.00 eq) was dissolved in DCM (1.00 mL) and TFA (0.30 mL), then stirred at 25° C. for 1 hour to give a yellow solution. The reaction mixture was concentrated under reduced pressure to give N-(1-((3S,6S,7aS,8aR,9aR)-6-amino-5-oxodecahydro-1H-cyclopropa[d]pyrrolo[1,2-a]azocine-3-carbonyl)-3-(pyridin-2-yl)azetidin-3-yl)-8-(2-(2,6-dioxopiperidin-3-yl)-1-oxoisoindolin-4-yl)oct-7-ynamide (0, 10 g, crude) as a yellow oil. LCMS (ESI) m/z=734.2

### Step 6: Preparation of S-(2-hydroxyethyl) butanethioate

To a solution of 2-mercaptoethanol (10 g, 0.13 mol, 1.0 eq) in DCM (100 mL) was added triethylamine (13 g, 0.13 mol, 1.0 eq) at 25° C., then cooled to −70° C. under N₂ protect. Butanoyl chloride (12 g, 0.11 mol, 0.90 eq) was added to the reaction at −70° C., then the mixture was warmed to 25° C. and stirred at 25° C. for 12 h to give a white suspension. The reaction mixture was quenched with water (100 mL), extracted with DCM (100 mL×3). The water phase was quenched with NaOCl aqueous solution (10% mass fraction) for 12 h. Combined organic layer was dried over Na₂SO₄, filtered and concentrated under reduced pressure to give a residue. The residue was purified by column chromatography to give S-(2-hydroxyethyl) butanethioate (13 g, 69% yield) as white oil. ¹H NMR (400 MHz, CHLOROFORM-d) δ ppm 4.20 (m, 2H) 2.75 (m, 2H) 2.25-2.34 (m, 2H) 1.66-1.72 (m, 2H) 0.94-0.98 (m, 3H)

### Step 7: Preparation of 4-nitrophenyl 5-((dichlorophosphoryl)difluoromethyl)benzo[b]thiophene-2-carboxylate

To a solution of (difluoro(2-((4-nitrophenoxy)carbonyl)benzo[b]thiophen-5-yl)methyl)phosphonic acid (0.20 g, 0.47 mmol, 1.0 eq) in DCM (4.00 mL) was added N,N-dimethylformamide (3.39 mg, 46 μmol, 0.10 eq) at 25° C., then cooled to 0° C., a solution of oxalic dichloride (0.23 g, 1.85 mmol, 4.00 eq) in DCM (2.00 mL) was added to the mixture. Then stirred at 40° C. for 1 hour to give a yellow solution. The reaction mixture was concentrated under reduced pressure to 4-nitrophenyl 5-((dichlorophosphoryl)difluoromethyl)benzo[b]thiophene-2-carboxylate (0.20 g, crude) as a yellow oil. LCMS (ESI) m/z=457.8 (MeOH quench)

### Step 8: Preparation of 4-nitrophenyl 5-((bis(2-(butyrylthio)ethoxy)phosphoryl)difluoromethyl)benzo[b]thiophene-2-carboxylate

To a solution of 4-nitrophenyl 5-((dichlorophosphoryl)difluoromethyl)benzo[b]thiophene-2-carboxylate (0.20 g, 0.43 mmol, 1.00 eq) in DCM (3.00 mL) was slowly dropwise a solution DIEA (0.22 g, 1.71 mmol, 4.00 eq) in DCM (3.00 mL) at −70° C. then a solution of S-(2-hydroxyethyl) butanethioate (0.19 g, 1.28 mmol, 3.00 eq) in DCM (4 mL) was slowly dropwise added to the mixture at −70° C., then the mixture was stirred at −70° C. for 5 min to give a yellow solution. The reaction mixture was purified by flash silica gel chromatography to give 4-nitrophenyl 5-((bis(2-(butyrylthio)ethoxy)phosphoryl)difluoromethyl)benzo[b]thiophene-2-carboxylate (61 mg, 21% yield) as a yellow oil. LCMS (ESI) m/z=690.1

### Step 9: Preparation of S,S′-(((((2-(((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of 4-nitrophenyl 5-((bis(2-(butyrylthio)ethoxy)phosphoryl)difluoromethyl)benzo[b]thiophene-2-carboxylate (60 mg, 86 μmol, 1.0 eq) in DCM (1.00 mL) was added 1H-1,2,3-benzotriazol-3-ium-1-olate amine (13 mg, 86 μmol, 1.00 eq) was stirred at 25° C. for 5 minutes. A solution of N-(1-((3S,6S,7aS,8aR,9aR)-6-amino-5-oxodecahydro-1H-cyclopropa[d]pyrrolo[1,2-a]azocine-3-carbonyl)-3-(pyridin-2-yl)azetidin-3-yl)-8-(2-(2,6-dioxopiperidin-3-yl)-1-oxoisoindolin-4-yl)oct-7-ynamide (64 mg, 86 μmol, 1.00 eq) and ethylbis(propan-2-yl)amine (45 mg, 0.35 mmol, 4.00 eq) in DCM (1.00 mL) was added to the mixture. Then the reaction was stirred at 25° C. for 1 hour to give a yellow solution. The reaction mixture was concentrated under reduced pressure to give a residue. The residue was purified by prep-HPLC (column: Welch Ultimate C18 150×25 mm×5 um; mobile phase: water(TFA)-ACN; B %: 45%-75%, 10 min). The solution was lyophilization to give S,S′-(((((2-(((3S,6S,7aS,8aR,9aR)-3-(3-(8-(2-(2,6-dioxopiperidin-3-yl)-1-oxoisoindolin-4-yl)oct-7-ynamido)-3-(pyridin-2-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 2, 8.30 mg, 7.47% yield) as a white solid. LCMS (ESI) m/z=643.0. ¹H NMR (400 MHz, CHLOROFORM-d) δ 9.02-9.53 (m, 1H), 8.62 (m, 1H), 8.13-8.28 (m, 2H), 8.04-8.12 (m, 1H), 7.92-8.03 (m, 2H), 7.76-7.88 (m, 2H), 7.65-7.74 (m, 11H), 7.40-7.60 (m, 3H), 5.21 (m, 3H), 4.35-4.47 (m, 3H), 4.16-4.34 (m, 7H), 3.02-3.16 (m, 5H), 2.39-2.49 (m, 3H), 2.21-2.36 (m, 10H), 2.18 (s, 4H), 1.53-1.73 (m, 9H), 1.23-1.47 (m, 4H), 0.90-0.99 (m, 7H), 0.78-0.89 (m, 2H), 0.02 (s, 1H).

### Preparation of ((2-(((3S,6S,7aS,8aR,9aR)-3-((rel-3S,4R)-3-cyano-4-(3-(4-(4-(2,6-dioxopiperidin-3-yl)phenoxy)butoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 3) and S,S′-(((((2-(((3S,6S,7aS,8aR,9aR)-3-((rel-3S,4R)-3-cyano-4-(3-(4-(4-(2,6-dioxopiperidin-3-yl)phenoxy)butoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 4)

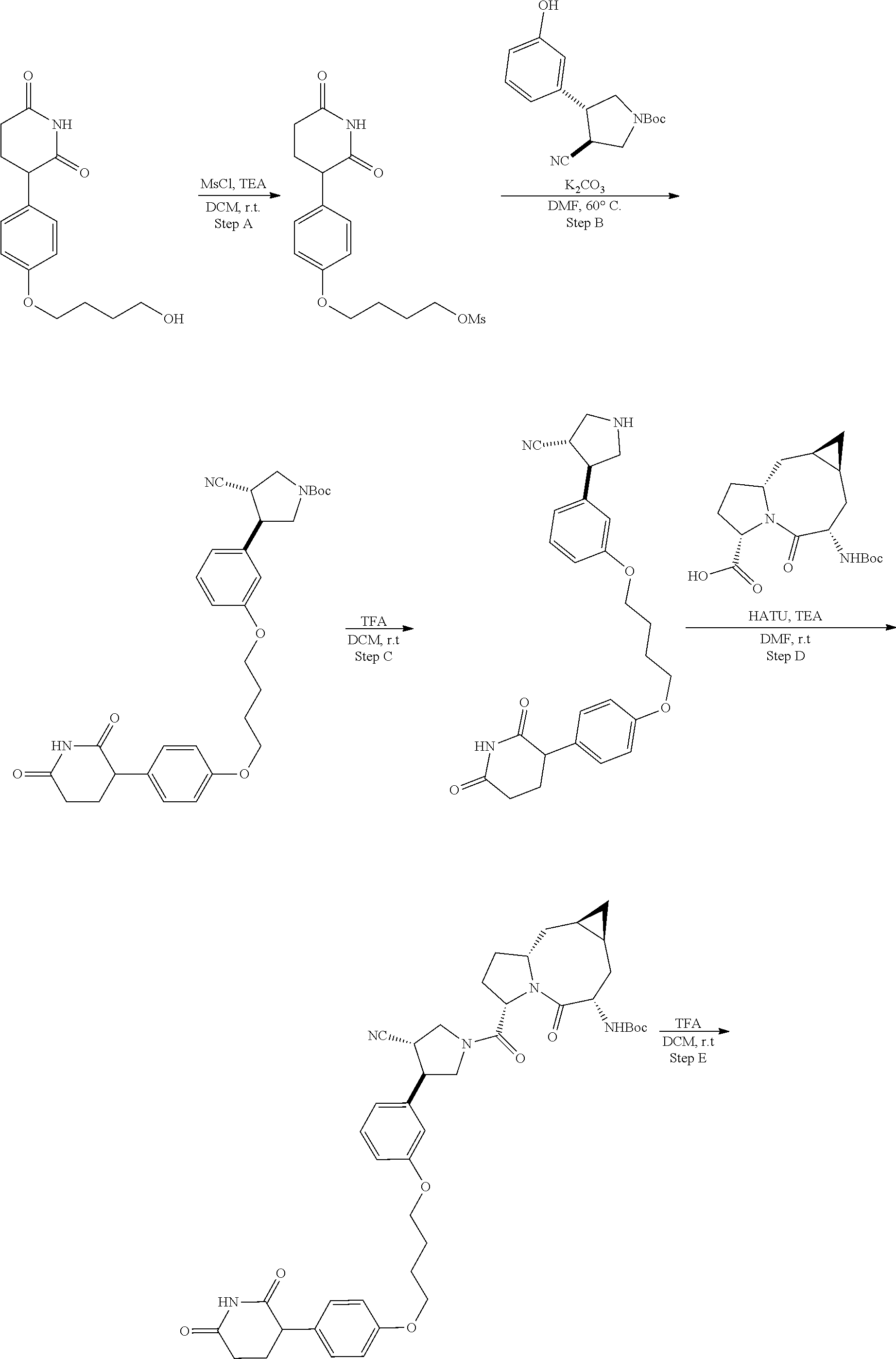

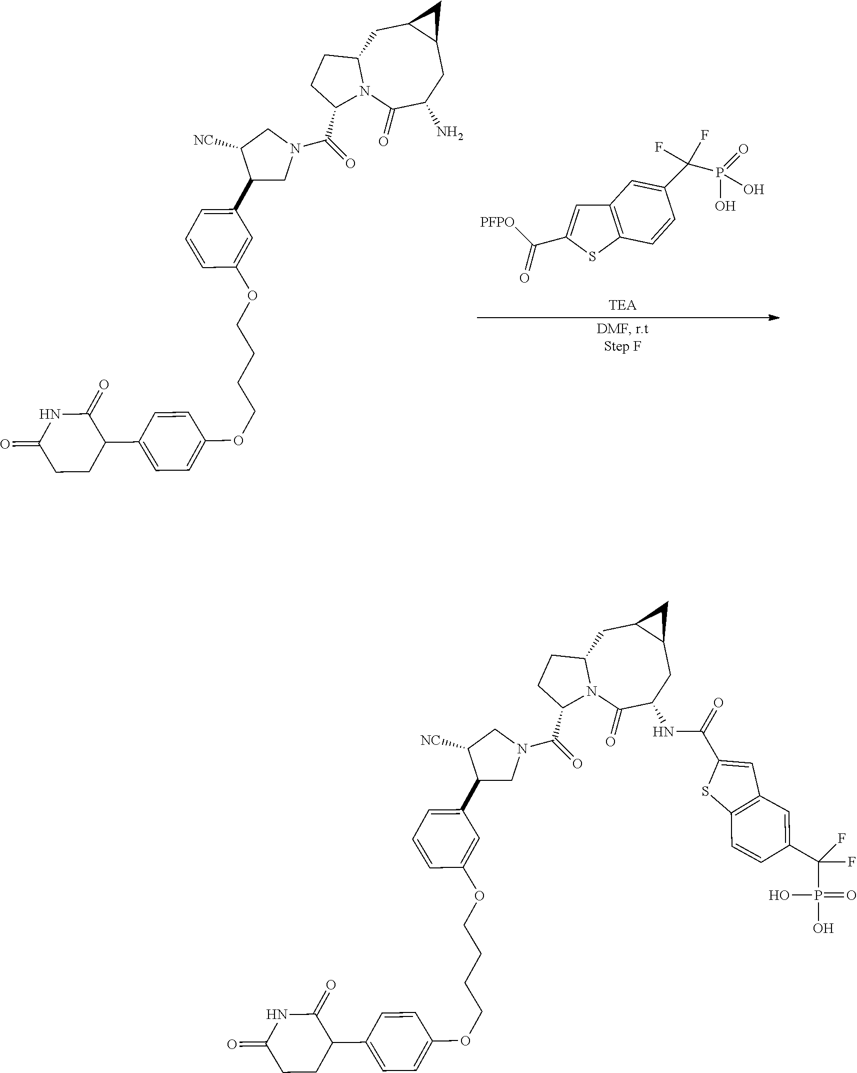

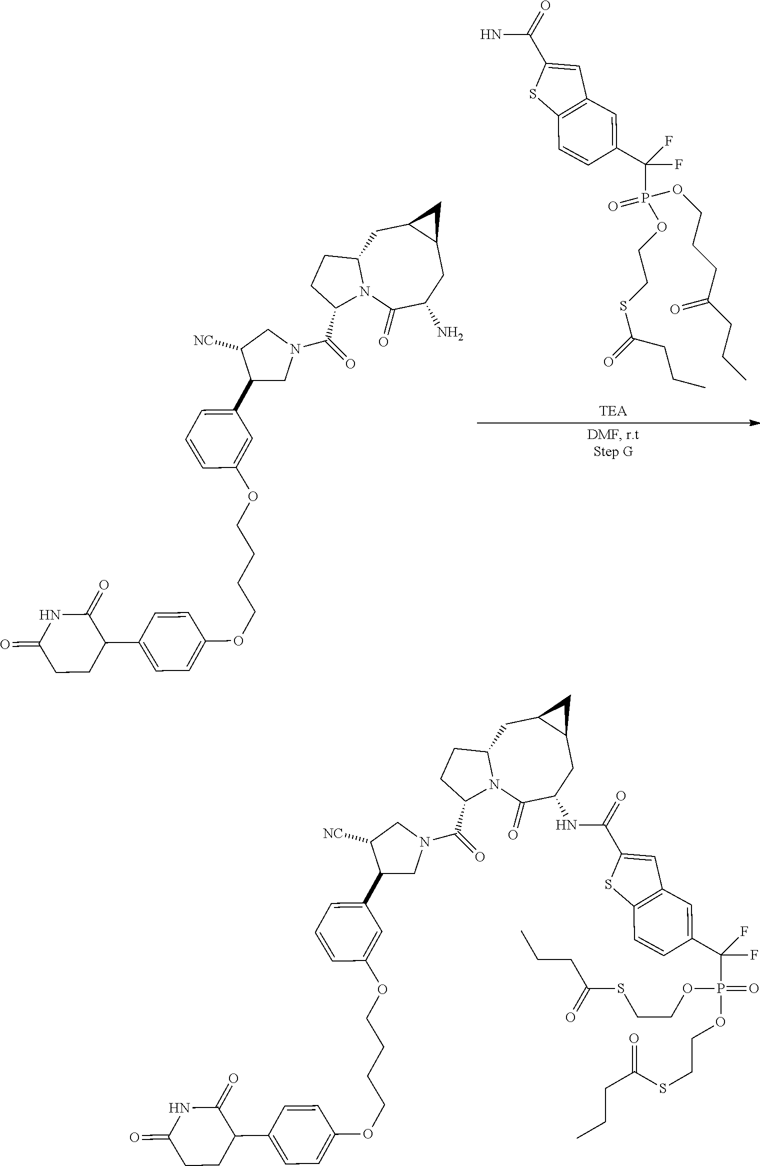

### STEP A: 4-(4-(2,6-dioxopiperidin-3-yl)phenoxy)butyl methanesulfonate

To a solution of 3-{4-[(4-hydroxybutyl)oxy]phenyl}hexahydropyridine-2,6-dione (600 mg, 2.16 mmol, 1.0 eq.) in DCM (15 mL) were added TEA (1.20 mL, 8.65 mmol) and MsCl (495 mg, 4.33 mmol) and then the resulting mixture was stirred at room temperature for 18 hrs under N₂. After completion, the reaction mixture was diluted with H₂O (30 mL) and extracted with EtOAc (30 mL×2). The combined organic layers was washed with brine (10 mL), dried over anhydrous Na₂SO₄, and concentrated under reduced pressure to get crude 4-{[4-(2,6-dioxohexahydropyridin-3-yl)phenyl]oxy}butyl methanesulfonate (600 mg, 1.69 mmol, 78%) as a white solid, which was used in next step directly without further purification. LCMS (ESI): m/z=356 [M+H]⁺.

### STEP B: rac-tert-butyl (3S,4R)-3-cyano-4-(3-(4-(4-(2,6-dioxopiperidin-3-yl)phenoxy)butoxy)phenyl)pyrrolidine-1-carboxylate

To a solution of 4-{[4-(2,6-dioxohexahydropyridin-3-yl)phenyl]oxy}butyl methanesulfonate (250 mg, 0.70 mmol, 1.0 eq.) and rac-2-methylpropan-2-yl 4-cyano-3-(3-hydroxyphenyl)tetrahydropyrrole-1-carboxylate (213 mg, 0.74 mmol, 1.2 eq.) in DMF (2 mL) was added K₂CO₃ (194 mg, 1.41 mmol, 2.0 eq.) and the resulting mixture was stirred at 60° C. for 18 hrs under N₂. After completion, the reaction mixture was concentrated under reduced pressure. The residue was purified by Biotage C18 column chromatography to get rac-tert-butyl (3S,4R)-3-cyano-4-(3-(4-(4-(2,6-dioxopiperidin-3-yl)phenoxy)butoxy)phenyl)pyrrolidine-1-carboxylate (108 mg, 0.20 mmol, 28%) as a white solid LCMS (ESI): m/z=548 [M+H]⁺.

### STEP C: rac-4-{3-[(4-{[4-(2,6-dioxohexahydropyridin-3-yl)phenyl]oxy}butyl)oxy]phenyl}tetrahydropyrrole-3-carbonitrile

To a solution of rac-tert-butyl (3S,4R)-3-cyano-4-(3-(4-(4-(2,6-dioxopiperidin-3-yl)phenoxy)butoxy)phenyl)pyrrolidine-1-carboxylate (108 mg, 0.20 mmol, 1.0 eq.) in DCM (4 mL) was added TFA (2 mL) and the resulting mixture was stirred at room temperature for 2 hrs under N₂. After completion, the reaction mixture was concentrated under reduced pressure to get crude rac-4-{3-[(4-{[4-(2,6-dioxohexahydropyridin-3-yl)phenyl]oxy}butyl)oxy]phenyl}tetrahydropyrrole-3-carbonitrile (90 mg, crude) as a white solid, which was used in next step directly without further purification. LCMS (ESI): m/z=448 [M+H]⁺.

### STEP D: tert-butyl ((3S,6S,7aS,8aR,9aR)-3-((rel-3S,4R)-3-cyano-4-(3-(4-(4-(2,6-dioxopiperidin-3-yl)phenoxy)butoxy)phenyl)pyrrolidine-1-carbonyl)-5-oxodecahydro-1H-cyclopropa[d]pyrrolo[1,2-a]azocin-6-yl)carbamate

To a solution of rac-4-{3-[(4-{[4-(2,6-dioxohexahydropyridin-3-yl)phenyl]oxy}butyl)oxy]phenyl}tetrahydropyrrole-3-carbonitrile (90 mg, 0.20 mmol, 1.0 eq.) and (3S,6S,7aS,8aR,9aR)-6-({[(2-methylprop-2-yl)oxy]carbonyl}amino)-5-oxo-1,2,3,5,6,7,7a,8a,9,9a-decahydrocyclopropa[1,2-d]pyrrolo[1,2-a]azocine-3-carboxylic acid (68 mg, 0.20 mmol, 1.0 eq.) in DMF (4 mL) were added HATU (84 mg, 0.22 mmol, 1.0 eq.) and TEA (0.1 mL, 1.01 mmol, 5 eq.). The resulting mixture was stirred at room temperature for 2 hrs under N₂. After completion, the reaction mixture was concentrated under reduced pressure. The residue was purified by Biotage® C18 column chromatography to get tert-butyl ((3S,6S,7aS,8aR,9aR)-3-((rel-3S,4R)-3-cyano-4-(3-(4-(4-(2,6-dioxopiperidin-3-yl)phenoxy)butoxy)phenyl)pyrrolidine-1-carbonyl)-5-oxodecahydro-1H-cyclopropa[d]pyrrolo[1,2-a]azocin-6-yl)carbamate (90 mg, 0.12 mmol, 58%) as a white solid. LCMS (ESI): m/z=768 [M+H]⁺.

### STEP E: (rel-3S,4R)-1-((3S,7aS,8aR,9aR)-6-amino-5-oxodecahydro-1H-cyclopropa[d]pyrrolo[1,2-a]azocine-3-carbonyl)-4-(3-(4-(4-(2,6-dioxopiperidin-3-yl)phenoxy)butoxy)phenyl)pyrrolidine-3-carbonitrile

To a solution of tert-butyl ((3S,6S,7aS,8aR,9aR)-3-((rel-3S,4R)-3-cyano-4-(3-(4-(4-(2,6-dioxopiperidin-3-yl)phenoxy)butoxy)phenyl)pyrrolidine-1-carbonyl)-5-oxodecahydro-1H-cyclopropa[d]pyrrolo[1,2-a]azocin-6-yl)carbamate (45 mg, 59 μmol, 1.0 eq.) in DCM (4 mL) was added TFA (1 mL) and the reaction mixture was stirred at room temperature for 2 hrs under N₂. After completion, the reaction mixture was concentrated under reduced pressure to get crude (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(4-(4-(2,6-dioxopiperidin-3-yl)phenoxy)butoxy)phenyl)pyrrolidine-3-carbonitrile (40 mg, quant.) as a white solid, which was used in next step directly without further purification. LCMS (ESI): m/z=668 [M+H]⁺.

### STEP F: ((2-(((3S,6S,7aS,8aR,9aR)-3-((rel-3S,4R)-3-cyano-4-(3-(4-(4-(2,6-dioxopiperidin-3-yl)phenoxy)butoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(4-(4-(2,6-dioxopiperidin-3-yl)phenoxy)butoxy)phenyl)pyrrolidine-3-carbonitrile (40 mg, 60 μmol, 1.0 eq.) and (difluoro(2-((perfluorophenoxy)carbonyl)benzo[b]thiophen-5-yl)methyl)phosphonic acid (29 mg, 60 μmol, 1.0 eq.) in DMF (2 mL) was added TEA (18 mg, 0.18 mmol, 3.0 eq.). The reaction mixture was stirred at room temperature for 8 hrs. After completion, the residue was purified by prep-HPLC to give ((2-(((3S,6S,7aS,8aR,9aR)-3-((rel-3S,4R)-3-cyano-4-(3-(4-(4-(2,6-dioxopiperidin-3-yl)phenoxy)butoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 3, 20 mg, 21 μmol, 35%) as white solid.

¹H NMR (400 MHz, DMSO-d6) δ 10.89-10.66 (m, 1H), 8.91-8.78 (m, 1H), 8.37-8.28 (m, 1H), 8.18-8.03 (m, 2H), 7.64-7.54 (m, 1H), 7.33-7.23 (m, 1H), 7.15-7.08 (m, 2H), 7.07-6.94 (m, 2H), 6.93-6.83 (m, 3H), 4.98-4.91 (m, 1H), 4.59-4.53 (m, 1H), 4.35-4.21 (m, 2H), 4.02-3.88 (m, 3H), 3.84-3.73 (m, 2H), 3.71-3.60 (m, 1H), 3.57-3.46 (m, 1H), 3.34-3.22 (m, 1H), 3.16-3.01 (m, 1H), 2.71-2.59 (m, 1H), 2.35-2.12 (m, 2H), 2.07-1.71 (m, 13H), 1.69-1.45 (m, 3H), 1.30-1.12 (m, 2H).

LCMS (ESI): m/z=958.7 [M+H]⁻.

### STEP G: S,S′-(((((2-(((3S,6S,7aS,8aR,9aR)-3-((rel-3S,4R)-3-cyano-4-(3-(4-(4-(2,6-dioxopiperidin-3-yl)phenoxy)butoxy)phenyl)pyrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(4-(4-(2,6-dioxopiperidin-3-yl)phenoxy)butoxy)phenyl)pyrrolidine-3-carbonitrile (40 mg, 60 μmol, 1 eq.) and 4-nitrophenyl 5-((bis(2-(butyrylthio)ethoxy)phosphoryl)difluoromethyl)benzo[b]thiophene-2-carboxylate (41 mg, 60 μmol, 1.0 eq.) in DMF (2 mL) was added TEA (18 mg, 0.18 mmol, 2.0 eq.). The reaction mixture was stirred at room temperature for 8 hrs. After completion, the residue was purified by prep-HPLC to give S,S′-(((((2-(((3S,6S,7aS,8aR,9aR)-3-((rel-3S,4R)-3-cyano-4-(3-(4-(4-(2,6-dioxopiperidin-3-yl)phenoxy)butoxy)phenyl)pyrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 4, 20 mg, 16 μmol, 27%) as white solid.

¹H NMR (400 MHz, DMSO-d6) δ 10.78 (s, 1H), 9.04-8.84 (m, 1H), 8.35 (t, J=6.8 Hz, 1H), 8.26-8.09 (m, 2H), 7.62-7.46 (m, 1H), 7.33-7.21 (m, 1H), 7.16-7.07 (m, 2H), 7.06-6.93 (m, 2H), 6.91-6.84 (m, 3H), 5.01-4.90 (m, 1H), 4.59 (s, 1H), 4.46-4.08 (m, 6H), 4.07-3.90 (m, 5H), 3.89-3.40 (m, 5H), 3.19-3.05 (m, 4H), 2.73-2.57 (m, 1H), 2.57-2.51 (m, 4H), 2.45 (d, J=4.1 Hz, 1H), 2.41-2.26 (m, 1H), 2.21-1.95 (m, 6H), 1.94-1.76 (m, 6H), 1.70-1.48 (m, 5H), 1.47-1.33 (m, 1H), 0.90-0.78 (m, 7H), 0.77-0.66 (m, 1H), 0.09-0.13 (m, 1H).

LCMS (ESI): m/z=1219.1 [M+H]

### Preparation of 3-(1-oxo-4-(8-(2-oxo-4-((R)-4-azaspiro[2.4]heptan-6-yl)pyridin-1(2H)-yl)oct-1-yn-1-yl)isoindolin-2-yl)piperidine-2,6-dione

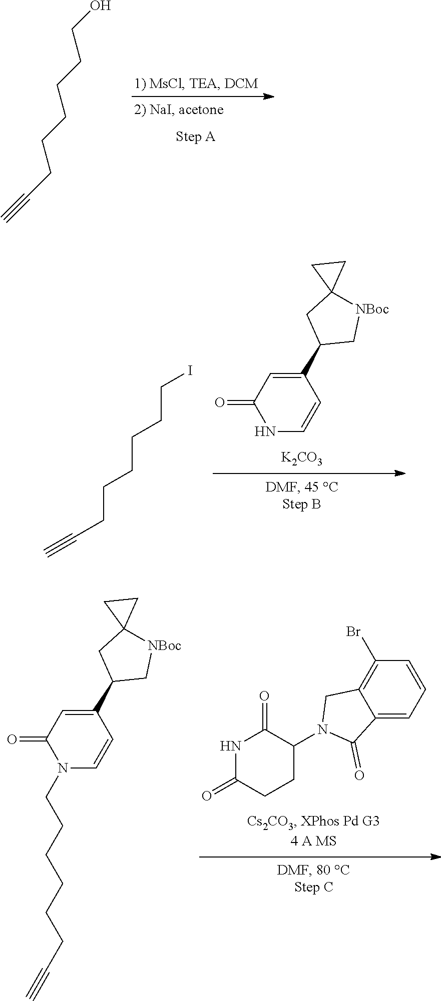

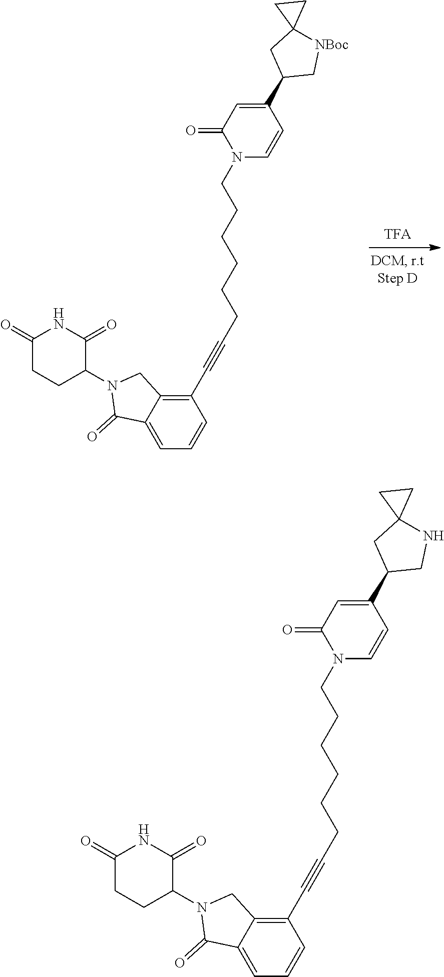 [SMILES: CC(C)(C)OC(=O)N1C[C@@H](c2ccn(CCCCCCC#Cc3cccc4c3CN(C3CCC(=O)NC3=O)C4=O)c(=O)c2)CC12CC2 ; O=C1CCC(N2Cc3c(C#CCCCCCCn4ccc([C@@H]5CNC6(CC6)C5)cc4=O)cccc3C2=O)C(=O)N1]

### STEP A: 8-Iodooct-1-yne

To a solution of oct-7-yn-1-ol (2 g, 15.8 mmol, 1.0 eq.) in DCM (15 mL) were added TEA (3.21 g, 31.6 mmol, 2.0 eq.) and MsCl (2.18 g, 19.0 mmol, 1.2 eq.) at 0° C. The reaction mixture was stirred at room temperature for 1 hr. After completion, the reaction mixture was diluted with H₂O (30 mL) and extracted with DCM (30 mL×2). The combined organic layers were washed with brine (20 mL), dried over anhydrous Na₂SO₄ and concentrated under reduced pressure to afford oct-7-yn-1-yl methanesulfonate (4.86 g, 15.8 mmol, 99%). To a solution of oct-7-yn-1-yl methanesulfonate (4.86 g, 15.8 mmol, 1.0 eq.) in 2-butanone (30 mL) was added NaI (7.14 g, 47.4 mmol, 3.0 eq.). The reaction mixture was stirred at 85° C. for 12 hrs. After completion, the reaction mixture was quenched with Na₂SO₃ (40 mL, saturated aqueous solution) and extracted with EtOAc (30 mL×2). The combined organic layers were washed with brine (20 mL), dried over anhydrous Na₂SO₄, and concentrated under reduced pressure. The residue was purified by flash column chromatography on silica gel to afford 8-iodooct-1-yne (3.1 g, 13.1 mmol, 83%) as a light oil.

¹H NMR (400 MHz, DMSO-d6) δ 3.30-3.23 (m, 2H), 2.76-2.67 (m, 1H), 2.18-2.10 (m, 2H), 1.81-1.69 (m, 2H), 1.47-1.31 (m, 6H).

### STEP B: tert-butyl (R)-6-(1-(oct-7-yn-1-yl)-2-oxo-1,2-dihydropyridin-4-yl)-4-azaspiro[2,4]heptane-4-carboxylate

To a solution of 8-iodooct-1-yne (245 mg, 1.04 mmol, 1.0 eq.) in DMF (4 mL) were added tert-butyl (R)-6-(2-oxo-1,2-dihydropyridin-4-yl)-4-azaspiro[2.4]heptane-4-carboxylate (300 mg, 1.04 mmol, 1.0 eq.) and K₂CO₃ (431 mg, 3.12 mmol, 3.0 eq.). The reaction mixture was stirred at 45° C. for 12 hrs. After completion, the reaction mixture was purified directly by Biotage® C18 column chromatography to afford tert-butyl (R)-6-(1-(oct-7-yn-1-yl)-2-oxo-1,2-dihydropyridin-4-yl)-4-azaspiro[2.4]heptane-4-carboxylate (260 mg, 0.65 mmol, 63%) as a light oil. LCMS (ESI): m/z=399 [M+H]⁺.

¹H NMR (400 MHz, DMSO-d6) δ 7.61 (d, J=7.0 Hz, 1H), 6.34-6.15 (m, 2H), 3.92-3.76 (m, 3H), 3.35-3.24 (m, 2H), 2.81-2.68 (m, 1H), 2.28-2.08 (m, 3H), 2.03-1.89 (m, 1H), 1.63-1.54 (m, 2H), 1.49-1.20 (m, 17H), 0.50 (s, 2H).

### STEP C: tert-butyl (6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carboxylate

To a solution of tert-butyl tert-butyl (R)-6-(1-(oct-7-yn-1-yl)-2-oxo-1,2-dihydropyridin-4-yl)-4-azaspiro[2.4]heptane-4-carboxylate (257 mg, 0.65 mmol, 1.1 eq.), 3-(4-bromo-1-oxoisoindolin-2-yl)piperidine-2,6-dione (190 mg, 0.59 mmol, 1.0 eq.), Cs₂CO₃ (575 mg, 1.76 mmol, 3.0 eq.) and 4 A molecular sieve (100 mg) in DMF (15 mL) were added CuI (22 mg, 0.12 mmol, 0.2 eq.) and Pd(dppf)Cl₂ (43 mg, 59 μmol, 0.1 eq.). And the reaction mixture was stirred at 80° C. overnight under N₂. The reaction mixture was diluted with H₂O (20 mL) and extracted with EtOAc (20 mL×2). The combined organic layers were washed with brine (10 mL), dried over anhydrous Na₂SO₄, and concentrated under reduced pressure. The residue was purified by C18 column chromatography to give tert-butyl (6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carboxylate (160 mg, 0.25 mmol, 42%) as a yellow solid LCMS (ESI): m/z=641 [M+H]⁺.

### STEP D: 3-(1-oxo-4-(8-(2-oxo-4-((R)-4-azaspiro[2.4]heptan-6-yl)pyridin-1(2H)-yl)oct-1-yn-1-yl)isoindolin-2-yl)piperidine-2,6-dione

To a solution of tert-butyl (6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carboxylate (160 mg, 0.25 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(1-oxo-4-(8-(2-oxo-4-((R)-4-azaspiro[2.4]heptan-6-yl)pyridin-1(2H)-yl)oct-1-yn-1-yl)isoindolin-2-yl)piperidine-2,6-dione (160 mg, quant.) as a yellow oil, which was used in next step directly without further purification. LCMS (ESI): m/z=541 [M+H]⁺.

### Preparation of ((2-(((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 5) and S,S′-(((((2-(((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 6)

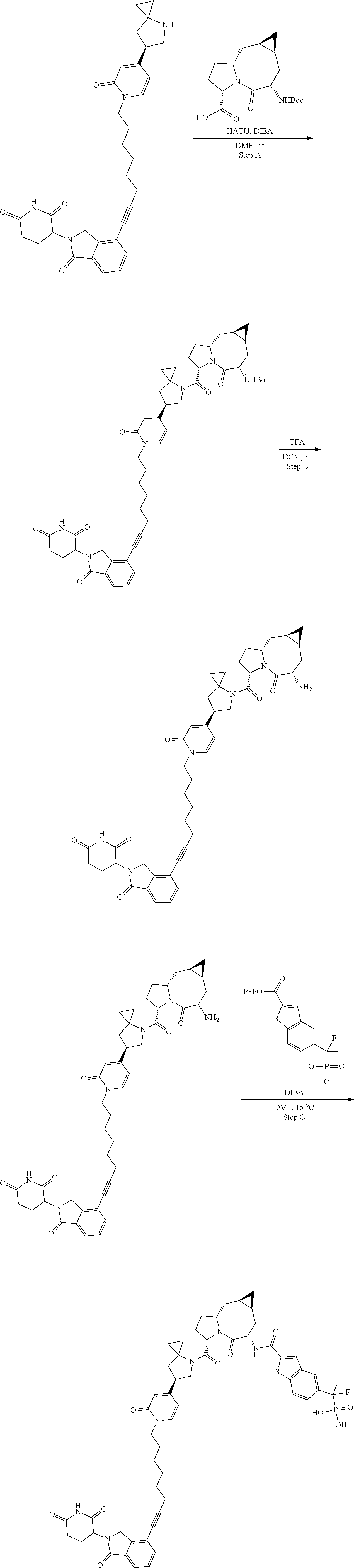

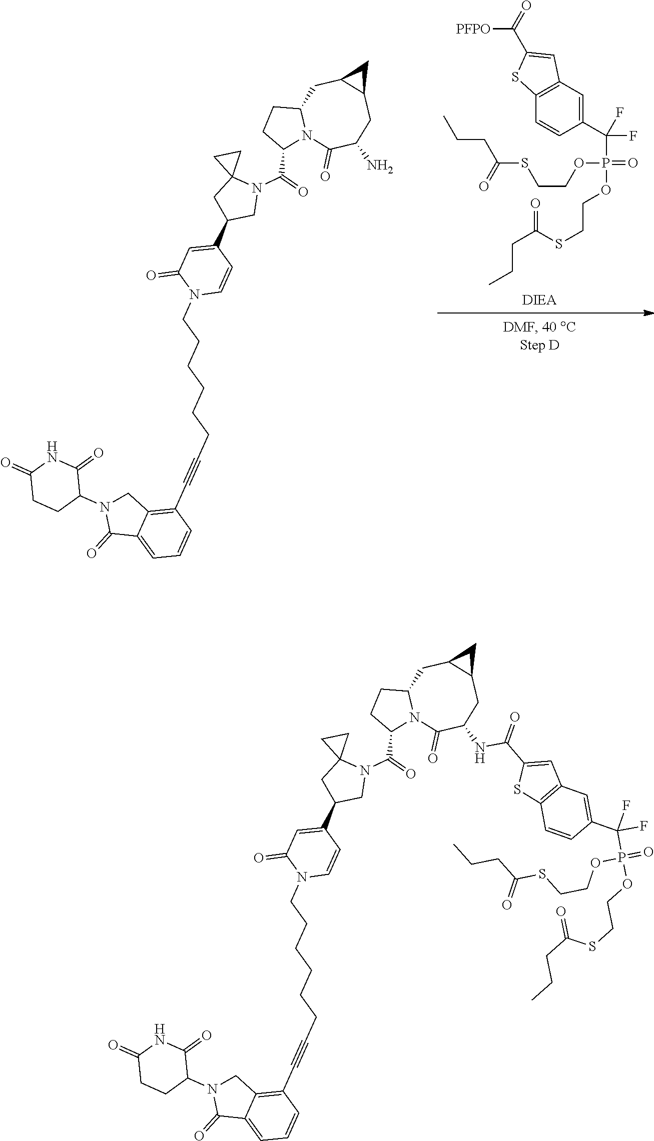

### STEP A: tert-butyl ((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (120 mg, 0.29 mmol, 1.2 eq.) and HATU (139 mg, 0.37 mmol, 1.5 eq.) in DMF(1 mL) were added 3-(1-oxo-4-(8-(2-oxo-4-((R)-4-azaspiro[2.4]heptan-6-yl)pyridin-1(2H)-yl)oct-1-yn-1-yl)isoindolin-2-yl)piperidine-2,6-dione (160 mg, 0.24 mmol, 1.0 eq.) and DIEA (95 mg, 0.73 mmol, 3 eq.). The resulting mixture was stirred at room temperature for 2 hrs under N₂. After completion, the reaction mixture was purified by Biotage® C18 column chromatography to get tert-butyl ((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (100 mg, 0.15 mmol. 56%) as a white solid. LCMS (ESI): m/z=861 [M+H]⁺.

### STEP B: 3-(4-(8-(4-((R)-4-((3S,6S 7aS,8aR,9aR)-6-amino-5-oxodecahydro-1H-cyclopropa[d]pyrrolo[1,2-a]azocine-3-carbonyl)-4-azaspiro[2,4]heptan-6-yl)-2-oxopyridin-1(2H)-yl)oct-1-yn-1-yl)-1-oxoisoindolin-2-yl)piperidine-2,6-dione

To a solution of tert-butyl ((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (100 mg, 0.15 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(4-(8-(4-(R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)oct-1-yn-1-yl)-1-oxoisoindolin-2-yl)piperidine-2,6-dione (80 mg, quant.) as a yellow oil, which was used in next step directly without further purification. LCMS (ESI): m/z=761 [M+H]⁺.

### STEP C: ((2-(((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of 3-(4-(8-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)oct-1-yn-1-yl)-1-oxoisoindolin-2-yl)piperidine-2,6-dione (40 mg, 52 μmol, 1.0 eq.) in DMF (1 mL) were added (difluoro(2-((perfluorophenoxy)carbonyl)benzo[b]thiophen-5-yl)methyl)phosphonic acid (25 mg, 52 μmol, 1.0 eq.) and TEA (16 mg, 157 μmol, 3.0 eq.). The resulting mixture was stirred at 40° C. for 2 hrs under N₂. After completion, the reaction mixture was concentrated under reduced pressure. The residue was purified by prep-HPLC to get ((2-(((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzol bithiophen-5-yl)difluoromethyl)phosphonic acid (Compound 5, 23.3 mg, 22 μmol, 42%) as a white solid.

¹H NMR (400 MHz, DMSO-d6) δ 10.98 (s, 1H), 8.88 (d, J=7.2 Hz, 1H), 8.34 (s, 1H), 8.17-8.04 (m, 2H), 7.72-7.55 (m, 4H), 7.51-7.43 (m, 1H), 6.31 (s, 1H), 6.22 (d, J=6.9 Hz, 1H), 5.12 (dd, J=13.2, 4.9 Hz, 1H), 4.96-4.90 (m, 1H), 4.55-4.51 (m, 1H), 4.46-4.41 (m, 1H), 4.32-4.28 (m, 1H), 4.17-4.12 (m, 1H), 4.03 (t, J=8.6 Hz, 1H), 3.83-3.72 (m, 3H), 3.38-3.30 (m, 1H), 3.03-2.83 (m, 1H), 2.67-2.55 (m, 1H), 2.44 (t, J=6.8 Hz, 3H), 2.36-2.30 (m, 1H), 2.17-1.97 (m, 7H), 1.90-1.77 (m, 2H), 1.67-1.49 (m, 6H), 1.43-1.35 (m, 3H), 1.30-1.23 (m, 3H), 0.87-0.71 (m, 2H), 0.50-0.36 (m, 2H), −0.01-−0.03 (m, 1H).(TFA salt)

LCMS (ESI): m/z=1051.0 [M+H]⁺.

### STEP D: S,S′-(((((2-(((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of 3-(4-(8-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)oct-1-yn-1-yl)-1-oxoisoindolin-2-yl)piperidine-2,6-dione (40 mg, 52 μmol, 1.0 eq.) in DMF (1 mL) were added 4-nitrophenyl 5-((bis(2-(butyrylthio)ethoxy)phosphoryl)difluoromethyl)benzo[b]thiophene-2-carboxylate (36 mg, 52 μmol, 1.0 eq.) and TEA (16 mg, 157 μmol, 3.0 eq.). The resulting mixture was stirred at room temperature for 2 hrs under N₂. After completion, the reaction mixture was purified by prep-HPLC to get S,S′-(((((2-(((3S,6S,7aS,8aR,9aR)-3-((6R)-6-(1-(8-(2-(2,6-dioxopiperidin-3-yl)-1-oxoisoindolin-4-yl)oct-7-yn-1-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 6, 38.3 mg, 29 μmol, 55%) as a white solid.

¹H NMR (400 MHz, DMSO-d6) δ 10.97 (s, 1H), 8.94 (d, J=7.3 Hz, 1H), 8.36 (s, 1H), 8.24-8.10 (m, 2H), 7.69 (d, J=7.4 Hz, 1H), 7.62-7.56 (m, 3H), 7.50 (t, J=7.6 Hz, 1H), 6.32 (s, 1H), 6.21 (d, J=6.5 Hz, 1H), 5.14-5.10 (m, 1H), 4.96-4.90 (m, 1H), 4.53-4.52 (m, 1H), 4.45-4.41 (m, 1H), 4.32-4.28 (m, 1H), 4.23-4.14 (m, 4H), 4.04 (t, J=8.4 Hz, 1H), 3.83-3.71 (m, 3H), 3.12 (t, J=5.7 Hz, 3H), 2.94-2.84 (m, 1H), 2.67-2.59 (m, 1H), 2.47-2.40 (m, 3H), 2.36-2.30 (m, 1H), 2.12-1.94 (m, 9H), 1.88-1.80 (m, 2H), 1.60-1.52 (m, 9H), 1.46-1.38 (m, 3H), 1.29-1.20 (m, 8H), 0.87-0.83 (m, 7H), 0.81-0.65 (m, 2H), 0.51-0.38 (m, 2H), 0.00-−0.03 (m, 1H).

LCMS (ESI): m/z=1311.9 [M+H]⁺.

### Preparation of rac-(3S,4R)-4-(3-((7-(2-(2,6-dioxopiperidin-3-yl)-1-oxoisoindolin-4-yl)hept-6-yn-1-yl)oxy)phenyl)pyrrolidine-3-carbonitrile

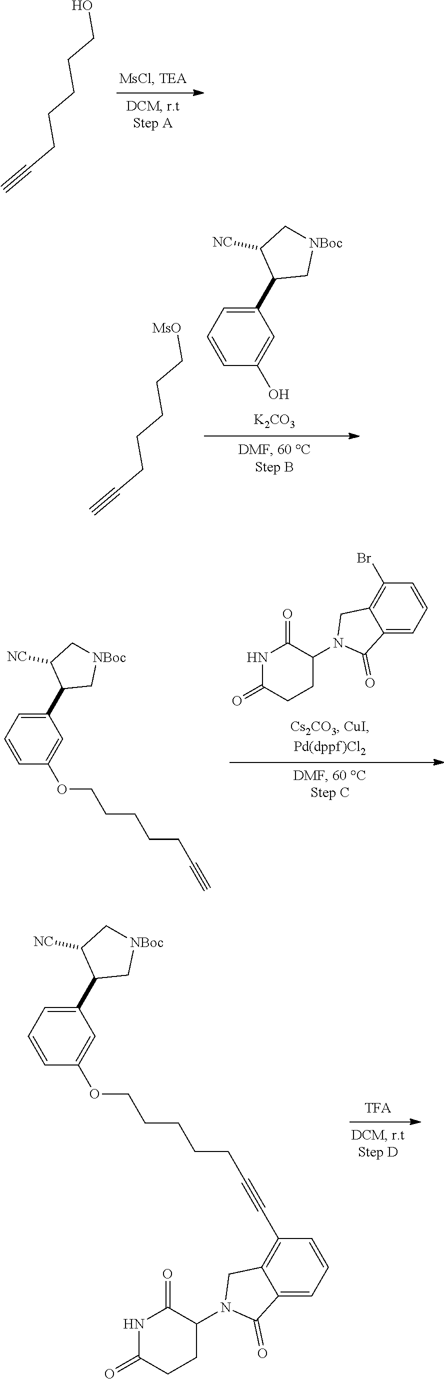

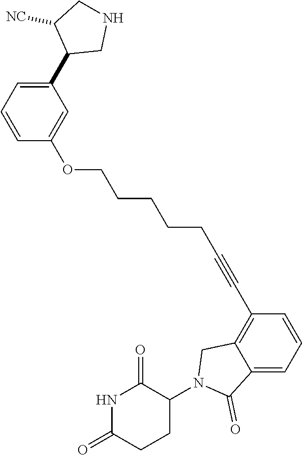

### STEP A: hept-6-yn-1-yl methanesulfonate

To a solution of hept-6-yn-1-ol (5.0 g, 44.6 mmol, 1.0 eq.) in DCM (60 mL) were added MsCl (10.0 g, 89.2 mmol, 2.0 eq.) and TEA (9.0 g, 89.2 mmol, 2.0 eq.). The resulting mixture was stirred at room temperature for 15 min under N₂. After completion, the reaction mixture was diluted with water (40 mL) and extracted with EtOAc (120 mL×3). The combined organic layer was washed with brine (100 mL×2), dried over anhydrous Na₂SO₄ and concentrated under reduced pressure to afford hept-6-yn-1-yl methanesulfonate (8 g, 42, mmol, 94%) as a colorless oil. ¹H NMR (400 MHz, DMSO-d6) δ 4.19 (t, J=6.4 Hz, 2H), 3.15 (s, 3H), 2.74 (t, J=2.5 Hz, 1H), 2.17 (td, J=6.7, 2.6 Hz, 2H), 1.73-1.62 (m, 2H), 1.51-1.40 (m, 4H).

### STEP B: rac-tert-butyl (3S,4R)-3-cyano-4-(3-(hept-6-yn-1-yloxy)phenyl)pyrrolidine-1-carboxylate

To a solution of hept-6-yn-1-yl methanesulfonate (800 mg, 4.2 mmol, 1.0 eq.) and rac-tert-butyl (3S,4R)-3-cyano-4(3-hydroxyphenyl)pyrrolidine-1-carboxylate (1.21 g, 4.2 mmol, 1.0 eq.) in DMF (10 mL) was added K₂CO₃ (1.15 g, 8.4 mmol, 2.0 eq.). The mixture was stirred at 60° C. for 12 hrs. After completion, the mixture was filtered and the filtrate was purified by Biotage C18 column chromatography to afford rac-tert-butyl (3S,4R)-3-cyano-4-(3-(hept-6-yn-1-yloxy)phenyl)pyrrolidine-1-carboxylate (850 mg, 2.2 mmol, 50%) as a colorless oil. LCMS (ESI): m/z=383 [M+H]⁺.

### STEP C: rac-tert-butyl (3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carboxylate

To a solution of rac-tert-butyl (3S,4R)-3-cyano-4-(3-(hept-6-yn-1-yloxy)phenyl)pyrrolidine-1-carboxylate (630 mg, 1.6 mmol, 1.0 eq.) and 3-(4-bromo-1-oxoisoindolin-2-yl)piperidine-2,6-dione (520 mg, 1.6 mmol, 1.0 eq.) in DMF (10 mL) were added Cs₂CO₃ (1.6 g, 4.8 mmol, 3.0 eq.). CuI (63 mg, 0.32 mmol, 0.2 eq.) and Pd(dppf)Cl₂ (241 mg, 0.2 mmol, 0.2 eq.). The mixture was purged and degassed with N₂ 3 times and then stirred at 60° C. for 12 hrs. After completion, the mixture was filtered and the filtrate was purified by Biotage C18 column chromatography to afford rac-tert-butyl (3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carboxylate (340 mg, 0.54 mmol, 31%) as a gray solid. LCMS (ESI): m/z=625 [M+H]⁺.

### STEP D: rac-(3S,4R)-4-(3-((7-(2-(2,6-dioxopiperidin-3-yl)-1-oxoisoindolin-4-yl)hept-6-yn-1-yl)oxy)phenyl)pyrrolidine-3-carbonitrile

To a solution of rac-tert-butyl (3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carboxylate (170 mg, 0.27 mmol, 1.0 eq.) in DCM (1.8 mL) was added TFA (0.6 mL). The resulting mixture was stirred at room temperature for 2 hrs. After completion, the mixture was concentrated under reduced pressure to give rac-(3S,4R)-4-(3-((7-(2-(2,6-dioxopiperidin-3-yl)-1-oxoisoindolin-4-yl)hept-6-yn-1-yl)oxy)phenyl)pyrrolidine-3-carbonitrile (138 mg, quant.) as a yellow oil, which was directly used without further purification. LCMS (ESI): m/z=525 [M+H]⁺.

### Preparation of ((2-(((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 7) and S,S′-(((((2-(((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 8)

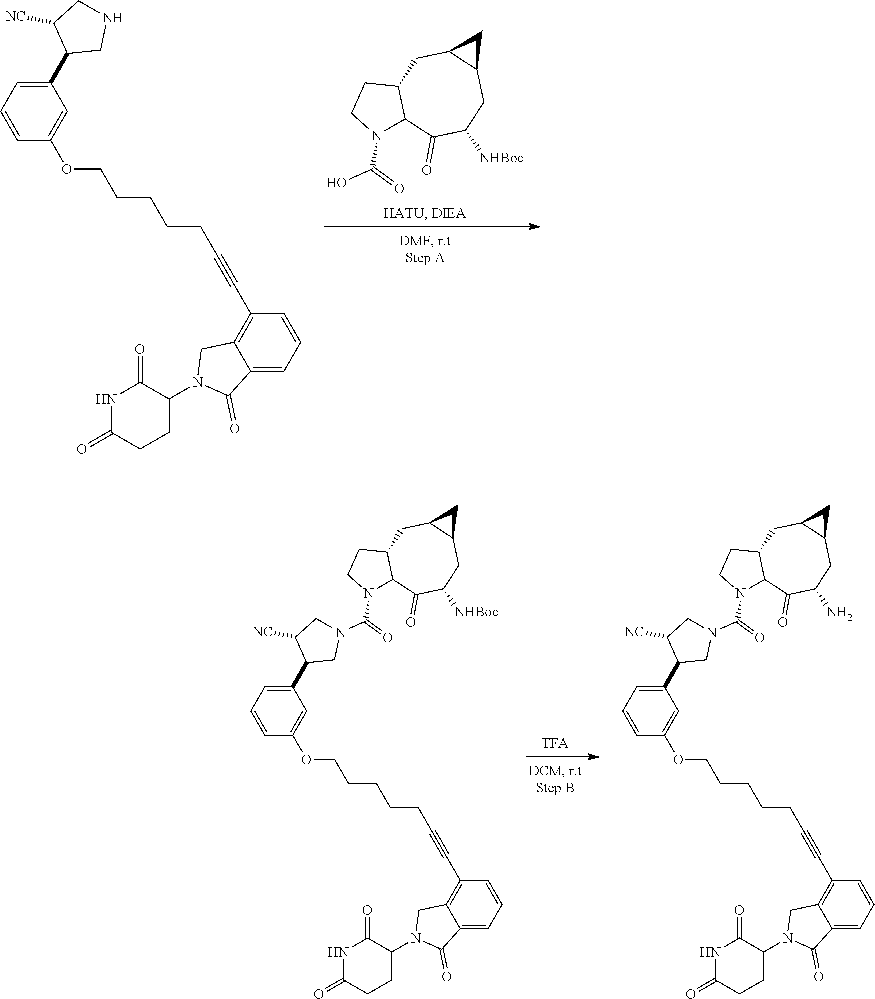

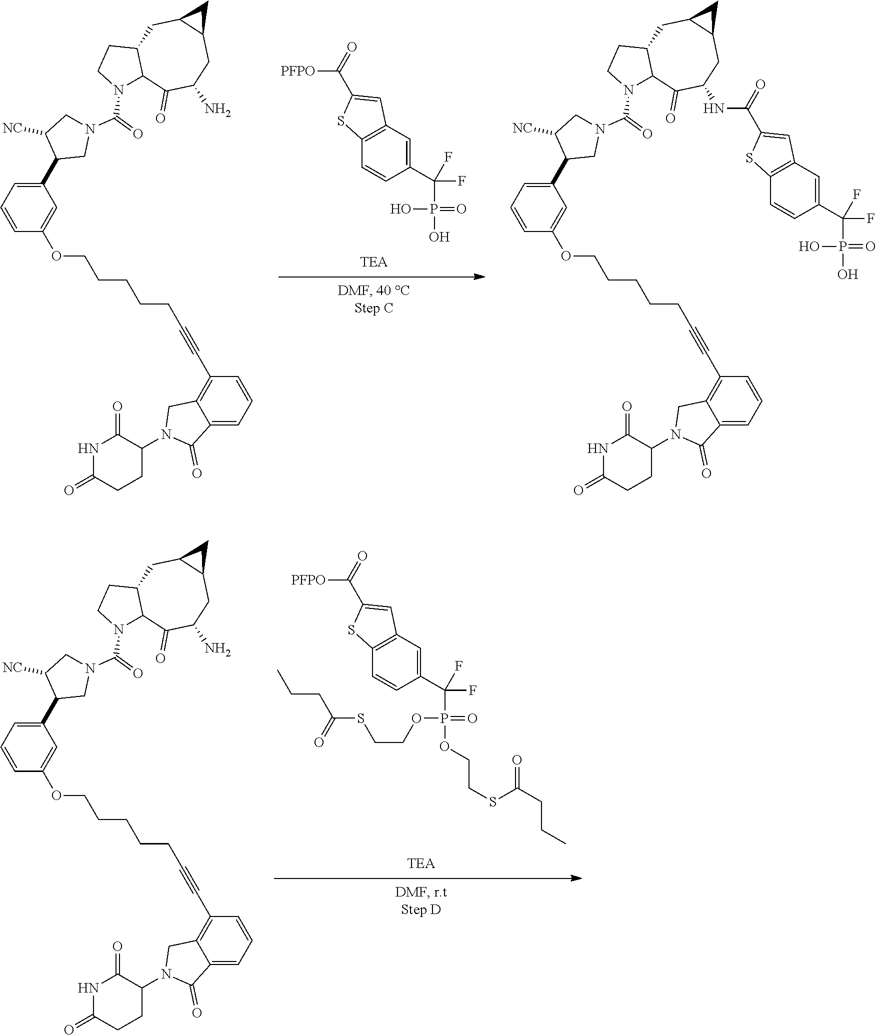

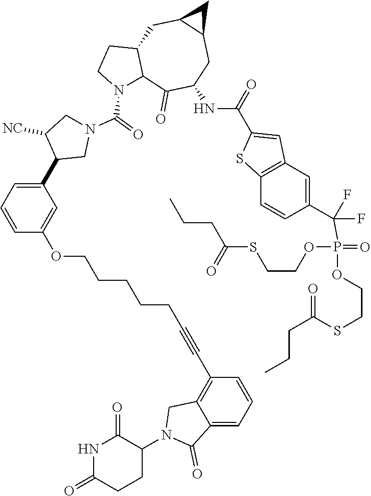

### STEP A: tert-butyl ((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (170 mg, 0.50 mmol, 1.2 eq.) and HATU (239 mg, 0.63 mmol, 1.5 eq.) in DMF (1 mL) were added rac-(3S,4R)-4-(3-((7-(2-(2,6-dioxopiperidin-3-yl)-1-oxoisoindolin-4-yl)hept-6-yn-1-yl)oxy)phenyl)pyrrolidine-3-carbonitrile (220 mg, 0.42 mmol, 1.0 eq.) and DIEA (162 mg, 1.26 mmol, 3 eq.). The resulting mixture was stirred at room temperature for 2 hrs under N₂. After completion, the reaction mixture was purified by Biotage® C18 column chromatography to get tert-butyl ((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamate (210 mg, 0.25 mmol, 59%) as a white solid. LCMS (ESI): m/z=845 [M+H]⁺.

### STEP B: (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile

To a solution of tert-butyl ((3S,6S,7aS,8aR,9aR)-3-(rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamate (210 mg, 0.25 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile (200 mg, quant.) as a yellow oil, which was used in next step directly without further purification. LCMS (ESI): m/z=745 [M+H]⁺.

### STEP C: ((2-(((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile (100 mg, 134 μmol. 1.0 eq.) in DMF (1 mL) were added (difluoro(2-((perfluorophenoxy)carbonyl)benzo[b]thiophen-5-yl)methyl)phosphonic acid (55 mg, 134 μmol, 1.0 eq.) and TEA (36 mg, 403 μmol, 3.0 eq.). The resulting mixture was stirred at 40° C. for 2 hrs under N₂. After completion, the reaction mixture was concentrated under reduced pressure. The residue was purified by prep-HPLC to get ((2-(((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 7, 44.2 mg, 42 μmol, 31%) as a white solid.

¹H NMR (400 MHz, DMSO-d6) δ 10.98 (s, 1H), 8.91-8.79 (m, 1H), 8.33 (t, J=6.3 Hz, 1H), 8.19-8.03 (m, 2H), 7.70 (d, J=7.5 Hz, 1H), 7.65-7.56 (m, 2H), 7.54-7.47 (i, 1H), 7.31-7.22 (m, 1H), 7.05-6.92 (m, 2H), 6.90-6.83 (m, 1H), 5.17-5.08 (m, 1H), 4.99-4.91 (m, 1H), 4.70-4.49 (m, 2H), 4.47-4.41 (m, 1H), 4.34-4.28 (m, 1H), 4.24-4.08 (m, 2H), 4.04-3.93 (m, 3H), 3.92-3.73 (m, 2H), 3.67-3.60 (m, 1H), 3.52-3.44 (m, 1H), 3.33-3.16 (m, 1H), 2.95-2.82 (m, 1H), 2.68-2.54 (m, 1H), 2.44-2.30 (m, 2H), 2.26-1.92 (m, 6H), 1.92-1.81 (m, 2H), 1.79-1.73 (m, 2H), 1.66-1.54 (m, 4H), 1.45-1.36 (m, 1H), 0.87-0.71 (m, 2H), 0.03-0.01 (m, 1H).

LCMS (ESI): m/z=1035.4 [M+H]+.

### STEP D: S,S′-(((((2-(((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-((7-(2-(2,6-dioxopiperidin-3-yl)-1-oxoisoindolin-4-yl)hept-6-yn-1-yl)oxy)phenyl)pyrrolidine-3-carbonitrile (100 mg, 134 μmol. 1.0 eq.) in DMF (1 mL) were added 4-nitrophenyl 5-((bis(2-(butyrylthio)ethoxy)phosphoryl)difluoromethyl)benzo[b]thiophene-2-carboxylate (64 mg, 93 μmol, 1.0 eq.) (92 mg, 134 μmol, 1.0 eq.) and TEA (36 mg, 402 μmol, 3.0 eq.). The resulting mixture was stirred at room temperature for 2 hrs under N₂. After completion, the reaction mixture was purified by prep-HPLC to afford S,S′-(((((2-(((3S,6S,7aS,8aR,9aR)-3-((rel-3S,4R)-3-cyano-4-(3-((7-(2-(2,6-dioxopiperidin-3-yl)-1-oxoisoindolin-4-yl)hept-6-yn-1-yl)oxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 8, 49.8 mg, 38 μmol, 28%) as a white solid.

¹H NMR (400 MHz, DMSO-d6) δ 10.99 (s, 1H), 8.94 (dd, J=19.6, 7.3 Hz, 1H), 8.36 (t, J=6.5 Hz, 1H), 8.24-8.13 (m, 2H), 7.73-7.67 (m, 1H), 7.64-7.56 (m, 2H), 7.54-7.47 (m, 11H), 7.30-7.23 (m, 1H), 7.04-6.93 (m, 2H), 6.89-6.83 (m, 1H), 5.17-5.09 (m, 1H), 5.02-4.90 (m, 1H), 4.64-4.54 (m, 1H), 4.48-4.40 (m, 1H), 4.34-4.28 (m, 1H), 4.25-4.07 (m, 6H), 4.03-3.92 (m, 3H), 3.85-3.72 (m, 1H), 3.68-3.59 (m, 1H), 3.54-3.44 (m, 1H), 3.31-3.17 (m, 1H), 3.16-3.10 (m, 4H), 2.95-2.84 (m, 1H), 2.56-2.52 (m, 5H), 2.45-2.33 (m, 2H), 2.22-1.94 (m, 6H), 1.93-1.83 (m, 2H), 1.81-1.71 (m, 2H), 1.64-1.51 (m, 8H), 1.45-1.36 (m, 1H), 1.31-1.25 (m, 1H), 0.92-0.80 (m, 8H), 0.77-0.69 (m, 1H), 0.03-0.01 (m, 1H).

LCMS (ESI): m/z=1296.2 [M+H]⁺.

### Preparation of 3-(4-((5-(2-oxo-4-((R)-4-azaspiro[2.4]heptan-6-yl)pyridin-1(2H)-yl)pentyl)oxy)phenyl)piperidine-2,6-dione

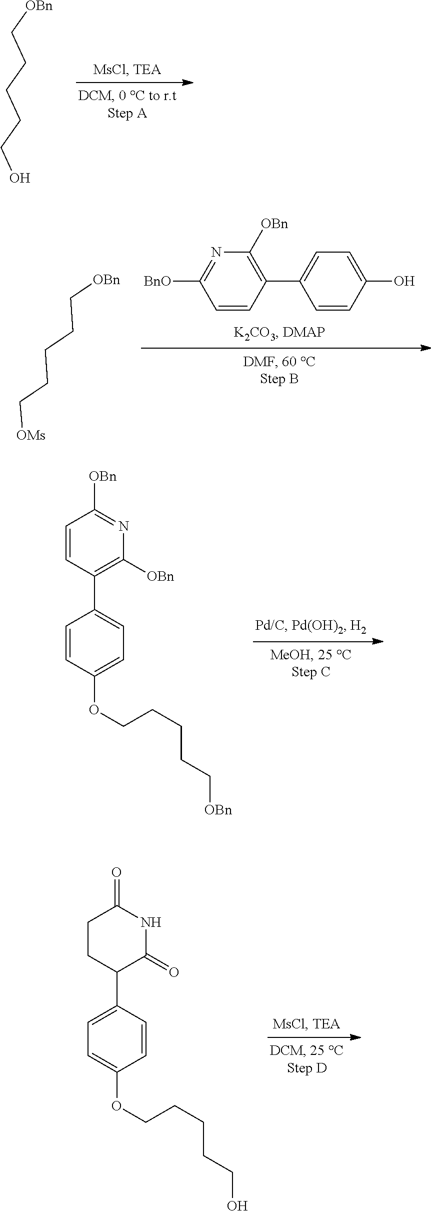

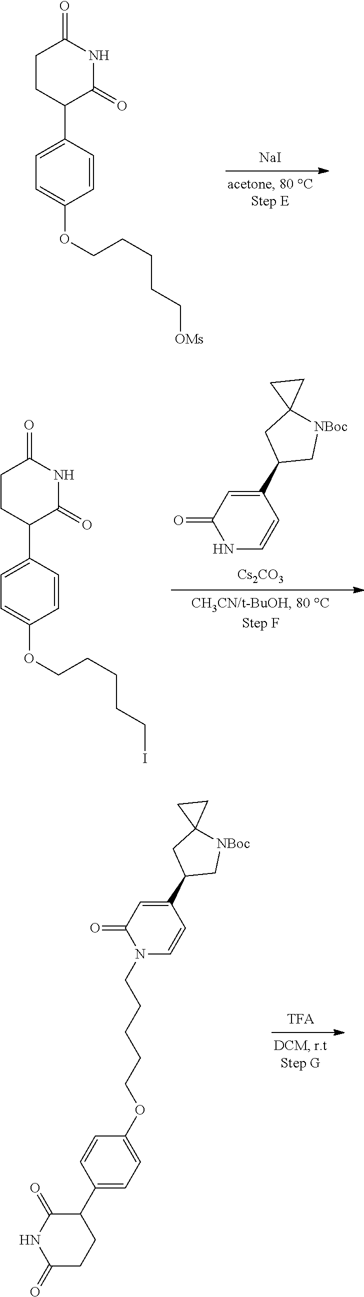

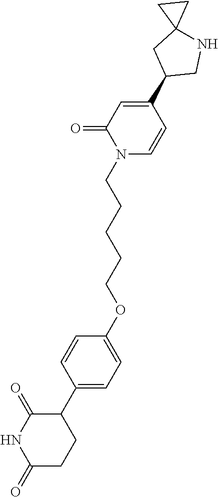 [SMILES: O=C1CCC(c2ccc(OCCCCCn3ccc([C@@H]4CNC5(CC5)C4)cc3=O)cc2)C(=O)N1]

### STEP A: 5-(benzyloxy)pentyl methanesulfonate

To a solution of 5-(benzyloxy)pentan-1-ol (4 g, 20.6 mmol, 1 eq.) and TEA (4.2 g, 41.22 mmol, 2 eq.) in DCM (60 mL) was added MsCl (2.6 g, 22.8 mmol, 1.1 eq.) dropwise at 0° C. The reaction mixture was warmed up to room temperature and stirred for 0.5 hrs under N₂ atmosphere. After completion, the reaction mixture was diluted with water (100 mL) and extracted with DCM (100 mL×3). The combined organic layer was washed with brine (100 mL), dried over anhydrous Na₂SO₄ and concentrated under reduced pressure to give 5-(benzyloxy)pentyl methanesulfonate (5.5 g, quant.) as a colorless oil. LCMS (ESI) m/z=273 [M+H]⁺.

### STEP B: 2,6-bis(benzyloxy)-3-(4-((5-(benzyloxy)pentyl)oxy)phenyl)pyridine

To a solution of 5-(benzyloxy)pentyl methanesulfonate (3 g, 11.0 mmol. 1 eq.) and 4-(2,6-bis(benzyloxy)pyridin-3-yl)phenol (4.22 g, 11.03 mmol, 1.0 eq.) in DMF (100 mL) were added DMAP (134 mg, 1.10 mmol, 0.1 eq.) and K₂CO₃ (3.04 g, 22.1 mmol, 2 eq.). The reaction mixture was stirred at 60° C. for 48 hrs under N₂ atmosphere. After completion, the reaction mixture was diluted with water (100 mL) and extracted with EtOAc (100 mL×3). The combined organic layer was washed with brine (100 mL), dried over anhydrous Na₂SO₄ and concentrated under reduced pressure. The residue was purified by column chromatography on silica gel to give 2,6-bis(benzyloxy)-3-(4-((5-(benzyloxy)pentyl)oxy)phenyl)pyridine (3.95 g, 7.06 mmol, 64%) as a colorless oil. LCMS (ESI) m/z=560 [M+H]⁺.

### STEP C: 3-(4-((5-hydroxypentyl)oxy)phenyl)piperidine-2,6-dione

To a solution of 2,6-bis(benzyloxy)-3-(4-((5-(benzyloxy)pentyl)oxy)phenyl)pyridine (3.95 g, 7.06 mmol, 1 eq.) in MeOH (150 mL) were added Pd/C (400 mg) and Pd(OH)₂ (400 mg) at 25° C. under N₂. The suspension was degassed under vacuum and purged with H₂ several times. The resulting mixture was stirred at room temperature for 16 hrs. After completion, the suspension was filtered through a pad of Celite®, the filter cake was washed with MeOH (50 mL). The combined filtrates were concentrated to dryness to give 3-(4-((5-hydroxypentyl)oxy)phenyl)piperidine-2,6-dione (1.5 g, 5.15 mmol, 73%) as a white solid. LCMS (ESI) m/z=292 [M+H]⁺.

### STEP D: 5-(4-(2,6-dioxopiperidin-3-yl)phenoxy)phenyl methanesulfonate

To a solution of 3-(4-((5-hydroxypentyl)oxy)phenyl)piperidine-2,6-dione (1.5 g, 5.15 mmol, 1 eq.) in DCM (50 ml) were added TEA (1.0 g, 10.3 mmol, 2 eq.) and MsCl (587 mg, 5.66 mmol, 1.1 eq.). The reaction mixture was stirred at 25° C. for 1 hr under N₂ atmosphere. After completion, the reaction mixture was diluted with water (10 mL) and extracted with DCM (20 mL×3). The combined organic layer was washed with brine (10 mL), dried over anhydrous Na₂SO₄ and concentrated under reduced pressure to give 5-(4-(2,6-dioxopiperidin-3-yl)phenoxy)pentyl methanesulfonate (1.7 g, 4.61 mmol, 89%) as a white solid. LCMS (ESI) m/z=370 [M+H]⁺.

### STEP E: 3-(4-((5-iodopentyl)oxy)phenyl)piperidine-2,6-dione

To a solution of 5-(4-(2,6-dioxopiperidin-3-yl)phenoxy)pentyl methanesulfonate (1.7 g, 4.61 mmol, 1 eq.) in acetone (20 mL) was added NaI (2.1 g, 14.1 mmol, 3.0 eq.) at 25° C. The reaction mixture was stirred at 80° C. for 2 hrs under N₂ atmosphere. After completion, Na₂S₂O₃ in H₂O was added to the reaction solution until the yellow color faded away. The aqueous phase was extracted with EtOAc (10 mL×2). The combined organic layers were washed with brine (50 mL), dried over Na₂SO₄ and concentrated under reduced pressure to give crude product. The residue was purified by column chromatography on silica gel to give 3-(4-((5-iodopentyl)oxy)phenyl)piperidine-2,6-dione (1.5 g, 3.74 mmol, 82%) as a white solid. LCMS (ESI) m/z=402 [M+H]⁺.

### STEP F: tert-butyl (6R)-6-(1-(5-(4-(2,6-dioxopiperidin-3-yl)phenoxy)pentyl)-2-oxo-1,2-dihydropyridin-4-yl)-4-azaspiro[2.4]heptane-4-carboxylate and tert-butyl (6R)-6-(2-((5-(4-(2,6-dioxopiperidin-3-yl)phenoxy)pentyl)oxy)pyridin-4-yl)-4-azaspiro[2.4]heptane-4-carboxylate

To a solution of 3-(4-((5-iodopentyl)oxy)phenyl)piperidine-2,6-dione (1 g, 2.49 mmol, 1 eq.) and tert-butyl (R)-6-(2-oxo-1,2-dihydropyridin-4-yl)-4-azaspiro[2.4]heptane-4-carboxylate (723 mg, 2.49 mml, 1 eq.) in CH₃CN (20 mL) and t-BuOH (10 mL) was added and Cs₂CO₃ (2.04 g, 6.23 mmol, 2.5 eq.) at 25° C. The reaction mixture was stirred at 80° C. for 2 hrs under N₂ atmosphere. The mixture was purified by C18 column to afford tert-butyl (6R)-6-(1-(5-(4-(2,6-dioxopiperidin-3-yl)phenoxy)pentyl)-2-oxo-1,2-dihydropyridin-4-yl)-4-azaspiro[2.4]heptane-4-carboxylate (180 mg, 0.32 mmol, 13%) and tert-butyl (6R)-6-(2-((5-(4-(2,6-dioxopiperidin-3-yl)phenoxy)pentyl)oxy)pyridin-4-yl)-4-azaspiro[2.4]heptane-4-carboxylate (69 mg, 0.12 mmol, 5%) both isomers as white solid. LCMS (ESI) m/z=564 [M+H]⁺.

### STEP G: 3-(4-((5-(2-oxo-4-((R)-4-azaspiro[2.4]heptan-6-yl)pyridin-1(2H)-yl)pentyl)oxy)phenyl)piperidine-2,6-dione

To a solution of tert-butyl (6R)-6-(1-(5-(4-(2,6-dioxopiperidin-3-yl)phenoxy)pentyl)-2-oxo-1,2-dihydropyridin-4-yl)-4-azaspiro[2.4]heptane-4-carboxylate (90 mg, 0.16 mmol, 1 eq.) in DCM (2 mL) was added TFA (1 mL) and the resulting mixture was stirred at room temperature for 0.5 hrs. After completion, the reaction mixture was concentrated under reduced pressure to give 3-(4-((5-(2-oxo-4-((R)-4-azaspiro[2.4]heptan-6-yl)pyridin-1(2H)-yl)pentyl)oxy)phenyl)piperidine-2,6-dione (74 mg, quant) as a yellow oil LCMS (ESI) m/z=464 [M+H]⁺.

### Preparation of S,S′-(((((2-(((3S,6S,7aS,8aR,9aR)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 9) and ((2-(((3S,6S,7aS,8aR,9aR)-3-((6R)-4 (1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 10)

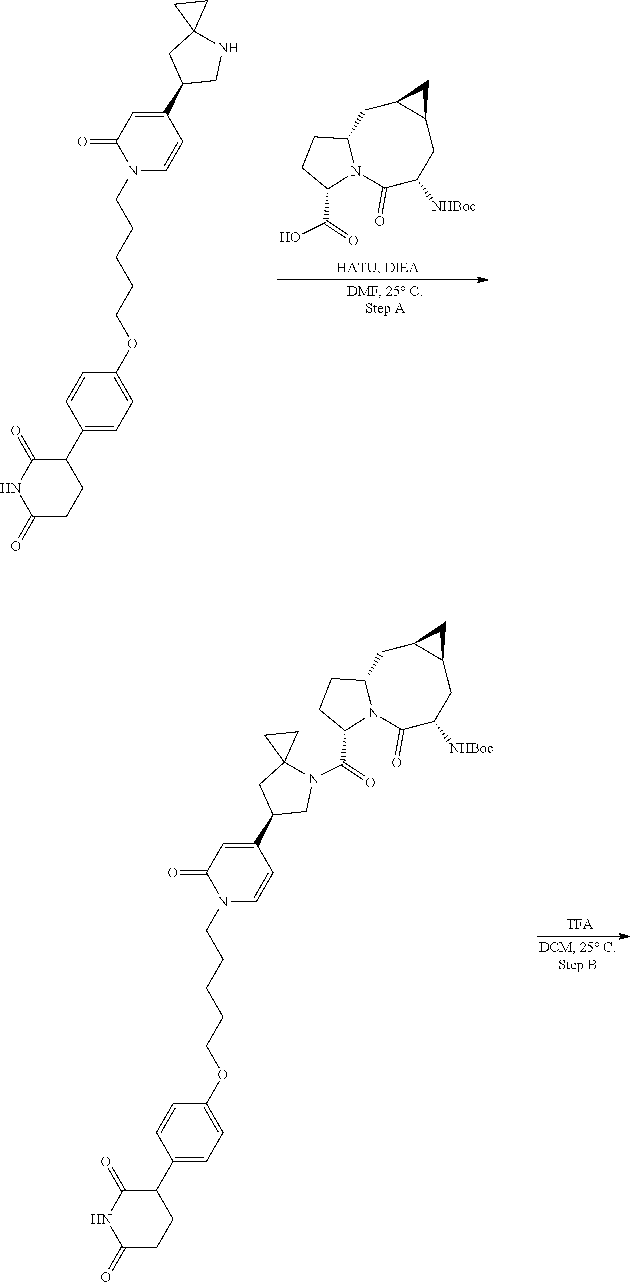 [SMILES: O=C1CCC(c2ccc(OCCCCCn3ccc([C@@H]4CNC5(CC5)C4)cc3=O)cc2)C(=O)N1 ; CC(C)(C)OC(=O)N[C@H]1C[C@@H]2C[C@@H]2C[C@H]2CC[C@@H](C(=O)O)N2C1=O ; CC(C)(C)OC(=O)N[C@H]1C[C@@H]2C[C@@H]2C[C@H]2CC[C@@H](C(=O)N3C[C@@H](c4ccn(CCCCCOc5ccc(C6CCC(=O)NC6=O)cc5)c(=O)c4)CC34CC4)N2C1=O]

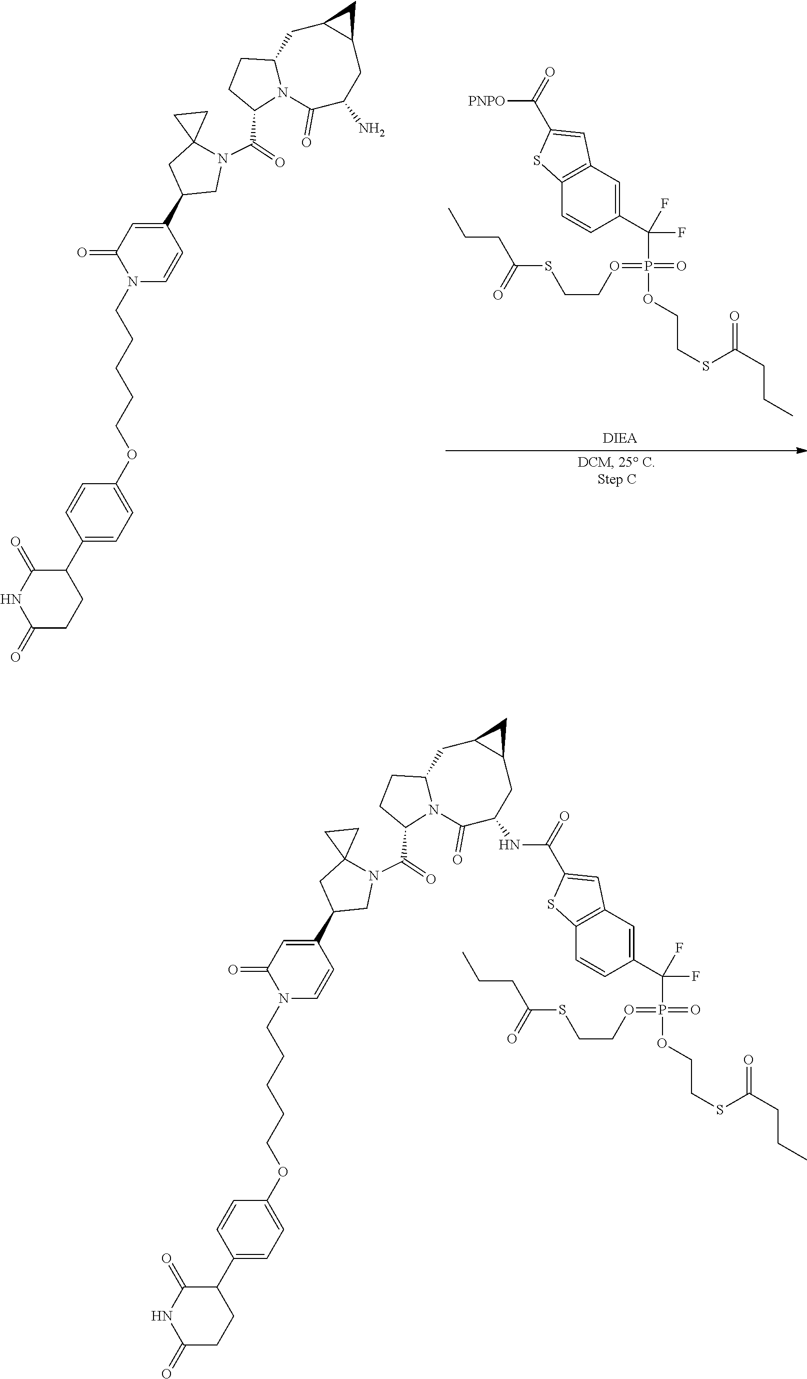

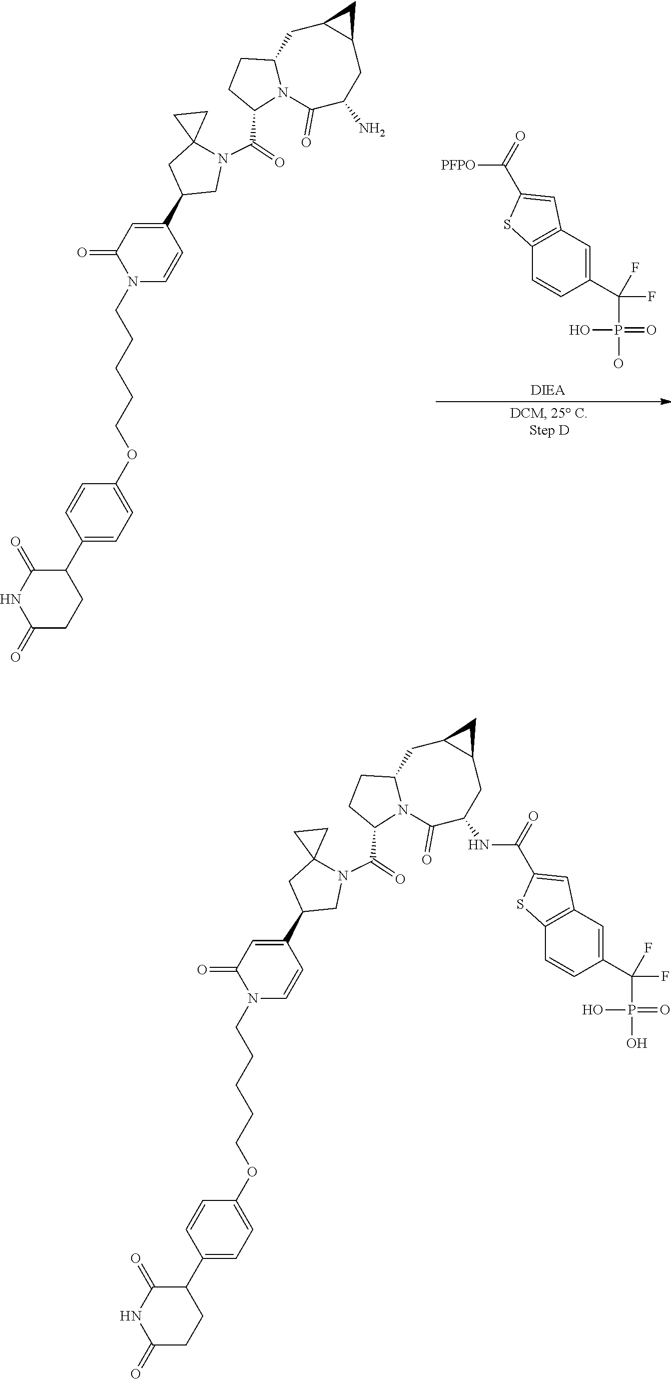

### STEP A: tert-butyl ((3S,6S,7aS,8aR,9aR)-3-((6R)-6-(1-(5-(4-(2,6-dioxopiperidin-3-yl)phenoxy)pentyl)-2-oxo-1,2-dihydropyridin-4-yl)-4-azaspiro[2,4]heptane-4-carbonyl)-5-oxodecahydro-1H-cyclopropra[d]pyrrolo[1,2-a]azocin-6-yl)carbamate

A solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (100 mg, 0.30 mmol, 1 eq.), DIEA (194 mg, 1.5 mmol, 5 eq.) and HATU (137 mg, 0.36 mmol, 1.2 eq.) in DMF (1 mL) was stirred at room temperature for 20 min. Then a solution of 3-(4-((5-((4-((R)-4-azaspiro[2.4]heptan-6-yl)pyridin-2-yl)oxy)pentyl)oxy)phenyl)piperidine-2,6-dione (139 mg, 0.30 mmol, 1 eq.) in DMF (1 ml) was added. The reaction mixture was stirred at room temperature for 1 hr. The reaction mixture was purified by C18 column to afford tert-butyl ((3S,6S,7aS,8aR,9aR)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (108 mg, 0.14 mmol, 46%) as a white solid LCMS (ESI) m/z=784 [M+H]⁺.

### STEP B: 3-(4-((5-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione

To a solution of tert-butyl ((3S,6S,7aS,8aR,9aR)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (40 mg, 51 μmol, 1.0 eq.) in dry DCM (5 mL) was added TFA (1 mL) at 25° C. The mixture was stirred at 25° C. for 2 hrs. After completion, the reaction mixture was concentrated under reduced pressure to give 3-(4-((5-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione (35 mg, quant.) as a yellow oil, which was used in next step without further purification. LCMS (ESI): m/z=684.6 [M+H]⁺.

### STEP C: S,S′-(((((2-(((3S,6S,7aS,8aR,9aR)-3-((6R)-6-(1-(5-(4-(2,6-dioxopiperidin-3-yl)phenoxy)phenyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a mixture of 3-(4-((5-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione (40 mg, 58 μmol, 1.0 eq.) and DIEA (23 mg, 0.18 mmol, 3.0 eq.) in DMF (3 mL) were added 4-nitrophenyl 5-((bis(2-(butyrylthio)ethoxy)phosphoryl)difluoromethyl)benzo[b]thiophene-2-carboxylate (40 mg, 58 μmol, 1.1 eq.) and DMAP (4 mg, 29 μmol, 0.5 eq.). The mixture was stirred at 25° C. for 30 min. After completion, the reaction mixture was purified by prep-HPLC to give S,S′-(((((2-(((3S,6S,7aS,8aR,9aR)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 9, (14.3 mg, 12 μmol, 20%) as a white solid.

¹H NMR (400 MHz, DMSO-d6) δ 10.79 (s, 1H), 8.96 (d, J=7.5 Hz, 1H), 8.37 (s, 1H), 8.24-8.15 (m, 2H), 7.60 (d, J=6.8 Hz, 2H), 7.10 (d, J=8.6 Hz, 2H), 6.85 (d, J=8.6 Hz, 2H), 6.35 (s, 1H), 6.24 (dd. J=7.1, 1.5 Hz, 1H), 4.94 (q, J=7.8 Hz, 1H), 4.54 (s, 1H), 4.25-4.11 (m, 5H), 4.05 (t, J=8.6 Hz, 1H), 3.91 (t, J=6.3 Hz, 2H), 3.87-3.73 (m, 4H), 3.14 (t, J=5.8 Hz, 4H), 2.72-2.59 (m, 1H), 2.55 (d, J=7.3 Hz, 3H), 2.52 (s, 2H), 2.50-2.43 (m, 1H), 2.40-2.30 (m, 1H), 2.18-1.94 (m, 9H), 1.90-1.78 (m, 2H), 1.73-1.60 (m, 5H), 1.58-1.50 (m, 5H), 1.38 (d, J=6.6 Hz, 3H), 0.91-0.79 (m, 7H), 0.74-0.70 (m, 1H), 0.51-0.39 (m, 2H), 0.05-4.05 (m, 1H).

LCMS (ESI): m/z=1234.3 [M+H]⁺.

### STEP D: ((2-(((3S,6S 7a,8aR,9aR)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of 3-(4-((5-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)pentyl)oxy)phenyl)piperidine-2,6-dione (35 mg, 51 μmol, 1.0 eq.) in DMF (2 mL) were added perfluorophenyl 5-((bis(2-(butyrylthio)ethoxy)phosphoryl)difluoromethyl)benzo[b]thiophene-2-carboxylate (22 mg, 51 μmol, 0.9 eq.) and TEA (15 mg, 153 μmol, 3.0 eq.). The resulting mixture was stirred at room temperature for 2 hrs under N₂. After completion, the reaction mixture was concentrated under reduced pressure. The residue was purified by prep-HPLC to get ((2-(((3S,6S,7aS,8aR,9aR)-3-((6R)-6-(1-(5-(4-(2,6-dioxopiperidin-3-yl)phenoxy)pentyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 10, 11.1 mg, 11 μmol, 22%) as a white solid.

¹H NMR (400 MHz, DMSO-d6) δ 10.78 (s, 1H), 8.88 (d, J=7.4 Hz, 1H), 8.34 (s, 1H), 8.15-8.07 (m, 2H), 7.59 (t, J=7.1 Hz, 2H), 7.09 (d, J=8.6 Hz, 2H), 6.84 (d, J=8.7 Hz, 2H), 6.33 (s, 1H), 6.24-6.19 (m, 1H), 4.97-4.89 (m, 1H), 4.56-4.51 (m, 1H), 4.18-4.11 (m, 1H), 4.07-4.01 (m, 1H), 3.93-3.88 (m, 2H), 3.85-3.73 (m, 4H), 3.39-3.30 (m, 1H), 2.69-2.59 (m, 1H), 2.49-2.42 (m, 1H), 2.38-2.30 (m, 1H), 2.16-1.96 (m, 8H), 1.90-1.77 (m, 2H), 1.73-1.52 (m, 6H), 1.41-1.30 (m, 3H), 0.86-0.70 (m, 2H), 0.51-0.36 (m, 2H), 0.03-−0.06 (m, 2H).

LCMS (ESI): m/z=974.6[M+H]⁺.

### Preparation of ((2-(((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 11) and S,S′-(((((2-(((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 12)

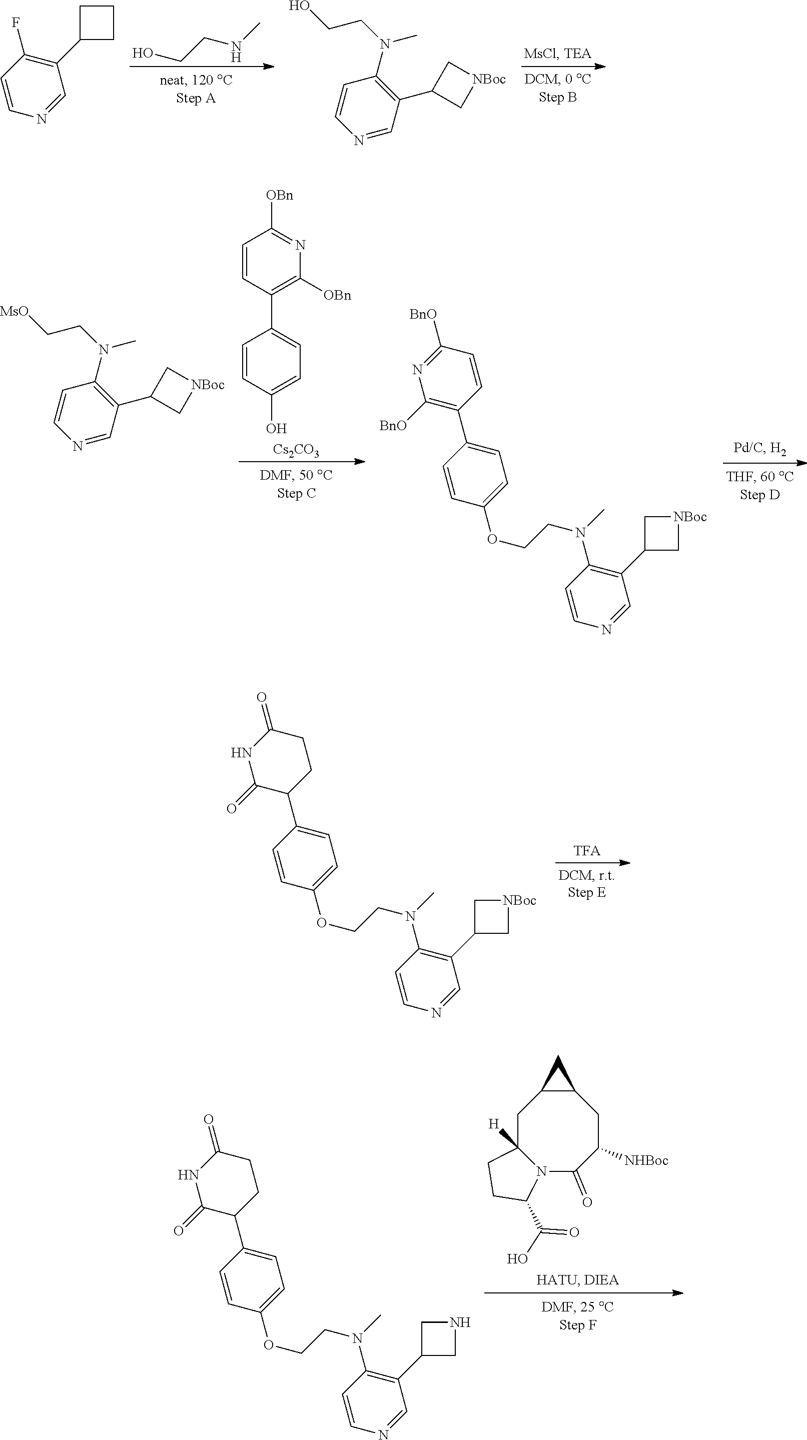

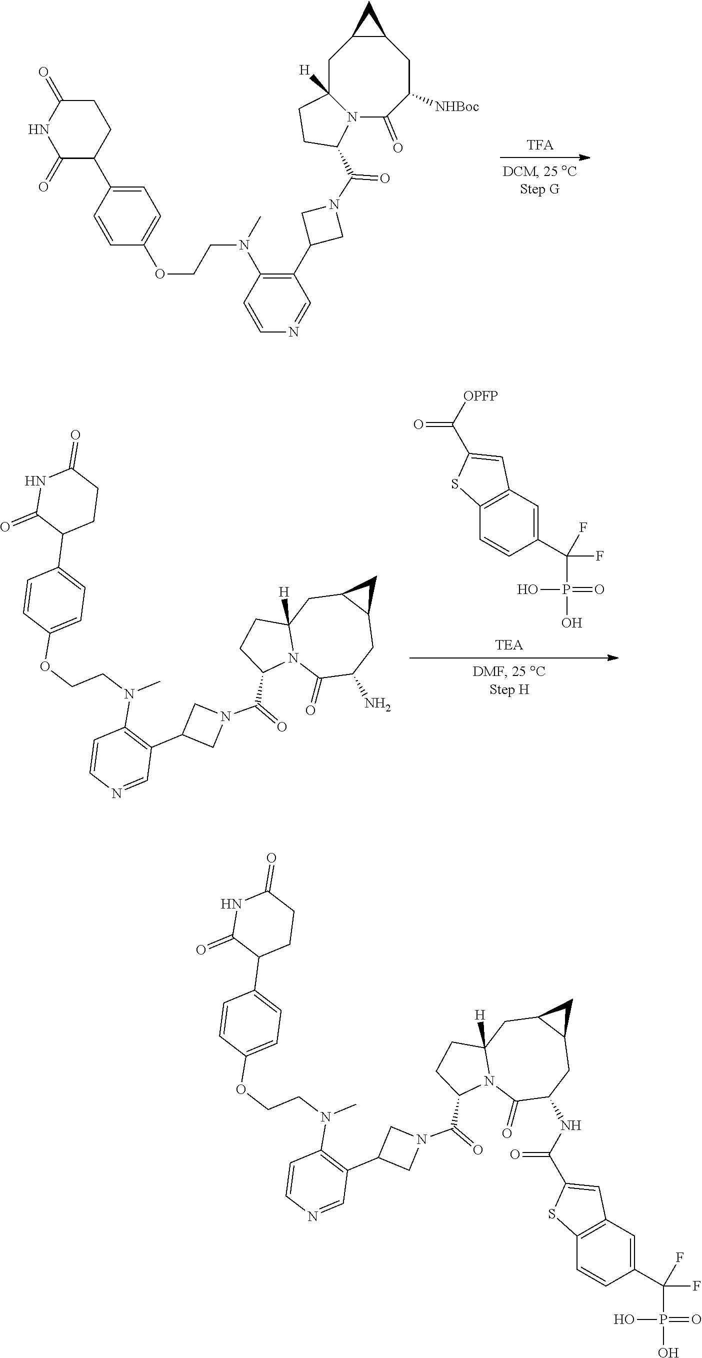

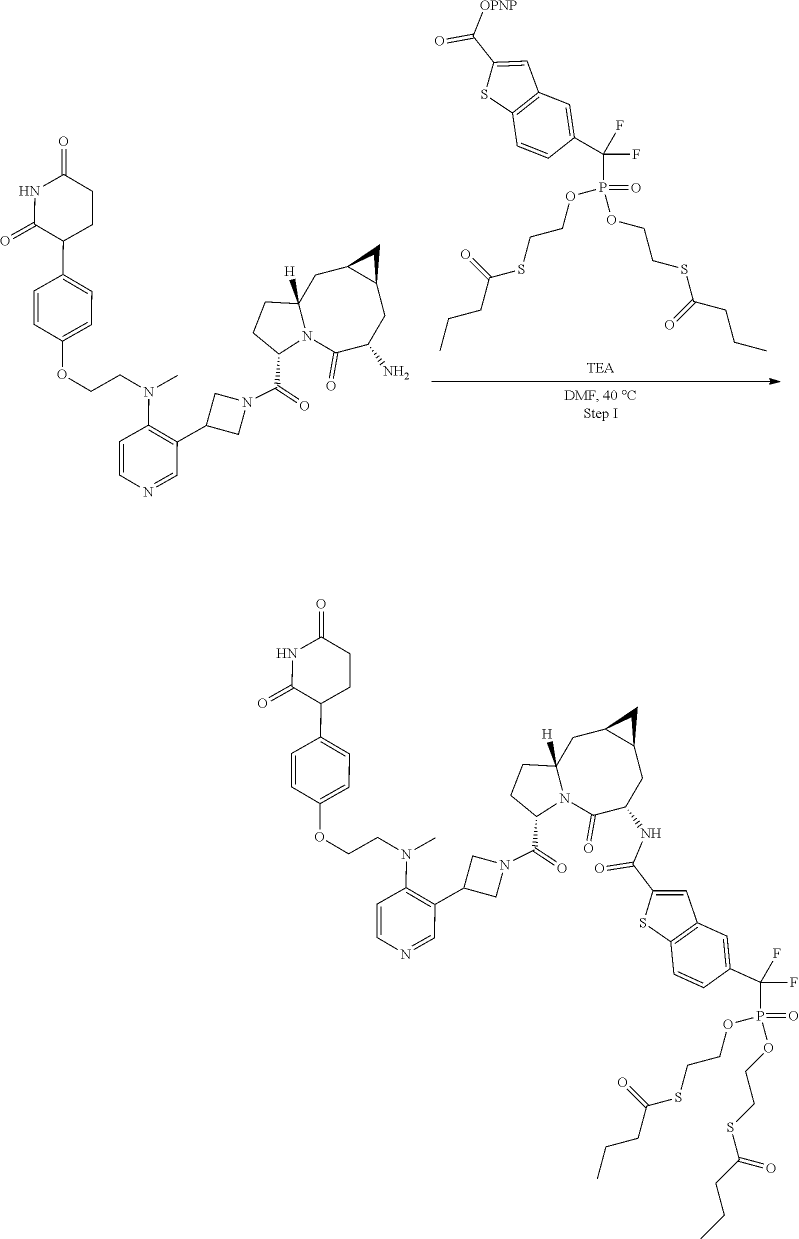

### STEP A: tert-butyl 3-(4-((2-hydroxyethyl)(methyl)amino)pyridin-3-yl)azetidine-1-carboxylate

2-Methylpropan-2-yl 3-(4-fluoropyridin-3-yl) azetidine-1-carboxylate (500 mg, 1.98 mmol, 1.0 eq) was added to a solution of 2-(methylamino) ethan-1-ol (1.5 g, 19.8 mmol, 10.0 eq.) at 25° C. The resulting mixture was heated to 120° C. and stirred for 12 his. After completion, the reaction mixture was purified by Biotage® C18 column chromatography to get tert-butyl 3-(4-((2-hydroxyethyl)(methyl)amino)pyridin-3-yl)azetidine-1-carboxylate (550 mg, 1.79 mmol, 90%) as a yellow oil. LCMS (ESI): m/z=308 [M+H]⁺.

¹H NMR (400 MHz, CDCl₃) δ 8.51 (s, 1H), 8.31 (d, J=5.6 Hz, 1H), 6.88 (d, J=5.6 Hz, 1H), 4.34 (t, J=8.1 Hz, 2H), 4.11-3.97 (m, 3H), 3.79 (t, J=5.7 Hz, 2H), 3.13 (t, J=5.7 Hz, 2H), 2.78 (s, 3H), 2.57 (br, 1H), 1.46 (s, 9H).

### STEP B: tert-butyl 3-(4-(methyl(2-((methylsulfonyl)oxy)ethyl)amino)pyridin-3-yl)azetidine-1-carboxylate

To a solution of tert-butyl 3-(4-((2-hydroxyethyl)(methyl)amino)pyridin-3-yl)azetidine-1-carboxylate (550 mg, 1.79 mmol, 1.0 eq.) in DCM (10 mL) were added TEA (0.5 mL, 3.58 mmol, 2.0 eq.) and MsCl (246 mg, 2.15 mmol, 1.2 eq.) at 0° C. and the resulting mixture was stirred at 0° C. for 1 hr. After completion, the reaction mixture was diluted with H₂O (10 mL) and extracted with DCM (20 mL×2). The combined organic layers was washed with brine (10 mL), dried over anhydrous Na₂SO₄ and concentrated under reduced pressure to afford crude tert-butyl 3-(4-(methyl(2-((methylsulfonyl)oxy)ethyl)amino)pyridin-3-yl)azetidine-1-carboxylate (550 mg, quant.) as a yellow oil, which was used in next step directly without further purification. LCMS (ESI): m/z=386 [M+H]⁺.

### STEP C: tert-butyl 3-(4-((2-(4-(2,6-bis(benzyloxy)pyridin-3-yl)phenoxy)ethyl)(methyl)amino)pyridin-3-yl)azetidine-1-carboxylate

To a solution of tert-butyl 3-(4-(methyl(2-((methylsulfonyl)oxy)ethyl)amino)pyridin-3-yl)azetidine-1-carboxylate (550 mg, 1.43 mmol, 1.0 eq) in DMF (10 mL) were added 4-(2,6-bis(benzyloxy)pyridin-3-yl)phenol (438 mg, 1.14 mmol, 0.8 eq.) and Cs₂CO₃ (930 mg, 2.85 mmol, 2.0 eq.) at 25° C. The mixture was heated to 50° C. and stirred for 12 hrs. After completion, the reaction mixture was diluted with H₂O (10 mL) and extracted with EtOAc (20 mL×2). The combined organic layers were washed with brine (10 mL), dried over anhydrous Na₂SO₄ and concentrated under reduced pressure. The residue was purified by flash column chromatography on silica gel to afford tert-butyl 3-(4-((2-(4-(2,6-bis(benzyloxy)pyridin-3-yl)phenoxy)ethyl)(methyl)amino)pyridin-3-yl)azetidine-1-carboxylate (550 mg, 0.82 mmol, 57%) as a yellow oil. LCMS (ESI): m/z=673 [M−H]⁺.

### STEP D: tert-butyl 3-(4-((2-(4-(2,6-dioxopiperidin-3-yl)phenoxy)ethyl)(methyl)amino)pyridin-3-yl)azetidine-1-carboxylate

To a solution of tert-butyl 3-(4-((2-(4-(2,6-bis(benzyloxy)pyridin-3-yl)phenoxy)ethyl)(methyl)amino)pyridin-3-yl)azetidine-1-carboxylate (400 mg, 0.60 mmol 1.0 eq.) in THF (30 mL) was added Pd/C (50 mg) under nitrogen. The suspension was degassed under vacuum and purged with H₂ several times. The resulting mixture was heated to 60° C. and stirred for 12 hrs under H₂ (15 Psi). After completion, the suspension was filtered through a pad of Celite®, the filter cake was washed with THF (40 mL). The combined filtrates were concentrated under reduced pressure. The residue was purified by Biotage® C18 column chromatography to give tert-butyl 3-(4-((2-(4-(2,6-dioxopiperidin-3-yl)phenoxy)ethyl)(methyl)amino)pyridin-3-yl)azetidine-1-carboxylate (220 mg, 0.45 mmol, 75%) as a yellow oil. LCMS (ESI): m/z=495 [M+H]⁺.

¹H NMR (400 MHz, CDCl₃) δ 8.49 (s, 1H), 8.34 (d, J=5.6 Hz, 1H), 7.98 (s, 1H), 7.11 (d, J=8.6 Hz, 2H), 6.88 (d, J=5.6 Hz, 1H), 6.80 (d, J=8.7 Hz, 2H), 4.38-4.32 (m, 2H), 4.12-4.01 (m, 5H), 3.75-3.69 (m, 1H), 3.41-3.35 (m, 2H), 2.85 (s, 3H), 2.79-2.61 (m, 2H), 2.31-2.16 (m, 2H), 1.46 (s, 9H).

### STEP E: 3-(4-(2-((3-(azetidin-3-yl)pyridin-4-yl)(methyl)amino)ethoxy)phenyl)piperidine-2,6-dione

To a solution of tert-butyl 3-(4-((2-(4-(2,6-dioxopiperidin-3-yl)phenoxy)ethyl)(methyl)amino)pyridin-3-yl)azetidine-1-carboxylate (100 mg, 0.20 mmol, 1.0 eq.) in dry DCM (6 mL) was added TFA (2 mL) at 25° C. The mixture was stirred at 25° C. for 30 min. After completion, the reaction mixture was concentrated under reduced pressure to give crude (3-{4-[(2-{[3-(azetidin-3-yl)pyridin-4-yl](methyl)amino}ethyl)oxy]phenyl}hexahydropyridine-2,6-dione (80 mg, quant.) as a yellow oil, which was used in next step directly without further purification. LCMS (ESI): m/z=395 [M+H]⁺.

### STEP F: tert-butyl ((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamate

To a mixture of (3S,6S,7aS,8aR,9aR)-6-({[(2-methylprop-2-yl)oxy]carbonyl}amino)-5-oxo-1,2,3,5,6,7,7a,8a,9,9a-decahydrocyclopropa[1,2-d]pyrrolo[1,2-a]azocine-3-carboxylic acid (75 mg, 0.22 mmol, 1.0 eq.) and HATU (85 mg, 0.22 mmol, 1.1 eq.) in DMF (3 mL) were added DIEA (131 mg, 1.01 mmol, 5.0 eq.) and 3-{4-[(2-{[3-(azetidin-3-yl)pyridin-4-yl](methyl)amino}ethyl)oxy]phenyl}hexahydropyridine-2,6-dione (80 mg, 0.20 mmol, 1.0 eq.). The resulting mixture was stirred at 25° C. for 30 min. After completion, the reaction mixture was purified directly by Biotage® C18 column chromatography to afford tert-butyl ((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamate (100 mg, 0.14 mmol, 69%) as a yellow oil. LCMS (ESI): m/z=715 [M+H]⁺.

### STEP G: 3-(4-(2-((3-(1-((3S,6S,7aS,8aR,9aR)-6-amino-5-oxodecahydro-1H-cyclopropa[d]pyrrolo[1,2-a]azocine-3-carbonyl)azetidin-3-yl)pyridin-4-yl)(methyl)amino)ethoxy)phenyl)piperidine-2,6-dione

To a solution of tert-butyl ((3S,6S,7aS,8aR,9aR)-3-(3-(4-((2-(4-(2,6-dioxopiperidin-3-yl)phenoxy-)ethyl)(methyl)amino)pyridin-3-yl)azetidine-1-carbonyl)-5-oxodecahydro-1H-cyclopropa[d]pyrrolo[1,2-a]azocin-6-yl)carbamate (100 mg, 0.14 mmol, 1.0 eq.) in dry DCM (3 mL) was added TFA (1 mL) at 25° C. The mixture was stirred at 25° C. for 30 min. After completion, the reaction mixture was concentrated under reduced pressure to give crude 3-(4-(2-((3-(1-((3S,6S,7aS,8aR,9aR)-6-amino-5-oxodecahydro-1H-cyclopropa[d]pyrrolo[1,2-a]azocine-3-carbonyl)azetidin-3-yl)pyridin-4-yl)(methyl)amino)ethoxy)phenyl)piperidine-2,6-dione (80 mg, crude) as a yellow oil, which was used in next step directly without further purification. LCMS (ESI): m/z=615 [M+H]⁺.

### STEP H: ((2-(((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamyl)benzo[b]thiophen-5-yl)difluoromethyl)phosphonic acid

To a mixture of 3-(4-(2-((3-(1-((3S,6S,7aS,8aR,9aR)-6-amino-5-oxodecahydro-1H-cyclopropa[d]pyrrolo[1,2-a]azocine-3-carbonyl)azetidin-3-yl)pyridin-4-yl)(methyl)amino)ethoxy)phenyl)piperidine-2,6-dione (40 mg, 65 μmol, 1.0 eq.) and TEA (20 mg, 195 μmol, 3.0 eq.) in DMF (3 mL) was added [difluoro(2-{[(2,3,4,5,6-pentafluorophenyl)oxy]carbonyl}benzo[b]thiophen-5-yl)methyl]phosphonic acid (29 mg, 61 μmol, 0.95 eq.). The mixture was stirred at 25° C. for 30 min. After completion, the reaction mixture was purified by prep-HPLC to give ((2-(((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 11, 15 mg, 17 μmol, 25%) as a white solid.

¹H NMR (400 MHz, DMSO-d6) δ 10.78 (s, 1H), 8.87 (t, J=6.4 Hz, 1H), 8.40-8.20 (m, 3H), 8.14-8.03 (m, 2H), 7.60 (d, J=8.6 Hz, 1H), 7.28-7.18 (m, 1H), 7.09-7.01 (m, 2H), 6.74 (d, J=8.6 Hz, 2H), 4.95-4.88 (m, 1H), 4.60-4.46 (m, 1H), 4.32-4.28 (m, 1H), 4.22-4.15 (m, 4H), 3.95-3.92 (m, 1H), 3.80-3.71 (m, 5H), 3.11 (d, J=6.6 Hz, 3H), 2.68-2.53 (m, 2H), 2.41-2.27 (m, 1H), 2.16-1.57 (m, 8H), 1.34 (s, 1H), 0.89-0.65 (m, 2H), 0.06-−0.06 (m, 1H).

LCMS (ESI): m/z=905.6 [M+H]⁺.

### STEP I: S,S′-(((((2-(((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a mixture of 3-{4-[(2-{[3-(1-{[(3S,6S,9aS)-6-amino-5-oxo-2,3,5,6,7,8,9,9a-octahydro-1H-pyrrolo[1,2-a]azepin-3-yl]carbonyl}azetidin-3-yl)pyridin-4-yl](methyl)amino}ethyl)oxy]phenyl}hexahydropyridine-2,6-dione (40 mg, 65 μmol, 1.0 eq.) and TEA (20 mg, 0.20 mmol, 3.0 eq.) in DMF (3 mL was added 4-nitrophenyl 5-((bis(2-(butyrylthio)ethoxy)phosphoryl)difluoromethyl)benzo[b]thiophene-2-carboxylate (45 mg, 65 μmol, 0.95 eq.). The mixture was stirred at 40° C. for 4 hrs. After completion, the reaction mixture was purified by prep-HPLC to give S,S′-(((((2-(((3S,6S,7aS,8aR,9aR)-3-(3-(4-((2-(4-(2,6-dioxopiperidin-3-yl)phenoxy)ethyl)(methyl)amino)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate. (Compound 12, 25.7 mg, 22 μmol, 32%) as a white solid.

¹H NMR (400 MHz, DMSO-d6) δ 10.78 (s, 1H), 8.98-8.86 (m, 1H), 8.40-8.36 (m, 1H), 8.34-8.27 (m, 2H), 8.24-8.11 (m, 2H), 7.61-7.56 (m, 1H), 7.29-7.21 (m, 1H), 7.09-7.01 (m, 2H), 6.78-6.67 (m, 2H), 4.97-4.56 (m, 2H), 4.48-4.27 (m, 2H), 4.25-4.08 (m, 9H), 4.01-3.80 (m, 4H), 3.19-3.06 (m, 7H), 2.70-2.59 (m, 1H), 2.58-2.51 (m, 4H), 2.46-2.24 (m, 2H), 2.17-1.77 (m, 8H), 1.68-1.50 (m, 5H), 1.41-1.30 (m, 1H), 0.89-0.67 (m, 8H), 0.06-−4.07 (m, 1H).

LCMS (ESI): m/z=1165.6 [M+H]⁺.

### Preparation of ((2-(((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 13) and S,S′-(((((2-(((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 14)

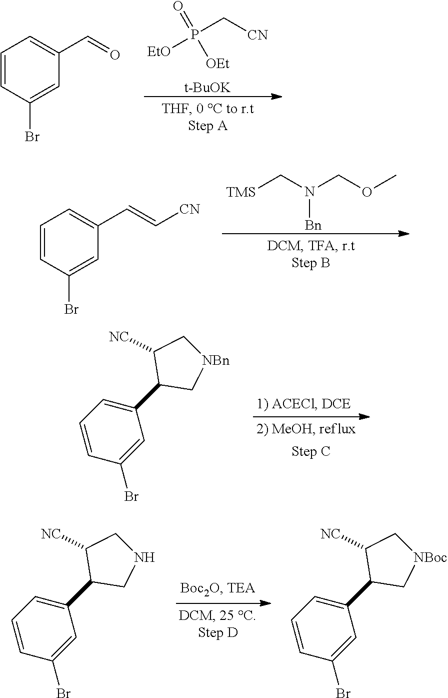

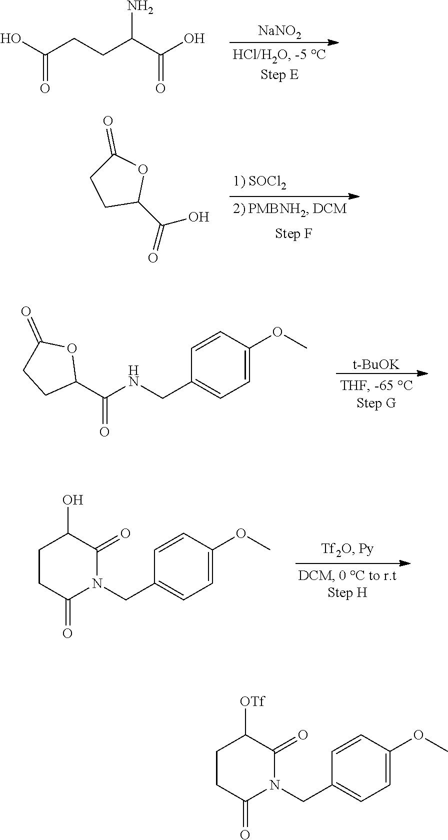

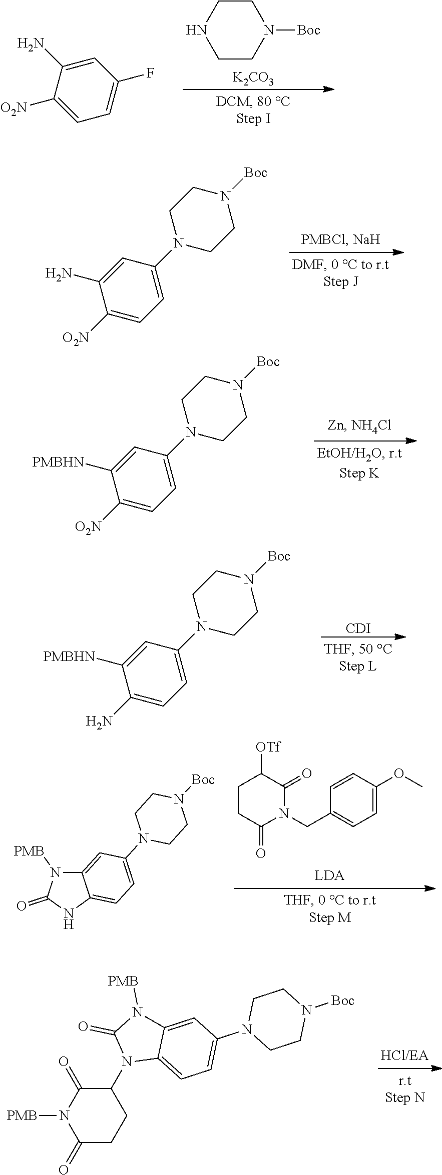

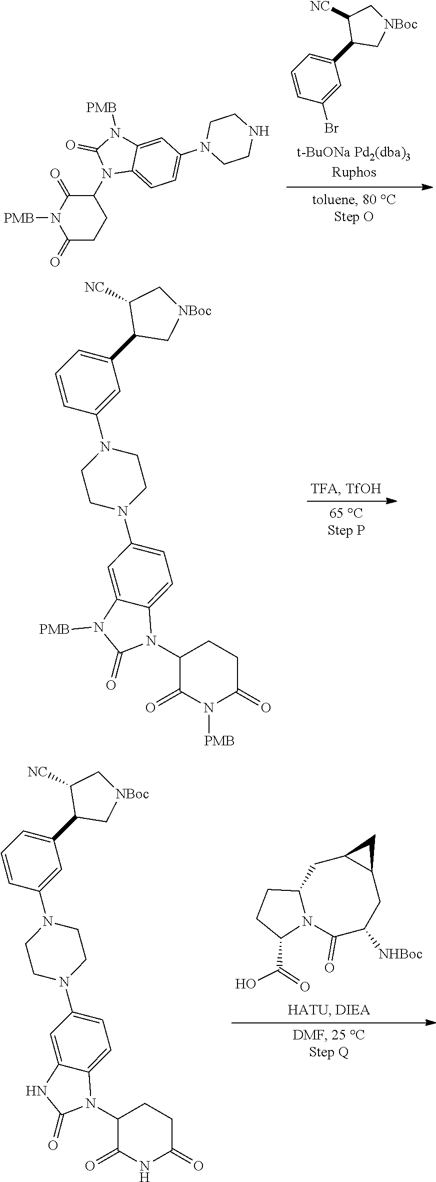

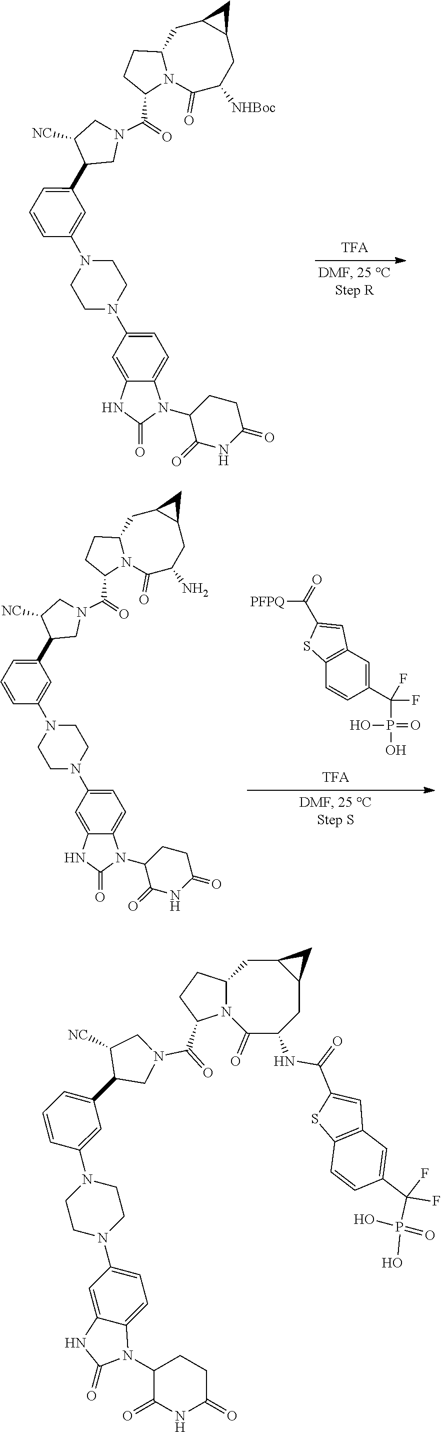

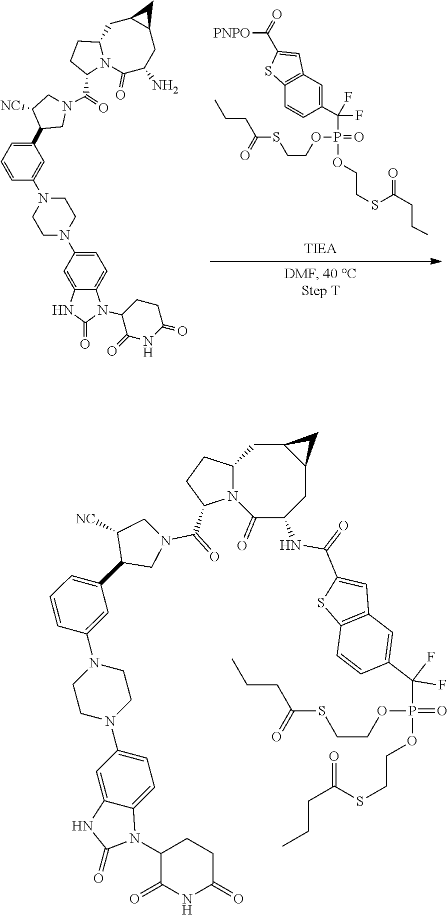

### STEP A: (E)-3-(3-bromophenyl) acrylonitrile

To a solution of diethyl (cyanomethyl)phosphonate (9.56 g. 54.0 mmol, 1 eq.) in THF (50 mL) was added (tert-butoxy) potassium (6.05 g, 54.0 mmol, 1 eq) at 0° C. The solution was stirred at room temperature for 1 hr, then 3-bromobenzaldehyde (10 g, 54.0 mmol, 1 eq.) was added to the solution, the solution was stirred at room temperature for additional 1 hr. After completion, H₂O (100 mL) was added to the solution, the solution was extracted with EtOAc (200 mL×3). The organic layers were combined and washed with brine (100 mL), dried over anhydrous Na₂SO₄, and concentrated under reduced pressure. The residue was purified by flash column chromatography on silica gel to afford (2E)-3-(3-bromophenyl)prop-2-enenitrile (10.0 g, 4.0 mmol, 89%) as a yellow solid. LCMS (ESI): m/z=208 [M+H]⁺.

### STEP B: rac-(3S,4R)-1-benzyl-4-(3-bromophenyl)pyrrolidine-3-carbonitrile

To a solution of (2E)-3-(3-bromophenyl)prop-2-enenitrile (10 g, 48.0 mmol, 1 eq.) in DCM (100 mL) were added benzyl(methoxymethyl)[(trimethylsilyl)methyl]amine (11.3 g, 48.0 mmol, 1 eq.) and TEA (2 mL). The solution was stirred at room temperature overnight. After completion, the reaction mixture was concentrated under reduced pressure. The residue was purified by C18 column to afford rac-1-benzyl-4-(3-bromophenyl)pyrrolidine-3-carbonitrile (4.0, 11.7 mmol, 25%) as a yellow oil. LCMS (ESI): m/z=341 [M+H]⁺

### STEP C: rac-(3S,4R)-4-(3-bromophenyl)pyrrolidine-3-carbonitrile

To a solution of rac-1-benzyl-4-(3-bromophenyl)pyrrolidine-3-carbonitrile (3 g, 8.79 mmol, 1 eq.) in DCE (30 mL) was added ACEC1 (6.27 g, 43.9 mmol, 5 eq.). The solution was stirred at 90° C. overnight. After completion, the reaction mixture concentrated under reduced pressure. The residue was dissolved in MeOH (100 mL) and stirred under reflux for 3 hrs. The mixture was concentrated under reduced pressure to afford crude rac-4-(3-bromophenyl)pyrrolidine-3-carbonitrile (2.0 g, 7.96 mmol, 91%) as a yellow oil. LCMS (ESI): m/z=251 [M+H]⁺.

### STEP D: rac-tert-butyl (3R,4S)-3-(3-bromophenyl)-4-cyanopyrrolidine-1-carboxylate

To a solution of rac-4-(3-bromophenyl)pyrrolidine-3-carbonitrile (0.5 g, 5.27 mmol, 1 eq.) in DCM (30 mL) were added Boc₂O (0.9, 11.9 mmol, 2 eq.) and TEA (1.8 g, 17.9 mmol, 3 eq.) The solution was stirred at 25° C. overnight. After completion, the reaction mixture concentrated under reduced pressure. The residue was purified by flash column chromatography on silica gel to afford crude rac-tert-butyl 3-(3-bromophenyl)-4-cyanopyrrolidine-1-carboxylate (1.5 g, 4.27 mmol, 72%) as a yellow oil. LCMS (ESI): m/z=351 [M+H]⁺.

### STEP E: 5-oxotetrahydrofuran-2-carboxylic acid

To a solution of glutamic acid (21 g, 143 mmol, 1.0 eq.) in H₂O (80 mL) and HCl (12 M, 21 mL) was added a solution of NaNO₂ (14.7 g, 2.13 mol, 15.0) in H₂O (40 mL) at −5° C. And the resulting mixture was stirred for 12 hrs. After completion, the mixture was concentrated under reduced pressure and then dissolved in EtOAc (50 mL). The suspension was filtered and the filter cake was washed with EtOAc (10 mL×3). The combined filtrate was dried over anhydrous Na₂SO₄, then concentrated under reduced pressure to afford crude 5-oxotetrahydrofuran-2-carboxylic acid (10 g, quant.) as a colorless oil, which was used directly in next step without further purification.

### STEP F: N-(4-methoxybenzyl)-5-oxotetrahydrofuran-2-carboxamide

To a solution of 5-oxotetrahydrofuran-2-carboxylic acid (0 g, 76.8 mmol, 1.0 eq.) in DCM (100 mL) was added SOCl₂ (45.5 g, 383 mmol, 5.0 eq.). The solution was stirred at room temperature for 2 hrs. then concentrated under vacuum. The residue was dissolved in DCM (100 mL), then 1-(4-methoxyphenyl)methanamine (10.5 g, 76.8 mmol, 1.0 eq.) and TEA (15.4 g, 153 mmol, 2.0 eq) were added to the solution. The solution was stirred at room temperature overnight. After completion, the mixture was concentrated under reduced pressure. The residue was purified by flash column chromatography on silica gel to afford N-[(4-methoxyphenyl)methyl]-5-oxooxolane-2-carboxamide (10.0 g, 40.1 mmol, 52%) as a yellow solid. LCMS (ESI): m/z=250 [M+H]⁺

### STEP G: 3-hydroxy-1-(4-methoxybenzyl)piperidine-2,6-dione

To a solution of N-[(4-methoxyphenyl)methyl]-5-oxooxolane-2-carboxamide (10 g, 40.1 mmol, 1 eq.) in THF (100 mL) was added (tert-butoxy)potassium (4.49 g, 40.1 mmol, 1 eq.) at −65° C. The solution was stirred at −65° C. for 4 hrs. After completion, the reaction mixture was quenched by adding H₂O (100 mL), then extracted with EtOAc (100 mL×3). The organic layers were combined and washed with brine (100 mL), dried over anhydrous Na₂SO₄, and concentrated under reduced pressure. The residue was purified by flash column chromatography on silica gel to afford 3-hydroxy-1-[(4-methoxy phenyl)methyl]piperidine-2,6-dione (5.0 g, 20.0 mmol, 50%) as a colorless oil LCMS (ESI): m/z=250 [M+H]⁺.

### STEP H: 1-(4-methoxybenzyl)-2,6-dioxopiperidin-3-yl trifluoromethanesulfonate

To a solution of 3-hydroxy-1-[(4-methoxyphenyl)methyl]piperidine-2,6-dione (5 g, 20.0 mmol, 1 eq.) in DCM (50 mL) were added pyridin (3.16 g, 40.0 mmol, 2 eq.) and trifluoromethanesulfonic acid anhydride (6.28 g, 20.0 mmol, 1 eq.) at 0° C. The resulting solution was stirred at room temperature overnight. After completion, the mixture was concentrated under reduced pressure. The residue was purified by flash column chromatography on silica gel to afford 1-[(4-methoxyphenyl)methyl]-2,6-dioxopiperidin-3-yl trifluoromethanesulfonate (4.0 g, 10.4 mmol, 52%) as a white solid. LCMS (ESI): m/z=382 [M+H]⁺.

### STEP I: tert-butyl 4-(3-amino-4-nitrophenyl)piperazine-1-carboxylate

To a solution of 5-fluoro-2-nitroaniline (3 g, 19.2 mmol, 1 eq.) in DMF (25 mL) were added K₂CO₃ (5.29 g, 38.4 mmol, 2 eq.) and tert-butyl piperazine-1-carboxy late (3.57 g, 19.2 mmol, 1 eq.). The solution was stirred at 80° C. overnight. After completion, the mixture was poured into H₂O (500 mL), the product was collected by filtration to afford tert-butyl 4-(3-amino-4-nitrophenyl)piperazine-1-carboxylate (5.00 g, 15.5 mmol, 81%) as a yellow solid. LCMS (ESI): m/z=323 [M+H]⁺.

### STEP J: tert-butyl 4-(3-((4-methoxybenzyl)amino)-4-nitrophenyl)piperazine-1-carboxylate

To a solution of tert-butyl 4-(3-amino-4-nitrophenyl)piperazine-1-carboxylate (3 g, 9.30 mmol, 1 eq.) in DMF (30 mL) was added NaH (60 wt %) (446 mg, 18.6 mmol, 2 eq.) at 0° C., the solution was stirred at 0° C. for 30 min, then 1-(chloromethyl)-4-methoxybenzene (1.45 g, 9.30 mmol, 1 eq.) was added to the solution. The reaction mixture was warmed up to room temperature and stirred for 1 hr. After completion, the reaction mixture was quenched by adding H₂O (100 mL), then extracted with EtOAc (100 mL×3). The organic layers were combined and washed with brine (100 mL), dried over anhydrous Na₂SO₄, and concentrated under reduced pressure. The residue was purified by flash column chromatography on silica gel to afford tert-butyl 4-(3-{[(4-methoxyphenyl)methyl]amino}-4-nitrophenyl)piperazine-1-carboxylate (3.0 g, 6.77 mmol, 73%) as a yellow solid. LCMS (ESI): m/z=443 [M+H]⁺.

### STEP K: tert-butyl 4-(4-amino-3-((4-methoxybenzyl)amino)phenyl)piperazine-1-carboxylate

To a solution of tert-butyl 4-(3-{[(4-methoxyphenyl)methyl]amino}-4-nitrophenyl)piperazine-1-carboxylate (kg, 4.51 mmol, 1 eq.) in EtOH (30 mL) and saturated NH₄Cl (30 mL, aq.) was added zinc (2.94 g, 45.1 mmol, 10 eq.), the solution was stirred at room temperature for 3 hrs. After completion, the suspension was filtered through a pad of Celite®, the filter cake was washed with MeOH (20 mL). The combined filtrates were concentrated under reduced pressure. The residue was dissolved in H₂O (30 mL) and extracted with EtOAc (50 mL×3). The organic layers were combined and washed with brine (100 mL), dried over anhydrous Na₂SO₄, and concentrated under reduced pressure. The residue was purified by flash column chromatography on silica gel to afford tert-butyl 4(4-amino-3-{[(4-methoxyphenyl)methyl]amino}phenyl)piperazine-1-carboxylate (1.80 g, 4.36 mmol, 97%) as a yellow solid. LCMS (ESI): m/z=413 [M+H]⁺.

### STEP L: tert-butyl 4-(3-(4-methoxybenzyl)-2-oxo-2,3-dihydro-1H-benzo[d]imidazol-5-yl)piperazine-1-carboxylate

To a solution of tert-butyl 4-(4-amino-3-{[(4-methoxyphenyl)methyl]amino}phenyl)piperazine-1-carboxylate (1.5 g, 3.63 mmol 1 eq.) in THF (20 mL) was added CDI (1.17 g, 7.26 mmol, 2 eq.). The solution was stirred at 50° C. for 1 hr. The mixture was concentrated under reduced pressure. The residue was purified by flash column chromatography on silica gel to afford tert-butyl 4-{3-[(4-methoxyphenyl)methyl]-2-oxo-2,3-dihydro-1H-1,3-benzodiazol-5-yl}piperazine-1-carboxylate (1.50 g, 3.42 mmol, 94%) as a yellow solid. LCMS (ESI): m/z=439 [M+H]⁺.

### STEP M: tert-butyl 4-(3-(4-methoxybenzyl)-2-oxo-2,3-dihydro-1H-benzo[d]imidazol-5-yl)piperazine-1-carboxylate

To a solution of tert-butyl 4-{3-[(4-methoxyphenyl)methyl]-2-oxo-2,3-dihydro-1H-1,3-benzodiazol-5-yl}piperazine-1-carboxylate (1 g, 2.28 mmol, 1 eq.) in THF (10 mL) was added LDA (1.2 mL, 2 M in THF, 1.05 eq.) at 0° C. and the mixture was stirred at 0° C. for 1 hr, then 1-[(4-methoxyphenyl)methyl]-2,6-dioxopiperidin-3-yl trifluoromethanesulfonate (869 mg, 2.28 mmol, 1 eq.) was added to the solution. The solution was warmed up to room temperature and stirred for 1 hr. After completion, the reaction mixture was quenched by adding H₂O (20 mL), then extracted with EtOAc (50 mL×3). The organic layers were combined and washed with brine (20 mL), dried over anhydrous Na₂SO₄, and concentrated under reduced pressure. The residue was purified by flash column chromatography on silica gel to afford tert-butyl 4-{3-(4-methoxyphenylmethyl-1-{1-[(4-methoxyphenyl)methyl]-2,6-dioxopiperidin-3-yl}-2-oxo-2,3-dihydro-1H-1,3-benzodiazol-5-yl}piperazine-1-carboxylate (1 g, 1.49 mmol, 16%) as a yellow solid. LCMS (ESI): m/z=670 [M+H]⁺.

### STEP N: 1-(4-methoxybenzyl)-3-(3-(4-methoxybenzyl)-2-oxo-5-(piperazin-1-yl)-2,3-dihydro-1H-benzo[d]imidazol-1-yl)piperidine-2,6-dione

A solution of tert-butyl 4-{3-[(4-methoxyphenyl)methyl]-1-{1-[(4-methoxyphenyl)methyl]-2,6-dioxopiperidin-3-yl}-2-oxo-2,3-dihydro-1H-1,3-benzodiazol-5-yl}piperazine-1-carboxylate (1 g, 1.49 mmol, 1 eq.) in HCl/Ethyl Acetate (2 mL, 3 M) was stirred at room temperature for 1 hr. After completion, the reaction mixture was concentrated under reduced pressure. The residue was dissolved in H₂O (10 mL), cooled down in an ice bath, then neutralized carefully with NaHCO₃ (aq.) until the pH was adjusted to pH=9. The resulting mixture was extracted with DCM (20 mL×3), and the combined organic layers were washed with brine (10 mL), dried over with anhydrous Na₂SO₄, then concentrated under reduced pressure to give crude 1-[(4-methoxyphenyl)methyl]-3-{3-[(4-methoxyphenyl)methyl]-2-oxo-5-(piperazin-1-yl)-2,3-dihydro-1H-1,3-benzodiazol-1-yl}piperidine-2,6-dione (800 mg, quant.) as a blue solid, which was used in next step directly without further purification. LCMS (ESI): m/z=570 [M+H]⁺.

### STEP O: tert-butyl (rel-3S,4R)-3-cyano-4-(3-(4-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperazin-1-yl)phenyl)pyrrolidine-1-carboxylate

To a solution of 1-[(4-methoxyphenyl)methyl]-3-{3-[(4-methoxyphenyl)methyl]-2-oxo-5-(piperazin-1-yl)-2,3-dihydro-1H-1,3-benzodiazol-1-yl}piperidine-2,6-dione (500 mg, 877 μmol, 1 eq.) in toluene (0 mL) were added rac-tert-butyl 3-(3-bromophenyl)-4-cyanopyrrolidine-1-carboxylate (308 mg, 877 μmol, 1 eq.), Pd₂(dba)₃ (80 mg, 88 μmol, 0.1 eq.), Ruphos (82 mg, 175 μmol, 0.2 eq.) and t-BuONa (168 mg, 1.75 mmol, 2 eq.). The solution was stirred at 80° C. for 2 hrs under N₂. After completion, the solution was concentrated under reduced pressure. The residue was purified by C18 column to afford tert-butyl (rel-3S,4R)-3-cyano-4-(3-(4-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperazin-1-yl)phenyl)pyrrolidine-1-carboxylate (100 mg, 119 μmol, 14%) as a white solid LCMS (ESI): m/z=840 [M+H]⁺.

### STEP P: (rel-3S,4R)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile

To a solution of tert-butyl (rel-3S,4R)-3-cyano-4-(3-(4-(3-(4-methoxy benzyl)-1-(1-(4-methoxybenzyl)-2,6-dioxopiperidin-3-yl)-2-oxo-2,3-dihydro-1H-benzo[d]imidazol-5-yl)piperazin-1-yl)phenyl)pyrrolidine-1-carboxylate (300 mg, 357 μmol, 1 eq.) in TEA (3 mL) was added trifluoromethanesulfonic acid (686 mg, 7.14 mmol, 20 eq.). The solution was stirred at 65° C. for 2 hrs. After completion, the solution was concentrated under reduced pressure. The residue was purified by C18 column to afford (rel-3S,4R)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (80 mg, 160 μmol, 45%) as a white solid. LCMS (ESI): m/z=500 [M+H]⁺.

### STEP Q: tert-butyl ((3S,6S 7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamate

To a mixture of 2-methylpropan-2-yl {[(3S,6S,7aS,8aR,9aR)-3-(methoxycarbonyl)-5-oxo-1,2,3,5,6,7,7a,8a,9,9a-decahydrocyclopropa[1,2-d]pyrrolo[1,2-a]azocin-6-yl]amino}methanoate (23 mg, 66 μmol, 1.1 eq.) and HATU (25 mg, 66 μmol, 1.1 eq.) in DMF (3 mL) were added DIEA (39 mg, 0.30 mmol, 5.0 eq.) and rac-4-(3-{4-[1-(2,6-dioxohexahydropyridin-3-yl)-2-oxo-3H-benzo[d]imidazol-5-yl]piperazin-1-yl}phenyl)tetrahydropyrrole-3-carbonitrile (30 mg, 66 μmol, 1.0 eq). The resulting mixture w as stirred at 25° C. for 30 min. After completion, the reaction mixture was purified directly by Biotage® C18 column chromatography to afford tert-butyl ((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamate (40 mg, 49 μmol, 81%) as a yellow oil. LCMS (ESI): m/z=820 [M+H]⁺.

### STEP R: (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile

To a solution of tert-butyl ((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbon 1)-5-oxodecahydro-1H-cyclopropa[d]pyrrolo[1,2-a]azocin-6-yl)carbamate (20 mg, 24 μmol, 1.0 eq.) in dry DCM (3 mL) was added TFA (1 mL) at 25° C. The mixture was stirred at 25° C. for 30 min. After completion, the reaction mixture was concentrated under reduced pressure to give (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (15 mg, crude) as a yellow oil, which was used in next step without further purification. LCMS (ESI): m/z=720 [M+H]⁺.

### STEP S: ((2-(((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a mixture of (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (16, 20 mg, 28 μmol, 1.0 eq) and TEA (84 mg, 83 μmol, 3.0 eq.) in DMF (3 mL) was added (difluoro(2-((perfluorophenoxy)carbonyl)benzo[b]thiophen-5-yl)methyl)phosphonic acid (12 mg, 25 μmol, 0.9 eq.). The mixture was stirred at 25° C. for 30 min. After completion, the reaction mixture was purified by prep-HPLC to give ((2-(((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (TFA salt) (Compound 13, 12 mg, 12 μmol, 43%) as a white solid

¹H NMR (400 MHz, DMSO-d6) δ 11.07 (s, 1H), 10.98 (s, 1H), 8.93-8.80 (m, 1H), 8.36-8.30 (m, 1H), 8.17-8.10 (m, 1H), 8.10-8.03 (m, 1H), 7.63-7.55 (m, 1H), 7.30-7.22 (m, 1H), 7.16-7.07 (m, 1H), 7.03-6.94 (m, 2H), 6.90-6.80 (m, 3H), 5.31-5.24 (m, 1H), 4.97-4.91 (m, 1H), 4.63-4.58 (m, 2H), 4.20-4.17 (m, 1H), 3.99-3.94 (m, 1H), 3.84-3.77 (m, 1H), 3.71-3.63 (m, 1H), 3.56-3.47 (m, 1H), 3.43-3.27 (m, 8H), 2.98-2.83 (m, 1H), 2.75-2.57 (m, 2H), 2.40-2.28 (m, 1H), 2.20-1.80 (m, 8H), 1.71-1.58 (m, 1H), 1.48-1.33 (m, 1H), 0.90-0.70 (m, 2H), 0.06-0.00 (m, 1H).

LCMS (ESI): m/z=1010.5 [M+H]⁺.

### STEP T: S,S′-(((((2-(((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a mixture of (rei-3S,4R)-1-((3S,6S,7aS,8aR,9aR)-6-amino-5-oxodecahydro-1H-cyclopropa[d]pyrrolo[1,2-a]azocine-3-carbonyl)-4-(3-(4-(1-(2,6-dioxopiperidin-3-yl)-2-oxo-2,3-dihydro-1H-benzo[d]imidazol-5-yl)piperazin-1-yl)phenyl)pyrrolidine-3-carbonitrile (7 mg, 10 μmol, 1.0 eq) and TEA (30 mg, 30 μmol, 3.0 eq) in DMF (3 mL) was added 4-nitrophenyl 5-((bis(2-(butyrylthio)ethoxy)phosphoryl)difluoromethyl)benzo[b]thiophene-2-carboxylate (5 mg, 10 μmol, 0.9 eq.). The mixture was stirred at 40° C. for 4 hrs. After completion, the reaction mixture was purified by prep-HPLC to give S,S′-(((((2-(((3S,6S,7aS,8aR,9aR)-3-((rel-3S,4R)-3-cyano-4-(3-(4-(1-(2,6-dioxopiperidin-3-yl)-2-oxo-2,3-dihydro-1H-benzo[d]imidazol-5-yl)piperazin-1-yl)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 14, 1.9 mg, 1 μmol, 15%) as a white solid.

¹H NMR (400 MHz, DMSO-d6). δ 11.05 (s, 1H), 10.81 (s, 1H), 8.94 (dd, J=24.8, 7.5 Hz, 1H), 8.41-8.31 (m, 1H), 8.25-8.11 (m, 2H), 7.63-7.54 (m, 1H), 7.26-7.05 (m, 2H), 6.97-6.80 (m, 3H), 6.70-6.63 (m, 2H), 5.34-5.19 (m, 1H), 5.02-4.87 (m, 1H), 4.66-4.52 (m, 1H), 4.45-3.49 (m, 10H), 3.19-3.08 (m, 8H), 2.94-2.83 (m, 1H), 2.73-2.54 (m, 4H), 2.39-2.25 (m, 2H), 2.19-1.80 (m, 9H), 1.73-1.31 (m, 9H), 0.90-0.81 (m, 8H), 0.78-0.66 (m, 1H), 0.05-−0.06 (m, 1H).

LCMS (ESI): m/z=1270.3 [M+H]⁺.

### Preparation of trans-tert-butyl 3-cyano-4-(3-hydroxyphenyl)pyrrolidine-1-carboxylate

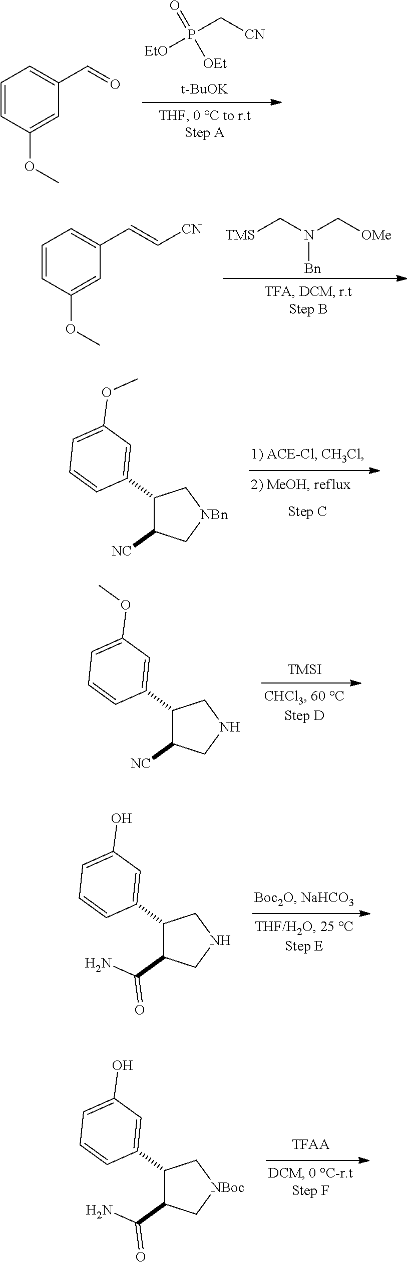

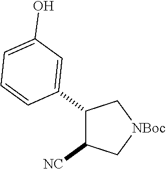 [SMILES: CC(C)(C)OC(=O)N1C[C@@H](C#N)[C@H](c2cccc(O)c2)C1]

### STEP A. (E)-3-(3-methoxyphenyl)acrylonitrile

To a solution of diethyl (cyanomethyl)phosphonate (13.7 g, 77.2 mmol, 1.05 eq.) in THF (200 mL) was added r-BuOK (9.06 g, 80.9 mmol, 1.1 eq) at 0° C., the solution was stirred for 30 min, then 3-methoxybenzaldehyde (10 g, 73.5 mmol, 1.0 eq.) was added to the solution. The solution was stirred at room temperature for 30 min. After completion, H₂O (10) mL) was added to the solution, the solution was extracted with EtOAc (200 mL×3). The organic layers were combined and washed with brine (100 mL), dried over anhydrous Na₂SO₄, and concentrated under reduced pressure. The residue was purified by flash column chromatography on silica gel to afford (E)-3-(3-methoxyphenyl)acrylonitrile (1.5 g, 72.3 mmol, 94%) as a white solid. LC-MS (ESI) m/z=160 [M+H]⁺.

### STEP B: trans-1-benzyl-4-(3-methoxyphenyl)pyrrolidine-3-carbonitrile

To a solution of N-benzyl-1-methoxy-N-((trimethylsilyl)methyl)methanamine (16 g, 69.2 mmol, 1.0 eq.) in DCM (10 mL) were added (E)-3-(3-methoxyphenyl)acrylonitrile (11 g, 69.2 mmol, 1.0 eq.) and TFA (1.58 g, 13.8 mmol, 0.2 eq.). The solution was stirred at room temperature overnight. After completion, the reaction mixture was concentrated under reduced pressure. The residue was purified by flash column chromatography on silica gel to afford trans-1-benzyl-4-(3-methoxyphenyl)pyrrolidine-3-carbonitrile (13.1 g, 44.9 mmol, 65%) as a yellow oil. LC-MS (ESI) m/z=293 [M+H]⁺.

### STEP C: trans-4-(3-methoxyphenyl)pyrrolidine-3-carbonitrile

To a solution of trans-1-benzyl-4-(3-methoxyphenyl)pyrrolidine-3-carbonitrile (10.7 g, 36.6 mmol, 1.0 eq.) in CHCl₃ (110 mL) were added ACEC1 (26.2 g, 183 mmol, 5.0 eq.), the resulting mixture was stirred at 80° C. for 16 hrs. After completion, the reaction mixture concentrated under reduced pressure. The residue was dissolved in MeOH (100 mL) and stirred at 75° C. for 3 hrs. The mixture was concentrated under reduced pressure to afford trans-4-(3-methoxyphenyl)pyrrolidine-3-carbonitrile (7.4 g, crude) as a yellow oil. LCMS (ESI): m/z=203.2 [M+H]⁺.

### STEP D: trans-4-(3-hydroxyphenyl)pyrrolidine-3-carboxamide

To a solution of trans-4-(3-methoxyphenyl)pyrrolidine-3-carbonitrile (7.4 g, 36.6 mmol, 1.0 eq.) in CHCl₃ (80 mL) were added TMSI (22 g, 110 mmol, 3.0 eq.), the resulting mixture was stirred at 60° C. for 16 hrs. After completion, the reaction mixture concentrated under reduced pressure to afford trans-4-(3-hydroxyphenyl)pyrrolidine-3-carboxamide (7.54 g, crude) as a yellow solid. LCMS (ESI): m/z=207.2 [M+H]⁺.

### STEP E: trans-tert-butyl 3-carbamoyl-4-(3-hydroxyphenyl)pyrrolidine-1-carboxylate

To a solution of trans-4-(3-hydroxyphenyl)pyrrolidine-3-carboxamide (7.2 g, 34.9 mmol, 1.0 eq.) and NaHCO₃ (16.1 g, 191.5 mmol, 5.5 eq.) in THF/H₂O (200 mL/40 mL) was added Boc₂O (16.6 g, 76.6 mmol, 2.2 eq.) at 25° C. The resulting mixture was stirred for 16 hrs. After completion, the reaction mixture was filtered and the filtrate was concentrated under reduced pressure. The residue was purified by flash column chromatography on silica gel to afford trans-tert-butyl 3-carbamoyl-4-(3-hydroxyphenyl)pyrrolidine-1-carboxylate (7.0 g, 22.9 mmol, 66%) as a yellow oil. LCMS (ESI): m/z=307.2 [M+H]⁺.

### STEP F: trans-tert-butyl 3-cyano-4-(3-hydroxyphenyl)pyrrolidine-1-carboxylate

To a solution of trans-tert-butyl 3-carbamoyl-4-(3-hydroxyphenyl)pyrrolidine-1-carboxylate (6.8 g, 22.2 mol, 1.0 eq.) in DCM (100 mL) were added TFAA (6.07 g, 28.9 mol, 1.3 eq.) dropwise at 0° C. After addition, the resulting mixture was warmed up to room temperature and stirred for 16 hrs. After completion, the reaction mixture was concentrated under reduced pressure. The residue was purified by C18 column chromatography to afford trans-tert-butyl 3-cyano-4-(3-hydroxyphenyl)pyrrolidine-1-carboxylate (5.0 g, 17.4 mmol, 78%) as a yellow oil. LCMS (ESI): m/z=289.2 [M+H]⁺.

### Preparation of ((2-(((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic add (Compound 15) and S,S′-(((((2-(((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 16)

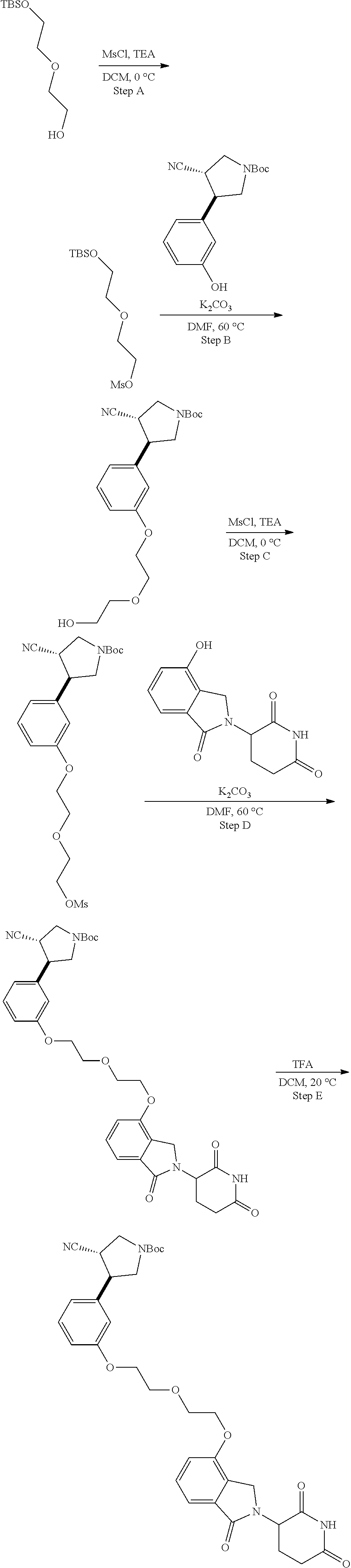

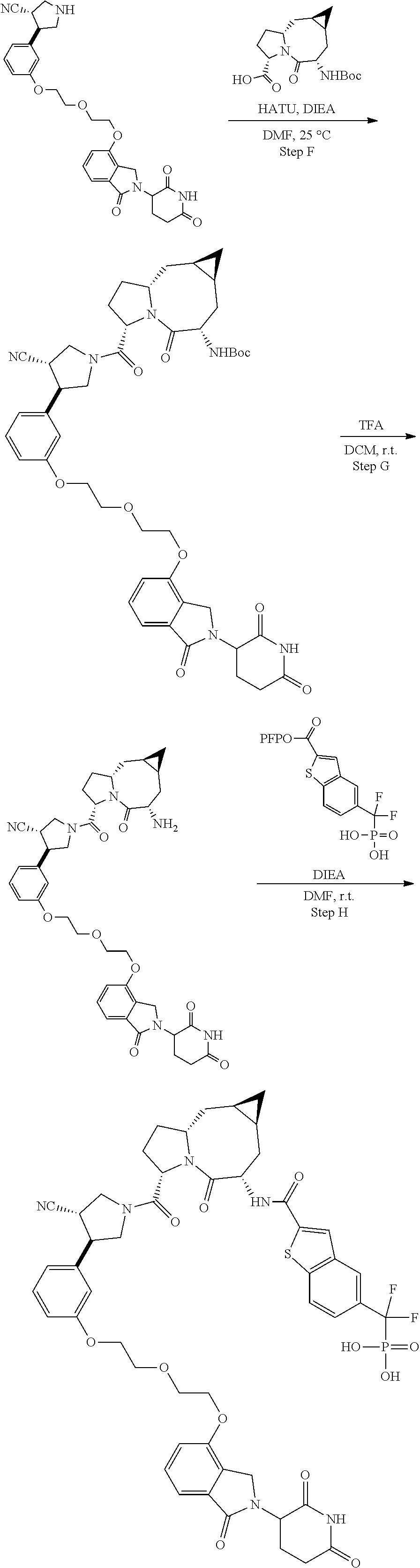

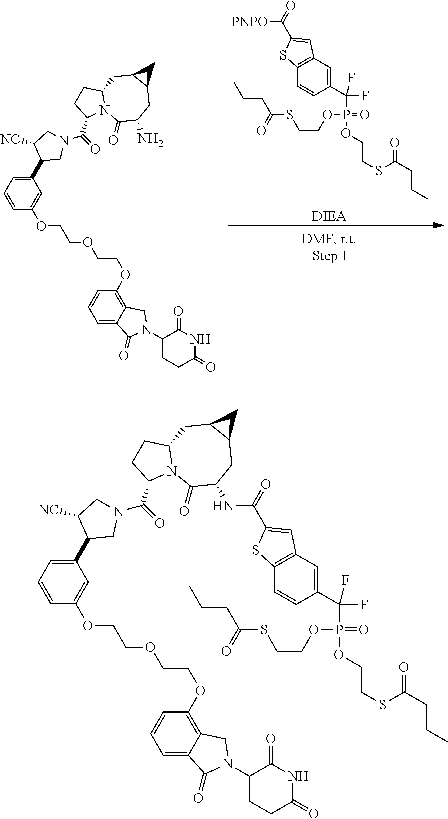

### STEP A: 2-(2-((tert-butyldimethylsilyl)oxy)ethoxy)ethyl methansulfonate

To a solution of 2-(2-((tert-butyldimethylsilyl)oxy)ethoxy)ethan-1-ol (500 mg, 2.26 mmol, 1.0 eq.) and TEA (342 mg, 3.38 mmol, 1.5 eq.) in DCM (10 mL) was added MsCl (387 mg, 3.38 mmol, 1.5 eq.) dropwise. The reaction mixture was stirred at 0° C. for 1 hr. After completion, the reaction mixture was diluted with H₂O (30 mL) and extracted with DCM (30 mL×2). The combined organic layers were washed with brine (10 mL), dried over anhydrous Na₂SO₄, then concentrated under reduced pressure to give 2-(2-((tert-butyldimethylsilyl)oxy)ethoxy)ethyl methanesulfonate (500 mg, 1.67 μmol, 74%) as a yellow liquid, which was used in next step directly without further purification. LCMS (ESI): m/z=299 [M+H]⁺.

### STEP B: rac-tert-butyl (3S,4R)-3-cyano-4-(3-(2-(2-hydroxyethoxy)ethoxy)phenyl)pyrrolidine-1-carboxylate

To a solution of 2-(2-((tert-butyldimethylsilyl)oxy)ethoxy)ethyl methanesulfonate (500 mg, 1.67 mmol, 1.0 eq.) and trans-tert-butyl 3-cyano-4-(3-hydroxyphenyl)pyrrolidine-1-carboxylate (3.5 g, 1.67 mmol, 1.0 eq.) in DMF (10 mL) was added K₂CO₃ (692 mg, 5.01 mmol, 3.0 eq.). The reaction mixture was stirred at 60° C. for 16 hrs. After completion, the reaction mixture was diluted with H₂O (30 mL) and extracted with EtOAc (30 mL×2). The combined organic layers were washed with brine (10 mL), dried over anhydrous Na₂SO₄, The residue was purified by flash column chromatography on silica gel to afford rac-tert-butyl (3S,4R)-3-cyano-4-(3-(2-(2-hydroxyethoxy)ethoxy)phenyl)pyrrolidine-1-carboxylate (260 mg, 690 μmol, 41%) as a yellow oil. LCMS (ESI): m/z=399 [M+Na]⁺.

### STEP C: rac-tert-butyl (3S,4R)-3-cyano-4-(3-(2-(2-((methylsulfonyl)oxy)ethoxy)ethoxy)phenyl)pyrrolidine-1-carboxylate

To a solution of tert-butyl (3S,4R)-3-cyano-4-(3-(2-(2-hydroxyethoxy)ethoxy)phenyl)pyrrolidine-1-carboxylate (260 mg, 690 μmol, 1.0 eq.) and TEA (104 mg, 1.03 mmol, 1.5 eq.) in DCM (5 mL) was added MsCl (117 mg, 1.03 mmol, 1.5 eq.) dropwise. The reaction mixture was stirred at 0° C. for 1 hr. After completion, the reaction mixture was diluted with H₂O (10 mL) and extracted with DCM (10 mL×2). The combined organic layers were washed with brine (10 mL), dried over anhydrous Na₂SO₄, then concentrated under reduced pressure to give crude rac-tert-butyl (3S,4R)-3-cyano-4-(3-(2-(2-((methylsulfonyl)oxy)ethoxy)ethoxy)phenyl)pyrrolidine-1-carboxylate (300 mg, quant.) as a yellow liquid, which was used in next step directly without further purification. LCMS (ESI): m/z=399 [M+H-56]⁺.

### STEP D: rac-tert-butyl (3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carboxylate

To a solution of rac-tert-butyl (3S,4R)-3-cyano-4-(3-(2-(2-((methylsulfonyl)oxy)ethoxy)ethoxy)phenyl)pyrrolidine-1-carboxylate (300 mg, 660 μmol. 1.0 eq.) and 3-(4-hydroxy-1-oxoisoindolin-2-yl)piperidine-2,6-dione (6.17 g, 660 μmol, 1.0 eq.) in DMF (10 mL) was added K₂CO₃ (273 mg, 1.98 mmol, 3.0 eq.). The reaction mixture was stirred at 60° C. for 16 hrs. After completion, the reaction mixture was diluted with H₂O (30 mL) and extracted with EtOAc (30 mL×2). The combined organic layers were washed with brine (10 mL), dried over anhydrous Na₂SO₄ and concentrated under reduced pressure. The residue was purified by Biotage®, C18 column chromatography to get rac-tert-butyl (3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carboxylate (180 mg, 0.29 mmol, 44%) as a white solid. LCMS (ESI): m/z=619 [M+H]⁺.

### STEP E: rac-(3S,4R)-4-(3-(2-(2-((2-(2,6-dioxopiperidin-3-yl)-1-oxoisoindolin-4-yl)oxy)ethoxy)ethoxy)phenyl)pyrrolidine-3-carbonitrile

To a solution of rac-tert-butyl (3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carboxylate (180 mg, 0.29 mmol, 1.0 eq.) in DCM (2 mL) was added TFA (0.6 mL) and the resulting mixture was stirred at room temperature for 2 hrs. After completion, the reaction mixture was concentrated under reduced pressure to get crude rac-(3S,4R)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (150 mg, quant.) as a colorless oil, which was used in next step directly without further purification. LCMS (ESI): m/z=519 [M+H]⁺.

### STEP F: tert-butyl ((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-JR-cyclopropa[d]pyrrolo[1,2-a]azocin-6-yl)carbamate

To a mixture of (3S,6S,7aS,8aR,9aR)-6-({[(2-methylprop-2-yl)oxy]carbonyl}amino)-5-oxo-1,2,3,5,6,7,7a,8a,9,9a-decahydrocyclopropa[1,2-dipyrrolo[1,2-a]azocine-3-carboxylic acid (59 mg, 0.17 mmol, 1 eq.) and HATU (79 mg, 0.21 mmol, 1.2 eq.) in DMF (2 mL) were added DIEA (67 mg, 0.52 mmol, 3.0 eq.) and rac-4-[3-({2-[(2-{[2-(2,6-dioxohexahydropyridin-3-yl)-1-oxo-2,3-dihydro-1H-isoindol-4-yl]oxy}ethyl)oxy]ethyl}oxy)phenyl]tetrahydropyrrole-3-carbonitrile (6, 90 mg, 0.17 mmol, 1.0 eq.). The resulting mixture was stirred at 25° C. for 2 hrs. After completion, the reaction mixture was purified directly by Biotage® C18 column chromatography to afford tert-butyl ((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamate (7, 50 mg, 60 μmol, 34%) as a white solid. LCMS (ESI): m/z=839 [M+H]⁺.

### STEP G: (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile

To a solution of tert-butyl ((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamate (50 mg, 60 μmol, 1.0 eq.) in DCM (5 mL) was added TFA (1.5 mL) and the resulting mixture was stirred at room temperature for 2 hrs. After completion, the reaction mixture was concentrated under reduced pressure to get crude (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (40 mg, quant.) as a colorless oil, which was used in next step directly without further purification. LCMS (ESI): m/z=739 [M+H]⁺.

### STEP H: ((2-(((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (20 mg, 27 μmol, 1.0 eq.) and [difluoro(2-{[(2,3,5,6-tetrafluoro-4-nitrophenyl)oxy]carbonyl}benzo[b]thiophen-5-yl)methyl]phosphonic acid (14 mg, 27 μmol, 1.0 eq.) in DMF (1 mL) was added DIEA (14 mg, 0.11 mmol, 4.0 eq.). The reaction mixture was stirred at room temperature for 2 hrs. After completion, the residue was purified by prep-HPLC to give ((2-(((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 15, 14 mg, 14 μmol, 50%) as a white solid.

¹H NMR (400 MHz, DMSO-d6) δ 10.97 (s, 1H), 8.98-8.72 (m, 1H), 8.39-8.21 (m, 1H), 8.14-8.01 (m, 2H), 7.65-7.52 (m, 1H), 7.52-7.39 (m, 1H), 7.34-7.18 (m, 3H), 7.06-6.95 (m, 2H), 6.92-6.83 (m, 1H), 5.22-5.03 (m, 1H), 5.01-4.86 (m, 1H), 4.65-4.52 (m, 1H), 4.38-4.30 (m, 1H), 4.22-4.08 (m, 4H), 3.99-3.92 (m, 1H), 3.84-3.74 (m, 4H), 3.69-3.58 (m, 2H), 3.55-3.43 (m, 2H), 3.30-3.20 (m, 1H), 2.98-2.83 (m, 1H), 2.78-2.53 (m, 1H), 2.45-1.75 (m, 11H), 1.70-1.55 (m, 1H), 1.46-1.33 (m, 11H), 0.87-0.71 (m, 2H), 0.02-0.00 (m, 1H).

LCMS (ESI): m/z=1029.6 [M+H]⁺.

### STEP I: S,S′-(((((2-(((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of (rel-3S,4R)-1-((3S,6S,7aS,8aR,9aR)-6-amino-5-oxodecahydro-1H-cyclopropa[d]pyrrolo[1,2-a]azocine-3-carbonyl)-4-(3-(2-(2-((2-(2,6-dioxopiperidin-3-yl)-1-oxoisoindolin-4-yl)oxy)ethoxy)ethoxy)phenyl)pyrrolidine-3-carbonitrile (20 mg, 27 μmol, 1.0 eq.) and 4-nitrophenyl 5-((bis(2-(butyrylthio)ethoxy)phosphoryl)difluoromethyl)benzo[b]thiophene-2-carboxylate (20 mg, 27 μmol, 1.0 eq.) in DMF (2 mL) was added DIEA (14 mg, 108 μmol, 4.0 eq.). The reaction mixture was stirred at room temperature for 2 hrs. After completion, the residue was purified by prep-HPLC to give S,S′-(((((2-(((3S,6S,7aS,8aR,9aR)-3-((rel-3S,4R)-3-cyano-4-(3-(2-(2-((2-(2,6-dioxopiperidin-3-yl)-1-oxoisoindolin-4-yl)oxy)ethoxy)ethoxy)phenyl)pyrrolidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 16, 20 mg, 16 μmol, 57%) as a white solid

¹H NMR (400 MHz, DMSO-d6) 11.11-9.97 (m, 1H), 9.09-8.81 (m, 1H), 8.39-8.28 (m, 1H), 8.28-8.11 (m, 2H), 7.63-7.54 (m, 1H), 7.50-7.41 (m, 1H), 7.35-7.14 (m, 3H), 7.08-6.93 (m, 2H), 6.92-6.83 (m, 1H), 5.21-5.05 (m, 1H), 5.03-4.88 (m, 1H), 4.66-4.48 (m, 1H), 4.39-4.31 (m, 1H), 4.31-4.02 (m, 11H), 4.00-3.91 (m, 1H), 3.90-3.55 (m, 7H), 3.53-3.43 (m, 1H), 3.30-3.20 (m, 1H), 3.17-3.09 (m, 4H), 2.96-2.82 (m, 1H), 2.60-2.52 (m, 4H), 2.40-2.30 (m, 2H), 2.20-1.80 (m, 8H), 1.72-1.61 (m, 1H), 1.59-1.51 (m, 4H), 1.45-1.35 (m, 1H), 0.89-0.70 (m, 7H), 0.06-0.00 (m, 1H).

LCMS (ESI): m/z=1290.1 [M+H]⁺.

### Preparation of ((2-(((3S,6S,7aS,8aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 17) and S,S′-(((((2-(((3S,6S,7aS,8aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 18)

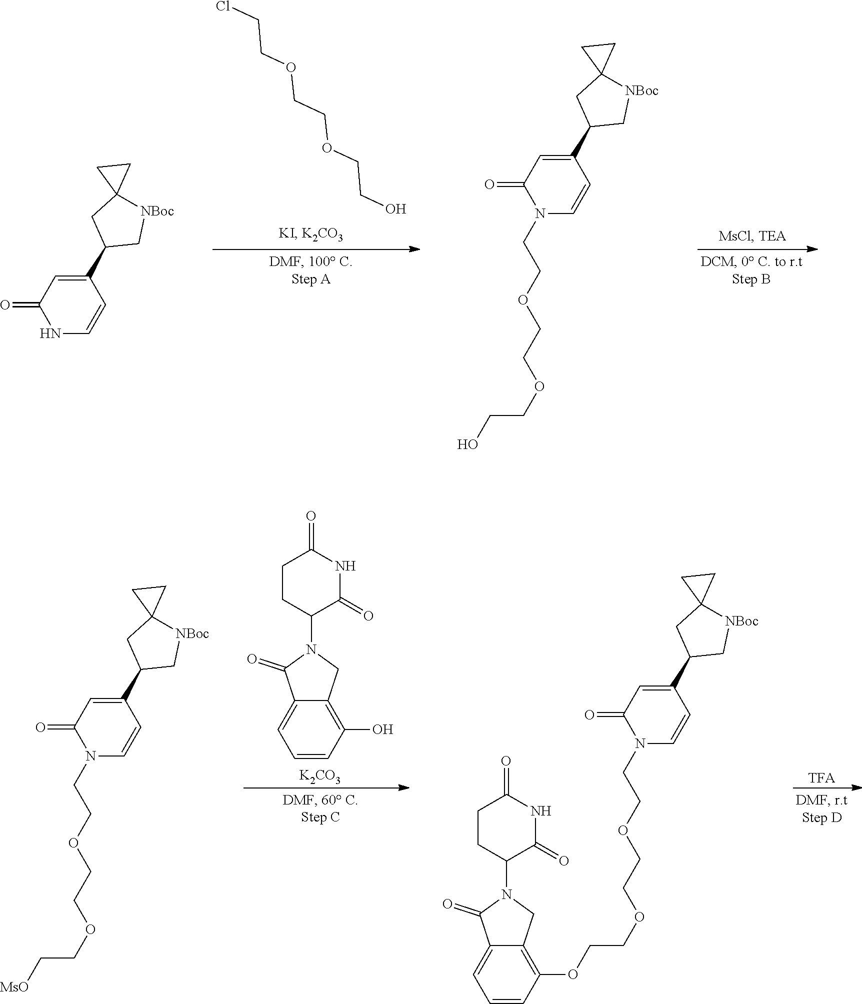

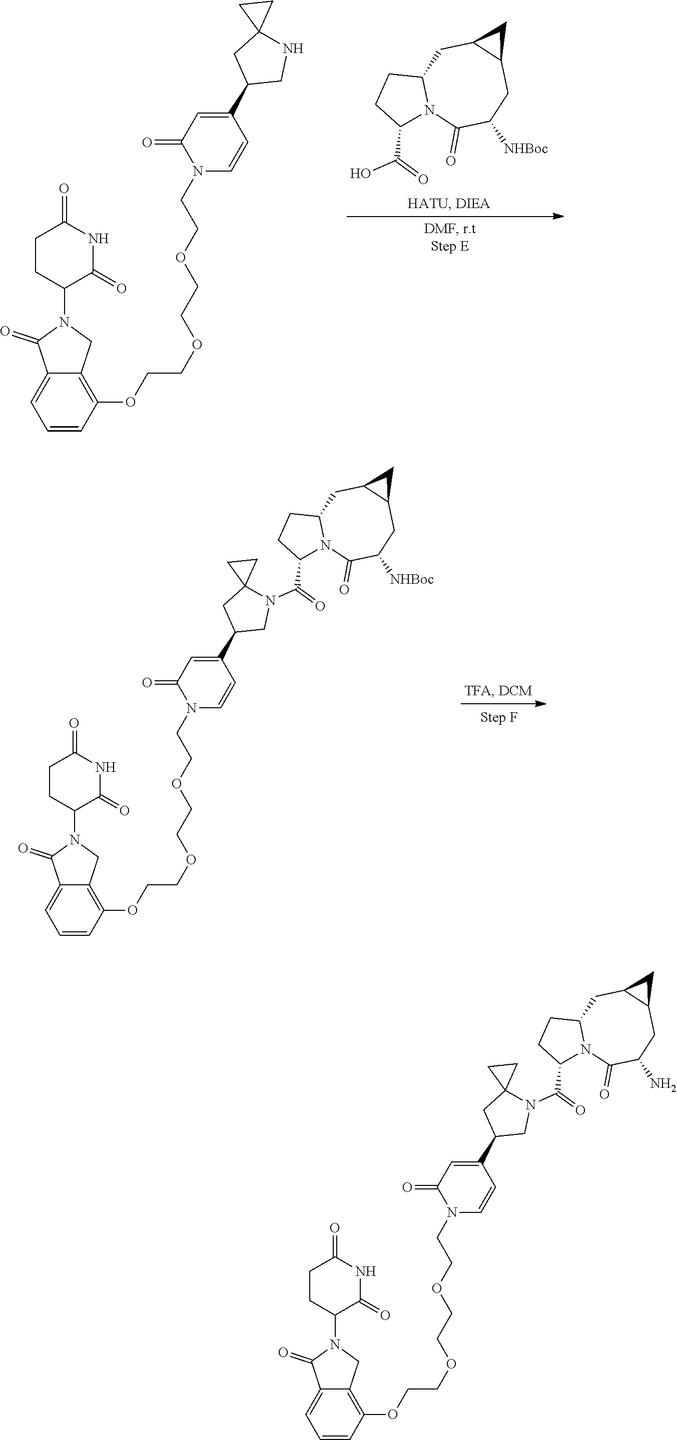 [SMILES: O=C1CCC(N2Cc3c(OCCOCCOCCn4ccc([C@@H]5CNC6(CC6)C5)cc4=O)cccc3C2=O)C(=O)N1 ; CC(C)(C)OC(=O)N[C@H]1C[C@@H]2C[C@@H]2C[C@H]2CC[C@@H](C(=O)O)N2C1=O ; CC(C)(C)OC(=O)N[C@H]1C[C@@H]2C[C@@H]2C[C@H]2CC[C@@H](C(=O)N3C[C@@H](c4ccn(CCOCCOCCOc5cccc6c5CN(C5CCC(=O)NC5=O)C6=O)c(=O)c4)CC34CC4)N2C1=O ; N[C@H]1C[C@@H]2C[C@@H]2C[C@H]2CC[C@@H](C(=O)N3C[C@@H](c4ccn(CCOCCOCCOc5cccc6c5CN(C5CCC(=O)NC5=O)C6=O)c(=O)c4)CC34CC4)N2C1=O]

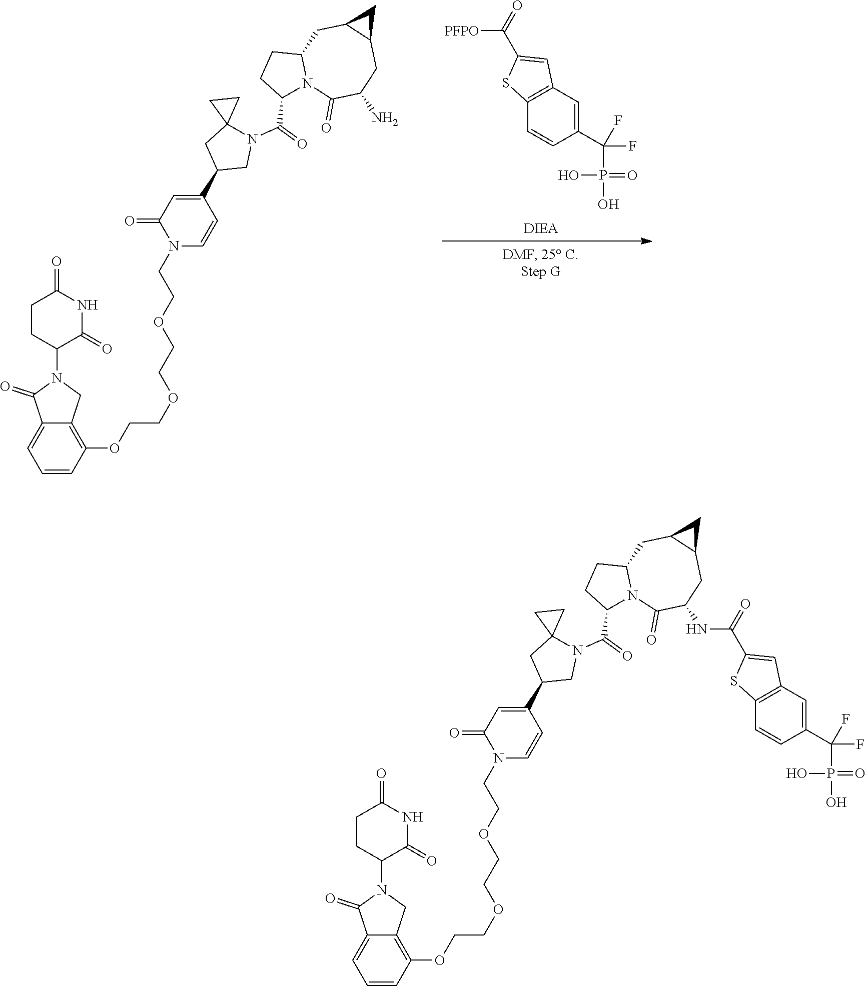

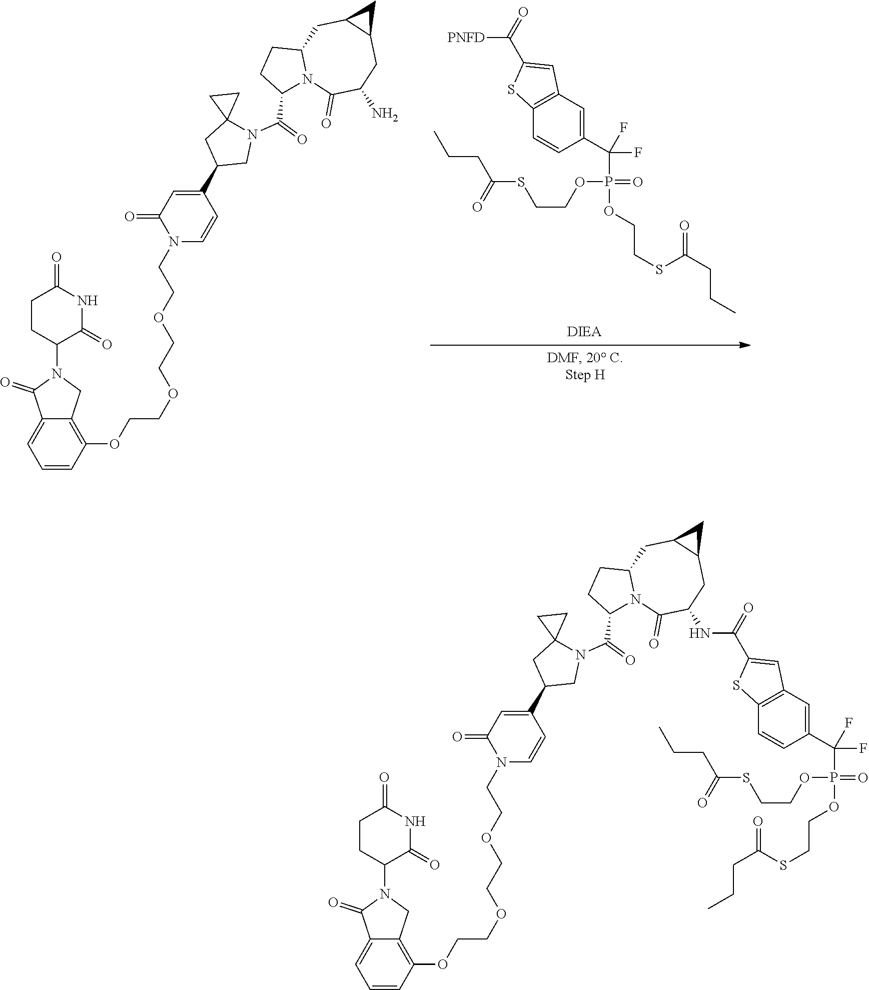

### STEP A: tert-butyl (R)-6-(1-(2-(2-(2-hydroxyethoxy)ethoxy)ethyl)-2-oxo-1,2-dihydropyridin-4-yl)-4-azaspiro[2,4]heptane-4-carboxylate

To a solution of tert-butyl (R)-6-(2-oxo-1,2-dihydropyridin-4-yl)-4-azaspiro[2.4]heptane-4-carboxylate (700 mg, 2.4 mmol, 1.0 eq.) in DMF (10 mL) were added 2-(2-(2-chloroethoxy)ethoxy)ethan-1-ol (490 mg, 2.9 mmol, 1.2 eq.), KI (200 mg, 1.2 mmol. 0.5 eq.) and K₂CO₃ (670 mg, 4.8 mmol, 2.0 eq.). The reaction mixture was stirred at 100° C. for 2 hrs. After completion, the reaction mixture was purified directly by Biotage® C18 column chromatography to afford tert-butyl tert-butyl (R)-6-(1-(2-(2-(2-hydroxyethoxy)ethoxy)ethyl)-2-oxo-1,2-dihydropyridin-4-yl)-4-azaspiro[2.4]heptane-4-carboxylate (400 mg, 0.95 mmol, 39%) as a yellow solid. LCMS (ESI): m/z=423 [M+H]⁺.

### STEP B: tert-butyl (R)-6-(1-(2-(2-(2-((methylsulfonyl)oxy)ethoxy)ethoxy)ethyl)-2-oxo-1,2-dihydropyridin-4-yl)-4-azaspiro[2.4]heptane-4-carboxylate

To a solution of tert-butyl (R)-6-(1-(2-(2-(2-hydroxyethoxy)ethoxy)ethyl)-2-oxo-1,2-dihydropyridin-4-yl)-4-azaspiro[2.4]heptane-4-carboxylate (400 mg, 0.95 mmol, 1.0 eq.) in DCM (5 mL) were added TEA (192 mg, 1.9 mmol, 2.0 eq.) and MsCl (130 mg, 1.14 mmol, 1.2 eq.) at 0° C. After addition, the resulting mixture was warmed up to room temperature and stirred for 1 hr. After completion, the reaction mixture was diluted with H₂O (10 mL), then extracted with EtOAc (15 mL×3). The organic layers were combined and washed with brine (10 mL), dried over anhydrous Na₂SO₄, and concentrated under reduced pressure. The residue was purified by flash column chromatography on silica gel to afford tert-butyl (R)-6-(1-(2-(2-(2-((methylsulfonyl)oxy)ethoxy)ethoxy)ethyl)-2-oxo-1,2-dihydropyridin-4-yl)-4-azaspiro[2.4]heptane-4-carboxylate (330 mg, 0.66 mmol, 69%) as a colorless oil. LCMS (ESI): m/z=501 [M+H]⁺.

### STEP C: tert-butyl (6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carboxylate

To a solution of tert-butyl (R)-6-(1-(2-(2-(2-((methylsulfonyl)oxy)ethoxy)ethoxy)ethyl)-2-oxo-1,2-dihydropyridin-4-yl)-4-azaspiro[2.4]heptane-4-carboxylate (330 mg, 0.66 mmol, 1.0 eq.) and K₂C₀ (324 mg, 1.98 mmol, 3.0 eq.) in DMF (5 mL) was added 3-(4-hydroxy-1-oxoisoindolin-2-yl)piperidine-2,6-dione (174 mg, 0.66 mmol, 1.0 eq.) at room temperature. After addition, the resulting mixture was heated to 60° C. and stirred for overnight. After completion, the reaction mixture was diluted with H₂O (10 mL), then extracted with EtOAc (15 mL×3). The organic layers were combined and washed with brine (10 mL), dried over anhydrous Na₂SO₄, and concentrated under reduced pressure. The residue was purified by flash column chromatography on silica gel to afford tert-butyl (6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carboxylate (340 mg, 0.51 mmol, 77%) as a white solid. LCMS (ESI): m/z=665 [M+H]⁺.

### STEP D: 3-(1-oxo-4-(2-(2-(2-(2-oxo-4-((R)-4-azaspiro[2.4]heptan-6-yl)pyridin-1(2H)-yl)ethoxy)ethoxy)ethoxy)isoindolin-2-yl)piperidine-2,6-dione

To a solution of tert-butyl tert-butyl (6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carboxylate (340 mg, 0.51 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(1-oxo-4-(2-(2-(2-(2-oxo-4-((R)-4-azaspiro[2.4]heptan-6-yl)pyridin-1(2H)-yl)ethoxy)ethoxy)ethoxy)isoindolin-2-yl)piperidine-2,6-dione (400 mg, quant.) as a yellow oil, which was used in next step directly without further purification. LCMS (ESI): m/z=565 [M+H]⁺.

### STEP E: tert-butyl ((3S,6S 7aS,8aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (144 mg, 0.43 mmol, 1.2 eq.) and HATU (202 mg, 0.53 mmol, 1.5 eq.) in DMF(2 mL) were added 3-(1-oxo-4-(2-(2-(2-(2-oxo-4-((R)-4-azaspiro[2.4]heptan-6-yl)pyridin-1(2H)-yl)ethoxy)ethoxy)ethoxy)isoindolin-2-yl)piperidine-2,6-dione (200 mg, 0.35 mmol, 1.0 eq.) and DIEA (137 mg, 1.06 nmol, 3.0 eq.). The resulting mixture was stirred at room temperature for 2 hrs under N₂. After completion, the reaction mixture was purified by Biotage® C18 column chromatography to get tert-butyl ((3S,6S,7aS,8aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (6, 90 mg, 0.1 mmol, 23%) as a white solid LCMS (ESI): m/z=885 [M+H]⁺.

### STEP F: 3-(4-(2-(2-(2-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)ethoxy)ethoxy)ethoxy)-1-oxoisoindolin-2-yl)piperidine-2,6-dione

To a solution of tert-butyl ((3S,6S,7aS,8aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (90 mg, 0.1 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(4-(2-(2-(2-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)ethoxy)ethoxy)ethoxy)-1-oxoisoindolin-2-yl)piperidine-2,6-dione (90 mg, quant.) as a yellow oil, which was used in next step directly without further purification LCMS (ESI): m/z=785 [M+H]⁺.

### STEP G: ((2-(((3S,6S 7aS, aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of 3-(4-(2-(2-(2-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)ethoxy)ethoxy)ethoxy)-1-oxoisoindolin-2-yl)piperidine-2,6-dione (45 mg, 57 μmol, 1.0 eq.) in DMF (1 mL) were added (difluoro(2-((perfluorophenoxy)carbonyl)benzo[b]thiophen-5-yl)methyl)phosphonic acid (27 mg, 57 μmol, 1.0 eq.) and DIEA (22 mg, 172 μmol, 3.0 eq.). The resulting mixture was stirred at 25° C. for 2 hrs under N₂. After completion, the reaction mixture was concentrated under reduced pressure. The residue was purified by prep-HPLC to get ((2-(((3S,6S,7aS,8aR,9aR)-3-((6R)-6-(1-(2-(2-2(2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 17, 13 mg, 12 μmol, 21%) as a white solid.

¹H NMR (400 MHz, DMSO-d6) δ 10.96 (s, 1H), 8.88 (d, J=7.4 Hz, 1H), 8.34 (s, 1H), 8.16-8.05 (m, 2H), 7.58 (d, J=8.6 Hz, 1H), 7.52-7.42 (m, 2H), 7.31 (d, J=7.4 Hz, 1H), 7.21 (d, J=8.1 Hz, 1H), 6.32 (s, 1H), 6.13 (d, J=7.0 Hz, 1H), 5.12-5.07 (m, 1H), 4.96-4.89 (m, 1H), 4.49-4.45 (m, 1H), 4.35-4.31 (m, 1H), 4.22-4.15 (m, 4H), 4.01-3.93 (m, 3H), 3.75-3.57 (m, 5H), 3.49 (s, 4H), 3.32-3.26 (m, 1H), 2.95-2.85 (m, 1H), 2.68-2.55 (m, 1H), 2.46-2.42 (m, 1H), 2.41-2.28 (m, 2H), 2.11-1.91 (m, 7H), 1.85-1.72 (m, 3H), 1.66-1.49 (m, 2H), 1.38-1.32 (m, 1H), 0.85-0.72 (m, 2H), 0.50-0.45 (m, 2H).

LCMS (ESI): m/z=1075.1 [M+H]+.

### STEP H: S,S′-(((((2-(((3S,6S,7aS,8aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of 3-(4-(2-(2-(2-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)ethoxy)ethoxy)ethoxy)-1-oxoisoindolin-2-yl)piperidine-2,6-dione (7, 45 mg, 57 μmol, 1.0 eq.) in DMF (1 mL) were added 4-nitrophenyl 5-((bis(2-(butyrylthio)ethoxy)phosphoryl)difluoromethyl)benzo[b]thiophene-2-carboxylate (39 mg, 57 μmol, 1.0 eq.) and TEA (20 mg, 172 μmol, 3.0 eq.). The resulting mixture was stirred at room temperature for 2 hrs under N₂. After completion, the reaction mixture was purified by prep-HPLC to get S,S′-(((((2-(((3S,6S,7aS,8aR,9aR)-3-((6R)-6-(1-(2-(2-(2-((2-(2,6-dioxopiperidin-3-yl)-1-oxoisoindolin-4-yl)oxy)ethoxy)ethoxy)ethyl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 18, 14.9 mg, 11 μmol, 19%) as a white solid.

¹H NMR (400 MHz, DMSO-d6) δ 10.96 (s, 1H), 8.95 (d, J=7.5 Hz, 1H), 8.36 (s, 1H), 8.22-8.13 (m, 2H), 7.58 (d, J=8.6 Hz, 1H), 7.50-7.44 (m, 2H), 7.31 (d, J=7.5 Hz, 1H), 7.21 (d, J=8.1 Hz, 1H), 6.32 (s, 1H), 6.13 (d, J=7.0 Hz, 1H), 5.12-5.07 (m, 1H), 4.96-4.90 (m, 1H), 4.50-4.45 (m, 1H), 4.35-4.31 (m, 1H), 4.22-4.11 (m, 8H), 3.97-3.96 (m, 3H), 3.74-3.66 (m, 3H), 3.59 (t, J=5.2 Hz, 2H), 3.48 (s, 4H), 3.13 (t, J=6.0 Hz, 4H), 2.95-2.85 (m, 1H), 2.67-2.59 (m, 1H), 2.55-2.53 (m, 3H), 2.46-2.24 (m, 3H), 2.14-1.93 (m, 8H), 1.87-1.75 (m, 2H), 1.60-1.48 (m, 6H), 1.40-1.32 (m, 1H), 0.90-0.83 (m, 7H), 0.81-0.71 (m, 2H), 0.41 (s, 2H), 0.01-−0.00 (m, 1H).

LCMS (ESI): m/z=1336.5 [M+H]⁺.

### Preparation of ((2-(((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 19) and S,S′-(((((2-(((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 20)

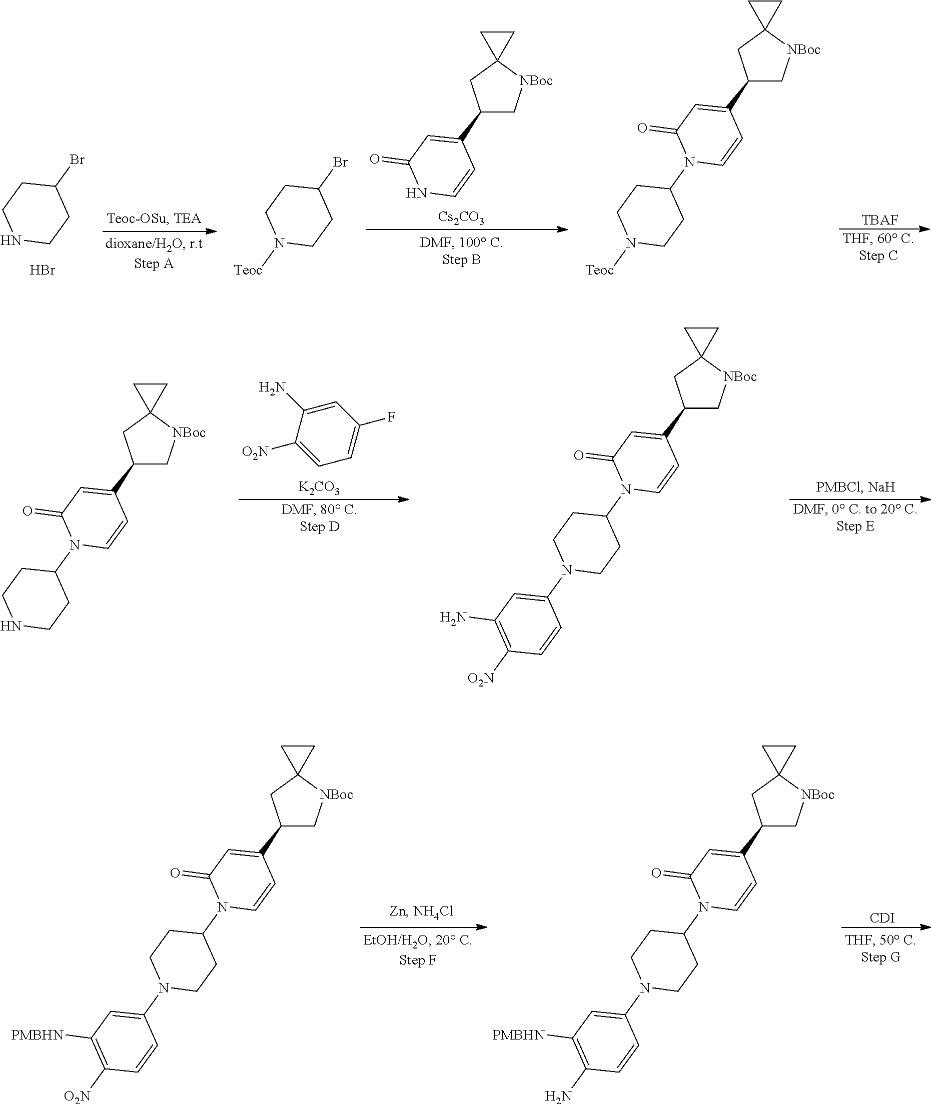

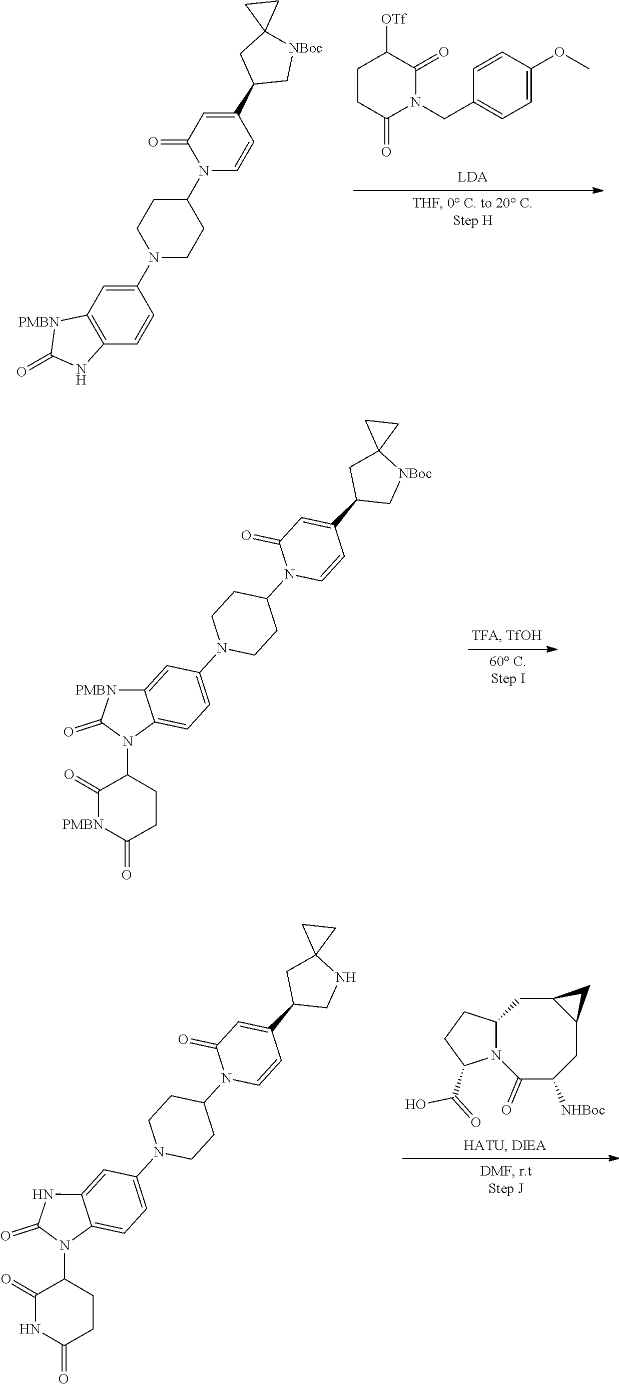

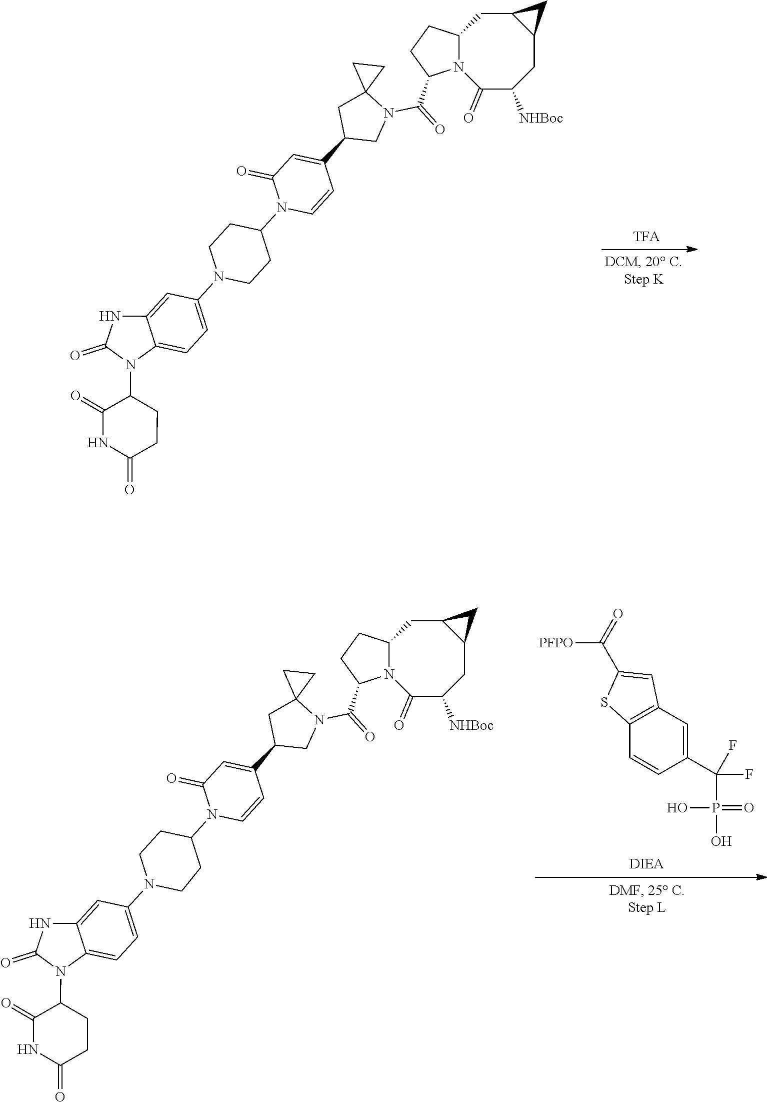

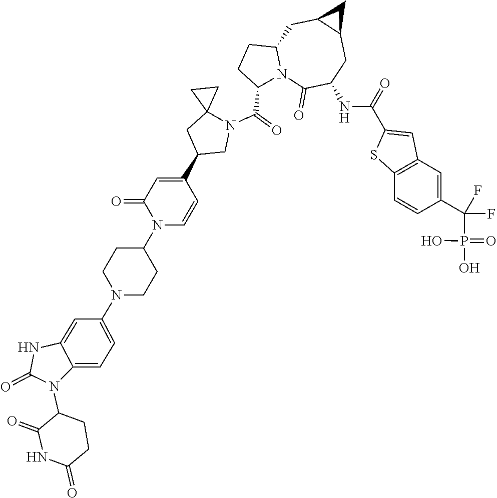 [SMILES: O=C1CCC(n2c(=O)[nH]c3cc(N4CCC(n5ccc([C@@H]6CN(C(=O)[C@@H]7CC[C@@H]8C[C@H]9C[C@H]9C[C@H](NC(=O)c9cc%10cc(C(F)(F)P(=O)(O)O)ccc%10s9)C(=O)N87)C7(CC7)C6)cc5=O)CC4)ccc32)C(=O)N1]

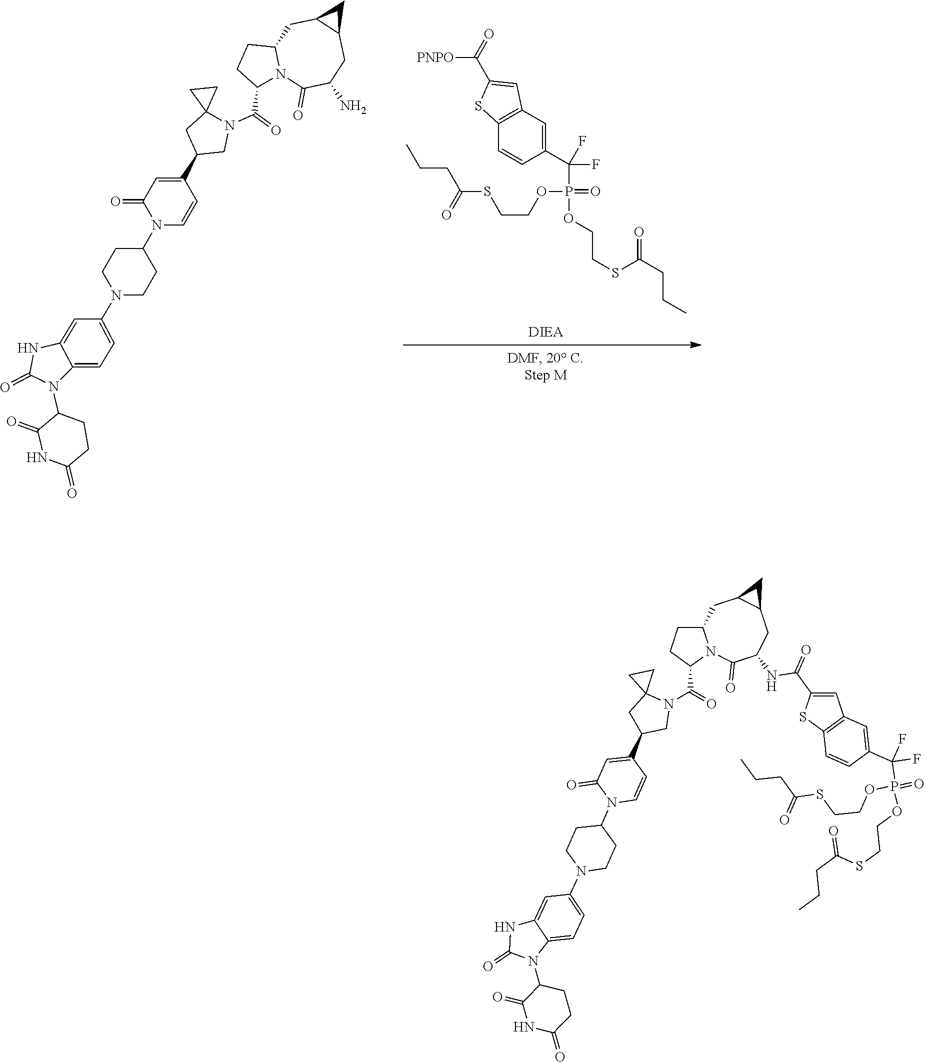

### STEP A: 2-(trimethylsilyl)ethyl 4-bromopiperidine-1-carboxylate

To a solution of 4-bromopiperidine hydrobromide (9 g, 37.0 mmol, 1 eq.) and TEA (7.8 mL, 55.5 mmol, 1.5 eq.) in dioxane/H₂O (100 mL/100 mL) was added 2,5-dioxopyrrolidin-1-yl (2-(trimethylsilyl)ethyl) carbonate (12.5 g, 48.1 mmol, 1.3 eq.) at 20° C. The solution was stirred at room temperature for 12 hrs. After completion, H₂O (300 mL) was added to the solution, the solution was extracted with EtOAc (200 mL×3). The organic layers were combined and washed with brine (300 mL) dried over anhydrous Na₂SO₄, and concentrated under reduced pressure. The residue was purified by flash column chromatography on silica gel to afford 2-(trimethylsilyl)ethyl 4-bromopiperidine-1-carboxylate (10 g, 32.6 mmol, 88%) as a colorless oil. LC-MS (ESI) m/z=308 [M+H]⁺.

### STEP B: tert-butyl (R)-6-(2-oxo-1-(1-((2-(trimethylsilyl)ethoxy)carbonyl)piperidin-4-yl)-1,2-dihydropyridin-4-yl)-4-azaspiro[2.4]heptane-4-carboxylate

A solution of tert-butyl (R)-6-(2-oxo-1,2-dihydropyridin-4-yl)-4-azaspiro[2.4]heptane-4-carboxylate (1 g, 3.45 mmol, 1.0 eq.), 2-(trimethylsilyl)ethyl 4-bromopiperidine-1-carboxylate (5.2 g, 17.3 mmol, 5 eq.) and Cs₂CO₃ (2.2 g, 6.9 mmol, 2 eq.) in DMF (10 mL) was stirred at 100° C. overnight. After completion, H₂O (300 mL) was added to the solution, the solution was extracted with EtOAc (200 mL×3). The organic layers were combined and washed with brine (300 mL), dried over anhydrous Na₂SO₄, and concentrated under reduced pressure. The residue was purified by flash column chromatography on silica gel to afford tert-butyl (R)-6-(2-oxo-1-(1-((2-(trimethylsilyl)ethoxy)carbonyl)piperidin-4-yl)-1,2-dihydropyridin-4-yl)-4-azaspiro[2.4]heptane-4-carboxylate (600 mg, 34%) as a yellow oil. LC-MS (ESI) m/z=518 [M+H]⁺.

### STEP C: tert-butyl (R)-6-(2-oxo-1-(piperidin-4-yl)-1,2-dihydropyridin-4-yl)-4-azaspiro[2.4]heptane-4-carboxylate

A solution of tert-butyl (R)-6-(2-oxo-4-(1-((2-(trimethylsilyl)ethoxy)carbonyl)piperidin-4-yl)-1,2-dihydropyridin-4-yl)-4-azaspiro[2.4]heptane-4-carboxylate (600 mg, 1.2 mmol, 1.0 eq.) and TBAF (909.1 mg, 3.6 mmol, 3 eq.) in THF (10 mL) was stirred at 60° C. for 12 hrs. After completion, the reaction mixture concentrated under reduced pressure to afford tert-butyl (R)-6-(2-oxo-1-(piperidin-4-yl)-1,2-dihydropyridin-4-yl)-4-azaspiro[2.4]heptane-4-carboxylate (270 mg, crude) as a yellow oil, which was used in the next step without further purification. LCMS (ESI): m/z=374 [M+H]⁺.

### STEP D: tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate

A solution of tert-butyl (R)-6-(2-oxo-1-(piperidin-4-yl)-1,2-dihydropyridin-4-yl)-4-azaspiro[2.4]heptane-4-carboxylate (270 mg, 0.72 mmol, 1.0 eq.), 5-fluoro-2-nitroaniline (113 mg, 0.72 mmol, 1.0 eq.) and K₂CO₃ (299 mg, 2.16 mmol, 3 eq.) in DMF (3 mL) was stirred at 80° C. for 16 hrs. After completion. H₂O (50 mL) was added to the solution, the solution was extracted with EtOAc (50 mL×3). The organic layers were combined and washed with brine (50 mL), dried over anhydrous Na₂SO₄, and concentrated under reduced pressure. The residue was purified by flash column chromatography on silica gel to afford tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 76%) as a yellow solid. LCMS (ESI): m/z=510 [M+H]⁺.

### STEP E: tert-butyl (R)-6-(1-(1-(3-amino-4-nitrophenyl)piperidin-4-yl)-2-oxo-1,2-dihydropyridin-4-yl)-4-azaspiro[2,4]heptane-4-carboxylate

To a solution of tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 0.55 mmol, 1.0 eq.) in DMF was added 60% NaH (44 mg, 1.1 mmol, 2 eq.) at 0° C. To the reaction mixture was added PMBCl (94.4 mg, 0.6 mmol, 1.1 eq.). The reaction mixture was stirred at 20° C. for 0.5 hrs. After completion, H₂O (50 mL) was added to the solution, the solution was extracted with EtOAc (50 mL×3). The organic layers were combined and washed with brine (50 mL), dried over anhydrous Na₂SO₄, and concentrated under reduced pressure. The residue was purified by flash column chromatography on silica gel to afford tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 81%) as a yellow oil. LCMS (ESI): m/z=630 [M+H]⁺.

### STEP F: tert-butyl (R)-6-(1-(1-(4-amino-3-((4-methoxybenzyl)amino)phenyl)piperidin-4-yl)-2-oxo-1,2-dihydropyridin-4-yl)-4-azaspiro[2.4]heptane-4-carboxylate

A solution of tert-butyl (R)-6-(1-(1-(3-amino-4-nitrophenyl)piperidin-4-yl)-2-oxo-1,2-dihydropyridin-4-yl)-4-azaspiro[2.4]heptane-4-carboxylate (280 mg, 0.44 mmol, 1.0 eq.) and Zn (284 mg, 4.4 mmol, 10 eq) in EtOH/sat.NH₄Cl (5 mL/2 mL) was stirred at 20° C. for 0.5 hrs. After completion, the reaction mixture was concentrated under reduced pressure. The residue was purified by C18 column chromatography to afford tert-butyl (R)-6-(1-(1-(4-amino-3-((4-methoxybenzyl)amino)phenyl)piperidin-4-yl)-2-oxo-1,2-dihydropyridin-4-yl)-4-azaspiro[2.4]heptane-4-carboxylate (180 mg, 0.3 mmol, 68%) as a yellow oil. LCMS (ESI): m/z=600 [M+H]⁺.

### STEP G: tert-butyl (R)-6-(1-(1-(3-(4-methoxybenzyl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate

A solution of tert-butyl (R)-6-(1-(1-(4-amino-3-((4-methoxybenzyl)amino)phenyl)piperidin-4-yl)-2-oxo-1,2-dihydropyridin-4-yl)-4-azaspiro[2.4]heptane-4-carboxylate (180 mg, 0.3 mmol, 1.0 eq.) and CDI (100 mg, 0.6 mmol, 2 eq.) in THF (5 mL) was stirred at 50° C. for 2 hrs. After completion, the reaction mixture was diluted with H₂O (30 mL), then extracted with EtOAc (25 mL×3). The organic layers were combined and washed with brine (20 mL), dried over anhydrous Na₂SO₄, and concentrated under reduced pressure. The residue was purified by flash column chromatography on silica gel to afford tert-butyl (R)-6-(1-(1-(3-(4-methoxybenzyl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (140 mg, 0.22 mmol, 74%) as a yellow oil. LCMS (ESI): m/z=626 [M+H]⁺.

### STEP H: tert-butyl (R)-6-(1-(1-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate

To a solution of tert-butyl (R)-6-(1-(1-(3-(4-methoxybenzyl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (140 mg, 0.22 nmol, 1.0 eq.) in THF (5 mL) was added LDA (0.11 mL, 0.22 mmol, 1.0 eq.) at 0° C. under N₂. The reaction mixture was stirred at 0° C. for 0.5 hrs. To the reaction mixture was added 1-(4-methoxybenzyl)-2,6-dioxopiperidin-3-yl trifluoromethanesulfonate (172 mg, 0.44 mmol, 2 eq.), The reaction was warmed up to 20° C. and stirred for 1 hr. After completion, the reaction mixture was diluted with H₂O (30 mL), extracted with EtOAc (20 mL×3). The organic layers were combined and washed with brine (50 mL), dried over anhydrous Na₂SO₄, and concentrated under reduced pressure. The residue was purified by flash column chromatography on silica gel to afford tert-butyl (6R)-6-(1-(1-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (170 mg, 0.20 mmol, 90%) as a yellow solid. LCMS (ESI): m/z=857 [M+H]⁺.

### STEP I: 3-(2-oxo-5-(4-(2-oxo-4-((R)-4-azaspiro[2.4]heptan-6-yl)pyridin-1(2H)-yl)piperidin-1-yl)-2,3-dihydro-1H-benzo[d]imidazol-1-yl)piperidine-2,6-dione

A solution of tert-butyl (6R)-6-(1-(1-(3-(4-methoxybenzyl)-1-(1-(4-methoxybenzyl)-2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carboxylate (170 mg, 0.20 mmol, 1.0 eq.) and TfOH (60 mg, 0.40 mmol, 2.0 eq.) in TFA (2 mL) was heated at 60° C. and stirred for 12 hrs. After completion, the reaction mixture was concentrated under reduced pressure. The residue was purified by C18 column to afford 3-(2-oxo-5-(4-(2-oxo-4-((R)-4-azaspiro[2.4]heptan-6-yl)pyridin-1(2H)-yl)piperidin-1-yl)-2,3-dihydro-1H-benzo[d]imidazol-1-yl)piperidine-2,6-dione (100 mg, 0.13 mmol, 66%) as a white solid. LCMS (ESI): m/z=517 [M+H]⁺.

### STEP J: tert-butyl ((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (17 mg, 0.05 mmol, 1 eq.), DIEA (32 mg, 0.25 mmol, 5 eq) and HATU (38 mg, 79 μmol, 1.2 eq.) in DMF (2 mL) was added 3-(2-oxo-5-(4-(2-oxo-4-((R)-4-azaspiro[2.4]heptan-6-yl)pyridin-1(2H)-yl)piperidin-1-yl)-2,3-dihydro-1H-benzo[d]imidazol-1-yl)piperidine-2,6-dione (25 mg, 0.05 mmol, 1 eq.). The reaction mixture was stirred at room temperature for 1 hr. After completion, the reaction mixture was purified by C18 column to afford tert-butyl ((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (28 mg, 33 μmol, 60%) as a white solid. LCMS (ESI): m/z=837 [M+H]⁺.

### STEP K: 3-(5-(4-(4-((R)-4-((3S,6S 7aS,8aR,9aR)-6-amino-1-oxodecahydro-1H-cyclopropa[d]pyrrolo[1,2-a]azocine-3-carbonyl)-4-azaspiro[2,4]heptan-6-yl)-2-oxopyridin-1(2H)-yl)piperidin-1-yl)-2-oxo-2,3-dihydro-1H-benzo[d]imidazol-1-yl)piperidin-2,6-dione

A solution of tert-butyl ((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamate (28 mg, 0.03 mmol, 1.0 eq.) in DCM/TFA (3 mL/1 mL) was stirred at 20° C. for 1 hr. The reaction mixture was concentrated to give 3-(5-(4-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)piperidin-1-yl)-2-oxo-2,3-dihydro-1H-benzo[d]imidazol-1-yl)piperidine-2,6-dione (24 mg, crude) as a yellow solid, which was used in the next step without further purification. LCMS (ESI): m/z=737 (M+H)⁺.

### STEP L: ((2-(((3S,6S,7aS,8aR,9aR)-3-((6R)-6-(1-(1-(1-(2,6-dioxopiperidin-3-yl)-2-oxo-2,3-dihydro-1H-benzo/d/imidazol-5-yl)piperidin-4-yl)-2-oxo-1,2-dihydropyridin-4-yl)-4-azaspiro[2,4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

A solution of 3-(5(4-(4-((R)-4-((3S,6S,7aS,8aR,9aR)-6-amino-5-oxodecahydro-1H-cyclopropa[d]pyrrolo[1,2-a]azocine-3-carbonyl)-4-azaspiro[2.4]heptan-6-yl)-2-oxopyridin-1(2H)-yl)piperidin-1-yl)-2-oxo-2,3-dihydro-1H-benzo[d]imidazol-1-yl)piperidine-2,6-dione (12 mg, 16 μmol, 1.0 eq.), DIEA (13 mg, 100 μmol, 6 eq.) and (difluoro(2-((perfluorophenoxy)carbonyl)benzo[b]thiophen-5-yl)methyl)phosphonic acid (8 mg, 16 μmol, 1.0 eq.) in DMF (1 mL) was stirred at 25° C. for 0.5 hrs. After completion, the reaction mixture was purified by prep-HPLC to afford ((2-(((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 19, 4 mg, 4 μmol, 24%) as a white solid.

¹H NMR (400 MHz, DMSO-d6) δ 11.28-10.93 (m, 2H), 9.08-8.72 (m, 1H), 8.32 (s, 1H), 8.16-7.95 (m, 3H), 7.63-7.50 (m, 1H), 7.26-6.89 (m, 4H), 6.80 (s, 1H), 5.43-5.16 (m, 2H), 5.02-4.83 (m, 1H), 4.60-4.49 (m, 1H), 4.20-4.07 (m, 2H), 3.83-3.76 (m, 1H), 3.54-3.47 (m, 2H), 3.01-2.83 (m, 2H), 2.75-2.58 (m, 3H), 2.38-2.28 (m, 1H), 2.23-1.73 (m, 13H), 1.66-1.20 (m, 6H), 0.91-0.64 (m, 2H), 0.54-0.35 (m, 2H).

LCMS (ESI): m/z=1027 [M+H]⁺.

### STEP M: S,S′-(((((2-(((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2,4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

A solution of ((2-(((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (12 mg, 16 μmol, 1.0 eq.), DIEA (13 mg, 100 μmol, 6 eq.) and 4-nitrophenyl 5-((bis(2-(butyrylthio)ethoxy)phosphoryl)difluoromethyl)benzo[b]thiophene-2-carboxylate (11 mg, 16 μmol, 1 eq.) was stirred at 20° C. for 2 hrs. After completion, the reaction mixture was purified by prep-HPLC to afford S,S′-(((((2-(((3S,6S,7aS,8aR,9aR)-3-((6R)-6-(1-(1-(1-(2,6-dioxopiperidin-3-yl)-2-oxo-2,3-dihydro-1H-benzo[d]imidazol-5-yl)piperidin-4-yl)-2-oxo-1,2-dihydropyridin-4-yl)-4-azaspiro[2.4]heptane-4-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 20, 4 mg, 3 μmol, 19%) as a white solid.

¹H NMR (400 MHz, DMSO-d6) δ 11.05 (s, 1H), 10.78 (s, 1H), 9.12-8.78 (m, 1H), 8.35 (s, 1H), 8.23-7.98 (m, 3H), 7.67-7.51 (m, 1H), 7.03-6.50 (m, 5H), 5.37-4.82 (m, 3H), 4.66-4.46 (m, 1H), 4.29-4.05 (m, 6H), 3.86-3.75 (m, 1H), 3.60-3.48 (m, 1H), 3.18-3.04 (m, 4H), 2.98-2.79 (m, 3H), 2.72-2.56 (m, 3H), 2.41-1.05 (m, 27H), 0.96-065 (m, 9H), 0.56-0.32 (m, 2H)

LCMS (ESI): m/z=1287 [M+H]⁺.

### Preparation of ((2-(((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 21) and S,S′-(((((2-(((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 22)

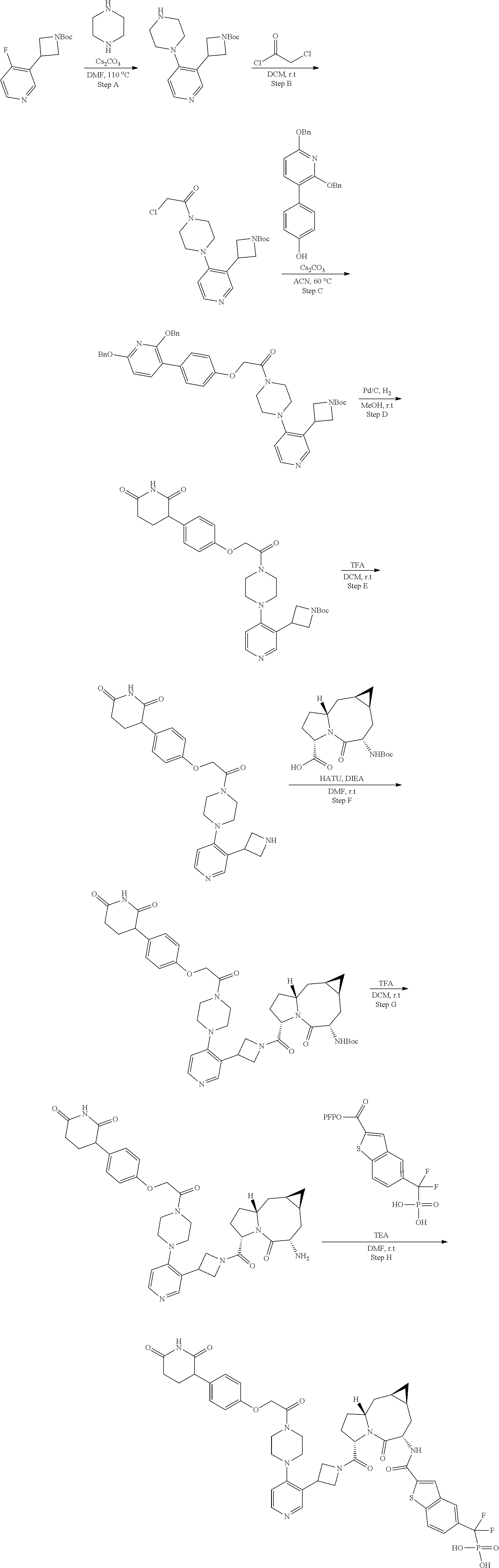

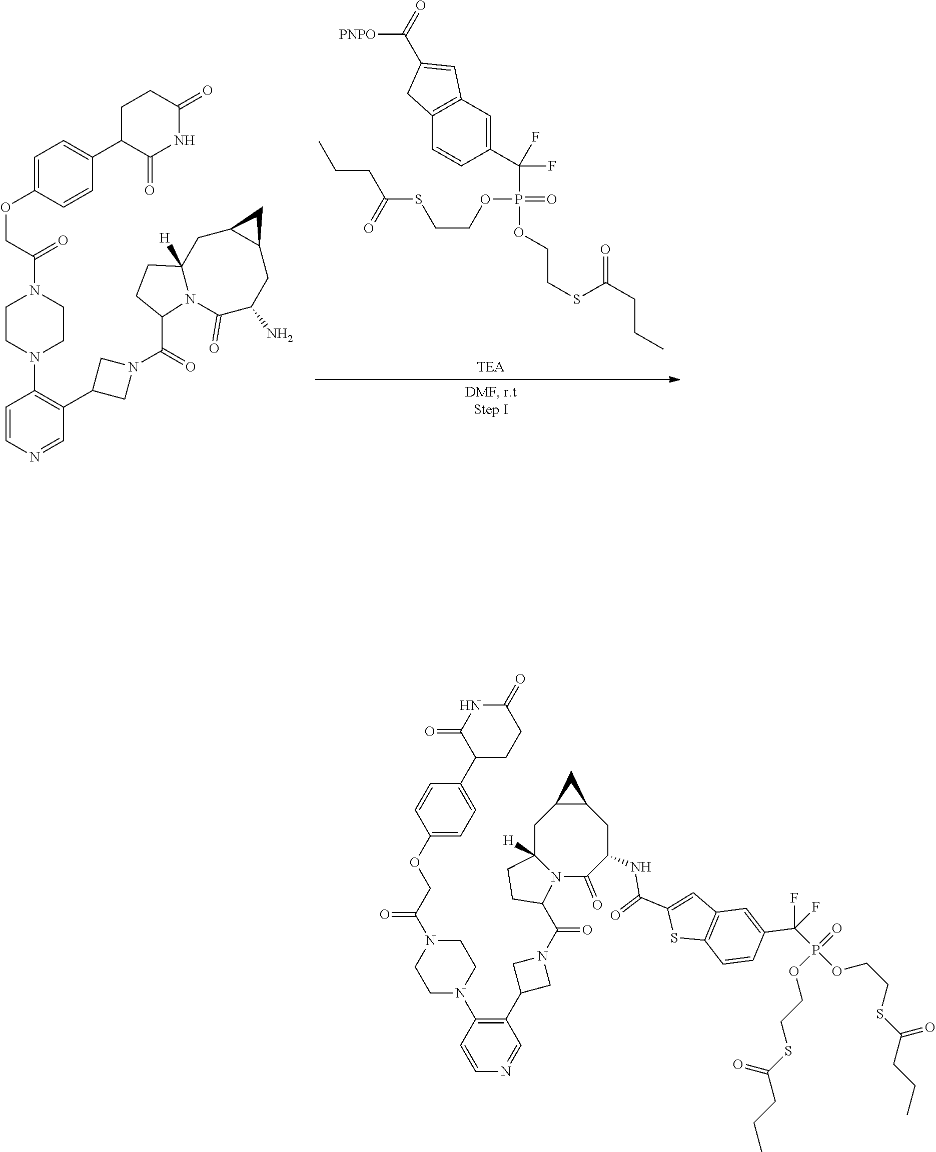

### STEP A: tert-butyl 3-(4-(piperazin-1-yl)pyridin-3-yl)azetidine-1-carboxylate

To a solution of tert-butyl 3-(4-fluoropyridin-3-yl)azetidine-1-carboxylate (1.0 g, 3.97 mmol, 1.0 eq.) in DMF (10 mL) were added piperazine (0.68 g, 7.94 mmol, 2.0 eq.) and Cs₂ (2.58 g, 7.94 mmol, 2.0 eq.). The resulting mixture was stirred at 110° C. overnight. After completion, The residue was purified by Biotage® C18 column chromatography to get tert-butyl 3-(4-(piperazin-1-yl)pyridin-3-yl)azetidine-1-carboxylate (0.9 g, 2.82 mmol, 21%) as a white solid. LCMS (ESI): m/z=319 [M+H]⁺.

### STEP B: tert-butyl 3-(4-(4-(2-chloroacetyl)piperazin-1-yl)pyridin-3-yl)azetidine-1-carboxylate

To a solution of tert-butyl 3-(4-(piperazin-1-yl)pyridin-3-yl)azetidine-1-carboxylate (0.9 g, 2.82 mmol, 1.0 eq.) in DCM (20 mL) was added 2-chloroacetyl chloride (0.35 g, 3.1 mmol, 1 eq.) and the resulting mixture was stirred at room temperature for 2 hrs under N₂. After completion, the reaction mixture was concentrated under reduced pressure. The residue was purified by Biotage® C18 column chromatography to get tert-butyl 3-(4-(piperazin-1-yl)pyridin-3-yl)azetidine-1-carboxylate (0.5 g, 1.26 mmol, 45%) as a white solid. LCMS (ESI): m/z=395 [M+H]⁺.

### STEP C: tert-butyl 3-(4-(4-(2-(4-(2,6-bis(benzyloxy)pyridin-3-yl)phenoxy)acetyl)piperazin-1-yl)pyridin-3-yl)azetidine-1-carboxylate

To a solution of tert-butyl 3-(4-(piperazin-1-yl)pyridin-3-yl)azetidine-1-carboxylate (0.5 g, 1.26 mmol, 1 eq) in ACN (5 mL) were added 4-(2,6-bis(benzyloxy)pyridin-3-yl)phenol (483 mg, 1.26 mmol, 1.0 eq.) and Cs₂CO₃ (822 mg, 2.52 mmol, 2 eq.). The resulting mixture was stirred at 60° C. for 6 hrs under N₂. After completion, the reaction mixture was concentrated under reduced pressure. The residue, was purified by Biotage® C18 column chromatography to get tert-butyl 3-(4-(4-(2-(4-(2,6-bis(benzyloxy)pyridin-3-yl)phenoxy)acetyl)piperazin-1-yl)pyridin-3-yl)azetidine-1-carboxylate (450 mg, 0.62 mmol, 42%) as a white solid. LCMS (ESI): m/z=742 [M+H]⁺.

### STEP D: tert-butyl 3-(4-(4-(2-(4-(2,6-dioxopiperidin-3-yl)phenoxy)acetyl)piperazin-1-yl)pyridin-3-yl)azetidine-1-carboxylate

To a solution of tert-butyl 3-(4-(4-(2-(4-(2,6-bis(benzyloxy)pyridin-3-yl)phenoxy)acetyl)piperazin-1-yl)pyridin-3-yl)azetidine-1-carboxylate (450 mg, 0.62 mmol, 1.0 eq.) in MeOH (20 mL) was added Pd/C (20 mg) under nitrogen. The suspension was degassed under vacuum and purged with H₂ several times. The resulting mixture was stirred at room temperature for 3 hrs. After completion, the suspension was filtered through a pad of Celite®, the filter cake was washed with MeOH (10 mL). The combined filtrates were concentrated to dryness to give tert-butyl 3-(4-(4-(2-(4-(2,6-dioxopiperidin-3-yl)phenoxy)acetyl)piperazin-1-yl)pyridin-3-yl)azetidine-1-carboxylate (200 mg, 0.35 mmol, 58%) as a white solid. LCMS (ESI): m/z=564 [M+H]⁺.

### STEP E: 3-(4-(2-(4-(3-(azetidin-3-yl)pyridin-4-yl)piperazin-1-yl)-2-oxoethoxy)phenyl)piperidine-2,6-dione

To a solution of tert-butyl 3-(4-(4-(2-(4-(2,6-dioxopiperidin-3-yl)phenoxy)acetyl)piperazin-1-yl)pyridin-3-yl)azetidine-1-carboxylate (200 mg, 0.35 mmol, 1.0 eq.) in DCM (10 mL) was added TFA (1 mL) and the reaction mixture was stirred at room temperature for 4 hrs under N₂. After completion, the reaction mixture was concentrated under reduced pressure to get crude 3-(4-(2-(4-(3-(azetidin-3-yl)pyridin-4-yl)piperazin-1-yl)-2-oxoethoxy)phenyl)piperidine-2,6-dione (180 mg, quant.) as a white solid, which was used in next step directly without further purification. LCMS (ESI): m/z=464 [M+H]⁺.

### STEP F: tert-butyl ((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamate

To a solution of (3S,6S,7aS,8aR,9aR)-6-((tert-butoxycarbonyl)amino)-5-oxodecahydro-1H-cyclopropa[d]pyrrolo[1,2-a]azocine-3-carboxylic acid (78 mg, 0.23 mmol, 1.2 eq.) and HATU (110 mg, 0.29 mmol, 1.5 eq.) in DMF(2 mL) were added 3-(4-(2-(4-(3-(azetidin-3-yl)pyridin-4-yl)piperazin-1-yl)-2-oxoethoxy)phenyl)piperidine-2,6-dione (90 mg, 0.19 mmol, 1.0 eq.) and DIEA (74 mg, 0.57 mmol, 3.0 eq.). The resulting mixture was stirred at room temperature for 2 hrs under N₂. After completion, the reaction mixture was purified by Biotage® C18 column chromatography to get tert-butyl ((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamate (80 mg, 0.1 mmol, 54%) as a white solid. LCMS (ESI): m/z=770 [M+H]⁺.

### STEP G: 3-(4-(2-(4-(3-(1-((3S,7aS,8aR,9aR)-6-amino-5-oxodecahydro-1H-cyclopropa[d]pyrrolo[1,2-a]azocine-3-carbonyl)azetidin-3-yl)pyridin-4-yl)piperazin-1-yl)-2-oxoethoxy)phenyl)piperidine-2,6-dione

To a solution of tert-butyl ((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamate (80 mg, 0.1 mmol, 1.0 eq.) in DCM (5 mL) was added TFA (2 mL) and the reaction mixture was stirred at room temperature for 2 hrs under N₂. After completion, the reaction mixture was concentrated under reduced pressure to get crude 3-(4-(2-(4-(3-(1-((3S,6S,7aS,8aR,9aR)-6-amino-5-oxodecahydro-1H-cyclopropa[d]pyrrolo[1,2-a]azocine-3-carbonyl)azetidin-3-yl)pyridin-4-yl)piperazin-1-yl)-2-oxoethoxy)phenyl)piperidine-2,6-dione (80 mg, quant.) as a yellow oil, which was used in next step directly without further purification. LCMS (ESI): m/z=670 [M+H]⁺.

### STEP H: ((2-(((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid

To a solution of 3-(4-(2-(4-(3-(1-((3S,6S,7aS,8aR,9aR)-6-amino-5-oxodecahydro-1H-cyclopropa[d]pyrrolo[1,2-a]azocine-3-carbonyl)azetidin-3-yl)pyridin-4-yl)piperazin-1-yl)-2-oxoethoxy)phenyl)piperidine-2,6-dione (40 mg, 50 μmol, 1.0 eq.) in DMF (1 mL) were added (difluoro(2-((perfluorophenoxy)carbonyl)benzo[b]thiophen-5-yl)methyl)phosphonic acid (24 mg, 50 μmol, 1.0 eq.) and TEA (18 mg, 150 μmol, 3.0 eq.). The resulting mixture was stirred at room temperature for 2 hrs under N₂. After completion, the reaction mixture was concentrated under reduced pressure. The residue was purified by prep-HPLC to get ((2-(((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphonic acid (Compound 21, 6.7 mg, 7 μmol, 14%) as a white solid.

¹H NMR (400 MHz, DMSO-d6) δ 10.79 (s, 1H), 8.91-8.78 (m, 1H), 8.59-8.37 (m, 2H), 8.31 (d, J=9.8 Hz, 1H), 8.09 (dd, J=20.2, 8.2 Hz, 2H), 7.59 (d, J=8.6 Hz, 1H), 7.30 (t, J=6.6 Hz, 1H), 7.11 (t, J=8.2 Hz, 2H), 6.88 (t, J=8.3 Hz, 2H), 4.94-4.82 (m, 3H), 4.70-4.48 (m, 1H), 4.39-4.27 (m, 2H), 4.23-4.14 (m, 2H), 4.02-3.94 (m, 1H), 3.83-3.78 (m, 1H), 2.75-2.58 (m, 2H), 2.43-227 (m, 2H), 2.24-1.94 (m, 8H), 1.91-1.82 (m, 2H), 1.71-1.60 (m, 1H), 1.49-1.32 (m, 2H), 1.29-1.18 (m, 4H), 0.92-0.66 (m, 3H), 0.09-0.06 (m, 11H).

LCMS (ESI): m/z==974.2 [M+H]⁻.

### STEP I: S,S′-(((((2-(((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate

To a solution of 3-(4-(2-(4-(3-(1-((3S,6S,7aS,8aR,9aR)-6-amino-5-oxodecahydro-1H-cyclopropa[d]pyrrolo[1,2-a]azocine-3-carbonyl)azetidin-3-yl)pyridin-4-yl)piperazin-1-yl)-2-oxoethoxy)phenyl)piperidine-2,6-dione (40 mg, 50 μmol, 1.0 eq.) in DMF (1 mL) were added 4-nitrophenyl 5-((bis(2-(butyrylthio)ethoxy)phosphoryl)difluoromethyl)benzo[b]thiophene-2-carboxylate (34 mg, 50 μmol, 1.0 eq.) and TEA (18 mg, 150 μmol, 3.0 eq.). The resulting mixture was stirred at room temperature for 2 hrs under N₂. After completion, the reaction mixture was purified by prep-HPLC to get S,S′-(((((2-(((3S,6S,7aS,8aR,9aR)-3-(3-(4-(4-(2-(4-(2,6-dioxopiperidin-3-yl)phenoxy)acetyl)piperazin-1-yl)pyridin-3-yl)azetidine-1-carbonyl)-5-oxodecahydro-1H-cyclopropa[d]pyrrolo[1,2-a]azocin-6-yl)carbamoyl)benzo[b]thiophen-5-yl)difluoromethyl)phosphoryl)bis(oxy))bis(ethane-2,1-diyl)) dibutanethioate (Compound 22, 8.4 mg, 7 μmol, 14%) as a white solid.

¹H NMR (400 MHz, DMSO-d6) δ 10.79 (s, 1H), 9.10-8.92 (m, 1H), 8.56-8.46 (m, 1H), 8.39-8.30 (m, 2H), 8.23-8.13 (m, 2H), 7.58 (d, J=8.6 Hz, 1H), 7.11 (t, J=8.8 Hz, 2H), 7.01-6.94 (m, 1H), 6.88 (t, J=7.9 Hz, 2H), 4.98-4.91 (m, 1H), 4.86-4.78 (m, 2H), 4.72-4.55 (m, 1H), 4.42-4.37 (m, 1H), 4.35-4.26 (m, 1H), 4.24-4.09 (m, 6H), 3.97-3.88 (m, 1H), 3.81-3.74 (m, 1H), 3.68-3.55 (m, 4H), 3.13 (t, J=6.3 Hz, 4H), 2.96-2.80 (m, 4H), 2.68-2.60 (m, 1H), 2.57-2.53 (m, 3H), 2.49-2.42 (m, 1H), 2.38-2.29 (m, 1H), 2.25-1.95 (m, 7H), 1.91-1.79 (m, 2H), 1.68-1.49 (m, 5H), 1.43-1.34 (m, 1H), 1.30-1.25 (m, 1H) 0.88-0.70 (m, 8H), 0.06-−0.05 (m, 1H).

LCMS (ESI): m/z=1234.2 [M+H]⁺.

### Biochemical and Cellular Assays

### STAT3 Fluorescence Polarization (FP) Assay

An FP assay was developed to determine IC₅₀ values for test substances. Recombinant STAT3 protein (STAT3(G127-I722)) at 25 nM was combined with a fluorescently labeled, phosphotyrosine peptide probe (5-FAM-GpYLPQTV-NH₂) at 2 nM in FP buffer (10 mM HEPES pH 7.4, 50 mM NaCl. 1 mM EDTA, 0.05% Tween 20, 2 mM DTT). 50 μL of STAT3-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 1. For STAT3 FP assay, A=≤100 nM; B=>100-1000 nM; C=>1-10 μM; and D=>10 μM.

### STAT6 Fluorescence Polarization (FP) Assay

An FP assay was developed to determine IC₅₀ values for test substances. Recombinant STAT6 protein (STAT6(W123-T658)) at 250 nM was combined with a fluorescently labeled, phosphotyrosine peptide probe (5-FAM-ApYKPFQDLI-NH₂) at 2 nM in FP buffer (10 mM HEPES pH 7.4, 50 mM NaCl, 1 mM EDTA, 0.05% Tween 20, 2 mM DTT). 50 μL of STAT6-probe mixture was added to serial diluted compounds in black, 96-well plates (Greiner BioOne 655076) to a final concentration of 1% DMSO. Reaction components were mixed, and FP w as measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 1. For STAT6 FP assay. A=≤300 nM; B=>300-3000 nM; C=>3-30 μM; and D=>30 μM.

TABLE 1

| Cmp# | STAT3 | STAT6 |
|---|---|---|
| 1 | A | C |
| 3 | C | B |
| 5 | B | B |
| 7 | B | B |
| 10 | B | A |
| 11 | A | C |
| 13 | B | A |
| 15 | B | A |
| 17 | C | B |
| 19 | B | B |
| 21 | A | C |

### MSD-pSTAT-PBMC Assay

### Materials:

Cryopreserved Peripheral Blood Mononuclear Cells (PBMC) are from AllCells. Recombinant Human 114 and IL-6 are from Peprotech. Mouse monoclonal anti-STAT3 antibody, rabbit monoclonal anti-pY705-STAT3 antibody, rabbit monoclonal Anti-pY641-STAT6 antibody, and lysis buffer are from Cell Signaling Technology (CST). Mouse monoclonal anti-STAT6 antibody is from BioLegend. Assay plates, blocker, and anti-rabbit secondary antibody are from Meso Scale Discovery (MSD).

### Assay Method:

Cryopreserved PBMCs were thawed out and allowed to recover overnight in IMDM+10% heat-inactivated FBS prior to plating 50,000 (STAT3) or 25,000 (STAT6) cells per well in 96-well U-bottom tissue culture plates. Cells were treated with compound for 3 hrs, then stimulated with 10 ng/mL IL-6 (STAT3) or 1 ng/mL IL-4 (STAT6) for 10 min. Cells were then spun down and washed with ice-cold PBS prior to lysing the cell pellet with 1× lysis buffer (CST) with 1× HALT protease and phosphatase inhibitor cocktail (Thermo). Lysates were transferred to and incubated overnight at 4° C. with shaking in QuickPlex 96-well high bind assay plates (MSD) pre-coated overnight with 30 μL per well of 0.6 μg/mL mouse monoclonal anti-STAT3 antibody (STAT3) or 2 μg/mL mouse monoclonal anti-STAT6 antibody (STAT6) in 1×PBS, and pre-blocked for 1 hour with 3% Blocker-A (MSD). Captured protein in the assay plates were then washed and probed with 25 μL per well of 0.25 μg/ml rabbit monoclonal anti-pY705-STAT3 antibody (STAT3) or 0.18 μg/ml rabbit monoclonal Anti-pY641-STAT6 antibody (STAT6) in 1% Blocker-A for 1 hour at room temperature with shaking, then washed and probed with 25 ul per well of 1 ug/ml Sulfo-TAG Labeled Goat Anti-Rabbit Antibody (MSD) in 1% Blocker-A for 1 hour at room temperature with shaking. Assay plates were then washed and 150 μL of 1× Read Buffer-T (MSD) was added to each well prior to reading on an SQ120 MSD Plate Reader. Assay signal from each sample was subtracted by the signal from unstimulated control wells and normalized to DMSO control wells, IC₅₀ values were calculated using Graph Pad Prism Dose-Response Nonlinear Regression with variable slope. Results are shown in Table 2. For both pSTAT3 and pSTAT6 A=≤100 nM; B=>100-1000 nM; C=>1000-3000 nM; and D=>3 μM.

TABLE 2

| Cmp# | pSTAT3 | pSTAT6 |
|---|---|---|
| 2 | A | D |
| 3 | | D |
| 4 | | B |
| 5 | | B |
| 6 | | A |
| 7 | | D |
| 8 | | A |
| 10 | | C |
| 9 | | A |
| 13 | | B |
| 15 | | D |
| 16 | | A |
| 17 | | D |
| 18 | | B |
| 14 | | A |
| 19 | | D |
| 20 | | B |
| 1 | D | |
| 11 | D | |
| 12 | A | |
| 21 | D | |
| 22 | A | |

### MSD-tSTAT-PBMC Assay

### Materials:

Cryopreserved Peripheral Blood Mononuclear Cells (PBMC) for STAT3 assay are from AllCells and for STAT6 assay are from STEMCELL Technologies. Mouse monoclonal anti-STAT6 antibody is from BioLegend. The rabbit monoclonal anti-STAT6 antibody and cell lysis buffer are from Cell Signaling Technologies. Total STAT3 assay kit is from Meso Scale Discovery (MSD). Assay plates, Blocker A, Blocker D-R, Blocker D-M, and anti-rabbit secondary antibody for the total STAT6 assay is from Meso Scale Discovery (MSD).

### Assay Method:

Cryopreserved PBMCs were thawed out and allowed to recover overnight in IMDM+10% heat-inactivated FBS and 1× Glutamax prior to plating 50,000 (STAT3) or 100,000 (STAT6) cells per well in a 96-well U-bottom tissue culture plate. Cells were treated with 10 uM compound for 20 hours, then spun down and washed with ice-cold PBS prior to lysing the cell pellet with 1× lysis buffer (CST) supplemented with IX HALT protease and phosphatase inhibitor cocktail (Thermo). Cells were then frozen at −80 C, thawed, and incubated with shaking at 4 C for 1 hour to allow cellular lysis. For the total STAT3 (tSTAT3) assay, assay ready plates (MSD) pre-blocked for 1 hour with 3% Blocker A (MSD) were used. Then, cell lysates were transferred to and incubated overnight at 4 C with shaking in QuickPlex 96-well high bind assay plates (MSD). For the total STAT6 (tSTAT6) assay, assay plates were pre-coated overnight with 30 ul per well of 2 ug/mL mouse monoclonal anti-STAT6 antibody (BioLegend) in IX PBS, and pre-blocked for 1 hour with 3% Blocker A (MSD). Then, cell lysates were transferred to and incubated for 72 h at 4 C with shaking in QuickPlex 96-well high bind assay plates (MSD). Following protein capture on the assay plates, plates were washed and probed with appropriate antibody. For the STAT3 assay, 25 ul SULFO-tag conjugated anti-total STAT3 antibody in 1% Blocker A/0.1% Blocker D-M/0.1% Blocker D-R (MSD) w as added and plates were incubated for 1 hr with shaking at room temperature. For the STAT6 assay, 25 ul per well of 0.25 ug/mL rabbit monoclonal anti-STAT6 antibody (CST) in 1% Blocker A was added and plates were incubated for 1 hour at room temperature with shaking, then washed and probed with 25 ul per well of 1 ug/mL Sulfo-TAG labeled Goat anti-Rabbit Antibody (MSD) in 1% Blocker A for 1 hour at room temperature with shaking. Assay plates were then washed and 150 ul of 1× Read Buffer-T (MSD) was added to each well prior to reading on an SQ120 MSD Plate Reader. Assay signal from each sample was subtracted by the signal from control wells and normalized to DMSO control wells to calculate percent control and percent degradation. Each condition was run in duplicate across two separate assays and values were averaged. Results are shown in Table 3. For both tSTAT3 and tSTAT6 A=>85% degradation; B=>70%-85% degradation; C=50%-70% degradation; and D=<50% degradation.

TABLE 3

| Cmp# | tSTAT3 | tSTAT6 |
|---|---|---|
| 3 | | A |
| 4 | | A |
| 5 | | A |
| 6 | | A |
| 7 | | C |
| 8 | | B |
| 10 | | A |
| 9 | | A |
| 13 | | A |
| 15 | | A |
| 16 | | B |
| 17 | | B |
| 18 | | C |
| 14 | | A |
| 19 | | B |
| 20 | | B |
| 1 | A | |
| 2 | A | |
| 11 | B | |
| 12 | A | |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the structural formula I: 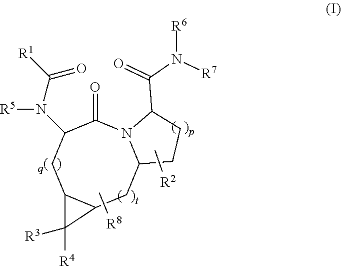
or a pharmaceutically acceptable salt thereof, wherein:
q is 0 or 1 and t is 0, 1, or 2, provided that at least one of q or t is 1;
p is 1 or 2;
R¹ is selected from an 8- to 10-membered fused bicyclic heteroaryl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an 8- to 10-membered fused bicyclic heterocyclyl substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}]; an aryl substituted with —CR^{1a}R^{2a}P(O)OR^{1b}OR^{2b}, CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], or —CR^{1a}R^{2a}P(O)[NH(AA)C(O)OR^{T}]][NH(AA)C(O)OR^{T}], —P(O)[OR^{1b}][NH(AA)C(O)OR^{T}]; and a —(C₂-C₄)alkenyl(aryl) wherein said aryl portion of —(C₂-C₄)alkenyl(aryl) is substituted with —CR^{1a}R^{2a}P(O)OR^{1b}OR^{2b}, —CR^{1a}R^{2a}P(O)[OR^{1b}][NH(AA)C(O)OR^{T}], —CR^{1a}R^{2a}P(O)[NHR^{Ty}][NH(AA)C(O)OR^{T}], —P(O)OR^{1b}OR^{2b}, —[P(O)[NHR^{Ty}][NH(AA)C(O)OR^{T}], —CR^{1a}R^{2a}P(O)[NH(AA)C(O)OR^{T}]][NH(AA)C(O)OR^{T}], or —P(O)[OR^{1b}][NH(AA)C(O)OR^{T}];
R^{1a} and R^{2a} are each independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O)—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, and (C₁-C₄)alkyl;
R⁵ and R⁶ are each independently selected from hydrogen, phenyl, and (C₁-C₄)alkyl;
R⁷ is selected from E, —R^{10A}E, (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, R¹¹ and/or 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, R¹² and/or 1 to 3 groups selected from R^{Q};
R⁸ is hydrogen or (C₁-C₄)alkyl;
R^{10A}, R^{10D}, R^{10C}, and R^{10D} are each independently a chemical spacer unit;
R¹¹ is selected from —NHC(O)R^{10B}E, —OR^{10B}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10B}E, —NHR^{10B}E, —[N(C₁-C₄)alkyl]R^{10B}E, or R^{10B}E, and wherein said 4- to 6-membered heterocyclyl is optionally substituted further with oxo;
R¹² is selected from —NHC(O)R^{10C}E, —OR^{10C}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10C}E, —NHR^{10C}E, —[N(C₁-C₄)alkyl]R^{10C}E, or R^{10C}E, and wherein said 4- to 6-membered heterocyclyl is optionally substituted further with oxo;
R¹³ is selected from —NHC(O)R^{10D}E, —OR^{10D}E, phenyl, (C₃-C₆)cycloalkyl, 5- to 6-membered heteroaryl, and 4- to 6-membered heterocyclyl each of said phenyl, (C₃-C₆)cycloalkyl, 5- to 6-membered heteroaryl, and 4- to 6-membered heterocyclyl being substituted with —OR^{10D}E, —NHR^{10D}E, —[N(C₁-C₄)alkyl]R^{10D}E, or R^{10D}E, and wherein said 4- to 6-membered heterocyclyl is optionally substituted further with oxo;
E is a chemical moiety that binds to E3 ligase;
AA is the residue of an alpha or beta natural or non-natural amino acid;
R^{T} and R^{Ty} are each independently selected from (C₁-C₄)alkyl, (C₁-C₄)alkyl-C(O)O(C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 9-membered monocyclic or bicyclic heterocyclyl, 5- to 10-membered monocyclic or bicyclic heteroaryl, (C₃-C₆)cycloalkyl, oxo, imino, —OR^{e}, —C(O)R^{g}, —C(O)OR^{e}, —NR^{c}C(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₃-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 9-membered monocyclic or bicyclic heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{M} and R^{J} are each independently selected from halo, (C₁-C₄)alkyl, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NR(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F} and R^{X} are each independently selected from halo, cyano, (C₁-C₄)alkyl, cyano(C₁-C₄)alkyl, (C₃-C₆)cycloalkyl, halo(C₁-C₄)alkyl, —(C₁-C₄)alkylC(O)NR^{c}R^{d}, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, 5- to 6-membered monocyclic heteroaryl —S(O)R^{e}R^{f}, —S(O)₂R^{f}, —S(O)═NH(C₁-C₄)alkyl, —S(O)NR^{e}R^{f}, —S(O)₂NR^{e}R^{f}, —C(O)OR^{c}, —NR^{c}C(O)R^{e}, —(C₁-C₄alkyl)C(O)R^{g}, —C(O)R^{g}, —C(O)NR^{c}R^{d}, NO₂, and —NR^{a}R^{b}, wherein said phenyl, said 4- to 6-membered heterocyclyl, and said phenyl for —(C₁-C₄)alkylphenyl are each optionally and independently substituted with, as valency permits 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, —(C₁-C₄)alkyl(C₁-C₄)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo;
R^{Z} is selected from halo, cyano, (C₁-C₄)alkyl, (C₃-C₆cycloalkyl), halo(C₁-C₄)alkyl, —(C₁-C₄)alkyl(C₁-C₄)alkoxy, hydroxy(C₁-C₄)alkyl, —(C₁-C₄)alkylphenyl, —(C₁-C₄)alkylheteroaryl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, —OR^{e}, oxo, imino, phenyl, 4- to 6-membered heterocyclyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{c}, —(C₁-C₄alkyl)C(O)R^{g}, —C(O)R^{g}, —(C₁-C₄alkyl)C(O)NR^{c}R^{d}, —C(O)NR^{c}R^{d}, —NO₂, and —NR^{a}R^{b}, wherein the (C₁-C₄)alkyl is optionally substituted with cyano, wherein said phenyl, said 4- to 6-membered heterocyclyl, and said phenyl for —(C₁-C₄)alkylphenyl are each optionally and independently substituted with, as valency permits R¹³ and/or 1 to 3 groups selected from halo, cyano, oxo, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{h}, R^{c}, R^{d}, R^{e}, R^{f}, R^{g}, and R^{h} are each independently selected from, as valency permits, hydrogen, (C₁-C₄)alkyl, (C₂-C₄)alkynyl, —(C₁-C₄)alkylphenyl, phenyl, (C₁-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

2. The compound of claim 1, or pharmaceutically acceptable salt thereof, wherein, R⁵ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with R² and/or, as valency permits, optionally further substituted with 1 to 3 groups selected from R^{Q}.

3. The compound of claim 1 or 2, or pharmaceutically acceptable salt thereof, wherein the compound is of the structural formula II:

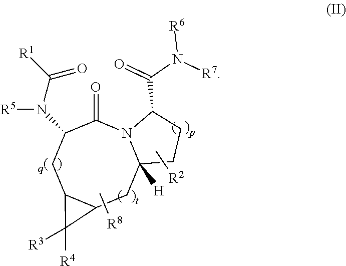

4. The compound of any one of claims 1 to 3, or pharmaceutically acceptable salt thereof, wherein q is 1.

5. The compound of any one of claims 1 to 4, or pharmaceutically acceptable salt thereof, wherein t is 1.

6. The compound of any one of claims 1 to 5, or pharmaceutically acceptable salt thereof, wherein p is 1.

7. The compound of any one of claims 1 to 6, or pharmaceutically acceptable salt thereof, wherein R² is hydrogen.

8. The compound of any one of claims 1 to 7, or pharmaceutically acceptable salt thereof, wherein the compound is of the structural formula III or IV:

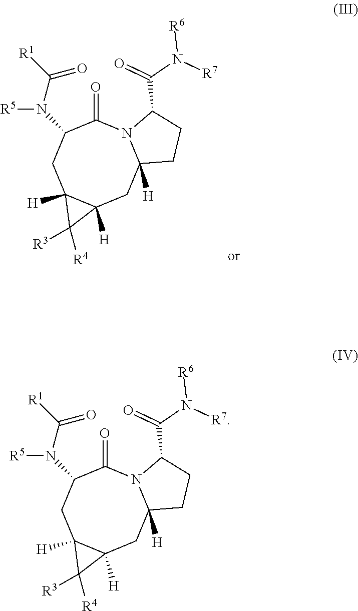

9. The compound of any one of claims 1 to 8, or pharmaceutically acceptable salt thereof, wherein R⁵ is hydrogen.

10. The compound of any one of claims 1 to 9, or pharmaceutically acceptable salt thereof, wherein R³ and R⁴ are each independently selected from hydrogen and halo.

11. The compound of any one of claims 1 to 10, or pharmaceutically acceptable salt thereof, wherein R³ and R⁴ are each hydrogen.

12. The compound of any one of claims 1 to 11, or pharmaceutically acceptable salt thereof, wherein R¹ is selected from 8- to 10-membered fused bicyclic heteroaryl and aryl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}].

13. The compound of any one of claims 1 to 12, or pharmaceutically acceptable salt thereof, wherein, R¹ is selected from benzothiophenyl and naphthalenyl, each of which are substituted with —CR^{1a}R^{2a}P(O)OR^{1b}OR^{2b}, —CR^{1a}R^{2a}P(O)[NHR^{Ty}][NH(AA)C(O)OR^{T}], —CR^{1a}R^{2a}P(O)[NH(AA)C(O)OR^{T}]][NH(AA)C(O)OR^{T}], or —CR^{1a}R^{2a}P(O)[OR^{1b}][NH(AA)C(O)OR^{T}].

14. The compound of any one of claims 1 to 13, or pharmaceutically acceptable salt thereof, wherein, R¹ is selected from,

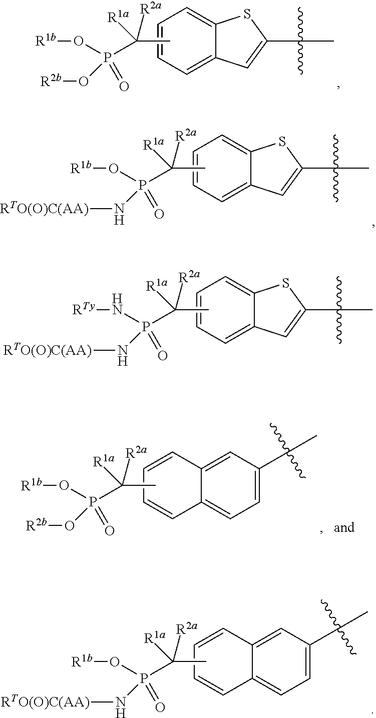

15. The compound of any one of claims 1 to 14, or pharmaceutically acceptable salt thereof, wherein, R¹ is

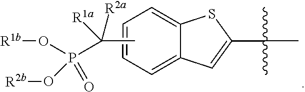

16. The compound of any one of claims 1 to 15, or pharmaceutically acceptable salt thereof, wherein R^{1a} and R^{2a} are each independently selected from hydrogen and fluoro.

17. The compound of any one of claims 1 to 16, or pharmaceutically acceptable salt thereof, wherein R^{1a} and R^{2a} are each hydrogen.

18. The compound of any one of claims 1 to 16, or pharmaceutically acceptable salt thereof, wherein R^{1a} is hydrogen and R^{2a} is fluoro.

19. The compound of any one of claims 1 to 16, or pharmaceutically acceptable salt thereof, wherein R^{1a} and R^{2a} are each fluoro.

20. The compound of any one of claims 1 to 19, or pharmaceutically acceptable salt thereof, wherein R^{1b} and R^{2b} are each independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkylphenyl]-C(O)O—[(C₁-C₄)alkyl], —[(C₁-C₄)alkyl]-OC(O)—[NH(AA)C(O)OR^{T}], —[(C₁-C₄)alkyl]-OC(O)—[(C₁-C₄)alkyl]-OH, —[(C₁-C₄)alkyl]-OC(O)O-[5- to 7-membered heterocyclyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)—[(C₁-C₄)alkyl]-OH, and phenyl.

21. The compound of any one of claims 1 to 20, or pharmaceutically acceptable salt thereof, wherein R^{1b} and R^{2b} are each independently selected from hydrogen, [(C₁-C₄)alkyl]-OC(O)—[(C₁-C₄)alkyl], —[(C₁-C₄)alkyl]-OC(O)O—[(C₁-C₄)alkyl]-O—[(C₁-C₄)alkyl], —[(C₁-C₄)alkyl]-OC(O)O—[(C₁-C₄)alkyl], and —[(C₁-C₄)alkyl]-SC(O)—[(C₁-C₄)alkyl].

22. The compound of any one of claims 1 to 21, or pharmaceutically acceptable salt thereof, wherein R^{1b} and R^{2b} are each —[(C₁-C₄)alkyl]-OC(O)—[(C₁-C₄)alkyl].

23. The compound of any one of claims 1 to 21, or pharmaceutically acceptable salt thereof, wherein R^{1b} and R^{2b} are hydrogen.

24. The compound of any one of claims 1 to 15 and 20, or pharmaceutically acceptable salt thereof, wherein —CR^{1a}R^{2a}P(O)OR^{1b}OR^{2b} is selected from

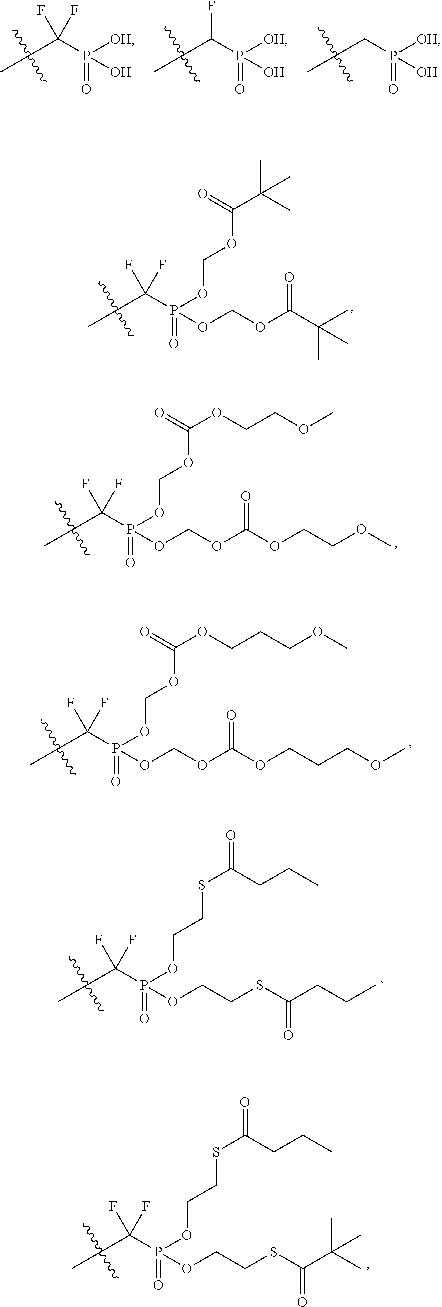

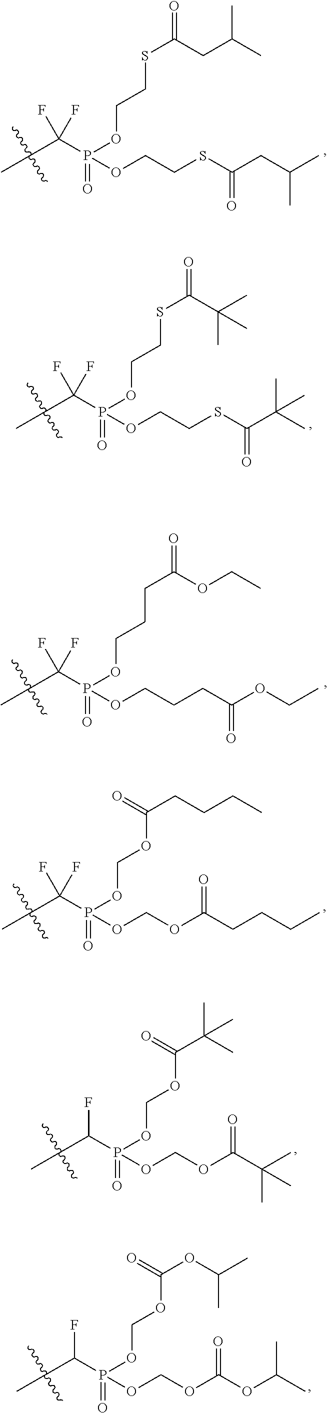 [SMILES: *C(F)(F)P(=O)(OCCSC(=O)CC(C)C)OCCSC(=O)CC(C)C ; *C(F)(F)P(=O)(OCCSC(=O)C(C)(C)C)OCCSC(=O)C(C)(C)C ; *C(F)(F)P(=O)(OCCCC(=O)OCC)OCCCC(=O)OCC ; *C(F)(F)P(=O)(OCOC(=O)CCCC)OCOC(=O)CCCC ; *C(F)P(=O)(OCOC(=O)C(C)(C)C)OCOC(=O)C(C)(C)C ; *C(F)P(=O)(OCOC(=O)OC(C)C)OCOC(=O)OC(C)C]

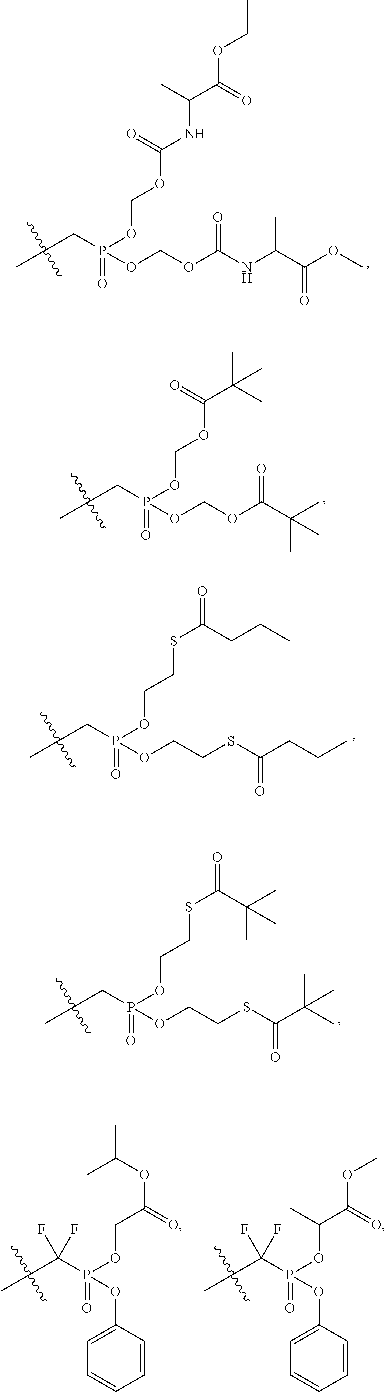 [SMILES: *CP(=O)(OCOC(=O)NC(C)C(=O)OC)OCOC(=O)NC(C)C(=O)OCC ; *CP(=O)(OCOC(=O)C(C)(C)C)OCOC(=O)C(C)(C)C ; *CP(=O)(OCCSC(=O)CCC)OCCSC(=O)CCC ; *CP(=O)(OCCSC(=O)C(C)(C)C)OCCSC(=O)C(C)(C)C ; *C(F)(F)P(=O)(OCC(=O)OC(C)C)Oc1ccccc1 ; *C(F)(F)P(=O)(Oc1ccccc1)OC(C)C(=O)OC]

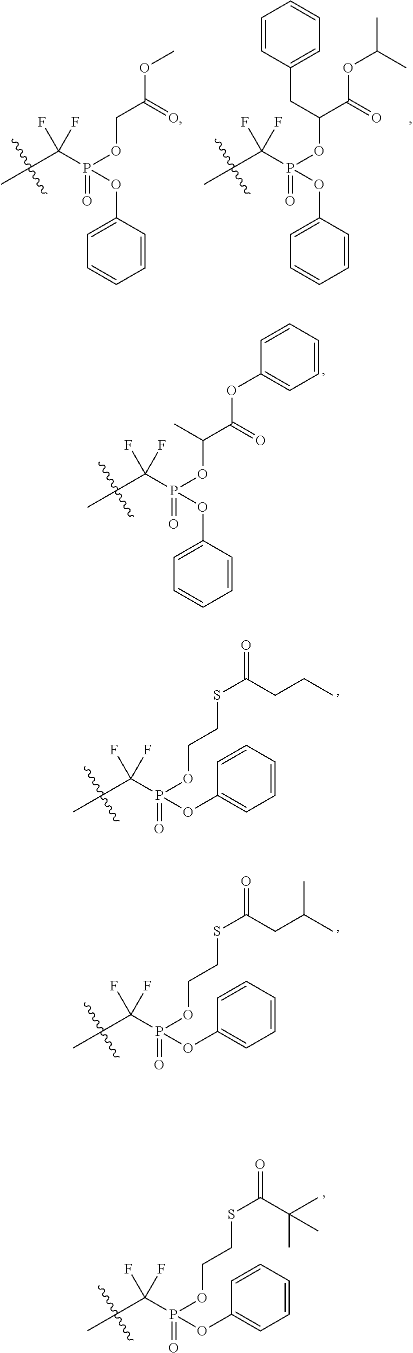 [SMILES: *C(F)(F)P(=O)(OCC(=O)OC)Oc1ccccc1 ; *C(F)(F)P(=O)(Oc1ccccc1)OC(Cc1ccccc1)C(=O)OC(C)C ; *C(F)(F)P(=O)(Oc1ccccc1)OC(C)C(=O)Oc1ccccc1 ; *C(F)(F)P(=O)(OCCSC(=O)CCC)Oc1ccccc1 ; *C(F)(F)P(=O)(OCCSC(=O)CC(C)C)Oc1ccccc1 ; *C(F)(F)P(=O)(OCCSC(=O)C(C)(C)C)Oc1ccccc1]

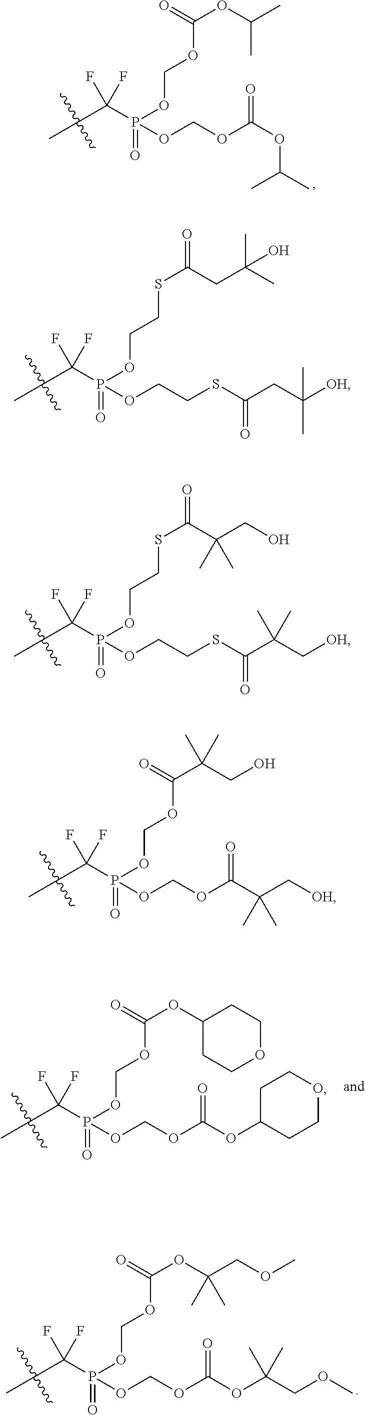

24. The compound of any one of claims 1 to 15 and 20, or pharmaceutically acceptable salt thereof, wherein —CR^{1a}R^{2a}P(O)OR^{1b}OR^{2b} is selected from

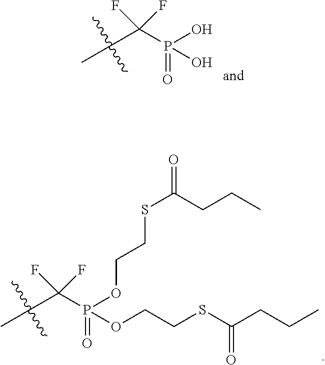 [SMILES: *C(F)(F)P(=O)(O)O ; *C(F)(F)P(=O)(OCCSC(=O)CCC)OCCSC(=O)CCC]

25. The compound of any one of claims 1 to 24, or pharmaceutically acceptable salt thereof, wherein R⁶ and R⁷ together with the nitrogen atom to which they are attached form a 4- to 9-membered monocyclic or bicyclic heterocyclyl substituted with R¹² and/or, as valency permits, optionally further substituted with 1 to 3 groups selected from R^{Q}.

26. The compound of any one of claims 1 to 25, or pharmaceutically acceptable salt thereof, wherein R⁶ and R⁷ together with the nitrogen atom to which they are attached form pyrrolidinyl, azetidinyl, or 4-azaspiro[2.4]heptanyl, each substituted with R¹² and/or, as valency permits, optionally further substituted with 1 to 3 groups selected from R^{Q}.

27. The compound of any one of claims 1 to 26, or pharmaceutically acceptable salt thereof, wherein R^{Q} is cyano or 5- to 7-membered heteroaryl.

28. The compound of any one of claims 1 to 27, or pharmaceutically acceptable salt thereof, wherein R^{Q} is cyano or pyridinyl.

29. The compound of any one of claims 1 to 28, or pharmaceutically acceptable salt thereof, wherein R¹² is selected from —NHC(O)R^{10C}E, —OR^{10C}E, phenyl, pyridinyl, and pyridin-2(1H)-one, each substituted with R^{10C}E, —OR^{10C}E, —NHR^{10C}E, or —[N(C₁-C₄)alkyl]R^{10C}E.

30. The compound of any one of claims 1 to 29, or pharmaceutically acceptable salt thereof, wherein R^{10C} is selected from (C₁-C₁₀)alkylene and (C₂-C₁₀)alkynelene each optionally substituted with one or more groups selected from halo and oxo, and wherein said (C₁-C₆)alkylene and (C₂-C₁₀)alkynelene may also be optionally interrupted by one or more heteroatoms selected from S, N, and O, and/or optionally interrupted by one or more rings selected from 5- to 7-membered heteroaryl, (C₃-C₆)cycloakyl, phenyl, and 4- to 7-membered heterocyclyl.

31. The compound of any one of claims 1 to 30, or pharmaceutically acceptable salt thereof, wherein R^{10C} is selected from (C₁-C₁₀)alkylene, (C₂-C₁₀)alkynelene, —(CH₂)C(O)NH[(CH₂)O]ᵥ(CH₂)ₙ, —(CH₂)O[(CH₂)O]ᵥ(CH₂)ₙ, —(C₂-C₈)alkynelene[4- to 7-membered heterocyclyl], 4- to 7-membered heterocyclyl, [(CH₂)O]ᵥ(CH₂)ₙ, and —(CH₂)ₙC(O)NH[4- to 7-membered heterocyclyl], wherein m, n, and v are each independently selected from 0, 1, 2, 3, 4, 5, and 6.

32. The compound of any one of claims 1 to 31, or pharmaceutically acceptable salt thereof, wherein R^{10C} is selected from:

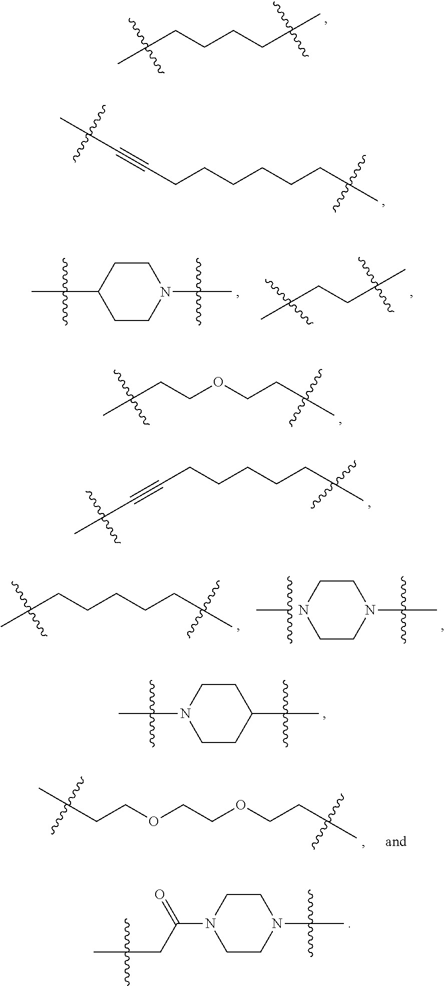

33. The compound of any one of claims 1 to 32, or pharmaceutically acceptable salt thereof, wherein E is selected from a CRBN, VHL, IAP, or MDM2 based E3 binder.

34. The compound of any one of claims 1 to 33, or pharmaceutically acceptable salt thereof, wherein E is selected from 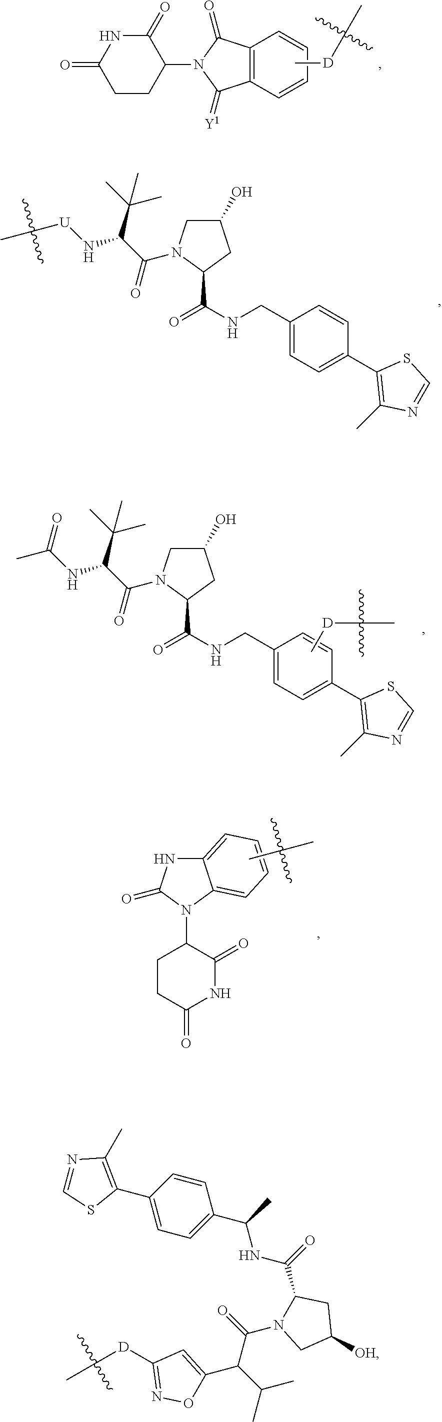 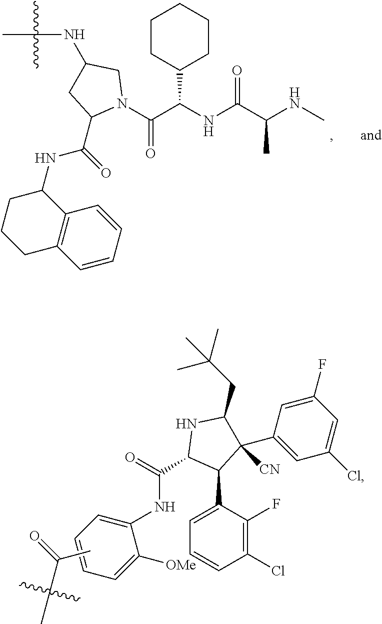
wherein:
Y and Y′ are each independently is O or CH₂;
D is O, NH, or a bond;
U is absent or C(O); and
R is halo or cyano.

35. The compound of any one of claims 1 to 34, or pharmaceutically acceptable salt thereof, wherein E is

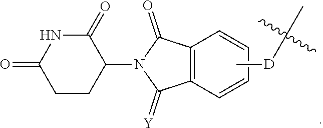

36. The compound of claim 1, or pharmaceutically acceptable salt thereof, wherein the compound is selected from

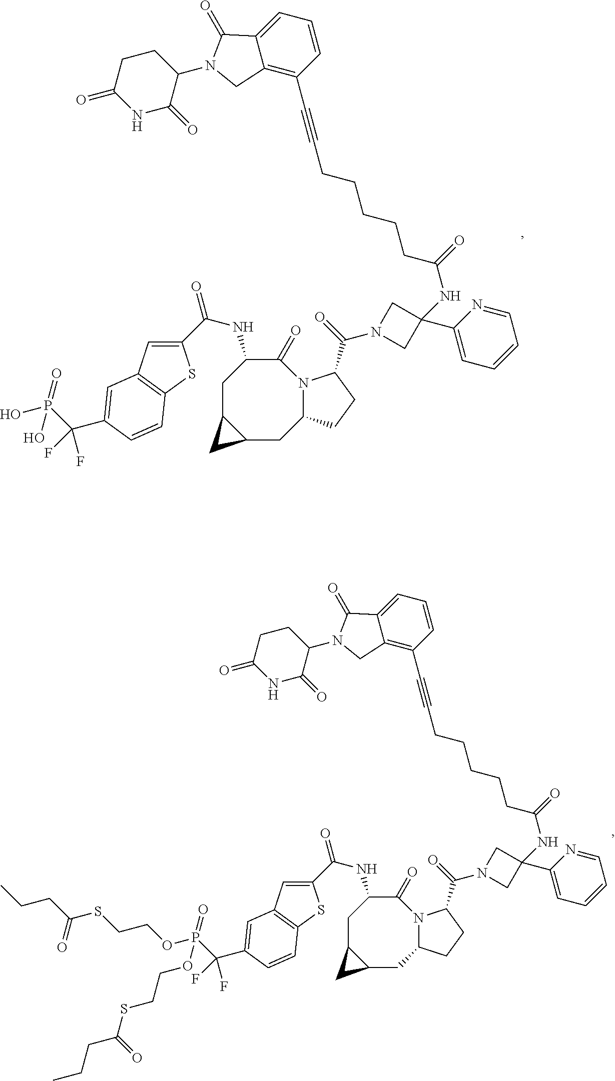 [SMILES: O=C1CCC(N2Cc3c(C#CCCCCCC(=O)NC4(c5ccccn5)CN(C(=O)[C@@H]5CC[C@@H]6C[C@H]7C[C@H]7C[C@H](NC(=O)c7cc8cc(C(F)(F)P(=O)(O)O)ccc8s7)C(=O)N65)C4)cccc3C2=O)C(=O)N1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5CC(NC(=O)CCCCCC#Cc6cccc7c6CN(C6CCC(=O)NC6=O)C7=O)(c6ccccn6)C5)N4C3=O)cc2c1]

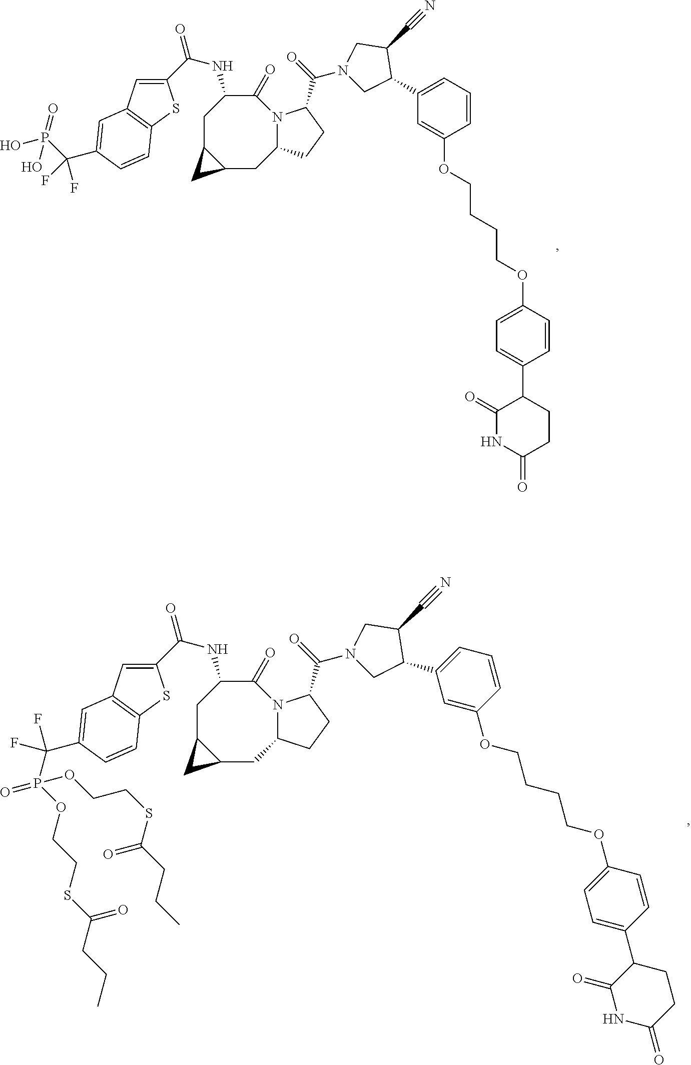 [SMILES: N#C[C@@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@H]1c1cccc(OCCCCOc2ccc(C3CCC(=O)NC3=O)cc2)c1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5C[C@@H](C#N)[C@H](c6cccc(OCCCCOc7ccc(C8CCC(=O)NC8=O)cc7)c6)C5)N4C3=O)cc2c1]

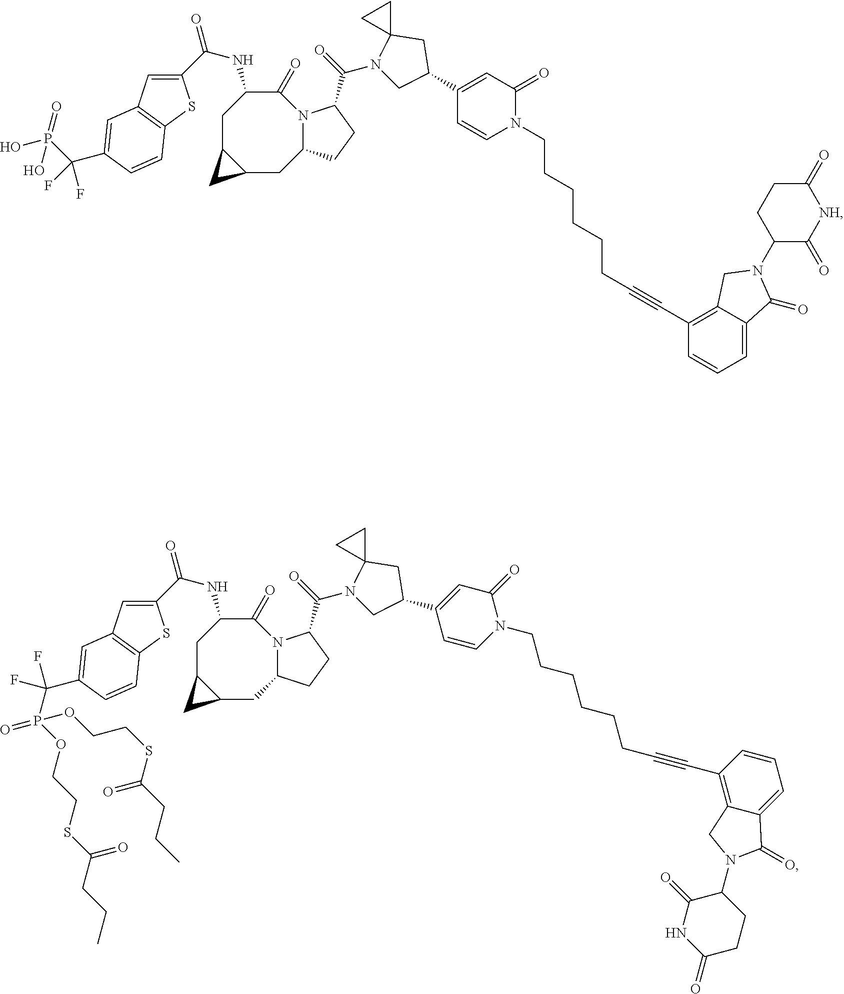 [SMILES: O=C1CCC(N2Cc3c(C#CCCCCCCn4ccc([C@@H]5CN(C(=O)[C@@H]6CC[C@@H]7C[C@H]8C[C@H]8C[C@H](NC(=O)c8cc9cc(C(F)(F)P(=O)(O)O)ccc9s8)C(=O)N76)C6(CC6)C5)cc4=O)cccc3C2=O)C(=O)N1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5C[C@@H](c6ccn(CCCCCCC#Cc7cccc8c7CN(C7CCC(=O)NC7=O)C8=O)c(=O)c6)CC56CC6)N4C3=O)cc2c1]

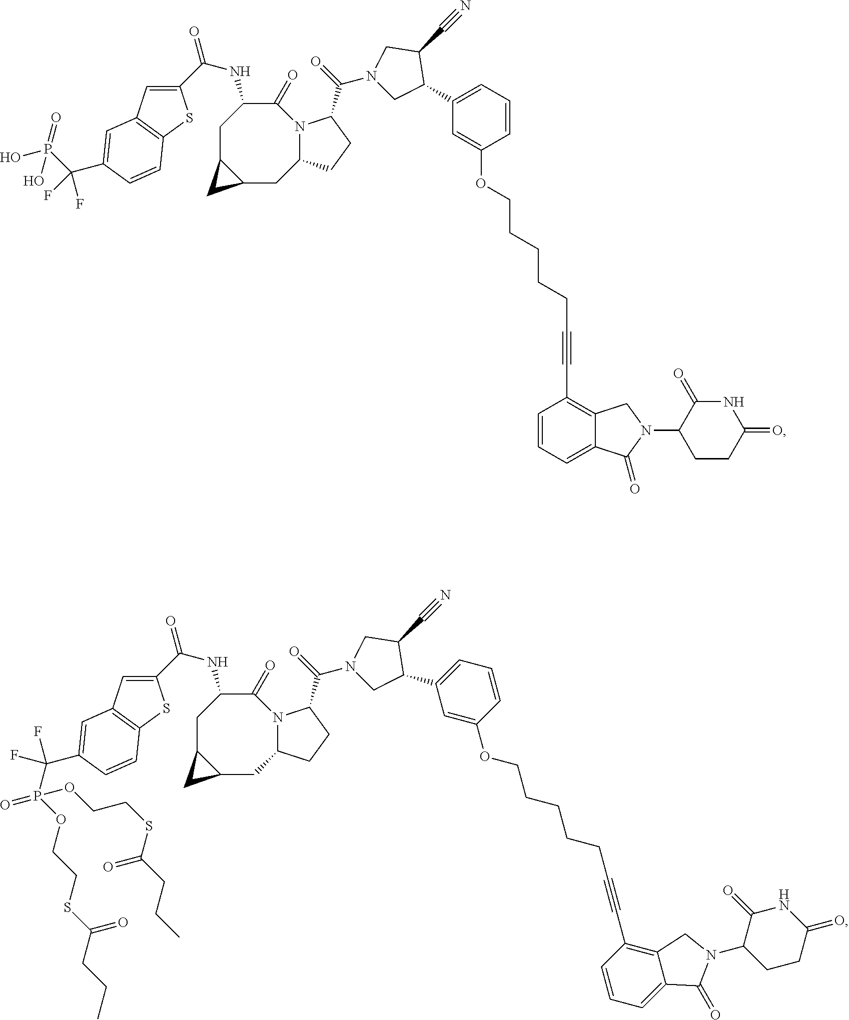 [SMILES: N#C[C@@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@H]1c1cccc(OCCCCCC#Cc2cccc3c2CN(C2CCC(=O)NC2=O)C3=O)c1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5C[C@@H](C#N)[C@H](c6cccc(OCCCCCC#Cc7cccc8c7CN(C7CCC(=O)NC7=O)C8=O)c6)C5)N4C3=O)cc2c1]

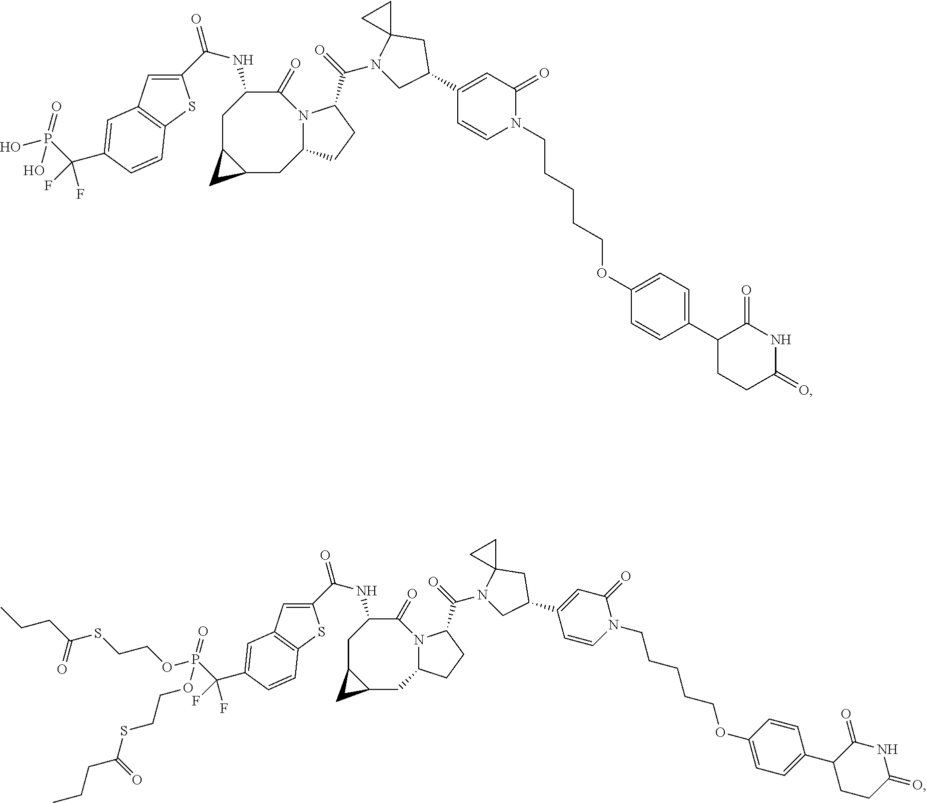 [SMILES: O=C1CCC(c2ccc(OCCCCCn3ccc([C@@H]4CN(C(=O)[C@@H]5CC[C@@H]6C[C@H]7C[C@H]7C[C@H](NC(=O)c7cc8cc(C(F)(F)P(=O)(O)O)ccc8s7)C(=O)N65)C5(CC5)C4)cc3=O)cc2)C(=O)N1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5C[C@@H](c6ccn(CCCCCOc7ccc(C8CCC(=O)NC8=O)cc7)c(=O)c6)CC56CC6)N4C3=O)cc2c1]

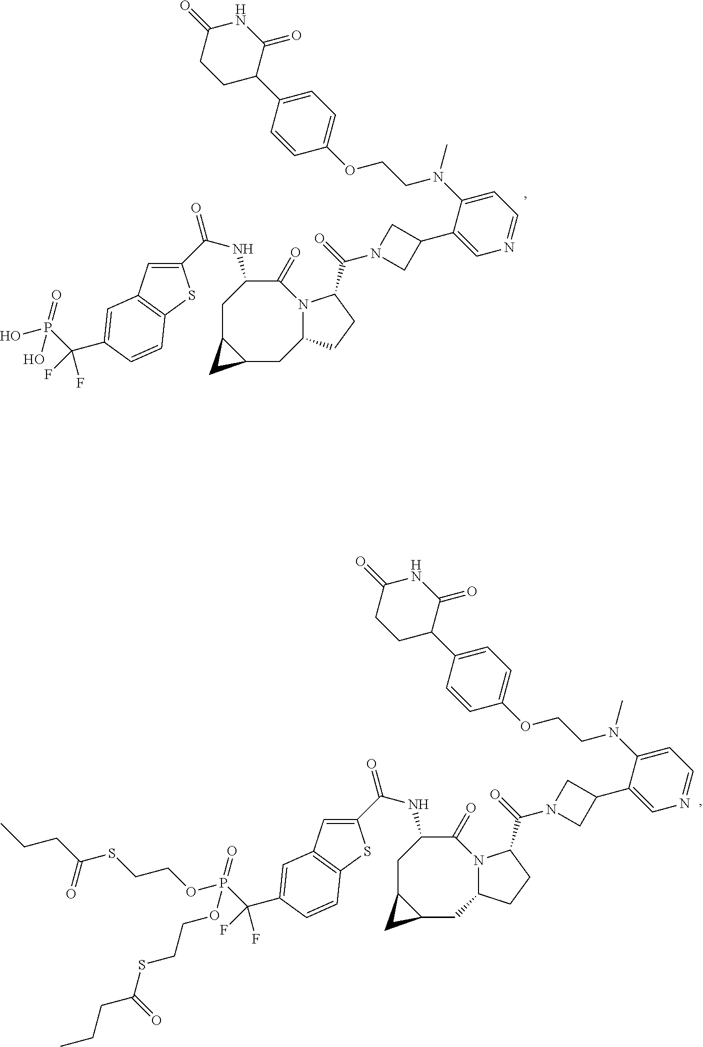 [SMILES: CN(CCOc1ccc(C2CCC(=O)NC2=O)cc1)c1ccncc1C1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5CC(c6cnccc6N(C)CCOc6ccc(C7CCC(=O)NC7=O)cc6)C5)N4C3=O)cc2c1]

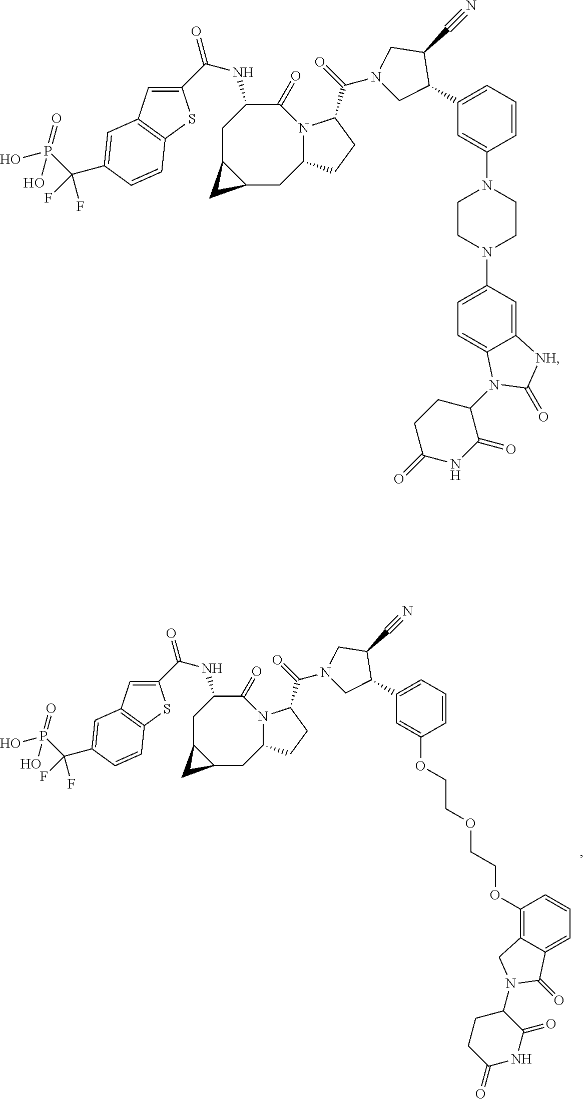 [SMILES: N#C[C@@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@H]1c1cccc(N2CCN(c3ccc4c(c3)[nH]c(=O)n4C3CCC(=O)NC3=O)CC2)c1 ; N#C[C@@H]1CN(C(=O)[C@@H]2CC[C@@H]3C[C@H]4C[C@H]4C[C@H](NC(=O)c4cc5cc(C(F)(F)P(=O)(O)O)ccc5s4)C(=O)N32)C[C@H]1c1cccc(OCCOCCOc2cccc3c2CN(C2CCC(=O)NC2=O)C3=O)c1]

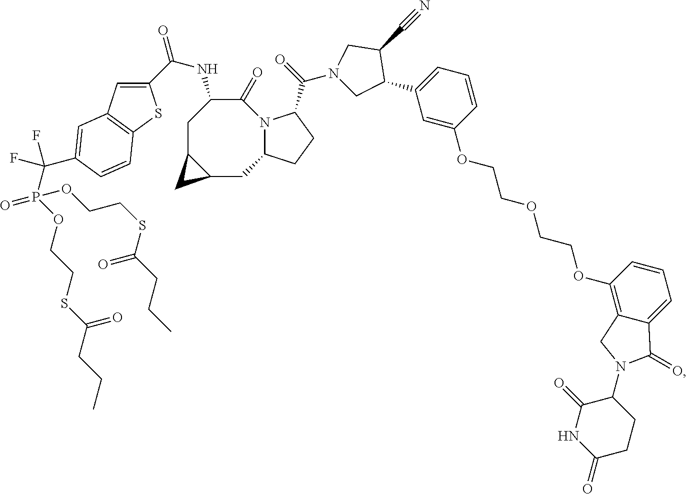 [SMILES: CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5C[C@@H](C#N)[C@H](c6cccc(OCCOCCOc7cccc8c7CN(C7CCC(=O)NC7=O)C8=O)c6)C5)N4C3=O)cc2c1]

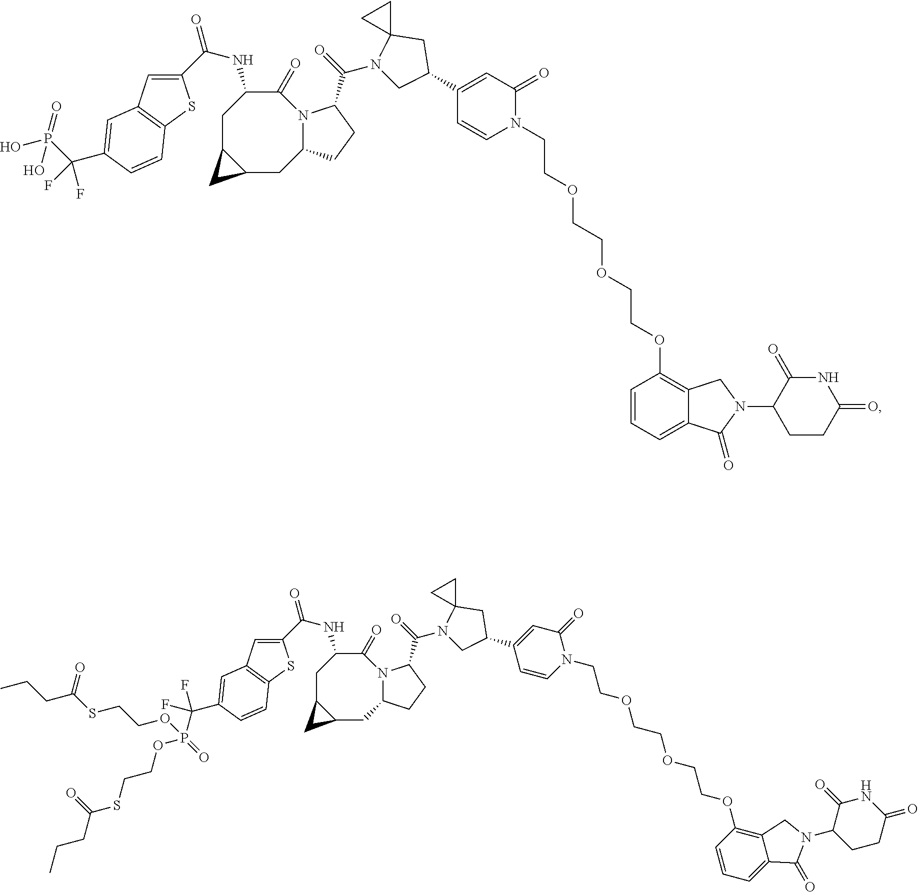 [SMILES: O=C1CCC(N2Cc3c(OCCOCCOCCn4ccc([C@@H]5CN(C(=O)[C@@H]6CC[C@@H]7C[C@H]8C[C@H]8C[C@H](NC(=O)c8cc9cc(C(F)(F)P(=O)(O)O)ccc9s8)C(=O)N76)C6(CC6)C5)cc4=O)cccc3C2=O)C(=O)N1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5C[C@@H](c6ccn(CCOCCOCCOc7cccc8c7CN(C7CCC(=O)NC7=O)C8=O)c(=O)c6)CC56CC6)N4C3=O)cc2c1]

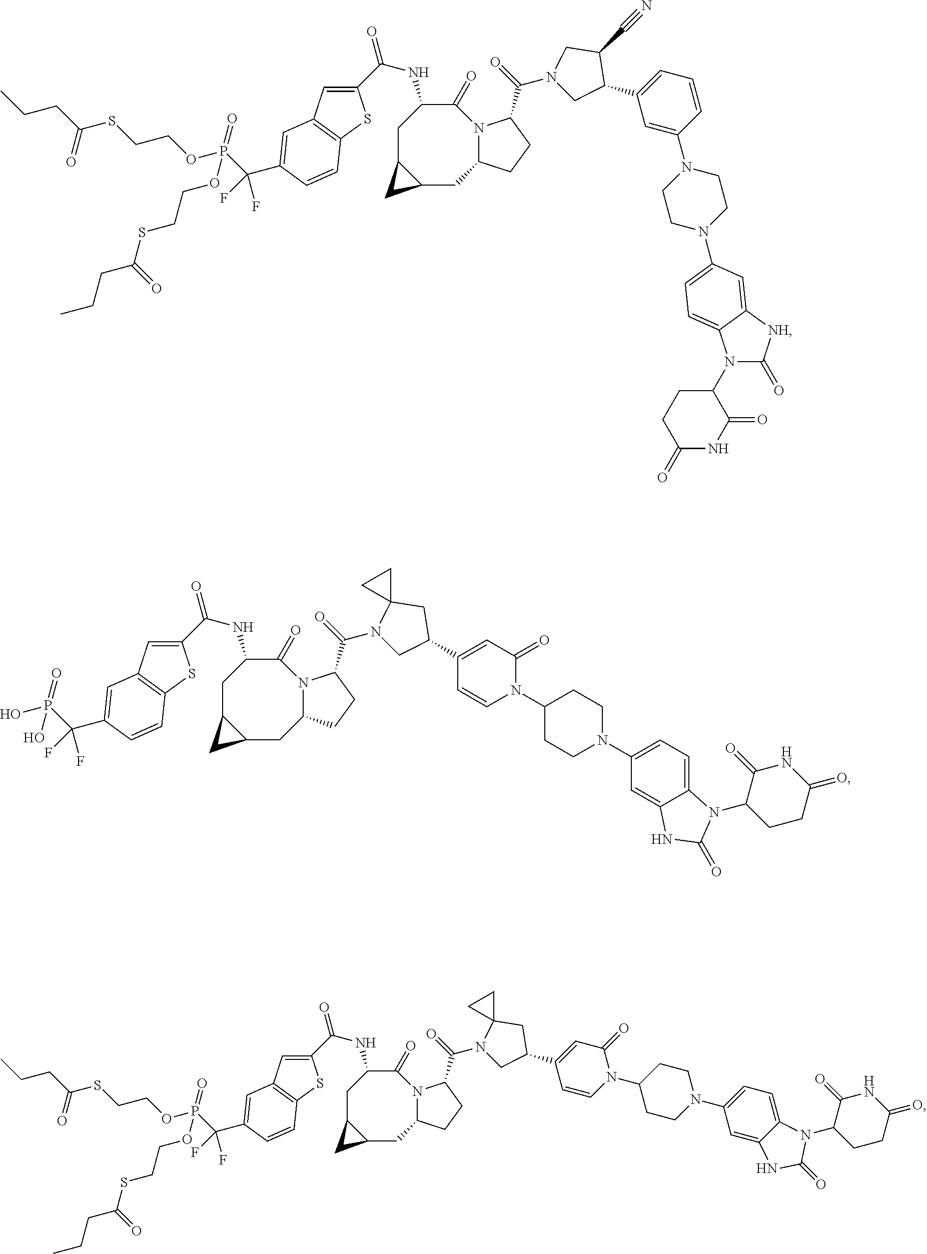

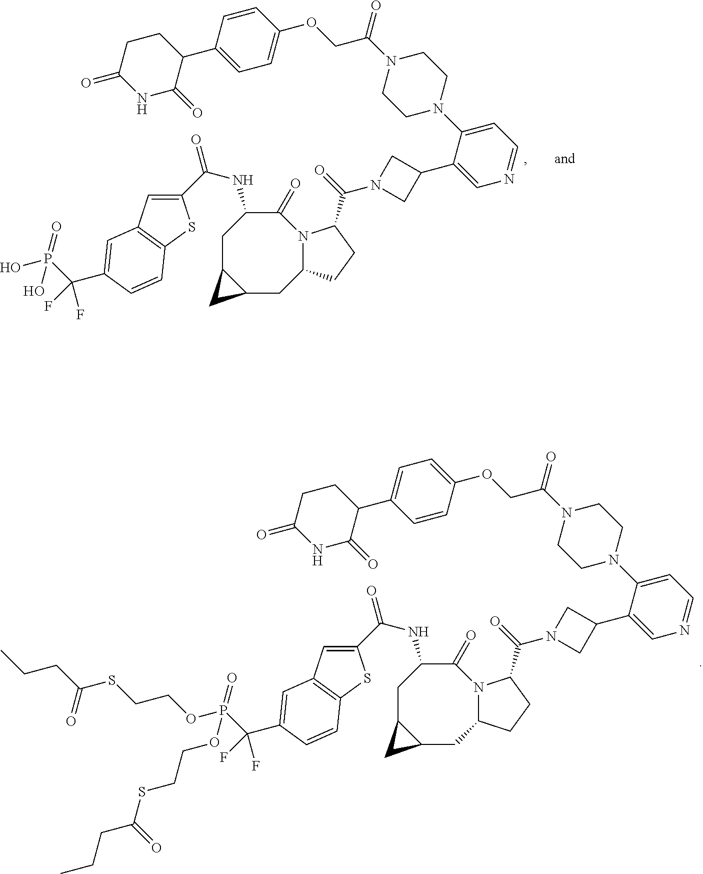 [SMILES: O=C1CCC(c2ccc(OCC(=O)N3CCN(c4ccncc4C4CN(C(=O)[C@@H]5CC[C@@H]6C[C@H]7C[C@H]7C[C@H](NC(=O)c7cc8cc(C(F)(F)P(=O)(O)O)ccc8s7)C(=O)N65)C4)CC3)cc2)C(=O)N1 ; CCCC(=O)SCCOP(=O)(OCCSC(=O)CCC)C(F)(F)c1ccc2sc(C(=O)N[C@H]3C[C@@H]4C[C@@H]4C[C@H]4CC[C@@H](C(=O)N5CC(c6cnccc6N6CCN(C(=O)COc7ccc(C8CCC(=O)NC8=O)cc7)CC6)C5)N4C3=O)cc2c1]

37. A pharmaceutically acceptable composition comprising the compound of any one of claims 1 to 36, or a pharmaceutically acceptable salt thereof; and a pharmaceutically acceptable carrier.

38. A method of treating a condition responsive to the modulation of STAT3 or STAT6 in a subject comprising administering to the subject a therapeutically effective amount of the compound of any one of claims 1 to 36, or a pharmaceutically acceptable salt thereof, or the pharmaceutically acceptable composition of claim 37.
